(19) United States
(12) Patent Application Publication
(10) Pub. No.: US 2005/0090398 A1
(43) Pub. Date: Apr. 28, 2005

(54) Novel tetrazole derivative useful as herbicides

(76) Inventors: Akihiko Yanagi, Tochigi (JP); Shinichi Narabu, Ibaraki (JP); Yoshihiro Yamaguchi, Kanagawa (JP); Toshio Goto, Tochigi (JP); Shinichi Shirakura, Tochigi (JP); Chieko Ueno, Tochigi (JP); Shin Nakamura, Tochigi (JP)

Correspondence Address: BAYER CROPSCIENCE LP;Patent Department, 100 BAYER ROAD, PITTSBURGH, PA, 15205-9741, US

(21) Appl. No.: 10/503,771

(22) PCT Filed: Jan. 27, 2003
(86) PCT No.: PCT/EP03/00772

(30) Foreign Application Priority Data
Feb. 8, 2002 (JP) 2002/32551

(51) Int. Cl. A01N 43/647; C07D 257/02

(52) U.S. Cl. 504/261; 548/252

(57) ABSTRACT

Compounds of the Formula (I) wherein T represents a group, Q represents a group, R¹, R², R³, R⁴, R⁵, R⁶, R⁷, T⁸, R⁹, R¹⁰, R¹¹ are as defined in the description, m represents 0, 1, 2 or 3, n represents 0 or 1, A represents alkylene, processes for preparation, their intermediates and their use in agriculture are described.

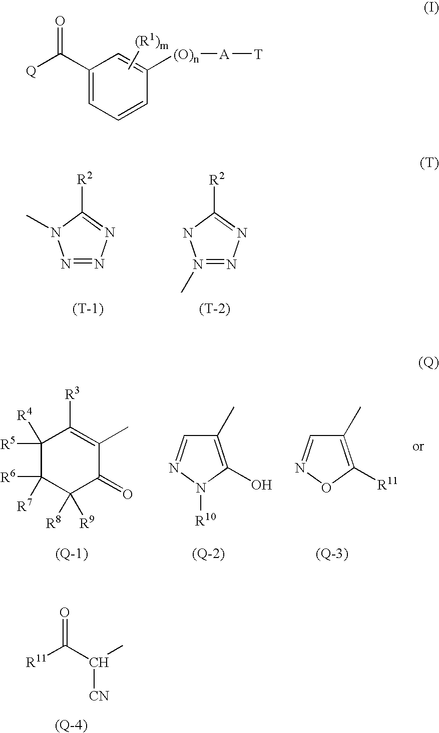

The present invention relates to novel tetrazole derivatives, to processes for their preparation, to their intermediates and to their use in agriculture, especially to their use as herbicides.

It is known that certain tetrazole derivatives show herbicidal activity (cf. JP 12275/1999, JP 21280/1999 etc.). Further, it is known, that certain heterocyclic derivatives act as herbicide (JP 114769/2001, WO 99/10327, WO 00/21924).

There have now been found novel tetrazole derivatives of the formula (I) 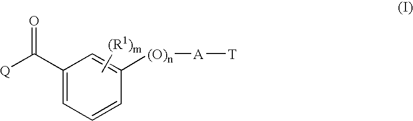
wherein
R¹ represents halogen, alkyl, haloalkyl, alkoxy, alkylthio, alkylsulfonyl, alkylsulfonyloxy, alkoxyalkyl, alkylthioalkyl, alkylsulfonylalkyl, nitro or cyano,
m represents 0, 1, 2 or 3,
R¹ may be identical or different to each other, when in represents 2 or 3,
n represents 0 or 1,
A represents alkylene,
T represents a group 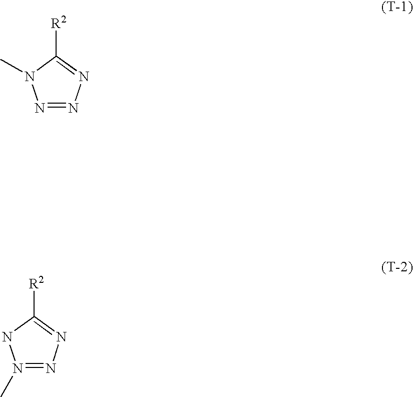
   in which
R² represents hydrogen, alkyl or cycloalkyl, which may be optionally substituted by alogen and alkyl, or
   represents alkenyl, alkynyl, haloalkyl, alkylthio, or phenyl which may be optionally substituted by halogen, alkyl, haloalkyl and nitro, and
Q represents a group 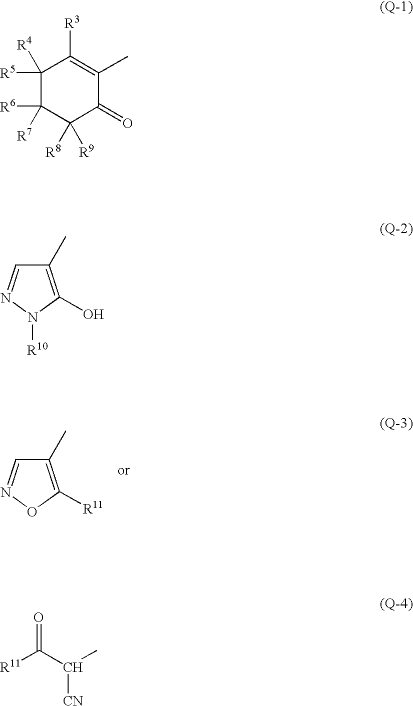
   wherein
R³ represents hydroxy, halogen or alkylcarbonyloxy, or represents alkylthio which may be optionally substituted by hydroxy, cyano, carboxy, alkoxycarbonyl and phenyl, or
   represents 5- or 6-membered heteroarylthio containing 1-2 hetero atom(s) selected from the group consisting of nitrogen, oxygen and sulfur, which may be optionally substituted by halogen and alkyl, and when R³ represents pyridylthio, said pyridylthio may form N-oxide, or
   represents phenylthio which may be optionally substituted by halogen, alkyl, alkoxy, haloalkyl and nitro, or represents phenylcarbonyloxy which may be optionally substituted by halogen and alkyl, or
   represents 1-pyrazolyl which may be optionally substituted or 1-imidazolyl which may be optionally substituted by halogen and alkyl, or represents 1,2,4-triazol-1-yl or 1H-tetrazol-1-yl,
R⁴, R⁵, R⁶, R⁷, R⁸ and R⁹ each independently represent hydrogen or alkyl, or
R⁴ may, together with R⁹, form an ethylene chain,
R¹⁰ represents alkyl, and
R¹¹ represents alkyl or cycloalkyl.

The compounds of the formula (I), according to the invention, can be obtained by a process in which
a) in case of preparing a compound of the formula (1) in which Q represents the group (Q-1) and R³ represents hydroxy compounds of the formula (II) 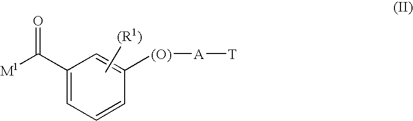
   wherein
R¹, m, n, A and T have the same definition as aforementioned, and
M¹ represents group 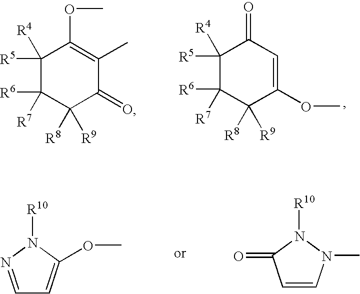
   in which
R⁴, R⁵, R⁶, R⁷, R⁸, R⁹ and R¹⁰ have the same definition as aforementioned,
are reacted to a rearrangement in the presence of inert solvents, and if appropriate, in the presence of a base and a cyanide, and if appropriate, in the presence of a phase-transfer catalyst, or
b) in case of preparing a compound of the formula (I) in which Q represents the group (Q-1) and R³ represents halogen, preferably chloro or bromo: compounds of the formula (Ib) 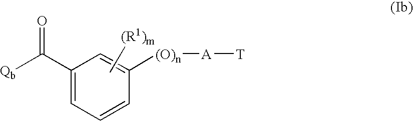
   wherein
R¹, m, n, A and T have the same definition as aforementioned, and
Q_{b} represents group 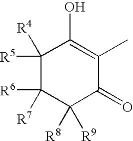
   in which
R⁴, R⁵, R⁶, R⁷, R⁸ and R⁹ have the same definition as aforementioned,
are reacted with a halogenating agent in the presence of inert solvents, or
c) in case of preparing a compound of the formula (I) in which
   Q represents the group (Q-1) and
   R³ represents alkylthio which may be optionally substituted or
      represents 5- or 6-membered heteroarylthio, or
      represents phenylthio which may be optionally substituted, or
      represents 1-pyrazolyl which may be optionally substituted or
      represents 1-imidazolyl which may be optionally substituted, or
      represents 1,2,4-triazol-1-yl or 1H-tetrazol-1-yl:
   compounds of the formula (1c) 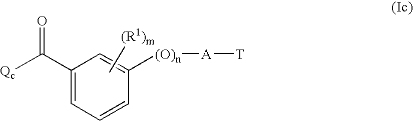
      wherein
R¹, m, n, A and T have the same definition as aforementioned, and
Q_{c} represents group 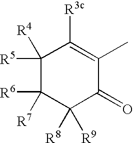
   in which
R⁴, R⁵, R⁶, R⁷, R⁸ and R⁹ have the same definition as aforementioned, and
R^{3c} represents chloro or bromo,
are reacted with compounds of the formula (III)
   R¹²—H (III)
   wherein
R¹² has the same definition as the above-mentioned R³ in the preparation process (c),
in the presence of inert solvents, and if appropriate, in the presence of an acid binding agent, or
d) in case of preparing a compound of the formula (I) in which Q represents the group (Q-1) and R³ represents alkylcarbonyloxy or phenylcarbonyloxy which may be optionally substituted:
   compounds of the formula (Ib) are reacted with compounds of the formula (IV) 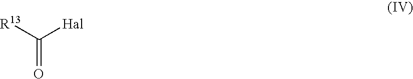
      wherein
R¹³ represents alkyl or phenyl which may be optionally substituted, and
Hal represents halogen, preferably chloro or bromo,
in the presence of inert solvents, and if appropriate, in the presence of an acid binding agent, or
e) in case of preparing a compound of the formula (I) in which Q represents the group (Q-2):
   compounds of the formula (IIe) 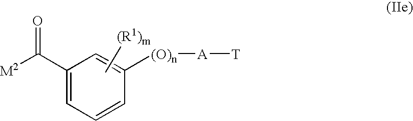
      wherein
R¹, m, n, A and T have the same definition as aforementioned, and
M² represents group 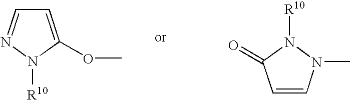
   in which
R¹⁰ has the same definition as aforementioned,
are reacted to a rearrangement in the presence of inert solvents,
and if appropriate, in the presence of a base, or
f) in case of preparing a compound of the formula (I) in which Q represents the group (Q-3):
compounds of the formula (V) 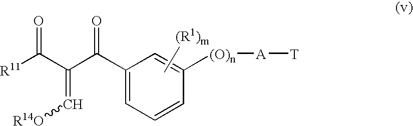
   wherein
R¹, m, n, A, T and R¹¹ have the same definition as aforementioned, and
R¹⁴ represents C₁₋₄ alkyl, preferably methyl or ethyl,
are reacted with hydroxylamine in the presence of inert solvents, and if appropriate, in the presence of a base, or
g) in case of preparing a compound of the formula (I) in which Q represents the group (Q-4):
   compounds of the formula (Ig) 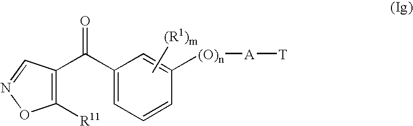
      wherein
R¹, m, n, A, T and R¹¹ have the same definition as aforementioned,
are reacted to a ring—opening in the presence of inert solvents, and if appropriate, in the presence of a base.

The tetrazole derivatives of the formula (I) provided by the present invention show a strong herbicidal action. The compounds of the formula (I) of the present invention unexpectedly show an extremely strong herbicidal action compared with the known compounds disclosed in the above-mentioned state of the art. They particularly show an extremely good effect as a selective herbicide for paddy rice that shows excellent herbicidal action against paddy field weeds and has no substantial phytotoxicity to paddy rice. The compound of the formula (I) show an even stronger herbicidal action it they are mixed with other herbicidal compounds or safeners as specifically mentioned later.

In the formulae used herein the following definitions are used unless otherwise specified:

“Halogen” represents fluoro, chloro, bromo or iodo, preferably represents fluoro, chloro or bromo.

“Alkyl” can be straight-chain or branched-chain. Alkyl preferably represents C₁₋₆ alkyl, and particularly preferably methyl, ethyl, n- or iso-propyl, n-, iso-, sec- or tert-butyl, n-, iso-, neo- or tert-pentyl, n- or iso-hexyl.

“Cycloalkyl” represents a cyclyc hydrocarbon moiety. It preferably represents cyclopropyl, cyclobutyl, cyclopentyl, cyclohexyl etc. These cycloalkyls may be optionally substituted by halogen or alkyl. When a plurality of substituents exist, they may be identical or different. As specific examples of such substituted cycloalkyl there can be mentioned 1-methylcyclopropyl, 1-ethylcyclopropyl, 1-n-propylcyclopropyl, 1-methyl-2-fluorocyclopropyl, 2-methylcyclopropyl, 2-fluoro-cyclopropyl, 1-methyl-2,2-difluorocyclopropyl, 1-methyl-2,2-dichlorocyclopropyl, 2,2-difluorocyclopropyl, 2-methylcyclopentyl, 1-methylcyclohexyl, 2-methyl-cyclohexyl, 3-methylcyclohexyl, 4-methylcyclohexyl, 2,3-dimethylcyclohexyl, 2,6-dimethylcyclohexyl, 2,5-dimethylcyclohexyl and so on.

“Alkenyl” represents a straight-chain or branched-chain hydrocarbon moiety having one or more carbon-carbon double bonds. It preferably represents vinyl, allyl, 1-methylallyl, 1,1-dimethylallyl, 2-butenyl, 2-pentenyl, 2-hexenyl and so on.

“Alkynyl” represents a straight-chain or branched-chain hydrocarbon-moiety having one or more carbon-carbon triple bonds, ethynyl, 2-propynyl, 1-methyl-2-propynyl, 1,1-dimethyl-2-propynyl, 2-butynyl, 2-pentynyl, 2-hexynyl and so on.

“Alkylene” can be straight-chain or branched-chain and includes, for example, methylene, ethylidene, ethylene, propylidene, methylethylene (propylene), trimethylene, ethylethylene, methyltrimethylene, 2-methyltrimethylene, tetramethylene and so on.

“Alkoxy” represents an Alkyl-O— group, whose alkyl part has the above-mentioned meaning. It preferably represents C₁₋₆ alkoxy, and particularly preferably methoxy, ethoxy, n- or iso-propoxy, n-, iso-, sec- or tert-butoxy, n-pentyloxy, n-hexyloxy.

“Alkylthio” represents an Alkyl-S— group, whose alkyl part has the above-mentioned meaning. It preferably represents C₁₋₆ alkylthio, and particularly preferably methylthio, ethylthio, n- or iso-propylthio, n-, iso-, sec- or tert-butylthio, n-pentylthio, n-hexylthio.

“Alkylsulfonyl” represents an Alkyl-SO₂— group, whose alkyl part has the above-mentioned meaning. It preferably represents C₁₋₆ alkylsulfonyl, and particularly preferably methylsulfonyl, ethylsulfonyl, n- or iso-propylsulfonyl, n-, iso-, sec- or tert-butylsulfonyl.

“Alkylsulfonyloxy” represents an Alkyl-SO₂—O— group, whose alkyl part has the above-mentioned meaning. It preferably represents C₁₋₄ alkylsulfonyloxy, and particularly preferably methylsulfonyloxy, ethylsulfonyloxy, n- or iso-propyl-sulfonyloxy, n-, iso-, sec- or tert-butylsulfonyloxy.

“Alkylcarbonyl” represents an Alkyl-CO— group, whose alkyl part has the above-mentioned meaning. It preferably represents C₁₋₆ alkylcarbonyl, and particularly preferably acetyl, propionyl, butyryl, isobutyryl, valeryl, isovaleryl, pivaloyl, n-pentylcarbonyl, n-hexylcarbonyl.

“Alkylcarbonyloxy” represents an Alkyl-CO₂— group, whose alkyl part has the above-mentioned meaning. It preferably represents C₁₋₆ alkylcarbonyloxy, and particularly preferably acetoxy, ethylcarbonyloxy, n- or iso-propylcarbonyloxy, n-, iso-, sec- or tert-butylcarbonyloxy, n-pentylcarbonyloxy, n-hexylcarbonyloxy.

“Alkoxyalkyl” represents alkyl substituted with alkoxy. It preferably represents C_{2-≢}(total carbon number) alkoxyalkyl, and particularly preferably methoxymethyl, 1-methoxyethyl, 2-methoxyethyl, 2-methoxy-1-methylethyl, methoxypropyl, methoxybutyl, methoxypentyl, ethoxymethyl, n- or iso-propoxymethyl, n-, iso-, sec- or tert-butoxymethyl.

“Alkylthioalkyl” represents alkyl substituted with alkylthio. It preferably represents C₂₋₆ (total carbon number) alkylthioalkyl, and particularly preferably methylthiomethyl, methylthioethyl, 1-methylthiopropyl, 2-methylthiopropyl, 1-methyl-2-methylthioethyl, methylthiobutyl, methylthiopentyl, ethylthiomethyl, n- or iso-propylthiomethyl, n-, iso-, sec- or tert-butylthiomethyl.

“Alkylsulfonylalkyl” represents alkyl substituted with alkylsulfonyl. It preferably represents C₂₋₆ (total carbon number) alkylsulfonylalkyl, and particularly preferably methylsulfonylmethyl, methylsulfonylethyl, 1-methylsulfonylpropyl, 2-methylsulfonylpropyl, 1-methyl-2-methylsulfonylethyl, methylsulfonylbutyl, methylsulfonylpentyl, ethylsulfonylmethyl, n- or iso-propylsulfonylmethyl, n-, iso-, sec- or tert-butylsulfonylmethyl.

“Haloalkyl” represents straight-chain or branched-chain alkyl, in which at least one hydrogen is halogen-substituted. It preferably represents C₁₋₄ alkyl substituted with 1-6 fluoro and/or chloro, difluoromethyl, trifluoromethyl, 2,2,2-trifluoroethyl, dichloromethyl, 2-chloro-1,1,2-trifluoroethyl, 3-fluoropropyl, 3-chloropropyl, 2,2,3,3,3-pentafluoropropyl, 1,2,2,3,3,3-hexafluoropropyl.

The haloalkyl part in “haloalkoxy” can be of the same definition as in the above-mentioned “haloalkyl”. It particularly preferably represents difluoromethoxy, trifluoromethoxy, 2-fluoroethoxy, 2-chloroethoxy, 2-bromoethoxy, 2,2,2-trifluoroethoxy, 3-chloropropoxy and so on.

As “5- or 6-membered heteroarylthio containing 1-2 hetero atom(s) selected from the group consisting of nitrogen, oxygen and sulfur” there can be mentioned, for example, thienylthio, thiazolylthio, oxazolylthio, pyridylthio, pyrimidylthio and so on. In case of pyridylthio, said pyridylthio can form an N-oxide.

Preferred substituents or preferred ranges of the radicals present in the formulae listed above and below are defined as follows:
R¹ preferably represents fluoro, chloro, bromo, iodo, C₁₋₆ alkyl, C₁₋₆ haloalkyl, C₁₋₆ alkoxy, C₁₋₄ alkylthio, C₁₋₆ alkylsulfonyl, C₁₋₆ alkylsulfonyloxy, C₂₋₆ alkoxyalkyl, C₂₋₆ alkylthioalkyl, C₂₋₆ alkylsulfonylalkyl, nitro or cyano.
m preferably represents 2 or 3.
A preferably represents C₁₋₄ alkylene.
R² preferably represents hydrogen, C₁₋₆ alkyl, C₃₋₆ cycloalkyl, C₂₋₆ alkenyl, C₂₋₆ alkynyl, C₁₋₆ haloalkyl, C₁₋₆ alkylthio, or phenyl which may be optionally substituted by chloro, C₁₋₄ alkyl, C₁₋₄ haloalkyl and nitro.
R³ preferably represents hydroxy, chloro, bromo, C₂₋₅ alkylcarbonyloxy, or C₁₋₆ alkylthio which may be optionally substituted by hydroxy, cyano, carboxy, C₂₋₅ alkoxycarbonyl and phenyl, or
   thienylthio, thiazolylthio, oxazolylthio, pyridylthio, 1-oxidopyridylthio or pyrimidylthio, optionally substituted by chloro-, bromo-, or C₁-C₄ alkyl, or phenylthio which may be optionally substituted by one or two substituents selected from the group consisting of fluoro, chloro, bromo, C₁₋₄ alkyl, C₁₋₄ alkoxy, C₁₋₄ haloalkyl and nitro, or
   phenylcarbonyloxy which may be optionally substituted by one or two substituents selected from the group consisting of chloro and C₁₋₄ alkyl, or 1-pyrazolyl or 1-imidazolyl which may be optionally substituted by one or two substituents selected from the group consisting of chloro, bromo and C₁₋₄ alkyl, or
   1,2,4-triazol-1-yl or 1H-tetrazol-1-yl.
R⁴, R⁵, R⁶, R⁷, R⁸ and R⁹ each independently preferably represent hydrogen or C₁₋₄ alkyl, or
R⁴ may, together with R⁹, form an ethylene chain.
R¹⁰ preferably represents C₁₋₄ alkyl.
R¹¹ preferably represents C₃₋₆ cycloalkyl.
R¹ particularly preferably represents chloro, bromo, methyl, trifluoromethyl, methoxy, methylthio, ethylthio, methylsulfonyl, ethylsulfonyl, n-propylsulfonyl, methylsulfonyloxy, methoxymethyl, methylthiomethyl, methylsulfonylmethyl, nitro or cyano.
m particularly preferably represents 2,
A particularly preferably represents C₁₋₄ alkylene.
R² particulary preferably represents hydrogen, methyl, ethyl, n-propyl, cyclopropyl, cyclopentyl, vinyl, allyl, ethynyl, trifluoromethyl, 2-chloroethyl, 3-bromopropyl, methylthio, ethylthio, n-propylthio, or phenyl which may be optionally substituted by chloro, methyl, ethyl, n-propyl, trifluoromethyl and nitro.
R³ particularly preferably represents hydroxy, chloro, acetoxy, tert-butyl-carbonyloxy, methylthio, ethylthio, n-propylthio, 2-hydroxyethylthio, 2-cyanoethylthio, carboxymethylthio, methoxycarbonylmethylthio, 2-(ethoxy-carbonyl)ethylthio, benzylthio, or
   2-thienylthio, 2-thiazolylthio, 2-oxazolylthio, 2-pyridylthio, 1-oxido-2-pyridylthio or 2-pyrimidylthio, optionally substituted by one or more substituents selected from the group consisting of chloro and methyl, or phenylthio which may be optionally substituted by a substituent selected from the group consisting of fluoro, chloro, methyl, ethyl, n-propyl, methoxy, trifluoromethyl and nitro, or
   represents phenylcarbonyloxy which may be optionally substituted by a substituent selected from the group consisting of chloro and methyl, or
   represents1-pyrazolyl or 1-imidazolyl which may be optionally substituted by one or two substituents selected from the group consisting of chloro and methyl, or
   represents 1,2,4-triazol-1-yl or 1H-tetrazol-1-yl.
R⁴, R⁵, R⁶, R⁷, R⁸ and R⁹ each independently, particularly preferably represent hydrogen or methyl, or
R⁴ may, together with R⁹, form an ethylene chain.
R¹⁰ particularly preferably represents methyl or ethyl.
R¹¹ particularly preferably represents cyclopropyl.

Compounds of the formula (I-1) 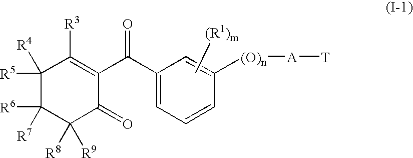
in which R¹, R³, R⁴, R⁵, R⁶, R⁷, R⁸, R⁹, m, n, A, T are each as defined above are particularly emphasised as being part of the invention.

Compounds of the formula (I-2) 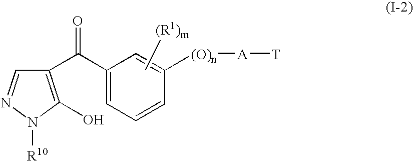
in which R¹, R¹⁰, m, n, A, T are each as defined above are particularly emphasised as being part of the invention.

Compounds of the formula (I-3) 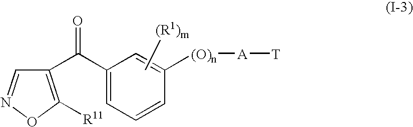
in which R¹, R¹¹, m, n, A, T are each as defined above are particularly emphasised as being part of the invention.

Compounds of the formula (I-4) 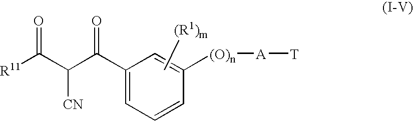
in which R¹, R¹¹, m, n, A, T are each as defined above are particularly emphasised as being part of the invention.

The abovementioned general or preferred radical definitions apply both to the end products of the formula (I) and also, correspondingly, to the starting materials or intermediates required in each case for the preparation. These radical definitions can be combined with one another at will, i.e. including combinations between the given preferred ranges.

Preference according to the invention is given to compounds of the formula (I) which contain a combination of the meanings mentioned above as being preferred.

Particular preference according to the invention is given to compounds of the formula (I) which contain a combination of the meanings listed above as being particularly preferred.

Very particular preference according to the invention is given to the compounds of the formula (I) which contain a combination of the meanings listed above as being very particularly preferred.

Process (a) can be illustrated by the following reaction formula when, for example, 3-oxo-1-cyclohexenyl 2,4-dichloro-3-[2-(1H-tetrazol-1-yl)ethoxy]benzoate and acetone cyanohydrin, as a cyanide, are used as the starting materials.

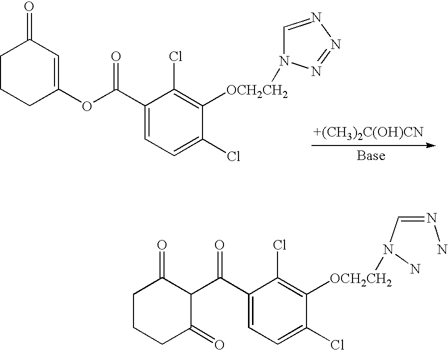

Pprocess (b) can be illustrated by the following reaction formula when, for example, 2-{2,4-dichloro-3-[2-(1H-tetrazol-1-yl)ethoxy]benzoyl}cyclohexan-1,3-dione and, for example, oxalyl dichloride, as a chlorinating agent, are used as the starting materials.

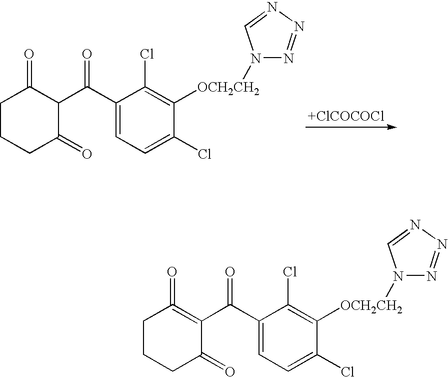

Process(c) can be illustrated by the following reaction formula when, for example, 3-chloro-2-{2,4-dichloro-3-[2-(1H-tetrazol-1-yl)ethoxy]benzoyl}-2-cyclohexen-1-one and thiophenol are used as the starting materials.

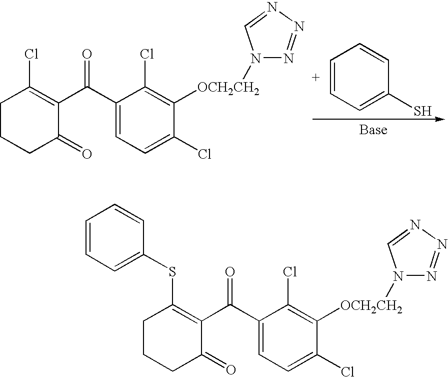

Process(d) can be illustrated by the following reaction formula when, for example, 2-{2,4-dichloro-3-[2-(1H-tetrazol-1-yl)ethoxy]benzoyl}-2-cyclohexan-1,3-dione and benzoyl chloride are used as the starting materials.

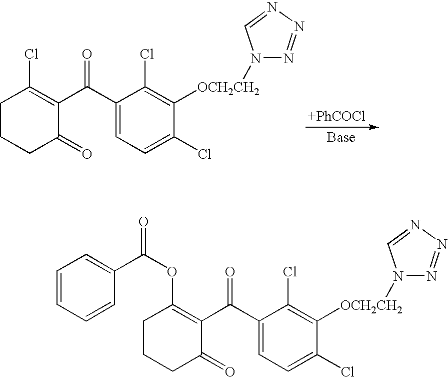

Process(e) can be illustrated by the following reaction formula when, for example, 5-{2,4-dichloro-3-[2-(1H-tetrazol-1-yl)ethoxy]benzoyloxy}-1-ethylpyrazole, and, for example, triethylamine as a base, are used as the starting materials.

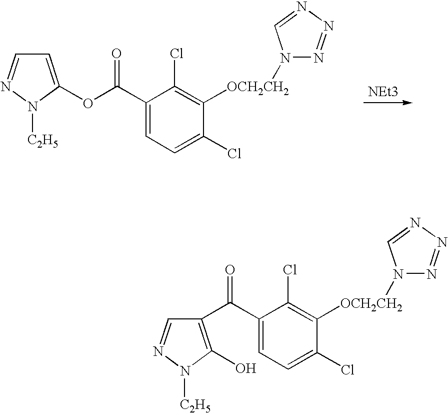

Process(f) can be illustrated by the following reaction formula when, for example, 3-cyclopropyl-1-{2,4-dichloro-3-[2-(1H-tetrazol-1-yl)ethoxy]phenyl}-2-ethoxymethylenepropan-1,3-dione and hydroxylamine are used as the starting materials.

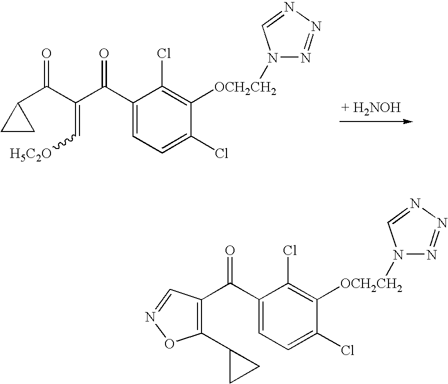

Process(g) can be illustrated by the following reaction formula when, for example, 5-cyclopropyl-4-{2,4-dichloro-3-[2-(1H-tetrazol-1-yl)ethoxy]benzoyl}isoxazole, and, for example, triethylamine as a base, are used as the starting materials.

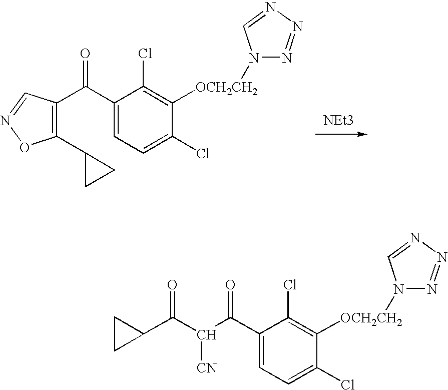

In case Q represents (Q-1) and R³ represents hydroxy in formula (I), this group has tautomers, as shown in the following (Q-1a), (Q-1b) and (Q-1c).

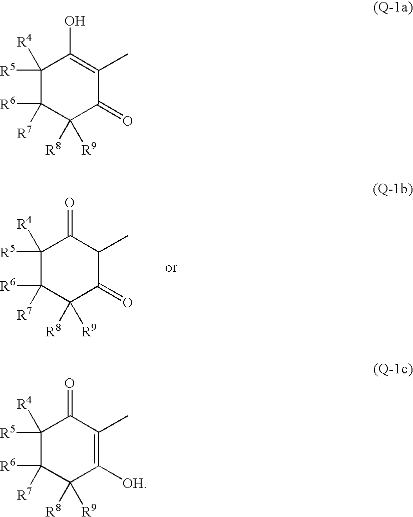

Consequently, when group Q represents the group (Q-1) and R³ represents hydroxy in compounds of the formula (I) of the present invention, the compounds of the formula (I) of the present invention can exist as a tautomers.

The compounds of the formula (II), starting material in the above-mentioned preparation process (a), are novel compounds. Compound (II) can be prepared by the process described in JP 222/1990, JP 173/1990, JP 6425/1990 etc., namely by reacting compounds of the formula (VI) 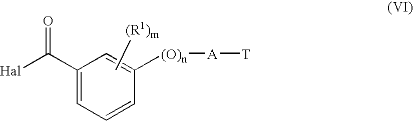
wherein
R¹, m, n, A and T have the same definition as aforementioned, and
Hal represents halogen, preferably chloro or bromo,
with compounds of the formula (VII)
   M¹-H (VII)
   wherein
M¹ has the same definition as aforementioned,
in an appropriate diluent, for example, dichloromethane in the presence of an appropriate condensing agent, for example, triethylamine.

The compounds of the above-mentioned formula (VI) include the known compounds described in U.S. Pat. No. 6,194,406, WO 97/22604 and can be prepared, for example, by reacting compounds of the formula (VIII) 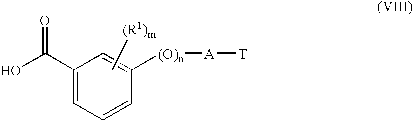
wherein
R¹, m, n, A and T have the same definition as aforementioned,
with a halogenating agent, for example, phosphorus oxychloride, phosphorus oxybromide, phosphorus trichloride, phosphorus tribromide, phosgene, oxalyl dichloride, thionyl chloride, thionyl bromide etc.

On the other hand, the compounds of the above-mentioned formula (VII) used as the starting materials in the preparation of the compounds of the above-mentioned formula (II) are per se known, can be obtained on the market, or can be prepared according to the process described in known literatures, (e.g. JP 6425/1990, JP 265415/1998, JP 265441/1998, JP 257974/1986 etc.

The compounds of the above-mentioned formula (VIII) include the known compounds described in U.S. Pat. No. 6,194,406, WO 97/22604 and can be easily prepared, for example, by hydrolyzing compounds of the formula (IX) 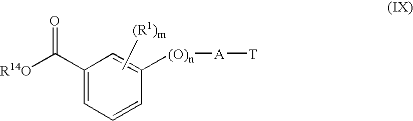
wherein
R¹, m, n, A, T and R¹⁴ have the same definition as aforementioned,
in an appropriate diluent, for example, hydrous dioxane, in the presence of an appropriate base, for example, sodium hydroxide.

The compounds of the above-mentioned formula (IX) include the known compounds described in U.S. Pat. No. 6,194,406, WO 97/22604 and can be easily prepared, for example, by reacting compounds of the formula (X)
T-H (X)
wherein
T has the same definition as aforementioned,
   with compounds of the formula (XI) 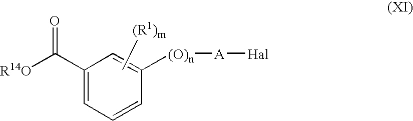
      wherein
R¹, m, n, A, Hal and R¹⁴ have the same definition as aforementioned,
in an appropriate diluent, for example, N,N-dimethylformamide, in the presence of an appropriate condensing agent, for example, potassium carbonate.

The compounds of the above-mentioned formula (X) are known.

The compounds of the above-mentioned formula (XI) include the known compounds described in JP 173/1990, JP 247891/1994, JP 206808/1995 and can be easily prepared, for example, by the processes described in the above-mentioned publications.

The compounds of the formula (II), the starting materials in the above-mentioned preparation process (a), can be prepared also from the compounds of the formula (XIII) by the process described in WO 93/18031.

As typical examples of the compounds of the formula (II) used as the starting materials in Process(a) the following can be mentioned:
3-oxo-1-cyclohexenyl 2,4-dichloro-3-[(1H-tetrazol-1-yl)methyl]benzoate,
3-oxo-1-cyclohexenyl 2,4-dichloro-3-[(2H-tetrazol-2-yl)methyl]benzoate,
3-oxo-1-cyclohexenyl 2,4-dichloro-3-[(5-methyl-tetrazol-1-yl)methyl]benzoate,
3-oxo-1-cyclohexenyl 2,4-dichloro-3-[(5-methyl-tetrazol-2-yl)methyl]benzoate,
3-oxo-1-cyclohexenyl 2,4-dibromo-3-[(1H-tetrazol-1-yl)methyl]benzoate,
3-oxo-1-cyclohexenyl 2,4-dibromo-3-[(2H-tetrazol-2-yl)methyl]benzoate,
3-oxo-1-cyclohexenyl 2-chloro-3-[(1H-tetrazol-1-yl)methyl]-4-methylsulfonylbenzoate,
3-oxo-1-cyclohexenyl 2-chloro-3-[(2H-tetrazol-2-yl)methyl]-4-methylsulfonylbenzoate,
3-oxo-1-cyclohexenyl 2-chloro-3-[(5-phenyl-tetrazol-1-yl)methyl]-4-methylsulfonylbenzoate,
3-oxo-1-cyclohexenyl 2-chloro-3-[(5-phenyl-tetrazol-2-yl)methyl]-4-methylsulfonylbenzoate,
3-oxo-1-cyclohexenyl 2-chloro-3-[(5-methylthio-tetrazol-1-yl)methyl]-4-methylsulfonylbenzoate,
3-oxo-1-cyclohexenyl 2-chloro-3-[(5-methylthio-tetrazol-2-yl)methyl]-4-methylsulfonylbenzoate,
4-{2,4-dichloro-3-[(5-methyl-tetrazol-1-yl)methyl]benzoyloxy}bicyclo[3.2.1]-3-octen-2-one,
4-{2,4-dichloro-3-[(5-methyl-tetrazol-2-yl)methyl]benzoyloxy}bicyclo[3.2.1]-3-octen-2-one,
3-oxo-1-cycloheptanyl 2-chloro-3-[(1H-tetrazol-1-yl)methyl]-4-methylsulfonylbenzoate,
3-oxo-1-cycloheptanyl 2-chloro-3-[(1H-tetrazol-2-yl)methyl]-4-methylsulfonylbenzoate,
5-{2,4-dichloro-3-[(1H-tetrazol-1-yl)methyl]benzoyloxy}-1-methylpyrazole,
5-{2,4-dichloro-3-[(2H-tetrazol-2-yl)methyl]benzoyloxy}-1-methylpyrazole,
5-{2-chloro-3-[(1H-tetrazol-1-yl)methyl]-4-methylsulfonylbenzoyloxy}-1-ethylpyrazole,
5-{2-chloro-3-[(2H-tetrazol-2-yl)methyl]-4-methylsulfonylbenzoyloxy}-1-ethylpyrazole,
3-oxo-1-cyclohexenyl 2,4-dichloro-3-[2-(1H-tetrazol-1-yl)ethyl]benzoate,
3-oxo-1-cyclohexenyl 2,4-dichloro-3-[2-(2H-tetrazol-2-yl)ethyl]benzoate,
3-oxo-1-cyclohexenyl 2-chloro-3-[2-(5-methyl-tetrazol-1-yl)propyloxy]-4-methylsulfonylbenzoate,
3-oxo-1-cyclohexenyl 2-chloro-3-[2-(5-methyl-tetrazol-2-yl)propyloxy]-4-methylsulfonylbenzoate,
3-oxo-1-cyclohexenyl 2,4-dichloro-3-[3-(1H-tetrazol-1-yl)propyloxy]benzoate,
3-oxo-1-cyclohexenyl 2,4-dichloro-3-[3-(2H-tetrazol-2-yl)propyloxy]benzoate,
3-oxo-1-cyclohexenyl 2,4-dichloro-3-[2-(1H-tetrazol-1-yl)ethyloxy]benzoate,
3-oxo-1-cyclohexenyl 2,4-dichloro-3-[3-(2H-tetrazol-2-yl)ethyloxy]benzoate,
3-oxo-1-cyclohexenyl 2,4-dichloro-3-[2-(5-methyl-tetrazol-1-yl)ethyloxy]benzoate,
3-oxo-1-cyclohexenyl 2,4-dichloro-3-[2-(5-methyl-tetrazol-2-yl)ethyloxy]benzoate,
3-oxo-1-cyclohexenyl 2,4-dibromo-3-[2-(1H-tetrazol-1-yl)ethyloxy]benzoate,
3-oxo-1-cyclohexenyl 2,4-dibromo-3-[2-(2H-tetrazol-2-yl)ethyloxy]benzoate,
3-oxo-1-cyclohexenyl 2-chloro-3-[2-(1H-tetrazol-1-yl)ethyloxy]-4-methylsulfonylbenzoate,
3-oxo-1-cyclohexenyl 2-chloro-3-[2-(2H-tetrazol-2-yl)ethyloxy]-4-methylsulfonylbenzoate,
3-oxo-1-cyclohexenyl 2-chloro-3-[2-(5-phenyl-tetrazol-1-yl)ethyloxy]-4-methylsulfonylbenzoate,
3-oxo-1-cyclohexenyl 2-chloro-3-[2-(5-phenyl-tetrazol-2-yl)ethyloxy]-4-methylsulfonylbenzoate,
3-oxo-1-cyclohexenyl 2-chloro-3-[2-(5-methylthio-tetrazol-1-yl)ethyloxy]-4-methylsulfonylbenzoate,
3-oxo-1-cyclohexenyl 2-chloro-3-[2-(5-methylthio-tetrazol-2-yl)ethyloxy]-4-methylsulfonylbenzoate,
4-{2,4-dichloro-3-[2-(5-methyl-tetrazol-1-yl)ethyloxy]benzoyloxy}bicyclo[3.2.1]-3-octen-2-one,
4-{2,4-dichloro-3-[2-(5-methyl-tetrazol-2-yl)ethyloxy]benzoyloxy}bicyclo[3.2.1]-3-octen-2-one,
3-oxo-1-cycloheptanyl 2,4-dichloro-3-[2-(1H-tetrazol-1-yl)ethyloxy]benzoate,
3-oxo-1-cycloheptanyl 2,4-dichloro-3-[2-(2H-tetrazol-2-yl)ethyloxy]benzoate,
5-{2,4-dichloro-3-[2-(1H-tetrazol-1-yl)ethyloxy]benzoyloxy}-1-methylpyrazole,
5-{2,4-dichloro-3-[2-(2H-tetrazol-2-yl)ethyloxy]benzoyloxy}-1-methylpyrazole,
5-{2-chloro-3-[2-(1H-tetrazol-1-yl)ethyloxy]-4-methylsulfonylbenzoyloxy}-1-ethylpyrazole,
5-{2-chloro-3-[2-(2H-tetrazol-2-yl)ethyloxy]-4-methylsulfonylbenzoyloxy}-1-ethylpyrazole, etc.

As typical examples of the compounds of the formula (VI) used as the starting materials in the preparation of the compounds of the formula (II) the following can be mentioned:
2,4-dichloro-3-[(1H-tetrazol-1-yl)methyl]benzoyl chloride,
2,4-dichloro-3-[(2H-tetrazol-2-yl)methyl]benzoyl chloride,
2,4-dichloro-3-[(5-methyl-tetrazol-1-yl)methyl]benzoyl chloride,
2,4-dichloro-3-[(5-methyl-tetrazol-2-yl)methyl]benzoyl chloride,
2,4-dibromo-3-[(1H-tetrazol-1-yl)methyl]benzoyl chloride,
2,4-dibromo-3-[(2H-tetrazol-2-yl)methyl]benzoyl chloride,
2-chloro-3-[(1H-tetrazol-1-yl)methyl]-4-methylsulfonylbenzoyl chloride,
2-chloro-3-[(2H-tetrazol-2-yl)methyl]-4-methylsulfonylbenzoyl chloride,
2-chloro-3-[(5-methyl-tetrazol-1-yl)methyl]-4-methylsulfonylbenzoyl chloride,
2-chloro-3-[(5-methyl-tetrazol-2-yl)methyl]-4-methylsulfonylbenzoyl chloride,
2-chloro-3-[(5-methylthio-tetrazol-1-yl)methyl]-4-methylsulfonylbenzoyl chloride,
2-chloro-3-[(5-methylthio-tetrazol-2-yl)methyl]-4-methylsulfonylbenzoyl chloride,
2,4-dichloro-3-[2-(1H-tetrazol-1-yl)ethyloxy]benzoyl chloride,
2,4-dichloro-3-[2-(1H-tetrazol-2-yl)ethyloxy]benzoyl chloride,
2,4-dichloro-3-[2-(5-methyl-tetrazol-1-yl)ethyloxy]benzoyl chloride,
2,4-dichloro-3-[2-(5-methyl-tetrazol-2-yl)ethyloxy]benzoyl chloride,
2,4-dichloro-3-[2-(5-methylthio-tetrazol-1-yl)ethyloxy]benzoyl chloride,
2,4-dichloro-3-[2-(5-methylthio-tetrazol-2-yl)ethyloxy]benzoyl chloride,
2,4-dibromo-3-[2-(1H-tetrazol-1-yl)ethyloxy]benzoyl chloride,
2,4-dibromo-3-[2-(2H-tetrazol-2-yl)ethyloxy]benzoyl chloride,
2-chloro-3-[2-(1H-tetrazol-1-yl)ethyloxy]-4-methylsulfonylbenzoyl chloride,
2-chloro-3-[2-(2H-tetrazol-2-yl)ethyloxy]-4-methylsulfonylbenzoyl chloride,
2-chloro-3-[2-(5-methyl-tetrazol-1-yl)ethyloxy]-4-methylsulfonylbenzoyl chloride,
2-chloro-3-[2-(5-methyl-tetrazol-2-yl)ethyloxy]-4-methylsulfonylbenzoyl chloride, etc.

As typical examples of the compounds of the formula (VIII) used as the starting materials in the preparation of the compounds of the formula (VI) the following can be mentioned:
2,4-dichloro-3-[(1H-tetrazol-1-yl)methyl]benzoic acid,
2,4-dichloro-3-[(2H-tetrazol-2-yl)methyl]benzoic acid,
2,4-dichloro-3-[(5-methyl-tetrazol-1-yl)methyl]benzoic acid,
2,4-dichloro-3-[(5-methyl-tetrazol-2-yl)methyl]benzoic acid,
2,4-dibromo-3-[(1H-tetrazol-1-yl)methyl]benzoic acid,
2,4-dibromo-3-[(2H-tetrazol-2-yl)methyl]benzoic acid,
2-chloro-3-[(1H-tetrazol-1-yl)methyl]-4-methylsulfonylbenzoic acid,
2-chloro-3-[(2H-tetrazol-2-yl)methyl]-4-methylsulfonylbenzoic acid,
2-chloro-3-[(5-methyl-tetrazol-1-yl)methyl]-4-methylsulfonylbenzoic acid,
2-chloro-3-[(5-methyl-tetrazol-2-yl)methyl]-4-methylsulfonylbenzoic acid,
2-chloro-3-[(5-methylthio-tetrazol-1-yl)methyl]-4-methylsulfonylbenzoic acid,
2-chloro-3-[(5-methylthio-tetrazol-2-yl)methyl]-4-methylsulfonylbenzoic acid,
2,4-dichloro-3-[2-(1H-tetrazol-1-yl)ethyloxy]benzoic acid,
2,4-dichloro-3-[2-(1H-tetrazol-2-yl)ethyloxy]benzoic acid,
2,4-dichloro-3-[2-(5-methyl-tetrazol-1-yl)ethyloxy]benzoic acid,
2,4-dichloro-3-[2-(5-methyl-tetrazol-2-yl)ethyloxy]benzoic acid,
2,4-dichloro-3-[2-(5-methylthio-tetrazol-1-yl)ethyloxy]benzoic acid,
2,4-dichloro-3-[2-(5-methylthio-tetrazol-2-yl)ethyloxy]benzoic acid,
2,4-dibromo-3-[2-(1H-tetrazol-1-yl)ethyloxy]benzoic acid,
2,4-dibromo-3-[2-(2H-tetrazol-2-yl)ethyloxy]benzoic acid,
2-chloro-3-[2-(1H-tetrazol-1-yl)ethyloxy]-4-methylsulfonylbenzoic acid,
2-chloro-3-[2-(2H-tetrazol-2-yl)ethyloxy]-4-methylsulfonylbenzoic acid,
2-chloro-3-[2-(5-methyl-tetrazol-1-yl)ethyloxy]-4-methylsulfonylbenzoic acid,
2-chloro-3-[2-(5-methyl-tetrazol-2-yl)ethyloxy]-4-methylsulfonylbenzoic acid, etc.

As typical examples of the compounds of the formula (IX) used as the starting materials in the preparation of the compounds of the formula (VIII) the following can be mentioned:
methyl 2,4-dichloro-3-[(1H-tetrazol-1-yl)methyl]benzoate,
methyl 2,4-dichloro-3-[(2H-tetrazol-2-yl)methyl]benzoate,
methyl 2,4-dichloro-3-[(5-methyl-tetrazol-1-yl)methyl]benzoate,
methyl 2,4-dichloro-3-[(5-methyl-tetrazol-2-yl)methyl]benzoate,
methyl 2,4-dibromo-3-[(1H-tetrazol-1-yl)methyl]benzoate,
methyl 2,4-dibromo-3-[(2H-tetrazol-2-yl)methyl]benzoate,
methyl 2-chloro-3-[(1H-tetrazol-1-yl)methyl]-4-methylsulfonylbenzoate,
methyl 2-chloro-3-[(2H-tetrazol-2-yl)methyl]-4-methylsulfonylbenzoate,
methyl 2-chloro-3-[(5-methyl-tetrazol-1-yl)methyl]-4-methylsulfonylbenzoate,
methyl 2-chloro-3-[(5-methyl-tetrazol-2-yl)methyl]-4-methylsulfonylbenzoate,
methyl 2-chloro-3-[(5-methylthio-tetrazol-1-yl)methyl]-4-methylsulfonylbenzoate,
methyl 2-chloro-3-[(5-methylthio-tetrazol-2-yl)methyl]-4-methylsulfonylbenzoate,
ethyl 2,4-dichloro-3-[2-(1H-tetrazol-1-yl)ethyloxy]benzoate,
ethyl 2,4-dichloro-3-[2-(2H-tetrazol-2-yl)ethyloxy]benzoate,
ethyl 2,4-dichloro-3-[2-(5-methyl-tetrazol-1-yl)ethyloxy]benzoate,
ethyl 2,4-dichloro-3-[2-(5-methyl-tetrazol-2-yl)ethyloxy]benzoate,
ethyl 2,4-dichloro-3-[2-(5-methylthio-tetrazol-1-yl)ethyloxy]benzoate,
ethyl 2,4-dichloro-3-[2-(5-methylthio-tetrazol-2-yl)ethyloxy]benzoate,
ethyl 2,4-dibromo-3-[2-(1H-tetrazol-1-yl)ethyloxy]benzoate,
ethyl 2,4-dibromo-3-[2-(2H-tetrazol-2-yl)ethyloxy]benzoate,
ethyl 2-chloro-3-[2-(1H-tetrazol-1-yl)ethyloxy]-4-methylsulfonylbenzoate,
ethyl 2-chloro-3-[2-(2H-tetrazol-2-yl)ethyloxy]-4-methylsulfonylbenzoate,
ethyl 2-chloro-3-[2-(5-methyl-tetrazol-2-yl)ethyloxy]-4-methylsulfonylbenzoate,
ethyl 2-chloro-3-[2-(5-methyl-tetrazol-2-yl)ethyloxy]-4-methylsulfonylbenzoate, etc.

The compounds of the formula (Ib), the starting materials in Process(b), are a part of the formula (I) of the present invention and can be easily prepared according to the above-mentioned preparation process (a).

As typical examples of the compounds of the formula (Ib) used as the starting materials in Process(b) the following compounds, which are included in the formula (1), can be mentioned:
2-{2,4-dichloro-3-[(1H-tetrazol-1-yl)methyl]benzoyl}cyclohexane-1,3-dione,
2-{2,4-dichloro-3-[(2H-tetrazol-2-yl)methyl]benzoyl}cyclohexane-1,3-dione,
2-{2,4-dibromo-3-[(5-methyl-tetrazol-1-yl)methyl]benzoyl}cyclohexane-1,3-dione,
2-{2,4-dibromo-3-[(5-methyl-tetrazol-2-yl)methyl]benzoyl}cyclohexane-1,3-dione,
2-{2-chloro-3-[(1H-tetrazol-1-yl)methyl]-4-methylsulfonylbenzoyl}cyclohexane-1,3-dione,
2-{2-chloro-3-[(1H-tetrazol-2-yl)methyl]-4-methylsulfonylbenzoyl}cyclohexane-1,3-dione,
2-{2-chloro-3-[(5-methylthio-tetrazol-1-yl)methyl]-4-methylsulfonylbenzoyl}cyclohexane-1,3-dione,
2-{2-chloro-3-[(5-methylthio-tetrazol-2-yl)methyl]-4-methylsulfonylbenzoyl}cyclohexane-1,3-dione,
3-{2-chloro-3-[(1H-tetrazol-1-yl)methyl]-4-methylsulfonylbenzoyl}bicyclo[3.2.1]-octane-2,4-dione,
3-{2-chloro-3-[(2H-tetrazol-2-yl)methyl]-4-methylsulfonylbenzoyl}bicyclo[3.2.1]-octane-2,4-dione,
2-{2,4-dichloro-3-[(1H-tetrazol-1-yl)methyl]benzoyl}cycloheptane-1,3-dione,
2-{2,4-dichloro-3-[(2H-tetrazol-2-yl)methyl]benzoyl}cycloheptane-1,3-dione,
2-{2,4-dichloro-3-[2-(1H-tetrazol-1-yl)ethyloxy]benzoyl}cyclohexane-1,3-dione,
2-{2,4-dichloro-3-[2-(2H-tetrazol-2-yl)ethyloxy]benzoyl}cyclohexane-1,3-dione,
2-{2,4-dibromo-3-[2-(1H-tetrazol-1-yl)ethyloxy]benzoyl}cyclohexane-1,3-dione,
2-{2,4-dibromo-3-[2-(2H-tetrazol-2-yl)ethyloxy]benzoyl}cyclohexane-1,3-dione,
2-{2-chloro-3-[2-(1H-tetrazol-1-yl)ethyloxy]-4-methylsulfonylbenzoyl}cyclohexane-1,3-dione,
2-{2-chloro-3-[2-(2H-tetrazol-2-yl)ethyloxy]-4-methylsulfonylbenzoyl}cyclohexane-1,3-dione,
2-{2-chloro-3-[2-(5-methylthio-tetrazol-1-yl)ethyloxy]-4-methylsulfonylbenzoyl}cyclohexane-1,3-dione,
2-{2-chloro-3-[2-(5-methylthio-tetrazol-2-yl)ethyloxy]-4-methylsulfonylbenzoyl}cyclohexane-1,3-dione,
3-{2,4-dichloro-3-[2-(1H-tetrazol-1-yl)ethyloxy]benzoyl}bicyclo[3.2.1]-octane-2,4-dione,
3-{2,4-dichloro-3-[2-(2H-tetrazol-2-yl)ethyloxy]benzoyl}bicyclo[3.2.1]-octane-2,4-dione,
2-{2-chloro-3-[2-(1H-tetrazol-1-yl)ethyloxy]-4-methylsulfonylbenzoyl}cycloheptane-1,3-dione,
2-{2-chloro-3-[2-(2H-tetrazol-2-yl)ethyloxy]-4-methylsulfonylbenzoyl}cycloheptane-1,3-dione, etc.

As halogenating agents used for the reaction with the compounds of the formula (Ib) in the preparation process (b) there can be mentioned, for example, thionyl chloride, thionyl bromide, oxalyl dichloride, oxalyl dibromide etc.

The compounds of the formula (Ic), the starting materials in Process(c), are a part of the formula (I) of the present invention and can be easily prepared according to the above-mentioned preparation process (a).

As typical examples of the compounds of the formula (Ic) used as starting materials in Process(c) the following compounds, which are included in the formula (I), can be mentioned:
3-chloro-2-{2,4-dichloro-3-[(1H-tetrazol-1-yl)methyl]benzoyl}-2-cyclohexen-1-one,
3-chloro-2-{2,4-dichloro-3-[(2H-tetrazol-2-yl)methyl]benzoyl}-2-cyclohexen-1-one,
3-chloro-2-{2,4-dibromo-3-[(5-methyl-tetrazol-1-yl)methyl]benzoyl}-2-cyclohexen-1-one,
3-chloro-2-{2,4-dibromo-3-[(5-methyl-tetrazol-2-yl)methyl]benzoyl}-2-cyclohexen-1-one,
3-chloro-2-{2-chloro-3-[(1H-tetrazol-1-yl)methyl]-4-methylsulfonylbenzoyl}-2-cyclohexen-1-one,
3-chloro-2-{2-chloro-3-[(2H-tetrazol-2-yl)methyl]-4-methylsulfonylbenzoyl}-2-cyclohexen-1-one,
3-chloro-2-{2-chloro-3-[(5-methylthio-tetrazol-1-yl)methyl]-4-methylsulfonyl-benzoyl}-2-cyclohexen-1-one,
3-chloro-2-{2-chloro-3-[(5-methylthio-tetrazol-2-yl)methyl]-4-methylsulfonyl-benzoyl}-2-cyclohexen-1-one,
4-chloro-3-{2-chloro-3-[(1H-tetrazol-1-yl)methyl]-4-methylsulfonylbenzoyl}bicyclo[3.2.1]-3-octen-1-one,
4-chloro-3-{2-chloro-3-[(2H-tetrazol-2-yl)methyl]-4-methylsulfonylbenzoyl}bicyclo[3.2.1]-3-octen-1-one,
3-chloro-2-{2,4-dichloro-3-[(1H-tetrazol-1-yl)methyl]benzoyl}-2-cyclohepten-1-one,
3-chloro-2-{2,4-dichloro-3-[(2H-tetrazol-2-yl)methyl]benzoyl}-2-cyclohepten-1-one,
3-chloro-2-{2,4-chloro-3-[2-(1H-tetrazol-1-yl)ethyloxy]benzoyl}-2-cyclohexen-1-one,
3-chloro-2-{2,4-dichloro-3-[2-(2H-tetrazol-2-yl)ethyloxy]benzoyl}-2-cyclohexen-1-one,
3-chloro-2-{2,4-dibromo-3-[2-(1H-tetrazol-1-yl)ethyloxy]benzoyl}-2-cyclohexen-1-one,
3-chloro-2-{2,4-dibromo-3-[2-(2H-tetrazol-2-yl)ethyloxy]benzoyl}-2-cyclohexen-1-one,
3-chloro-2-{2-chloro-3-[2-(1H-tetrazol-1-yl)ethyloxy]-4-methylsulfonylbenzoyl}-2-cyclohexen-1-one,
3-chloro-2-{2-chloro-3-[2-(2H-tetrazol-2-yl)ethyloxy]-4-methylsulfonylbenzoyl}-2-cyclohexen-1-one,
3-chloro-2-{2-chloro-3-[2-(5-methylthio-tetrazol-1-yl)ethyloxy]-4-methylsulfonyl-benzoyl}-2-cyclohexen-1-one,
3-chloro-2-{2-chloro-3-[2-(5-methylthio-tetrazol-2-yl)ethyloxy]-4-methylsulfonyl-benzoyl}-2-cyclohexen-1-one,
4-chloro-3-{2,4-dichloro-3-[2-(1H-tetrazol-1-yl)ethyloxy]benzoyl}bicyclo[3.2.1]-3-octen-2-one,
4-chloro-3-{2,4-dichloro-3-[2-(2H-tetrazol-2-yl)ethyloxy]benzoyl}bicyclo[3.2.1]-3-octen-2-one,
3-chloro-2-{2-chloro-3-[2-(1H-tetrazol-1-yl)ethyloxy]-4-methylsulfonylbenzoyl}-2-cyclohepten-1-one,
3-chloro-2-{2-chloro-3-[2-(2H-tetrazol-2-yl)ethyloxy]-4-methylsulfonylbenzoyl}cyclohepten-1-one, etc.

The compounds of the formula (IV), the starting materials in Process(d), are carbonyl halides well known in the field of organic chemistry. As their specific examples the following can be mentioned:
acetyl chloride,
propionyl chloride, isobutyryl chloride, pivaloyl chloride, benzoyl chloride, 2-methylbenzoyl chloride, 2,6-dichlorobenzoyl chloride, 2,6-dimethylbenzoyl chloride, etc.

The compounds of the formula (IIe), the starting materials in Process(c), are a part of the formula (II) and can be easily prepared according to the above-mentioned.

As typical examples of the compounds of the formula (IIe) the following compounds, which are included in the formula (II), can be mentioned:
5-{2,4-dichloro-3-[(1H-tetrazol-1-yl)methyl]benzoyloxy}-1-methylpyrazole,
5-{2,4-dichloro-3-[(2H-tetrazol-2-yl)methyl]benzoyloxy}-1-methylpyrazole,
5-{2-chloro-3-[(1H-tetrazol-1-yl)methyl]-4-methylsulfonylbenzoyloxy}-1-ethylpyrazole,
5-{2-chloro-3-[(2H-tetrazol-2-yl)methyl]-4-methylsulfonylbenzoyloxy}-1-ethylpyrazole,
5-{2,4-dichloro-3-[2-(1H-tetrazol-1-yl)ethyloxy]benzoyloxy}-1-methylpyrazole,
5-{2,4-dichloro-3-[2-(2H-tetrazol-2-yl)ethyloxy]benzoyloxy}-1-methylpyrazole,
5-{2-chloro-3-[2-(1H-tetrazol-1-yl)ethyloxy]-4-methylsulfonylbenzoyloxy}-1-ethylpyrazole,
5-{2-chloro-3-[2-(2H-tetrazol-2-yl)ethyloxy]-4-methylsulfonylbenzoyloxy}-1-ethylpyrazole, etc.

The compounds of the formula (V), the starting materials in the above-mentioned preparation process (f), are novel. They can be prepared by the process described in JP 202008/1993, namely by reacting compounds of the formula (XII) 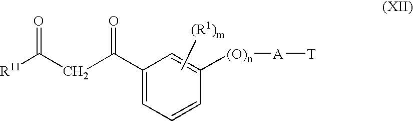
wherein
R¹, m, n, A, T and R¹¹ have the same definition as aforementioned,
with compounds of the formula (XI)
   HC(OR¹⁴)₃ (XIII)
   wherein
R¹⁴ has the same definition as aforementioned,
in an appropriate diluent, for example, acetic anhydride.

The compounds of formula (XII) are novel compounds. Compounds (XII) can be prepared by the process described in JP 202008/1993, namely by conducting a refluxing treatment of a compound of the formula (XIV) 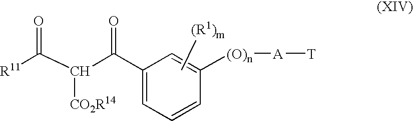
wherein
R¹, m, n, A, T, R¹¹ and R¹⁴ have the same definition as aforementioned,
in an appropriate diluent, for example, toluene under an appropriate acidic condition, for example, in the presence of p-toluenesulfonic acid monohydrate.

The compounds of formula (XIV) are novel compounds. Compounds (XIV) can be prepared by the process described in JP 202008/1993, namely by reacting a compound represented by formula (VI) with, for example, a complex obtained by treating a compound represented by the formula (XV) 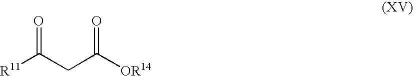
wherein
R¹¹ and R¹⁴ have the same definition as aforementioned,
with magnesium and carbon tetrachloride.

The compounds of the above-mentioned formula (XV) are available on the market and can be also prepared according to the process described in, for example, Journal of Organic Chemistry, Vol. 43, 2087 (1978).

As typical examples of the compounds of the formula (V) used as the starting materials in Process(f) the following can be mentioned:
3-cyclopropyl-1-{2,4-dichloro-3-[(1H-tetrazol-1-yl)methyl]phenyl}-2-ethoxypropane-1,3-dione,
3-cyclopropyl-1-{2,4-dichloro-3-[(2H-tetrazol-2-yl)methyl]phenyl}-2-ethoxypropane-1,3-dione,
3-cyclopropyl-1-{2-chloro-3-[(1H-tetrazol-1-yl)methyl]-4-methylsulfonylphenyl}-2-ethoxypropane-1,3-dione,
3-cyclopropyl-1-{2-chloro-3-[(2H-tetrazol-2-yl)methyl]-4-methylsulfonylphenyl}-2-ethoxypropane-1,3-dione,
3-cyclopropyl-1-{2,4-dichloro-3-[2-(1H-tetrazol-1-yl)ethyloxy]phenyl}-2-ethoxy-propane-1,3-dione,
3-cyclopropyl-1-{2,4-dichloro-3-[2-(2H-tetrazol-2-yl)ethyloxy]phenyl}-2-ethoxypropane-1,3-dione, etc.

The compounds of the formula (Ig), the starting materials in Process(g), are a part of the formula (I) of the present invention and can be easily prepared according to the above-mentioned preparation process (f).

As typical examples of the compounds of the formula (Ig) used as the starting materials in Process(g) the following compounds, which are included in the formula (I), can be mentioned:
5-cyclopropyl-4-{2,4-dichloro-3-[(1H-tetrazol-1-yl)methyl]benzoyl}isoxazole,
5-cyclopropyl-4-{2,4-dichloro-3-[(2H-tetrazol-2-yl)methyl]benzoyl}isoxazole,
5-cyclopropyl-4-{2-chloro-3-[(1H-tetrazol-1-yl)methyl]-4-methylsulfonylbenzoyl}isoxazole,
5-cyclopropyl-4-{2-chloro-3-[(2H-tetrazol-2-yl)methyl]-4-methylsulfonylbenzoyl}isoxazole,
5-cyclopropyl-4-{2,4-dichloro-3-[2-(1H-tetrazol-1-yl)ethyloxy]benzoyl}isoxazole,
5-cyclopropyl-4-{2,4-dichloro-3-[2-(2H-tetrazol-2-yl)ethyloxy]benzoyl}isoxazole, etc.

The compounds of the formulae (II), (V), (XII) and (XV) are either starting materials or intermediates in the processes (a)-(g), for preparing compounds of the formula (I). They are novel compounds and are represented collectively by formula (XVI) 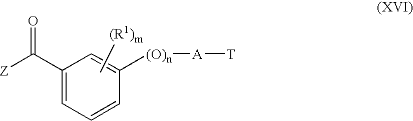
wherein
R¹, m, n, A and T have the same definition as aforementioned, and
Z represents group 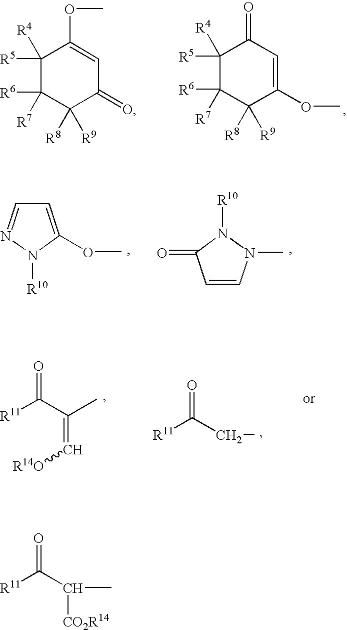
   in which
R⁴, R⁵, R⁶, R⁷, R⁸, R⁹, R¹⁰, R¹¹ and R¹⁴ have the same definition as aforementioned.

The reaction of process (a) can be conducted in an appropriate diluent. As the diluent used in that case there can be mentioned, for example, aliphatic, alicyclic and aromatic hydrocarbons (which may be optionally chlorinated), for example, toluene, dichloromethane, chloroform, 1,2-dichloroethane etc.; ethers, for example, ethyl ether, dimethoxyethane (DME), tetrahydrofuran (THF) etc.; ketones, for example, methyl isobutyl ketone (MIBK) etc.; nitriles, for example, acetonitrile etc.: esters, for example, ethyl acetate etc.; acid amides, for example, dimethylformamide (DMF) etc.

The process (a) can be conducted in the presence of a cyanide and a base and as the cyanide usable in that case there can be mentioned, for example, sodium cyanide, potassium cyanide, acetone cyanohydrin, hydrogen cyanide etc. As the base there can be mentioned, for example, as inorganic base, hydroxide, carbonate etc. of alkali metal and alkaline earth metal, for example, sodium carbonate, potassium carbonate, lithium hydroxide, sodium hydroxide, potassium hydroxide, calcium hydroxide etc.; and, as organic base, tertiary amines, dialkylaminoanilines, and pyridines, for example, triethylamine, pyridine, 4-dimethylaminopyridine (DMAP), 1,4-diazabicyclo[2,2,2]octane (DABCO) and 1,8-diazabicyclo[5,4,0]undec-7-ene (DBU) etc.

The process (a) can be also conducted by adding a phase transfer catalyst. As the phase transfer catalyst usable in that case there can be mentioned, for example, crown ethers, for example, dibenzo-18-crown-6,18-crown-6,15-crown-5 etc.

The process (a) can be conducted in a substantially wide range of temperature. However, the temperatures in a range of generally about −10 to about 80° C., preferably about 5 to about 40° C. are adequate. Although said reaction is conducted desirably under normal pressure, it can be conducted optionally under elevated pressure or under reduced pressure.

In conducting process (a), the aimed compounds of the formula (I) can be obtained, for example, by reacting 1 to 4 moles of triethylamine to 1 mole of a compound of the formula (II) in a diluent, for example, acetonitrile, in the presence of 0.01 to 0.5 moles of acetone cyanohydrin.

In conducting the preparation process (a), the aimed compounds of the formula (I) can be obtained by conducting a one-pot reaction starting from the compounds of formula (VIII) continuously without isolating the compounds of the formula (VI) and the compounds of the formula (II).

The reaction of the process (b) can be conducted in an appropriate diluent. As the diluent used in that case there can be mentioned, for example, aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile etc.; esters, for example, ethyl, acetate, amyl acetate etc.; acid amides, for example, dimethylformamide (DMF), dimethylacetamide (DMA), N-methylpyrrolidone, 1,3-dimethyl-2-imidazolidinone, hexamethyl phosphoric triamide (HMPA) etc.

The reaction of the process (b) can be conducted in a substantially wide range of temperature. However, the temperatures in a range of generally about −20 to about 100° C., preferably about 0 to about 50° C. are adequate. Although said reaction is conducted desirably under normal pressure, it can be conducted optionally under elevated pressure or under reduced pressure.

In conducting the preparation process (b), the aimed compounds of the formula (I) can be obtained, for example, by reacting 1 to 5 moles of oxalyl dichloride to 1 mole of a compound of the formula (Ib) in a diluent, for example, dichloromethane.

The reaction of the process (c) can be conducted in an appropriate diluent. As the diluent used in that case there can be mentioned, for example, aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; nitriles, for example, acetonitrile, propionitrile, acrylonitrile etc.; esters, for example, ethyl acetate, amyl acetate etc.; acid amides, for example, dimethylformamide (DMF), dimethylacetamide (DMA), N-methylpyrrolidone etc.; sulfones and sulfoxides, for example, dimethyl sulfoxide (DMSO), sulfolane etc.; bases, for example, pyridine etc.

The process (c) can be conducted in the presence of an acid binder. As the usable acid binder there can be mentioned, as inorganic base, hydrides,carbonates etc. of alkali metal, for example, sodium hydride, lithium hydride, sodium carbonate, potassium carbonate etc.; and as organic base, tertiary amines, dialkylaminoanilines, and pyridines, for example, triethylamine, 1,1,4,4tetramethyl ethylenediamine (TMEDA), pyridine, 4-dimethylaminopyridine (DMAP),1,4-diazabicyclo[2,2,2]-octane (DABCO), 1,8diazabicyclo[5,4,0]undec-7-ene (DBU) etc.

The reaction of the process (c) can be conducted in a substantially wide range of temperature. However, the temperatures in a range of generally about −20 to about 140° C., preferably about 0 to about 100° C. are adequate. Although said reaction is conducted desirably under normal pressure, it can be conducted optionally under elevated pressure or under reduced pressure.

In conducting the process (c), the aimed compounds of the formula (I) can be obtained, for example, by reacting 1 to 5 moles of a compound of the formula (III) to 1 mole of a compound of the formula (Ic) in a diluent, for example, tetrahydrofuran, in the presence of 1 to 5 moles of triethylamine.

The reaction of the process (d) can be conducted in an appropriate diluent. As the diluent used in that case there can be mentioned, for example, aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM) etc.; ketones, for example, acetone, methyl ethyl ketone (MEK), methyl isopropyl ketone, methyl isobutyl ketone (MIBK) etc.; esters, for example, ethyl acetate, amyl acetate etc.; acid amides, for example, dimethylformamide (DMF), dimethylacetamide (DMA), N-methylpyrrolidone, 1,3-dimethyl-2-imidazolidinone, hexamethyl phosphoric triamide (HMPA) etc.; sulfones and sulfoxides, for example, dimethyl sulfoxide (DMSO), sulfolane etc.; bases, for example, pyridine etc.

The process (d) can be conducted in the presence of an acid binder. As the usable acid binder there can be mentioned, as inorganic base, hydrides, carbonates etc. of alkali metal and alkaline earth metal, for example, sodium hydride, lithium hydride, sodium carbonate, potassium carbonate etc.; and as organic base, tertiary amines, dialkylaminoanilines, and pyridines, for example, triethylamine, 1,1,4,4-tetramethyl ethylenediamine (TMEDA), N,N-dimethylaniline, N,N-diethylaniline, pyridine, 4-dimethylaminopyridine (DMAP), 1,4-diazabicyclo[2,2,2]octane (DABCO), 1,8-di-azabicyclo[5,4,0]undec-7-ene (DBU) etc.

The process (d) can be conducted in a substantially wide range of temperature. However, the temperatures in a range of generally about −20 to about 140° C., preferably about 0 to about 100° C. are adequate. Although said reaction is conducted desirably under normal pressure, it can be conducted optionally under elevated pressure or under reduced pressure.

In conducting process (d), the aimed compounds of the formula (I) can be obtained, for example, by reacting 1-5 moles of a compound of the formula (IV) to 1 mole of a compound of the formula (Ib) in a diluent, for example, tetrahydrofuran, in the presence of triethylamine.

The reaction of the process (e) can be conducted in an appropriate diluent. As the diluent used in that case there can be mentioned, for example, ethers, for example, dioxane, tetrahydrofuran (THF) etc.; alcohols, for example, tert-amyl alcohol, tert-butyl alcohol etc.

The process (e) can be conducted in the presence of a base. As the base usable in that case there can be mentioned, as inorganic base, carbonate etc. of alkali metal, for example, sodium carbonate, potassium carbonate etc.; and as organic base, tertiary amines, for example, triethylamine, pyridine, 4-dimethylaminopyridine (DMAP) etc. The preparation process (e) can be conducted in a substantially wide range of temperature. However, the temperatures in a range of generally about 5 to about 200° C., preferably about 25 to about 130° C. are adequate. Although said reaction is conducted desirably under normal pressure, it can be conducted optionally under elevated pressure or under reduced pressure.

In conducting the process (e), the aimed compounds of the formula (I) can be obtained, for example, by reacting 0.5 to 2 moles of potassium carbonate to 1 mole of a compound of the formula (IIe) in a diluent, for example, dioxane.

The reaction of process (f) can be conducted in an appropriate diluent. As the diluent used in that case there can be mentioned, for example, aliphatic, alicyclic and aromatic hydrocarbons (may be optionally chlorinated), for example, toluene, dichloromethane, chloroform, 1,2-dichloroethane etc.; ethers, for example, tetrahydrofuran (THF) etc.; nitrites, for example, acetonitrile etc.; alcohols, for example, methanol, ethanol, isopropanol etc.

The process (f) can be conducted in the presence of a base. As the base usable in that case there can be mentioned, as inorganic base, acetate, carbonate, bicarbonate etc. of alkali metal and alkaline earth metal, for example, sodium acetate, sodium hydrogen carbonate, potassium hydrogen carbonate, sodium carbonate, potassium carbonate etc.; and as organic base, tertiary amines, dialkylaminoanilines and pyridines, for example, triethylamine, pyridine, 4-dimethylaminopyridine (DMAP) etc.

The process (f) can be conducted in a substantially wide range of temperature. However, the temperatures in a range of generally about −10 to about 100° C., preferably about 0 to about 50° C. are adequate. Although said reaction is conducted desirably under normal pressure, it can be conducted optionally under elevated pressure or under reduced pressure.

In conducting the process (f), the aimed compounds of the formula (I) can be obtained, for example, by reacting 1 to 1.5 moles of hydroxylamine hydrochloride to 1 mole of a compound of the formula (V) in a diluent, for example, ethanol, in the presence of 1 to 1.5 moles of sodium acetate.

In conducting the process (f), the aimed compounds of the formula (I) can be obtained by starting from the compounds of the formula (VI) to obtain the compounds of the formula (XII) by continuously reacting without isolating the compounds of the formula (XIV) and further by continuously reacting, starting from the compounds of the formula (XII) without isolating the compounds of the formula (V).

The reaction of the process (g) can be conducted in an appropriate diluent. As the diluent used in that case there can be mentioned, for example, water; aliphatic, alicyclic and aromatic hydrocarbons (may be optionally chlorinated), for example, benzene, toluene, xylene, dichloromethane, chloroform, carbon tetrachloride, 1,2-dichloroethane etc.; ethers, for example, ethyl ether, dioxane, dimethoxyethane (DME), tetrahydrofuran (THF) etc.; nitriles, for example, acetonitrile etc.; alcohols, for example, methanol, ethanol, isopropanol etc.; esters, for example, ethyl acetate etc.; acid amides, for example, dimethylformamide (DMF) etc.

The process (g) can be conducted in the presence of a base. As the base usable in that case there can be mentioned, as inorganic base, hydroxide, carbonate etc. of alkali metal and alkaline earth metal, for example, sodium carbonate, potassium carbonate, lithium hydroxide, sodium hydroxide, potassium hydroxide, calcium hydroxide etc.; and as organic base, alcoholates, tertiary amines, dialkylaminoanilines and pyridines, for example, triethylamine, 1,1,4,4-tetramethyl ethylenediamine (TMEDA), 4-dimethylaminopyridine (DMAP) etc.

The process (g) can be conducted in a substantially wide range of temperature. However, the temperatures in a range of generally about −10 to about 100° C., preferably about 0 to about 50° C. are adequate. Although said reaction is conducted desirably under normal pressure, it can be conducted optionally under elevated pressure or under reduced pressure.

In conducting the process (g), the aimed compounds of the formula (I) can be obtained, for example, by opening the ring of a compound of the formula (Ig) in a diluent, for example, dichloromethane, in the presence of 1 to 3 moles of triethylamine to 1 mole of the compound of the formula (Ig).

The compounds of formula (I) show excellent herbicidal activities against various weeds and can be used as herbicides. In the present specification weeds mean, in a broader sense, all plants that grow in locations where they are not desired. The compounds, according to the present invention, act as total or selective herbicide depending upon the applied concentration. The active compounds, according to the present invention, can be used, for example, between the following weeds and cultures.

Genera of the dicotyledonous weeds: Sinapis, Lepidium, Galium, Stellaria, Chenopodium, Urtica, Senecio, Amaranthus, Portulaca, Xanthium, Ipomoea, Polygonum, Ambrosia, Cirsium, Sonchus, Solanum, Rorippa, Lamium, Veronica, Datura, Viola, Galeopsis, Papaver, Centaurea, Galinsoga, Rotala, Lindernia etc.

Genera of the dicotyledonous cultures: Gossypium, Glycine, Beta, Daucus, Phaseolus, Pisum, Solanum, Linum, Ipomoea, Vicia, Nicotiana, Lycopersicon, Arachis, Brassica, Lactuca, Cucumis, Cucurbita etc.

Genera of the monocotyledonous weeds: Echinochloa, Setaria, Panicum, Digitaria, Phleum, Poa, Festuca, Eleusine, Lolium, Bromus, Avena, Cyperus, Sorghum, Agropyron, Monochoria, Fimbristylis, Sagittaria, Eleocharis, Scirpus, Paspalum, Ischaemum, Agrostis, Alopecurus, Cynodon etc.

Genera of the monocotyledonous cultures: Oryza, Zea, Triticum, Hordeum, Avena, Secale, Sorghum, Panicum, Saccharum, Ananas, Asparagus, Allium etc.

The active compounds of the formula (I), according to the present invention and mixed herbicidal compositions can be used particularly against paddy field weeds. As paddy field weeds that can be controlled by using the compounds, according to the present invention, and mixed herbicidal compositions there can be specifically mentioned, for example, Rotala indica Koehne, Lindernia Procumbens Philcox, Ludwigia prostrata Roxburgh, Potamogeton distinctus A. Benn, Elatine triandra Schk, Oenanthe javanica, Echinochloa oryzicola Vasing, Monochoria vaginalis Presl, Eleocharis acicularis L., Eleocharis Kuroguwai Ohwi, Cyperus difformis L., Cyperus serotinus Rottboel, Sagittaria pygmaea Miq, Alisma canaliculatum A. Br. Et Bouche, Scirpus juncoides Roxburgh etc. Moreover, the compounds of the formula (I) and mixed herbicidal compositions can be used against weeds that show resistance against sulfonylurea type herbicides. As said resistant weeds there can be specifically mentioned, for example, Rotala indica Koehne, Lindernia Procumbens Philcox, Lindernia dubia L. PENNEL, Lindernia var. dubia Pennell, Lindernia angustifolia Wettstein, Elatine triandra Schk, Monochoria korsakowii REGEL & MACK, Monochoria vaginalis Presl, Scirpus juncoides Roxburgh etc. The use of the active compounds of the formula (I) of the present invention and mixed herbicidal compositions, however, is not restricted to the use against these weeds but can be applied against other paddy field weeds and other weeds than the sulfonylurea type herbicide-resistant weeds in a similar manner.

The use of the compounds, according to the present invention, is not restricted to the above-mentioned plants, but can be applied against other plants in a similar manner. The active compounds, according to the present invention, can, depending upon the applied concentration, non-selectively control weeds and can be used, for example, on industrial terrain such as factories, rail tracks, paths, places with or without tree plantings. Moreover, the active compounds of the present invention can be used for controlling weeds in perennial cultures and can be applied, for example, in afforestations, decorative tree plantings, orchards, vineyards, citrus groves, nut orchards, banana plantations, coffee plantations, tea plantations, rubber plantations, oil palm plantations, cocoa plantations, soft fruit plantings, hop fields etc. Further, they can be applied for the selective weed control in annual cultures.

The active compounds, according to the present invention, and mixed herbicidal compositions can be made into customary formulation forms. As such formulation forms there can be mentioned, for example, solutions, wettable powders, emulsions, suspensions, powders, water dispersible granules, tablets, granules, suspo-emulsion concentrates, microcapsules in polymer substance, jumbo formulations etc.

These formulations can be prepared according to per se known methods, for example, by mixing the active compounds with extenders, namely liquid or solid diluents or carriers, and optionally with surface-active agents, namely emulsifiers and/or dispersants and/or foam-forming agents.

As liquid diluents or carriers there can be mentioned, for example, aromatic hydrocarbons (for example, xylene, toluene, alkylnaphthalene etc.), chlorinated aromatic or chlorinated aliphatic hydrocarbons (for example, chlorobenzenes, ethylene chlorides, methylene chloride etc.), aliphatic hydrocarbons [for example, cyclohexane etc. or paraffins (for example, mineral oil fractions etc.)], alcohols (for example, butanol, glycols etc.) and their ethers, esters etc., ketones (for example, acetone, methyl ethyl ketone, methyl isobutyl ketone, cyclohexanone etc.), strongly polar solvents (for example, dimethylformamide, dimethyl sulfoxide etc.), water etc. When water is used as extender, for example, organic solvents can be used as auxiliary solvents.

As solid diluents or carriers there can be mentioned, for example, ground natural minerals (for example, kaolin, clay, talc, chalk, quartz, attapulgite, montmorillonite, diatomaceous earth etc.), ground synthetic minerals (for example, highly dispersed silicic acid, alumina, silicates etc.) etc. As solid carriers for granules there can be mentioned crushed and fractionated rocks (for example, calcite, marble, pumice, sepiolite, dolomite etc.) synthetic granules of inorganic and organic meals, particles of organic materials (for example, saw dust, coconut shells, maize cobs, tobacco stalks etc.) etc.

As emulsifiers and/or foam-forming agents there can be mentioned, for example, nonionic and anionic emulsifiers [for example, polyoxyethylene fatty acid esters, polyoxyethylene fatty acid alcohol ethers (for example, alkylaryl polyglycol ethers, alkylsulfonates, alkylsulfates, arylsulfonates etc.)], albumin hydrolysis products etc.

Dispersants include, for example, lignin sulfite waste liquor, methyl cellulose etc.

Tackifiers can also be used in formulations (powders, granules, emulsifiable concentrates). As said tackifiers there can be mentioned, for example, carboxymethyl cellulose, natural and synthetic polymers (for example, gum Arabic, polyvinyl alcohol, polyvinyl acetate etc.).

Colorants can also be used. As said colorants there can be mentioned inorganic pigments (for example, iron oxide, titanium oxide, Prussian Blue etc,), organic dyestuffs such as alizarin dyestuffs, azo dyestuffs or metal phthalocyanine dyestuffs, and further traces nutrients such as salts of metals such as iron, manganese, boron, copper, cobalt, molybdenum, zinc etc.

Said formulations can contain the active compounds of the formula (I) in the range of generally 0.1 to 95% by weight, preferably 0.5 to 90% by weight.

The active compounds of the formula (I), according to the present invention, can be used for weed control as themselves or in their formulation forms. And the mixed herbicidal compositions with known herbicides can be previously prepared in the final formulation forms or can be prepared by tank mixing when they are used. As the herbicides that can be used as mixed herbicidal compositions in combination with the compounds of the formula (I) of the present invention there can be mentioned, for example, the following herbicides represented by common names as typical examples:
acetamide type herbicides: for example, pretilachlor, butachlor, thenylchlor, alachlor etc.;
amide type herbicides: for example, clomeprop, etobenzanid etc.;
benzofuran type herbicides: for example, benfuresate etc.;
indandione type herbicides: for example, indanofan etc.;
pyrazole type herbicides: for example, pyrazolate, benzofenap, pyrazoxifen etc.;
oxazinone type herbicides: for example, oxaziclomefone etc.;
sulfonylurea type herbicides: for example, bensulfuron-methyl, azimsulfuron, imazosulfuron, pyrazosulfuron-ethyl, cyclosulfamuron, ethoxysulfuron, halosulfuronmethyl etc.;
thiocarbamate type herbicides: for example, thiobencarb, molinate, pyributycarb etc.;
triazine type herbicides: for example, dimethametryn, simetryn etc.;
triazole type herbicides: for example, cafenstrole etc.;
quinoline type herbicides: for example, quinclorac etc.;
isoxazole type herbicides: for example, isoxaflutole etc.;
dithiophosphate type herbicides: for example, anilofos etc.;
oxyacetamide type herbicides: for example, mefenacet, flufenacet etc.;
tetrazolinone type herbicides: for example, fentrazamide etc.;
dicarboxyimide type herbicides: for example, pentoxazone etc.;
trione type herbicides: for example, sulcotrione, benzobicyclon etc.;
phenoxypropionate type herbicides: for example, cyhalofop-butyl etc.;
benzoic acid type herbicides: for example, pyrimenobac-methyl etc.;
diphenyl ether type herbicides: for example, chlomethoxyfen, oxyfluorfen etc.;
pyridine dicarbothioate type herbicides: for example, dithiopyr etc.;
phenoxy type herbicides: for example, MCPA, MCPB etc.;
urea type herbicides: for example, dymron, cumyluron etc.;
naphthalenedione type herbicides: for example, quinoclamine etc.;
isoxazolidinone type herbicides: for example, clomazone etc.

In addition to the above mentioned herbicides, the following herbicides shown in common names, for example,

Acetochlor, Acifluorfen (-sodium), Aclonifen, Alloxydim (-sodium), Ametryne, Amicarbazone, Amidochlor, Amidosulfuron, Amitrole, Asulam, Atrazine, Azafenidin, Beflubutamid, Benazolin (-ethyl), Bentazon, Benzfendizone, Benzoylprop (-ethyl), Bialaphos, Bifenox, Bispyribac (-sodium), Bromacil, Bromobutide, Bromofenoxim, Bromoxynil, Butafenacil (-allyl), Butenachlor, Butralin, Butroxydim, Butylate, Carbetamide, Carfentrazone (-ethyl), Chloramben, Chloridazon, Chlorimuron (-ethyl), Chlornitrofen, Chlorsulfuron, Chlorthiamid, Chlortoluron, Cinidon (-ethyl), Cinmethylin, Cinosulfuron, Clefoxydim, Clethodim, Clodinafop (-propargyl), Clopyralid, Cloransulam (-methyl), Cyanazine, Cybutryne, Cycloate, Cycloxydim, 2,4-D, 2,4-DB, Desmedipham, Diallate, Dicamba, Dichlobenil, Dichlorprop (—P), Diclofop (-methyl), Diclosulam, Diethatyl (-ethyl), Difenopenten (-ethyl), Difenzoquat, Diflufenican, Diflufenzopyr, Dikegulac (-sodium), Dimefuron, Dimepiperate, Dimethachlor, Dimethenamid (—P), Dimexyflam, Dinitramine, Diphenamid, Diquat (-dibromide), Diuron, Epropodan, EPTC, Esprocarb, Ethalfluralin, Ethametsulfuron (-methyl), Ethiozin, Ethofumesate, Ethoxyfen, Fenoxaprop (—P-ethyl), Flamprop (-M-isopropyl, -M-methyl), Flazasulfuron, Florasulam, Fluazifop (—P-butyl), Fluazolate, Flucarbazone (-sodium), Fluchloralin, Flumetsulam, Flumiclorac (-pentyl), Flumioxazin, Flumipropyn, Fluometuron, Fluorochloridone, Fluoroglycofen (-ethyl), Flupoxam, Flupropacil, Flurpyrsulfuron (-methyl, -sodium), Flurenol (-butyl), Fluridone, Fluroxypyr (-butoxypropyl, -meptyl), Flurprimidol, Flurtamone, Fluthiacet (-methyl), Fomesafen, Foramsulfuron, Glufosinate (-ammonium), Glyphosate (-ammonium, -isopropylammonium), Halosafen, Haloxyfop (-ethoxyethyl, —P-methyl), Hexazinone, Imazamethabenz (-methyl), Imazamethapyr, Imazamox, Imazapic, Imazapyr, Imazaquin, Imazethapyr, lodosulfuron (-methyl, -sodium), loxynil, Isopropalin, Isoproturon, Isouron, Isoxaben, Isoxachlortole, Isoxadifen (-ethyl), Isoxapyrifop, Ketospiradox, Lactofen, Lenacil, Linuron, Mecoprop (—P), Mesotrione, Metamitron, Metazachlor, Methabenzthiazuron, Methyldymron, Metobenzuron, Metobromuron, (S—) Metolachlor, Metosulam, Metoxuron, Metribuzin, Metsulfuron (-methyl), Monolinuron, Naproanilide, Napropamide, Neburon, Nicosulfuron, Norflurazon, Orbencarb, Oryzalin, Oxadiargyl, Oxadiazon, Oxasulfuron, Paraquat, Pelargonsaiure, Pendimethalin, Pendralin, Pethoxamid, Phenmedipham, Picolinafen, Piperophos, Primisulfuron (-methyl), Profluazol, Profoxydim, Prometryn, Propachlor, Propanil, Propaquizafop, Propisochlor, Propoxycarbazone (-sodium), Propyzamide, Prosulfocarb, Prosulfuron, Pyraflufen (-ethyl), Pyrazogyl, Pyribenzoxim, Pyridafol, Pyridate, Pyridatol, Pyriftalid, Pyrithiobac (-sodium), Quinmerac, Quizalofop (—P-ethyl, —P-tefuryl), Rimsulfuron, Sethoxydim, Simazine, Sulfentrazone, Sulfometuron (-methyl), Sulfosate, Sulfosulfuron, Tebutam, Tebuthiuron, Tepraloxydim, Terbuthylazine, Terbutryn, Thiazopyr, Thidiazimin, Thifensulfuron (-methyl), Tiocarbazil, Tralkoxydim, Triallate, Triasulfuron, Tribenuron (-methyl), Triclopyr, Tridiphane, Trifloxysulfuron, Trifluralin, Triflusulfuron (-methyl), Tritosulfuron.

Further, when the active compounds of the formula (I), according to the present invention, are mixed with safeners, phytotoxicity is reduced by the mixing and a broader weed controlling spectrum is provided and the application as a selective herbicide can be broadened.

As safeners there can be mentioned, for example, the following compounds represented by common names or development codes:

AD-67, BAS-145138, benoxacor, cloquintocet-mexyl, cyometrinil, 2,4-D, DKA-24, dichlormid, dymron, fenclorim, fenchlorazole-ethyl, flurazole, fluxofenim, flurazole, isoxadifen-ethyl, mefenpyr-diethyl, MG-191, naphthalic anhydride, oxabetrinil, PPG-1292, R-29148 etc.

The above-mentioned safeners and active compounds are also described in “The Pesticide Manual” published in 2000 by British Crop Protect Council.

Moreover, it is possible to further mix the above-mentioned safeners to a mixed herbicide composition consisting of a compound of the formula (I) of the present invention and an above-mentioned herbicide. By the mixing phytotoxicity is reduced and a broader weed controlling spectrum is provided and the application as a selective herbicide can be broadened.

Surprisingly, some of the mixed herbicide compositions consisting of a compound of the present invention and a known herbicide and/or a safener can show synergistic effects.

In case of using the active compounds of the formula (I), according to the present invention, they can be directly used as such or used in formulation forms such as ready-to-use solutions, emulsifiable concentrates, tablets, suspensions, powders, pastes or granules, or used in use forms prepared by further dilution. The active compounds, according to the present invention, can be applied by means of, for example, watering, spraying, atomizing, granule application etc.

The active compounds of the formula (I), according to the present invention, can be used at wither stages before and after germination of plants. They can be also mixed into the soil before sowing.

The application amount of the active compounds, according to the present invention, can be varied in a substantial range. It is fundamentally different depending upon the properties of effects to be desired. In case of using as herbicide, there can be mentioned application amounts in the range of, for example, about 0.01 to about 4 kg, preferably about 0.05 to about 3 kg as active compound per hectare.

Then the preparation and application of the compounds, according to the present invention, will be described more specifically by the following examples. The present invention, however, should not be restricted only to them in any way.

### SYNTHESIS EXAMPLE 1

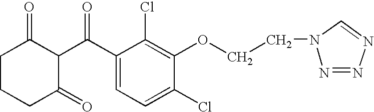

3-Oxo-1-cyclohexenyl 2,4-dichloro-3-[2-(1H-tetrazol-1-yl)ethoxy]benzoate (0.48 g) was dissolved in acetonitrile (15 ml), to which triethylamine (0.25 g) and acetone cyanohydrin (5 mg) were added and stirred at room temperature for 5 hours. After the solvent was distilled off, it was acidified by addition of diluted hydrochloric acid and extracted with dichloromethane (150 ml). The organic layer was washed with saturated aqueous solution of common salt and dried with anhydrous magnesium sulfate. Dichloromethane was distilled off to obtain the objective 2-{2,4-dichloro-3-[2-(1H-tetrazol-1-yl)ethoxy]benzoyl}cyclohexane-1,3-dione (0.41 g).

n^{D}₂₀: 1.5960

### SYNTHESIS EXAMPLE 2

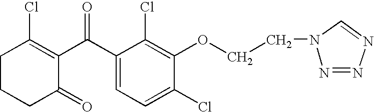

To a solution of 2-{2,4-dichloro-3-[2-(1H-tetrazol-1-yl)ethoxy]benzoyl}cyclohexane-1,3-dione (0.61 g) in dichloromethane (100 ml), oxalyl chloride (0.39 g) and 2 drops of N,N-dimethylformamide were added drop by drop under ice cooling. After that, the reaction solution was gradually warmed and refluxed for 3 hours. After the reaction, the solvent was distilled off and the obtained residue was purified by silica gel column chromatography (eluent:ethyl acetate:hexane=7:3) to obtain the objective 3-chloro-2-{2,4-dichloro-3-[2-(1H-tetrazol-1-yl)ethoxy]benzoyl}-2-cyclohexen-1-one (0.45 g).

Mp: 122-124° C.

### SYNTHESIS EXAMPLE 3

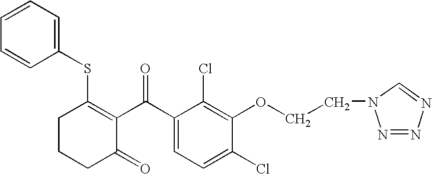

In tetrahydrofuran (7 ml) 3-chloro-2-{2,4-dichloro-3-[2-(1H-tetrazol-1-yl)ethoxy]-benzoyl}-2-cyclohexen-1-one (0.33 g) and thiophenol (0.10 g) were dissolved, to which triethylamine (0.10 g) in tetrahydrofuran (3 ml) was added drop at 5° C. by drop and stirred at room temperature for 4 hours. After the reaction, cold water was added thereto and the mixture was extracted with ethyl acetate (50 ml) and dried with anhydrous magnesium sulfate. After ethyl acetate was distilled off, the obtained residue was purified by silica gel column chromatography (eluent:ethyl acetate:hexane=2:1) to obtain the objective 2-{2,4-dichloro-3-[2-(1H-tetrazol-1-yl)ethoxy]-benzoyl}-3-phenylthio-2-cyclohexen-1-one (0.33 g).

Mp: 64-70° C.

### SYNTHESIS EXAMPLE 4

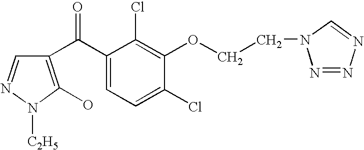

5-{2,4-Dichloro-3-[2-(1H-tetrazol-1-yl)ethoxy]benzoyloxy}-1-ethylpyrazole (0.58 g) was dissolved in acetonitrile (10 ml), to which triethylamine (0.30 g) and acetone cyanohydrin (4 mg) were added and stirred at room temperature for 5 hours. After the solvent was distilled off, it was acidified by addition of diluted hydrochloric acid and extracted with ethyl acetate (150 ml). The organic layer was washed with saturated aqueous solution of common salt and dried with anhydrous magnesium sulfate. Ethyl acetate was distilled off to obtain the objective 2-{2,4-dichloro-3-[2-(1H-tetrazol-1-yl)ethoxy]benzoyl}cyclohexane-1,3-dione (0.41 g).

Mp: 46-54° C.

The compounds obtained by operating in a similar manner as the preparation processes of the compounds of the formula (I) of the present invention exemplified in the above-mentioned Synthesis Examples 1-4, are shown, together with the compounds synthesized in Synthesis Examples 1-4, in the following Table 1 and Table 2.

Examples of the compounds of the formula (I) when they are represented by the formula 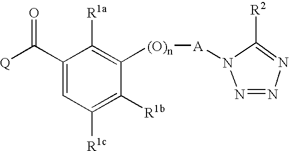
are shown in Table 1, and, when they are represented by the formula 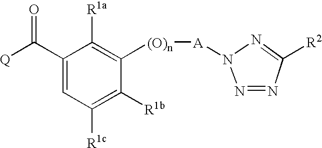
they are shown in Table 2.

The following abbreviations are used in table 1 and 2:

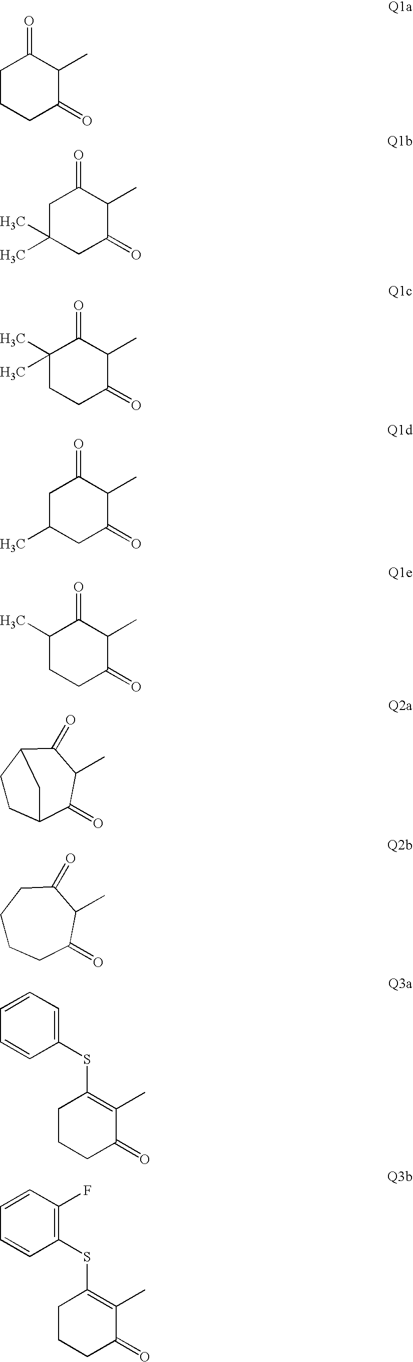

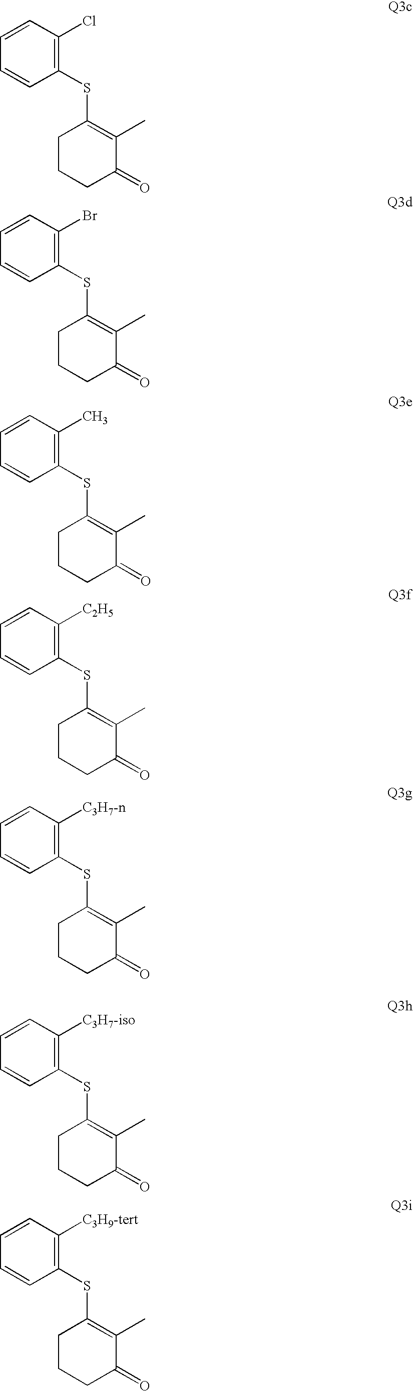

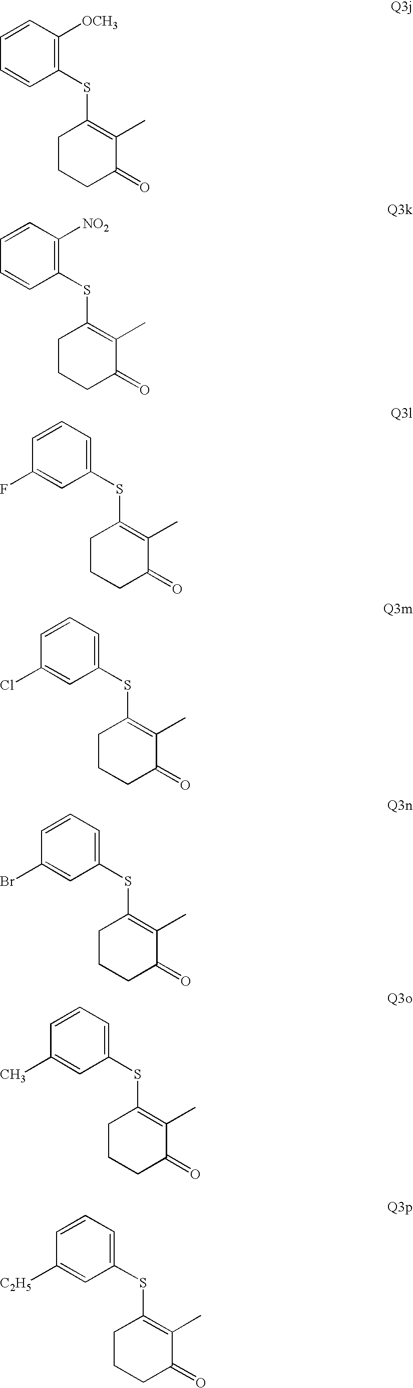

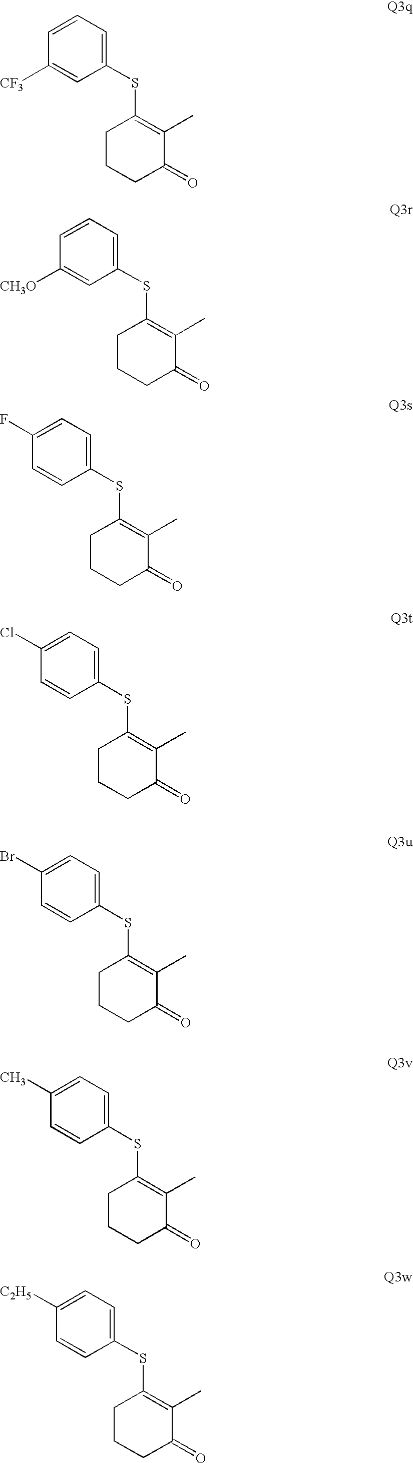

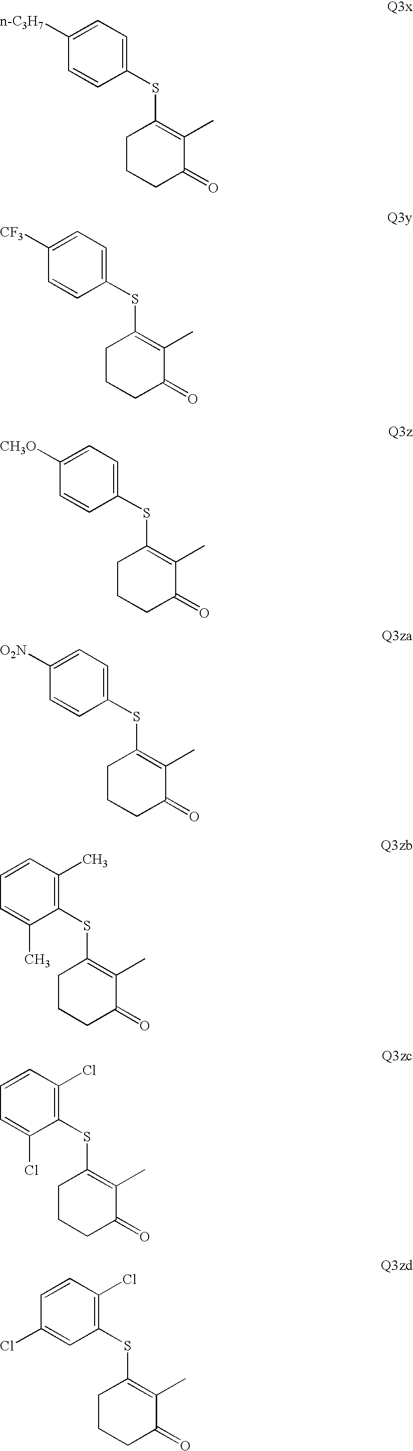

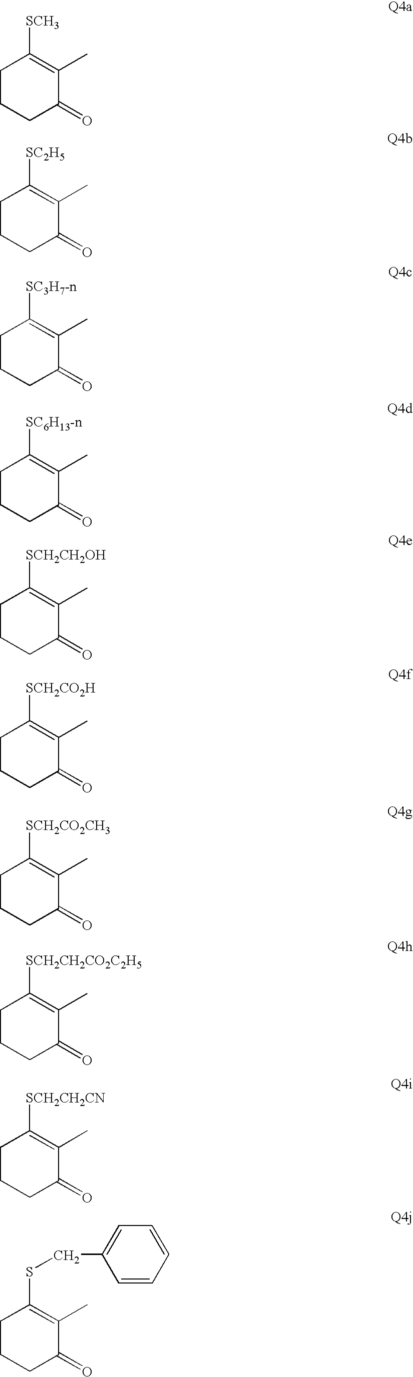

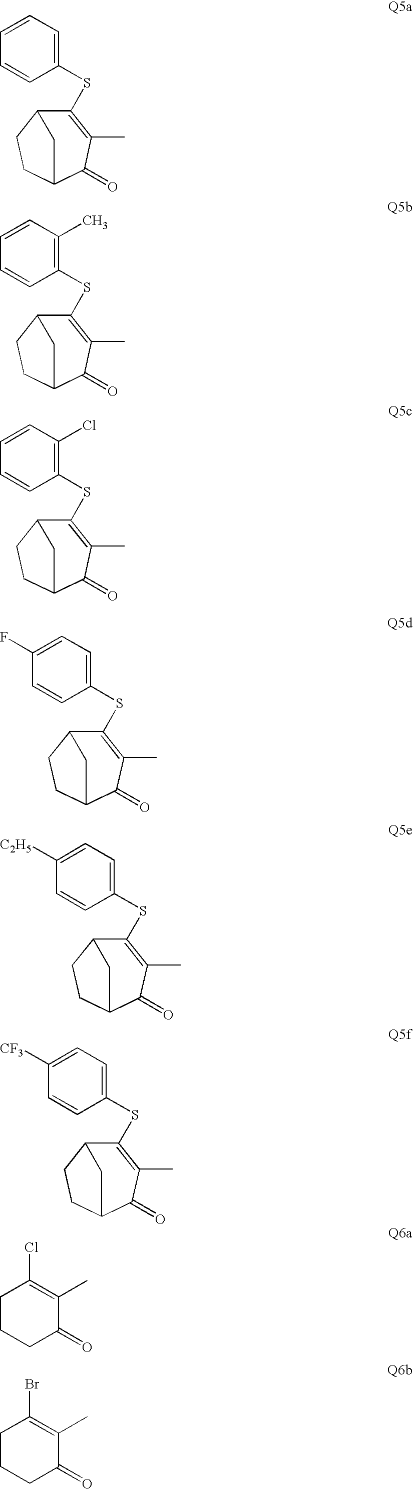

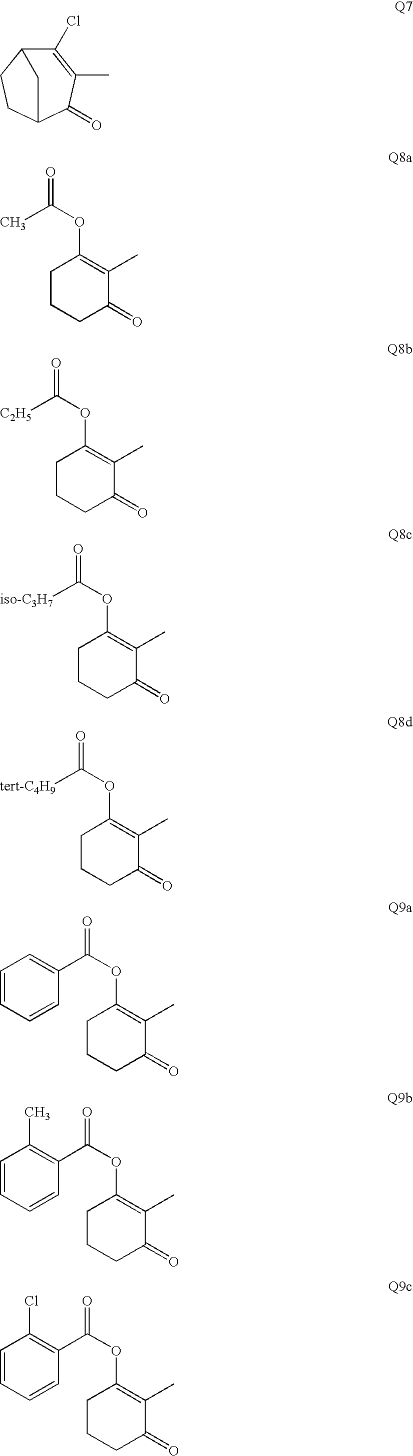

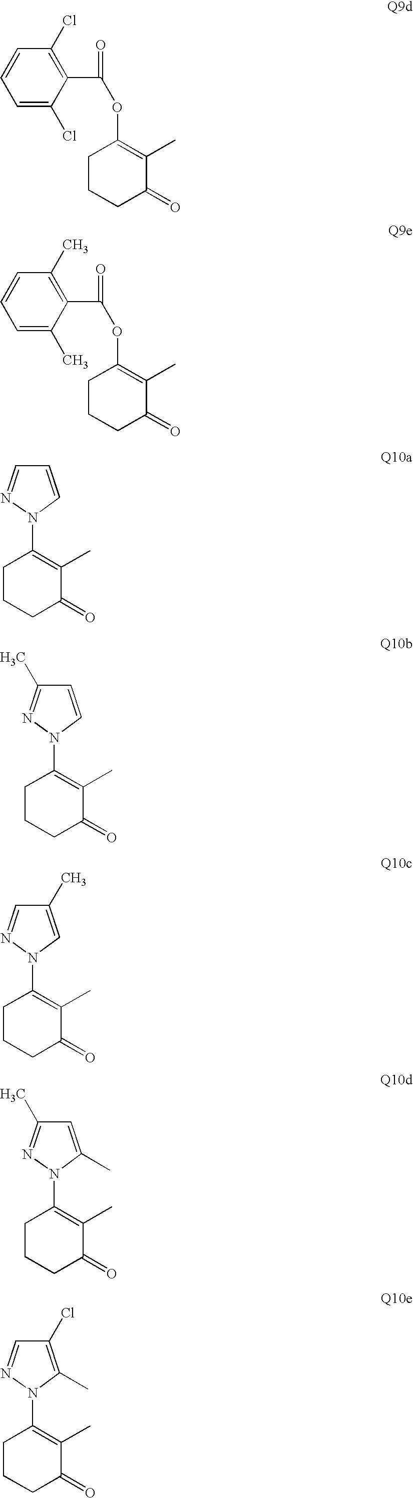

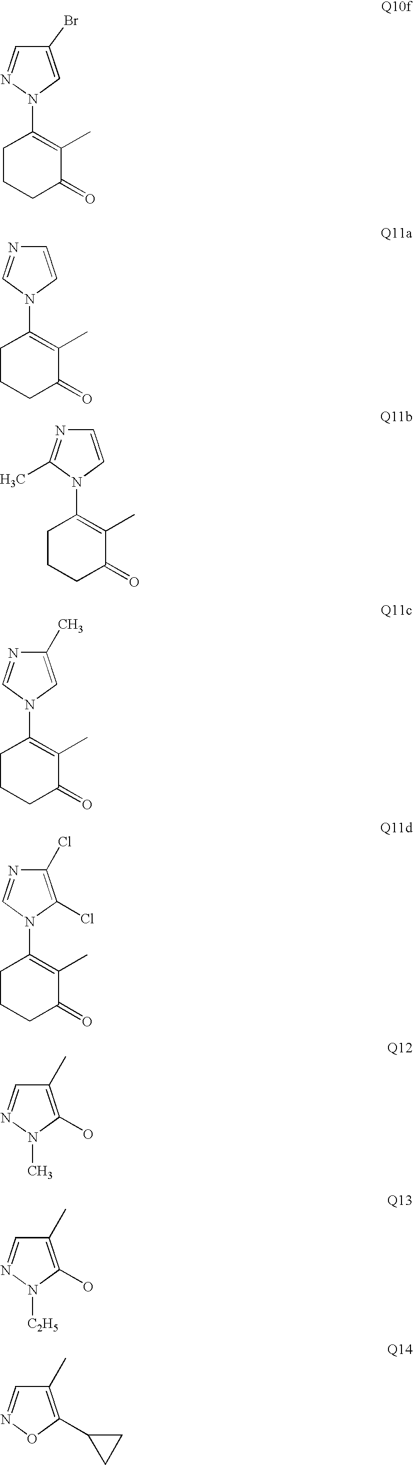

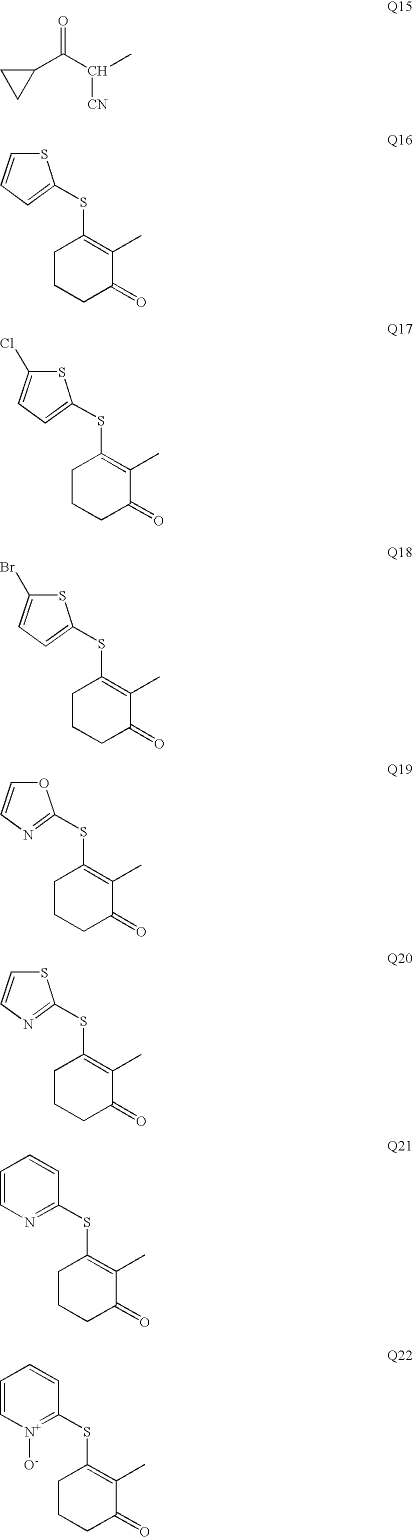

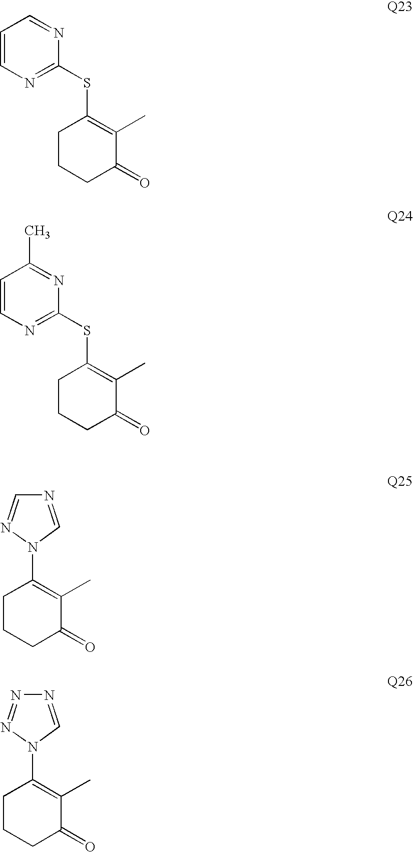

Further, Me represents methyl, Et represents ethyl, n-Pr represents n-propyl, i-Pr reprents isopropyl, n-Bu represents n-butyl, n-Pen represents n-pentyl, cyclo-Pro represents cyclopropyl, cyclo-Bu represents cyclobutyl, cyclo-Pen represents cyclopentyl, cyclo-Hex represents cyclohexyl, n-Hex represents n-hexyl, OMe represents methoxy, OEt represents ethoxy, SMe represents methylthio, SEt represents ethylthio, S-n-Pr represents n-propylthio, SO₂Me represents methylsulfonyl, SO₂Et represents ethylsulfonyl, SO₂Pr-n represents n-propylsulfonyl, OSO₂Me represents methylsulfonyloxy, OSO₂Et represents ethylsulfonyloxy, Ph represents phenyl, 2-Cl-Ph represents 2-chlorophenyl, 3-Cl-Ph represents 3-chlorophenyl, 2-Me-Ph represents 2-methylphenyl, 4-Me-Ph represents 4-methylphenyl, 3-Et-Ph represents 3-ethylphenyl, 4-n-Pr-Ph represents 4-(n-propyl)phenyl, 3-CF₃—Ph represents 3-trifluoromethylphenyl, 3-CH₂CF₃—Ph represents 3-(2,2,2-trifluoroethyl)phenyl and 3-NO₂—Ph represents 3-nitrophenyl.

TABLE 1

| Compd. | | | | | | Mp (° C.) |
|---|---|---|---|---|---|---|
| No. | R^{1a} | R^{1b} (R^{1c}) | (O)ₙ—A | R² | Q | or n_{D}²⁰ |
| 1. | H | Cl | OCH₂CH₂ | H | Q1a | |
| 2. | H | Cl | OCH₂CH₂ | H | Q2a | |
| 3. | F | Cl | CH₂ | H | Q1a | |
| 4. | F | Cl | OCH₂CH₂ | H | Q1a | |
| 5. | F | Cl | CH₂ | Me | Q1a | |
| 6. | Cl | F | CH₂ | H | Q1a | |
| 7. | Cl | F | CH₂ | Me | Q1a | |
| 8. | Cl | F | OCH₂CH₂ | H | Q1a | |
| 9. | Cl | F | OCH₂CH₂ | H | Q1b | |
| 10. | Cl | F | OCH₂CH₂ | H | Q1c | |
| 11. | Cl | F | OCH₂CH₂ | H | Q1d | |
| 12. | Cl | F | OCH₂CH₂ | H | Q1e | |
| 13. | Cl | F | OCH₂CH₂ | H | Q2a | |
| 14. | Cl | F | OCH₂CH₂ | H | Q2b | |
| 15. | Cl | F | OCH₂CH₂ | H | Q3a | |
| 16. | Cl | F | OCH₂CH₂ | H | Q3c | |
| 17. | Cl | F | OCH₂CH₂ | H | Q4a | |
| 18. | Cl | F | OCH₂CH₂ | H | Q5a | |
| 19. | Cl | F | OCH₂CH₂ | H | Q6a | |
| 20. | Cl | F | OCH₂CH₂ | H | Q8a | |
| 21. | Cl | F | OCH₂CH₂ | H | Q8d | |
| 22. | Cl | F | OCH₂CH₂ | H | Q9a | |
| 23. | Cl | F | OCH₂CH₂ | H | Q12 | |
| 24. | Cl | F | OCH₂CH₂ | H | Q13 | |
| 25. | Cl | F | OCH₂CH₂ | Me | Q1a | |
| 26. | Cl | F | OCH₂CH₂ | Me | Q2a | |
| 27. | Cl | F | OCH₂CH₂ | Me | Q2b | |
| 28. | Cl | F | OCH₂CH₂ | Me | Q3a | |
| 29. | Cl | F | OCH₂CH₂ | Me | Q6a | |
| 30. | Cl | F | OCH₂CH₂ | Me | Q8d | |
| 31. | Cl | F | OCH₂OH(Me) | H | Q1a | |
| 32. | Cl | F | OCH₂CH₂CH₂ | H | Q1a | |
| 33. | Cl | Cl | CH₂ | H | Q1a | 61-64 |
| 34. | Cl | Cl | CH₂ | H | Q1b | |
| 35. | Cl | Cl | CH₂ | H | Q1c | |
| 36. | Cl | Cl | CH₂ | H | Q1d | |
| 37. | Cl | Cl | CH₂ | H | Q1e | |
| 38. | Cl | Cl | CH₂ | H | Q2a | |
| 39. | Cl | Cl | CH₂ | H | Q2b | |
| 40. | Cl | Cl | CH₂ | H | Q3a | 179-180 |
| 41. | Cl | Cl | CH₂ | H | Q3b | |
| 42. | Cl | Cl | CH₂ | H | Q3c | |
| 43. | Cl | Cl | CH₂ | H | Q3d | |
| 44. | Cl | Cl | CH₂ | H | Q3e | |
| 45. | Cl | Cl | CH₂ | H | Q3f | |
| 46. | Cl | Cl | CH₂ | H | Q3g | |
| 47. | Cl | Cl | CH₂ | H | Q3h | |
| 48. | Cl | Cl | CH₂ | H | Q31 | |
| 49. | Cl | Cl | CH₂ | H | Q3j | |
| 50. | Cl | Cl | CH₂ | H | Q3k | |
| 51. | Cl | Cl | CH₂ | H | Q3l | |
| 52. | Cl | Cl | CH₂ | H | Q3m | |
| 53. | Cl | Cl | CH₂ | H | Q3n | |
| 54. | Cl | Cl | CH₂ | H | Q3o | |
| 55. | Cl | Cl | CH₂ | H | Q3p | |
| 56. | Cl | Cl | CH₂ | H | Q3q | |
| 57. | Cl | Cl | CH₂ | H | Q3r | |
| 58. | Cl | Cl | CH₂ | H | Q3s | |
| 59. | Cl | Cl | CH₂ | H | Q3t | |
| 60. | Cl | Cl | CH₂ | H | Q3u | |
| 61. | Cl | Cl | CH₂ | H | Q3v | |
| 62. | Cl | Cl | CH₂ | H | Q3w | |
| 63. | Cl | Cl | CH₂ | H | Q3x | |
| 64. | Cl | Cl | CH₂ | H | Q3y | |
| 65. | Cl | Cl | CH₂ | H | Q3z | |
| 66. | Cl | Cl | CH₂ | H | Q3za | |
| 67. | Cl | Cl | CH₂ | H | Q3zb | |
| 68. | Cl | Cl | CH₂ | H | Q3zc | |
| 69. | Cl | Cl | CH₂ | H | Q3zd | |
| 70. | Cl | Cl | CH₂ | H | Q4a | |
| 71. | Cl | Cl | CH₂ | H | Q4b | |
| 72. | Cl | Cl | CH₂ | H | Q4c | |
| 73. | Cl | Cl | CH₂ | H | Q4d | |
| 74. | Cl | Cl | CH₂ | H | Q5a | |
| 75. | Cl | Cl | CH₂ | H | Q5b | |
| 76. | Cl | Cl | CH₂ | H | Q5c | |
| 77. | Cl | Cl | CH₂ | H | Q6a | |
| 78. | Cl | Cl | CH₂ | H | Q6b | |
| 79. | Cl | Cl | CH₂ | H | Q7 | |
| 80. | Cl | Cl | CH₂ | H | Q8a | |
| 81. | Cl | Cl | CH₂ | H | Q8b | |
| 82. | Cl | Cl | CH₂ | H | Q8c | |
| 83. | Cl | Cl | CH₂ | H | Q8d | |
| 84. | Cl | Cl | CH₂ | H | Q9a | |
| 85. | Cl | Cl | CH₂ | H | Q9b | |
| 86. | Cl | Cl | CH₂ | H | Q9c | |
| 87. | Cl | Cl | CH₂ | H | Q9d | |
| 88. | Cl | Cl | CH₂ | H | Q10a | |
| 89. | Cl | Cl | CH₂ | H | Q11a | |
| 90. | Cl | Cl | CH₂ | H | Q12 | |
| 91. | GI | Cl | CH₂ | H | Q13 | |
| 92. | Cl | Cl | CH₂ | H | Q14 | |
| 93. | Cl | Cl | CH₂ | H | Q15 | |
| 94. | Cl | Cl | CH₂ | Me | Q1a | |
| 95. | Cl | Cl | CH₂ | Me | Q1b | |
| 96. | Cl | Cl | CH₂ | Me | Q1c | |
| 97. | Cl | Cl | CH₂ | Me | Q1d | |
| 98. | Cl | Cl | CH₂ | Me | Q1e | |
| 99. | Cl | Cl | CH₂ | Me | Q2a | |
| 100. | Cl | Cl | CH₂ | Me | Q2b | |
| 101. | Cl | Cl | CH₂ | Me | Q3a | |
| 102. | Cl | Cl | CH₂ | Me | Q3b | |
| 103. | Cl | Cl | CH₂ | Me | Q3c | |
| 104. | Cl | Cl | CH₂ | Me | Q3d | |
| 105. | Cl | Cl | CH₂ | Me | Q3e | |
| 106. | Cl | Cl | CH₂ | Me | Q3f | |
| 107. | Cl | Cl | CH₂ | Me | Q3g | |
| 108. | Cl | Cl | CH₂ | Me | Q3h | |
| 109. | Cl | Cl | CH₂ | Me | Q3i | |
| 110. | Cl | Cl | CH₂ | Me | Q3j | |
| 111. | Cl | Cl | CH₂ | Me | Q3k | |
| 112. | Cl | Cl | CH₂ | Me | Q31 | |
| 113. | Cl | Cl | CH₂ | Me | Q3m | |
| 114. | Cl | Cl | CH₂ | Me | Q3n | |
| 115. | Cl | Cl | CH₂ | Me | Q3o | |
| 116. | Cl | GI | CH₂ | Me | Q3p | |
| 117. | Cl | Cl | CH₂ | Me | Q3q | |
| 118. | Cl | Cl | CH₂ | Me | Q3r | |
| 119. | Cl | Cl | CH₂ | Me | Q3s | |
| 120. | Cl | Cl | CH₂ | Me | Q3t | |
| 121. | Cl | Cl | CH₂ | Me | Q3u | |
| 122. | Cl | Cl | CH₂ | Me | Q3v | |
| 123. | Cl | Cl | CH₂ | Me | Q3w | |
| 124. | Cl | Cl | CH₂ | Me | Q3x | |
| 125. | Cl | Cl | CH₂ | Me | Q3y | |
| 126. | Cl | Cl | CH₂ | Me | Q3z | |
| 127. | Cl | Cl | CH₂ | Me | Q3za | |
| 128. | Cl | Cl | CH₂ | Me | Q3zb | |
| 129. | Cl | Cl | CH₂ | Me | Q3zc | |
| 130. | Cl | Cl | CH₂ | Me | Q3zd | |
| 131. | Cl | Cl | CH₂ | Me | Q4a | |
| 132. | Cl | Cl | CH₂ | Me | Q4b | |
| 133. | Cl | Cl | CH₂ | Me | Q4c | |
| 134. | Cl | Cl | CH₂ | Me | Q4d | |
| 135. | Cl | Cl | CH₂ | Me | Q5a | |
| 136. | Cl | Cl | CH₂ | Me | Q6a | |
| 137. | Cl | Cl | CH₂ | Me | Q6b | |
| 138. | Cl | Cl | CH₂ | Me | Q7 | |
| 139. | Cl | Cl | CH₂ | Me | Q8a | |
| 140. | Cl | Cl | CH₂ | Me | Q8b | |
| 141. | Cl | Cl | CH₂ | Me | Q8c | |
| 142. | Cl | Cl | CH₂ | Me | Q8d | |
| 143. | Cl | Cl | CH₂ | Me | Q9a | |
| 144. | Cl | Cl | CH₂ | Me | Q9b | |
| 145. | Cl | Cl | CH₂ | Me | Q9c | |
| 146. | Cl | Cl | CH₂ | Me | Q9d | |
| 147. | Cl | Cl | CH₂ | Me | Q10a | |
| 148. | Cl | Cl | CH₂ | Me | Q11a | |
| 149. | Cl | Cl | CH₂ | Me | Q12 | |
| 150. | Cl | Cl | CH₂ | Me | Q13 | |
| 151. | Cl | Cl | CH₂ | Me | Q14 | |
| 152. | Cl | Cl | CH₂ | Me | Q15 | |
| 153. | Cl | Cl | CH₂ | Et | Q1a | |
| 154. | Cl | Cl | CH₂ | Et | Q1b | |
| 155. | Cl | Cl | CH₂ | Et | Q1c | |
| 156. | Cl | Cl | CH₂ | Et | Q1d | |
| 157. | Cl | Cl | CH₂ | Et | Q1e | |
| 158. | Cl | Cl | CH₂ | Et | Q2a | |
| 159. | Cl | Cl | CH₂ | Et | Q2b | |
| 160. | Cl | Cl | CH₂ | Et | Q3a | |
| 161. | Cl | Cl | CH₂ | Et | Q3c | |
| 162. | Cl | Cl | CH₂ | Et | Q4a | |
| 163. | Cl | Cl | CH₂ | Et | Q5a | |
| 164. | Cl | Cl | CH₂ | Et | Q6a | |
| 165. | Cl | Cl | CH₂ | Et | Q8a | |
| 166. | Cl | Cl | CH₂ | Et | Q8d | |
| 167. | Cl | Cl | CH₂ | Et | Q9a | |
| 168. | Cl | Cl | CH₂ | Et | Q12 | |
| 169. | Cl | Cl | CH₂ | Et | Q13 | |
| 170. | Cl | Cl | CH₂ | n-Pr | Q1a | |
| 171. | Cl | Cl | CH₂ | n-Pr | Q3a | |
| 172. | Cl | Cl | CH₂ | n-Pr | Q6a | |
| 173. | Cl | Cl | CH₂ | SMe | Q1a | 59-63 |
| 174. | Cl | Cl | CH₂ | SMe | Q1b | |
| 175. | Cl | Cl | CH₂ | SMe | Q1c | |
| 176. | Cl | Cl | CH₂ | SMe | Q1d | |
| 177. | Cl | Cl | CH₂ | SMe | Q1e | |
| 178. | Cl | Cl | CH₂ | SMe | Q2a | |
| 179. | Cl | Cl | CH₂ | SMe | Q2b | |
| 180. | Cl | Cl | CH₂ | SMe | Q3a | |
| 181. | Cl | Cl | CH₂ | SMe | Q3c | |
| 182. | Cl | Cl | CH₂ | SMe | Q4a | |
| 183. | Cl | Cl | CH₂ | SMe | Q5a | |
| 184. | Cl | Cl | CH₂ | SMe | Q6a | |
| 185. | Cl | Cl | CH₂ | SMe | Q8a | |
| 186. | Cl | Cl | CH₂ | SMe | Q8d | |
| 187. | Cl | Cl | CH₂ | SMe | Q9a | |
| 188. | Cl | Cl | CH₂ | SMe | Q12 | |
| 189. | Cl | Cl | CH₂ | SMe | Q13 | |
| 190. | Cl | Cl | CH₂CH₂ | H | Q1a | |
| 191. | Cl | Cl | CH₂CH₂ | H | Q1b | |
| 192. | Cl | Cl | CH₂CH₂ | H | Q1c | |
| 193. | Cl | Cl | CH₂CH₂ | H | Q1d | |
| 194. | Cl | Cl | CH₂CH₂ | H | Q1e | |
| 195. | Cl | Cl | CH₂CH₂ | H | Q2a | |
| 196. | Cl | Cl | CH₂CH₂ | H | Q2b | |
| 197. | Cl | Cl | CH₂CH₂ | H | Q3a | |
| 198. | Cl | Cl | CH₂CH₂ | H | Q3c | |
| 199. | Cl | Cl | CH₂CH₂ | H | Q4a | |
| 200. | Cl | Cl | CH₂CH₂ | H | Q5a | |
| 201. | Cl | Cl | CH₂CH₂ | H | Q6a | |
| 202. | Cl | Cl | CH₂CH₂ | H | Q8a | |
| 203. | Cl | Cl | CH₂CH₂ | H | Q8d | |
| 204. | Cl | Cl | CH₂CH₂ | H | Q9a | |
| 205. | Cl | Cl | CH₂CH₂ | H | Q12 | |
| 206. | Cl | Cl | CH₂CH₂ | H | Q13 | |
| 207. | Cl | Cl | CH₂CH₂ | Me | Q1a | |
| 208. | Cl | Cl | CH₂CH₂ | Me | Q2a | |
| 209. | Cl | Cl | CH₂CH₂ | Me | Q2b | |
| 210. | Cl | Cl | CH₂CH₂ | Me | Q3a | |
| 211. | Cl | Cl | CH₂CH₂ | Me | Q6a | |
| 212. | Cl | Cl | OCH₂CH₂ | H | Q1a | 1.5960 |
| 213. | Cl | Cl | OCH₂CH₂ | H | Q1b | 1.5710 |
| 214. | Cl | Cl | OCH₂CH₂ | H | Q1c | |
| 215. | Cl | Cl | OCH₂CH₂ | H | Q1d | 1.5758 |
| 216. | Cl | Cl | OCH₂CH₂ | H | Q1e | |
| 217. | Cl | Cl | OCH₂CH₂ | H | Q2a | 56-60 |
| 218. | Cl | Cl | OCH₂CH₂ | H | Q2b | 1.5830 |
| 219. | Cl | Cl | OCH₂CH₂ | H | Q3a | 64-70 |
| 220. | Cl | Cl | OCH₂CH₂ | H | Q3b | |
| 221. | Cl | Cl | OCH₂CH₂ | H | Q3c | |
| 222. | Cl | Cl | OCH₂CH₂ | H | Q3d | |
| 223. | Cl | Cl | OCH₂CH₂ | H | Q3e | |
| 224. | Cl | Cl | OCH₂CH₂ | H | Q3f | |
| 225. | Cl | Cl | OCH₂CH₂ | H | Q3g | |
| 226. | Cl | Cl | OCH₂CH₂ | H | Q3h | |
| 227. | Cl | Cl | OCH₂CH₂ | H | Q3i | |
| 228. | Cl | Cl | OCH₂CH₂ | H | Q3j | |
| 229. | Cl | Cl | OCH₂CH₂ | H | Q3k | |
| 230. | Cl | Cl | OCH₂CH₂ | H | Q31 | |
| 231. | Cl | Cl | OCH₂CH₂ | H | Q3m | |
| 232. | Cl | Cl | OCH₂CH₂ | H | Q3n | |
| 233. | Cl | Cl | OCH₂CH₂ | H | Q3o | |
| 234. | Cl | Cl | OCH₂CH₂ | H | Q3p | |
| 235. | Cl | Cl | OCH₂CH₂ | H | Q3q | |
| 236. | Cl | Cl | OCH₂CH₂ | H | Q3r | |
| 237. | Cl | Cl | OCH₂CH₂ | H | Q3s | |
| 238. | Cl | Cl | OCH₂CH₂ | H | Q3t | |
| 239. | Cl | Cl | OCH₂CH₂ | H | Q3u | |
| 240. | Cl | Cl | OCH₂CH₂ | H | Q3v | |
| 241. | Cl | Cl | OCH₂CH₂ | H | Q3w | |
| 242. | Cl | Cl | OCH₂CH₂ | H | Q3x | |
| 243. | Cl | Cl | OCH₂CH₂ | H | Q3y | |
| 244. | Cl | Cl | OCH₂CH₂ | H | Q3z | |
| 245. | Cl | Cl | OCH₂CH₂ | H | Q3za | |
| 246. | Cl | Cl | OCH₂CH₂ | H | Q3zb | |
| 247. | Cl | Cl | OCH₂CH₂ | H | Q3zc | |
| 248. | Cl | Cl | OCH₂CH₂ | H | Q3zd | |
| 249. | Cl | Cl | OCH₂CH₂ | H | Q4a | |
| 250. | Cl | Cl | OCH₂CH₂ | H | Q4b | |
| 251. | Cl | Cl | OCH₂CH₂ | H | Q4c | |
| 252. | Cl | Cl | OCH₂CH₂ | H | Q4d | |
| 253. | Cl | Cl | OCH₂CH₂ | H | Q5a | |
| 254. | Cl | Cl | OCH₂CH₂ | H | Q5b | |
| 255. | Cl | Cl | OCH₂CH₂ | H | Q5c | |
| 256. | Cl | Cl | OCH₂CH₂ | H | Q5d | |
| 257. | Cl | Cl | OCH₂CH₂ | H | Q5e | |
| 258. | Cl | Cl | OCH₂CH₂ | H | Q6a | 122-124 |
| 259. | Cl | Cl | OCH₂CH₂ | H | Q6b | |
| 260. | Cl | Cl | OCH₂CH₂ | H | Q7 | |
| 261. | Cl | Cl | OCH₂CH₂ | H | Q8a | |
| 262. | Cl | Cl | OCH₂CH₂ | H | Q8b | |
| 263. | Cl | Cl | OCH₂CH₂ | H | Q8c | |
| 264. | Cl | Cl | OCH₂CH₂ | H | Q8d | |
| 265. | Cl | Cl | OCH₂CH₂ | H | Q9a | |
| 266. | Cl | Cl | OCH₂CH₂ | H | Q9b | |
| 267. | Cl | Cl | OCH₂CH₂ | H | Q9c | |
| 268. | Cl | Cl | OCH₂CH₂ | H | Q9d | |
| 269. | Cl | Cl | OCH₂CH₂ | H | Q10a | |
| 270. | Cl | Cl | OCH₂CH₂ | H | Q11a | |
| 271. | Cl | Cl | OCH₂CH₂ | H | Q12 | |
| 272. | Cl | Cl | OCH₂CH₂ | H | Q13 | 46-54 |
| 273. | Cl | Cl | OCH₂CH₂ | H | Q14 | |
| 274. | Cl | Cl | OCH₂CH₂ | H | Q15 | |
| 275. | Cl | Cl | OCH₂CH₂ | Me | Q1a | 1.5908 |
| 276. | Cl | Cl | OCH₂CH₂ | Me | Q1b | 1.5475 |
| 277. | Cl | Cl | OCH₂CH₂ | Me | Q1c | |
| 278. | Cl | Cl | OCH₂CH₂ | Me | Q1d | 1.5741 |
| 279. | Cl | Cl | OCH₂CH₂ | Me | Q1e | |
| 280. | Cl | Cl | OCH₂CH₂ | Me | Q2a | |
| 281. | Cl | Cl | OCH₂CH₂ | Me | Q2b | |
| 282. | Cl | Cl | OCH₂CH₂ | Me | Q3a | |
| 283. | Cl | Cl | OCH₂CH₂ | Me | Q3b | |
| 284. | Cl | Cl | OCH₂CH₂ | Me | Q3c | |
| 285. | Cl | Cl | OCH₂CH₂ | Me | Q3d | |
| 286. | Cl | Cl | OCH₂CH₂ | Me | Q3e | |
| 287. | Cl | Cl | OCH₂CH₂ | Me | Q3f | |
| 288. | Cl | Cl | OCH₂CH₂ | Me | Q3g | |
| 289. | Cl | Cl | OCH₂CH₂ | Me | Q3h | |
| 290. | Cl | Cl | OCH₂CH₂ | Me | Q3i | |
| 291. | Cl | Cl | OCH₂CH₂ | Me | Q3j | |
| 292. | Cl | Cl | OCH₂CH₂ | Me | Q3k | |
| 293. | Cl | Cl | OCH₂CH₂ | Me | Q31 | |
| 294. | Cl | Cl | OCH₂CH₂ | Me | Q3m | |
| 295. | Cl | Cl | OCH₂CH₂ | Me | Q3n | |
| 296. | Cl | Cl | OCH₂CH₂ | Me | Q3o | |
| 297. | Cl | Cl | OCH₂CH₂ | Me | Q3p | |
| 298. | Cl | Cl | OCH₂CH₂ | Me | Q3q | |
| 299. | Cl | Cl | OCH₂CH₂ | Me | Q3r | |
| 300. | Cl | Cl | OCH₂CH₂ | Me | Q3s | |
| 301. | Cl | Cl | OCH₂CH₂ | Me | Q3t | |
| 302. | Cl | Cl | OCH₂CH₂ | Me | Q3u | |
| 303. | Cl | Cl | OCH₂CH₂ | Me | Q3v | |
| 304. | Cl | Cl | OCH₂CH₂ | Me | Q3w | |
| 305. | Cl | Cl | OCH₂CH₂ | Me | Q3x | |
| 306. | Cl | Cl | OCH₂CH₂ | Me | Q3y | |
| 307. | Cl | Cl | OCH₂CH₂ | Me | Q3z | |
| 308. | Cl | Cl | OCH₂CH₂ | Me | Q3za | |
| 309. | Cl | Cl | OCH₂CH₂ | Me | Q3zb | |
| 310. | Cl | Cl | OCH₂CH₂ | Me | Q3zc | |
| 311. | Cl | Cl | OCH₂CH₂ | Me | Q3zd | |
| 312. | Cl | Cl | OCH₂CH₂ | Me | Q4a | |
| 313. | Cl | Cl | OCH₂CH₂ | Me | Q4b | |
| 314. | Cl | Cl | OCH₂CH₂ | Me | Q4c | |
| 315. | Cl | Cl | OCH₂CH₂ | Me | Q4d | |
| 316. | Cl | Cl | OCH₂CH₂ | Me | Q5a | |
| 317. | Cl | Cl | OCH₂CH₂ | Me | Q5b | |
| 318. | Cl | Cl | OCH₂CH₂ | Me | Q5c | |
| 319. | Cl | Cl | OCH₂CH₂ | Me | Q6b | |
| 320. | Cl | Cl | OCH₂CH₂ | Me | Q7 | |
| 321. | Cl | Cl | OCH₂CH₂ | Me | Q8a | |
| 322. | Cl | Cl | OCH₂CH₂ | Me | Q8b | |
| 323. | Cl | Cl | OCH₂CH₂ | Me | Q8c | |
| 324. | Cl | Cl | OCH₂CH₂ | Me | Q8d | |
| 325. | Cl | Cl | OCH₂CH₂ | Me | Q9a | |
| 326. | Cl | Cl | OCH₂CH₂ | Me | Q9b | |
| 327. | Cl | Cl | OCH₂CH₂ | Me | Q9c | |
| 328. | Cl | Cl | OCH₂CH₂ | Me | Q9d | |
| 329. | Cl | Cl | OCH₂CH₂ | Me | Q10a | |
| 330. | Cl | Cl | OCH₂CH₂ | Me | Q11a | |
| 331. | Cl | Cl | OCH₂CH₂ | Me | Q12 | |
| 332. | Cl | Cl | OCH₂CH₂ | Me | Q13 | |
| 333. | Cl | Cl | OCH₂CH₂ | Me | Q14 | |
| 334. | Cl | Cl | OCH₂CH₂ | Me | Q15 | |
| 335. | Cl | Cl | OCH₂CH₂ | Et | Q1a | |
| 336. | Cl | Cl | OCH₂CH₂ | Et | Q1b | |
| 337. | Cl | Cl | OCH₂CH₂ | Et | Q1c | |
| 338. | Cl | Cl | OCH₂CH₂ | Et | Q1d | |
| 339. | Cl | Cl | OCH₂CH₂ | Et | Q1e | |
| 340. | Cl | Cl | OCH₂CH₂ | Et | Q2a | |
| 341. | Cl | Cl | OCH₂CH₂ | Et | Q2b | |
| 342. | Cl | Cl | OCH₂CH₂ | Et | Q3a | |
| 343. | Cl | Cl | OCH₂CH₂ | Et | Q3c | |
| 344. | Cl | Cl | OCH₂CH₂ | Et | Q4a | |
| 345. | Cl | Cl | OCH₂CH₂ | Et | Q5a | |
| 346. | Cl | Cl | OCH₂CH₂ | Et | Q6a | |
| 347. | Cl | Cl | OCH₂CH₂ | Et | Q8a | |
| 348. | Cl | Cl | OCH₂CH₂ | Et | Q8d | |
| 349. | Cl | Cl | OCH₂CH₂ | Et | Q9a | |
| 350. | Cl | Cl | OCH₂CH₂ | Et | Q12 | |
| 351. | Cl | Cl | OCH₂CH₂ | Et | Q13 | |
| 352. | Cl | Cl | OCH₂CH₂ | n-Pr | Q1a | |
| 353. | Cl | Cl | OCH₂CH₂ | n-Pr | Q3a | |
| 354. | Cl | Cl | OCH₂CH₂ | n-Pr | Q6a | |
| 355. | Cl | Cl | OCH₂CH₂ | SMe | Q7a | 1.6028 |
| 356. | Cl | Cl | OCH₂CH₂ | SMe | Q8a | |
| 357. | Cl | Cl | OCH₂CH₂ | SMe | Q6a | |
| 358. | Cl | Cl | OCH₂CH(Me) | H | Q1a | |
| 359. | Cl | Cl | OCH₂CH(Me) | H | Q1b | |
| 360. | Cl | Cl | OCH₂CH(Me) | H | Q1c | |
| 361. | Cl | Cl | OCH₂CH(Me) | H | Q1d | |
| 362. | Cl | Cl | OCH₂CH(Me) | H | Q1e | |
| 363. | Cl | Cl | OCH₂CH(Me) | H | Q2a | |
| 364. | Cl | Cl | OCH₂CH(Me) | H | Q2b | |
| 365. | Cl | Cl | OCH₂CH(Me) | H | Q3a | |
| 366. | Cl | Cl | OCH₂CH(Me) | H | Q3c | |
| 367. | Cl | Cl | OCH₂CH(Me) | H | Q4a | |
| 368. | Cl | Cl | OCH₂CH(Me) | H | Q5a | |
| 369. | Cl | Cl | OCH₂CH(Me) | H | Q6a | |
| 370. | Cl | Cl | OCH₂CH(Me) | H | Q8a | |
| 371. | Cl | Cl | OCH₂CH(Me) | H | Q8d | |
| 372. | Cl | Cl | OCH₂CH(Me) | H | Q9a | |
| 373. | Cl | Cl | OCH₂CH(Me) | H | Q12 | |
| 374. | Cl | Cl | OCH₂CH(Me) | H | Q13 | |
| 375. | Cl | Cl | OCH₂CH(Me) | Me | Q1a | |
| 376. | Cl | Cl | OCH₂CH(Me) | Me | Q3a | |
| 377. | Cl | Cl | OCH₂CH(Me) | Me | Q6a | |
| 378. | Cl | Cl | OCH₂CH₂CH₂ | H | Q1a | |
| 379. | Cl | Cl | OCH₂CH₂CH₂ | H | Q1b | |
| 380. | Cl | Cl | OCH₂CH₂CH₂ | H | Q1c | |
| 381. | Cl | Cl | OCH₂CH₂CH₂ | H | Q1d | |
| 382. | Cl | Cl | OCH₂CH₂CH₂ | H | Q1e | |
| 383. | Cl | Cl | OCH₂CH₂CH₂ | H | Q2a | |
| 384. | Cl | Cl | OCH₂CH₂CH₂ | H | Q2b | |
| 385. | Cl | Cl | OCH₂CH₂CH₂ | H | Q3a | |
| 386. | Cl | Cl | OCH₂CH₂CH₂ | H | Q3c | |
| 387. | Cl | Cl | OCH₂CH₂CH₂ | H | Q4a | |
| 388. | Cl | Cl | OCH₂CH₂CH₂ | H | Q5a | |
| 389. | Cl | Cl | OCH₂CH₂CH₂ | H | Q6a | |
| 390. | Cl | Cl | OCH₂CH₂CH₂ | H | Q8a | |
| 391. | Cl | Cl | OCH₂CH₂CH₂ | H | Q8d | |
| 392. | Cl | Cl | OCH₂CH₂CH₂ | H | Q9a | |
| 393. | Cl | Cl | OCH₂CH₂CH₂ | H | Q12 | |
| 394. | Cl | Cl | OCH₂CH₂CH₂ | H | Q13 | |
| 395. | Cl | Cl | OCH₂CH₂CH₂ | Me | Q1a | |
| 396. | Cl | Cl | OCH₂CH₂CH₂ | Me | Q3a | |
| 397. | Cl | Cl | OCH₂CH₂CH₂ | Me | Q6a | |
| 398. | Cl | Cl | CH(Me) | H | Q1a | |
| 399. | Cl | Cl | CH(Me)CH₂ | H | Q1a | |
| 400. | Cl | Cl | CH₂CH₂CH₂ | H | Q1a | |
| 401. | Cl | Cl | OCH₂ | H | Q1a | |
| 402. | Cl | Cl | OCH(Me)CH₂ | H | Q1a | |
| 403. | Cl | Cl(Me) | OCH₂CH₂ | H | Q1a | |
| 404. | Cl | Cl(Me) | OCH₂CH₂ | H | Q2a | |
| 405. | Cl | Cl(Me) | OCH₂CH₂ | H | Q2b | |
| 406. | Cl | Cl(Me) | OCH₂CH₂ | H | Q3a | |
| 407. | Cl | Cl(Me) | OCH₂CH₂ | H | Q6a | |
| 408. | Cl | Cl(Me) | OCH₂CH₂ | H | Q8d | |
| 409. | Cl | Cl(Me) | OCH₂CH₂ | Me | Q1a | |
| 410. | Cl | Cl(Me) | OCH₂CH₂ | Me | Q2a | |
| 411. | Cl | Cl(Me) | OCH₂CH₂ | Me | Q2b | |
| 412. | Cl | Cl(Me) | OCH₂CH₂ | Me | Q3a | |
| 413. | Cl | Cl(Me) | OCH₂CH₂ | Me | Q6a | |
| 414. | Cl | Cl(Me) | OCH₂CH₂ | Me | Q8d | |
| 415. | Cl | Br | CH₂ | H | Q1a | |
| 416. | Cl | Br | CF₂ | Me | Q1a | |
| 417. | Cl | Br | OCH₂CH₂ | H | Q1a | |
| 418. | Cl | Br | OCH₂CH₂ | H | Q1b | |
| 419. | Cl | Br | OCH₂CH₂ | H | Q1c | |
| 420. | Cl | Br | OCH₂CH₂ | H | Q1d | |
| 421. | Cl | Br | OCH₂CH₂ | H | Q1e | |
| 422. | Cl | Br | OCH₂CH₂ | H | Q2a | |
| 423. | Cl | Br | OCH₂CH₂ | H | Q2b | |
| 424. | Cl | Br | OCH₂CH₂ | H | Q3a | |
| 425. | Cl | Br | OCH₂CH₂ | H | Q3c | |
| 426. | Cl | Br | OCH₂CH₂ | H | Q4a | |
| 427. | Cl | Br | OCH₂CH₂ | H | Q5a | |
| 428. | Cl | Br | OCH₂CH₂ | H | Q6a | |
| 429. | Cl | Br | OCH₂CH₂ | H | Q8a | |
| 430. | Cl | Br | OCH₂CH₂ | H | Q8d | |
| 431. | Cl | Br | OCH₂CH₂ | H | Q9a | |
| 432. | Cl | Br | OCH₂CH₂ | H | Q12 | |
| 433. | Cl | Br | OCH₂CH₂ | H | Q13 | |
| 434. | Cl | Br | OCH₂CH₂ | Me | Q1a | |
| 435. | Cl | Br | OCH₂CH₂ | Me | Q3a | |
| 436. | Cl | Br | OCH₂CH₂ | Me | Q6a | |
| 437. | Cl | Br | OCH₂CH(Me) | H | Q1a | |
| 438. | Cl | Br | OCH₂CH₂CH₂ | H | Q1a | |
| 439. | Cl | I | OCH₂CH₂ | H | Q1a | |
| 440. | Cl | I | OCH₂CH₂ | Me | Q1a | |
| 441. | Cl | Me | OCH₂CH₂ | H | Q1a | |
| 442. | Cl | Me | OCH₂CH₂ | H | Q1b | |
| 443. | Cl | Me | OCH₂CH₂ | H | Q1c | |
| 444. | Cl | Me | OCH₂CH₂ | H | Q1d | |
| 445. | Cl | Me | OCH₂CH₂ | H | Q1e | |
| 446. | Cl | Me | OCH₂CH₂ | H | Q2a | |
| 447. | Cl | Me | OCH₂CH₂ | H | Q2b | |
| 448. | Cl | Me | OCH₂CH₂ | H | Q3a | |
| 449. | Cl | Me | OCH₂CH₂ | H | Q3c | |
| 450. | Cl | Me | OCH₂CH₂ | H | Q4a | |
| 451. | Cl | Me | OCH₂CH₂ | H | Q5a | |
| 452. | Cl | Me | OCH₂CH₂ | H | Q6a | |
| 453. | Cl | Me | OCH₂CH₂ | H | Q8a | |
| 454. | Cl | Me | OCH₂CH₂ | H | Q8d | |
| 455. | Cl | Me | OCH₂CH₂ | H | Q9a | |
| 456. | Cl | Me | OCH₂CH₂ | H | Q12 | |
| 457. | Cl | Me | OCH₂CH₂ | H | Q13 | |
| 458. | Cl | Me | OCH₂CH₂ | Me | Q1a | |
| 459. | Cl | Me | OCH₂CH₂ | Me | Q2a | |
| 460. | Cl | Me | OCH₂CH₂ | Me | Q2b | |
| 461. | Cl | Me | OCH₂CH₂ | Me | Q3a | |
| 462. | Cl | Me | OCH₂CH₂ | Me | Q6a | |
| 463. | Cl | Me | OCH₂CH₂ | Me | Q8d | |
| 464. | Cl | Me | OCH₂CH(Me) | H | Q1a | |
| 465. | Cl | Me | OCH₂CH₂CH₂ | H | Q1a | |
| 466. | Cl | CF₃ | OCH₂CH₂ | H | Q1a | |
| 467. | Cl | CF₃ | OCH₂CH₂ | H | Q2a | |
| 468. | Cl | CF₃ | OCH₂CH₂ | H | Q2b | |
| 469. | Cl | CF₃ | OCH₂CH₂ | H | Q3a | |
| 470. | Cl | CF₃ | OCH₂CH₂ | Me | Q1a | |
| 471. | Cl | CF₃ | OCH₂CH₂ | Me | Q3a | |
| 472. | Cl | CF₃ | OCH₂CH₂CH₂ | H | Q1a | |
| 473. | Cl | CF₃ | OCH₂CH₂CH₂ | Me | Q1a | |
| 474. | Cl | OMe | CH₂ | H | Q1a | 156-157 |
| 475. | Cl | OMe | CH₂ | H | Q3a | |
| 476. | Cl | OMe | CH₂ | Me | Q1a | |
| 477. | Cl | OSO₂Me | CH₂ | H | Q1a | |
| 478. | Cl | OSO₂Et | CH₂ | H | Q1a | |
| 479. | Cl | SMe | CH₂ | H | Q1a | |
| 480. | Cl | SMe | CH₂ | H | Q3a | |
| 481. | Cl | SMe | CH₂ | Me | Q1a | |
| 482. | Cl | SMe | OCH₂CH₂ | H | Q1a | |
| 483. | Cl | SMe | OCH₂CH₂ | H | Q2a | |
| 484. | Cl | SMe | OCH₂CH₂ | H | Q2b | |
| 485. | Cl | SMe | OCH₂CH₂ | H | Q3a | |
| 486. | Cl | SMe | OCH₂CH₂ | H | Q6a | |
| 487. | Cl | SMe | OCH₂CH₂ | H | Q8d | |
| 488. | Cl | SMe | OCH₂CH₂ | Me | Q1a | |
| 489. | Cl | SMe | OCH₂CH₂ | Me | Q3a | |
| 490. | Cl | SMe | OCH₂CH(Me) | H | Q1a | |
| 491. | Cl | SMe | OCH₂CH₂CH₂ | H | Q1a | |
| 492. | Cl | SEt | CH₂ | H | Q1a | |
| 493. | Cl | SEt | OCH₂CH₂ | H | Q1a | |
| 494. | Cl | SEt | OCH₂CH₂ | Me | Q1a | |
| 495. | Cl | SO₂Me | CH₂ | H | Q1a | 213-216 |
| 496. | Cl | SO₂Me | CH₂ | H | Q1b | |
| 497. | Cl | SO₂Me | CH₂ | H | Q1c | |
| 498. | Cl | SO₂Me | CH₂ | H | Q1d | |
| 499. | Cl | SO₂Me | CH₂ | H | Q1e | |
| 500. | Cl | SO₂Me | CH₂ | H | Q2a | |
| 501. | Cl | SO₂Me | CH₂ | H | Q2b | |
| 502. | Cl | SO₂Me | CH₂ | H | Q3a | 216-218 |
| 503. | Cl | SO₂Me | CH₂ | H | Q3b | |
| 504. | Cl | SO₂Me | CH₂ | H | Q3c | |
| 505. | Cl | SO₂Me | CH₂ | H | Q3d | |
| 506. | Cl | SO₂Me | CH₂ | H | Q3e | |
| 507. | Cl | SO₂Me | CH₂ | H | Q3f | |
| 508. | Cl | SO₂Me | CH₂ | H | Q3g | |
| 509. | Cl | SO₂Me | CH₂ | H | Q3h | |
| 510. | Cl | SO₂Me | CH₂ | H | Q3i | |
| 511. | Cl | SO₂Me | CH₂ | H | Q3j | |
| 512. | Cl | SO₂Me | CH₂ | H | Q3k | |
| 513. | Cl | SO₂Me | CH₂ | H | Q31 | |
| 514. | Cl | SO₂Me | CH₂ | H | Q3m | |
| 515. | Cl | SO₂Me | CH₂ | H | Q3n | |
| 516. | Cl | SO₂Me | CH₂ | H | Q3o | |
| 517. | Cl | SO₂Me | CH~ | H | Q3p | |
| 518. | Cl | SO₂Me | CH₂ | H | Q3q | |
| 519. | Cl | SO₂Me | CH₂ | H | Q3r | |
| 520. | Cl | SO₂Me | CH₂ | H | Q3s | |
| 521. | Cl | SO₂Me | CH₂ | H | Q3t | |
| 522. | Cl | SO₂Me | CH₂ | H | Q3u | |
| 523. | Cl | SO₂Me | CH₂ | H | Q3v | |
| 524. | Cl | SO₂Me | CH₂ | H | Q3w | |
| 525. | Cl | SO₂Me | CH₂ | H | Q3x | |
| 526. | Cl | SO₂Me | CH₂ | H | Q3y | |
| 527. | Cl | SO₂Me | CH₂ | H | Q3z | |
| 528. | Cl | SO₂Me | CH₂ | H | Q3za | |
| 529. | Cl | SO₂Me | CH₂ | H | Q3zb | |
| 530. | Cl | SO₂Me | CH₂ | H | Q3zc | |
| 531. | Cl | SO₂Me | CH₂ | H | Q3zd | |
| 532. | Cl | SO₂Me | CH₂ | H | Q4a | |
| 533. | Cl | SO₂Me | CH₂ | H | Q4b | |
| 534. | Cl | SO₂Me | CH₂ | H | Q4c | |
| 535. | Cl | SO₂Me | CH₂ | H | Q4d | |
| 536. | Cl | SO₂Me | CH₂ | H | Q5a | |
| 537. | Cl | SO₂Me | CH₂ | H | Q5b | |
| 538. | Cl | SO₂Me | CH₂ | H | Q5c | |
| 539. | Cl | SO₂Me | CH₂ | H | Q5d | |
| 540. | Cl | SO₂Me | CH₂ | H | QSe | |
| 541. | Cl | SO₂Me | CH₂ | H | Q6a | 82-85 |
| 542. | Cl | SO₂Me | CH₂ | H | Q6b | |
| 543. | Cl | SO₂Me | CH₂ | H | Q7 | |
| 544. | Cl | SO₂Me | CH₂ | H | Q8a | |
| 545. | Cl | SO₂Me | CH₂ | H | Q8b | |
| 546. | Cl | SO₂Me | CH₂ | H | Q8c | |
| 547. | Cl | SO₂Me | CH₂ | H | Q8d | |
| 548. | Cl | SO₂Me | CH₂ | H | Q9a | |
| 549. | Cl | SO₂Me | CH₂ | H | Q9b | |
| 550. | Cl | SO₂Me | CH₂ | H | Q9c | |
| 551. | Cl | SO₂Me | CH₂ | H | Q9d | |
| 552. | Cl | SO₂Me | CH₂ | H | Q10a | |
| 553. | Cl | SO₂Me | CH₂ | H | Q11a | |
| 554. | Cl | SO₂Me | CH₂ | H | Q12 | |
| 555. | Cl | SO₂Me | CH₂ | H | Q13 | |
| 556. | Cl | SO₂Me | CH₂ | H | Q14 | |
| 557. | Cl | SO₂Me | CH₂ | H | Q15 | |
| 558. | Cl | SO₂Me | CH₂ | Me | Q1a | 85-89 |
| 559. | Cl | SO₂Me | CH₂ | Me | Q1b | |
| 560. | Cl | SO₂Me | CH₂ | Me | Q1c | |
| 561. | Cl | SO₂Me | CH₂ | Me | Q1d | |
| 562. | Cl | SO₂Me | CH₂ | Me | Q1e | |
| 563. | Cl | SO₂Me | CH₂ | Me | Q2a | |
| 564. | Cl | SO₂Me | CH₂ | Me | Q2b | |
| 565. | Cl | SO₂Me | CH₂ | Me | Q3a | |
| 566. | Cl | SO₂Me | CH₂ | Me | Q3b | |
| 567. | Cl | SO₂Me | CH₂ | Me | Q3c | |
| 568. | Cl | SO₂Me | CH₂ | Me | Q3d | |
| 569. | Cl | SO₂Me | CH₂ | Me | Q3e | |
| 570. | Cl | SO₂Me | CH₂ | Me | Q3f | |
| 571. | Cl | SO₂Me | CH₂ | Me | Q3g | |
| 572. | Cl | SO₂Me | CH₂ | Me | Q3h | |
| 573. | Cl | SO₂Me | CH₂ | Me | Q3i | |
| 574. | Cl | SO₂Me | CH₂ | Me | Q3j | |
| 575. | Cl | SO₂Me | CH₂ | Me | Q3k | |
| 576. | Cl | SO₂Me | CH₂ | Me | Q31 | |
| 577. | Cl | SO₂Me | CH₂ | Me | Q3m | |
| 578. | Cl | SO₂Me | CH₂ | Me | Q3n | |
| 579. | Cl | SO₂Me | CH₂ | Me | Q3o | |
| 580. | Cl | SO₂Me | CH₂ | Me | Q3p | |
| 581. | Cl | SO₂Me | CH₂ | Me | Q3q | |
| 582. | Cl | SO₂Me | CH₂ | Me | Q3r | |
| 583. | Cl | SO₂Me | CH₂ | Me | Q3s | |
| 584. | Cl | SO₂Me | CH₂ | Me | Q3t | |
| 585. | Cl | SO₂Me | CH₂ | Me | Q3u | |
| 586. | Cl | SO₂Me | CH₂ | Me | Q3v | |
| 587. | Cl | SO₂Me | CH₂ | Me | Q3w | |
| 588. | Cl | SO₂Me | CH₂ | Me | Q3x | |
| 589. | Cl | SO₂Me | CH₂ | Me | Q3y | |
| 590. | Cl | SO₂Me | CH₂ | Me | Q3z | |
| 591. | Cl | SO₂Me | CH₂ | Me | Q3za | |
| 592. | Cl | SO₂Me | CH₂ | Me | Q3zb | |
| 593. | Cl | SO₂Me | CH₂ | Me | Q3zc | |
| 594. | Cl | SO₂Me | CH₂ | Me | Q3zd | |
| 595. | Cl | SO₂Me | CH₂ | Me | Q4a | |
| 596. | Cl | SO₂Me | CH₂ | Me | Q4b | |
| 597. | Cl | SO₂Me | CH₂ | Me | Q4c | |
| 598. | Cl | SO₂Me | CH₂ | Me | Q4d | |
| 599. | Cl | SO₂Me | CH₂ | Me | Q5a | |
| 600. | Cl | SO₂Me | CH₂ | Me | QSb | |
| 601. | Cl | SO₂Me | CH₂ | Me | QSc | |
| 602. | Cl | SO₂Me | CH₂ | Me | Q6b | |
| 603. | Cl | SO₂Me | CH₂ | Me | Q7 | |
| 604. | Cl | SO₂Me | CH₂ | Me | Q8a | |
| 605. | Cl | SO₂Me | CH₂ | Me | Q8b | |
| 606. | Cl | SO₂Me | CH₂ | Me | Q8c | |
| 607. | Cl | SO₂Me | CH₂ | Me | QSd | |
| 608. | Cl | SO₂Me | CH₂ | Me | Q9a | |
| 609. | Cl | SO₂Me | CH₂ | Me | Q9b | |
| 610. | Cl | SO₂Me | CH₂ | Me | Q9c | |
| 611. | Cl | SO₂Me | CH₂ | Me | Q9d | |
| 612. | Cl | SO₂Me | CH₂ | Me | Q10a | |
| 613. | Cl | SO₂Me | CH₂ | Me | Q11a | |
| 614. | Cl | SO₂Me | CH₂ | Me | Q12 | |
| 615. | Cl | SO₂Me | CH₂ | Me | Q13 | |
| 616. | Cl | SO₂Me | CH₂ | Et | Q1a | |
| 617. | Cl | SO₂Me | CH₂ | Et | Q1b | |
| 618. | Cl | SO₂Me | CH₂ | Et | Q1c | |
| 619. | Cl | SO₂Me | CH₂ | Et | Q1d | |
| 620. | Cl | SO₂Me | CH₂ | Et | Q1e | |
| 621. | Cl | SO₂Me | CH₂ | Et | Q2a | |
| 622. | Cl | SO₂Me | CH₂ | Et | Q2b | |
| 623. | Cl | SO₂Me | CH₂ | Et | Q3a | |
| 624. | Cl | SO₂Me | CH₂ | Et | Q3c | |
| 625. | Cl | SO₂Me | CH₂ | Et | Q4a | |
| 626. | Cl | SO₂Me | CH₂ | Et | Q5a | |
| 627. | Cl | SO₂Me | CH₂ | Et | Q6a | |
| 628. | Cl | SO₂Me | CH₂ | Et | Q8a | |
| 629. | Cl | SO₂Me | CH₂ | Et | Q8d | |
| 630. | Cl | SO₂Me | CH₂ | Et | Q9a | |
| 631. | Cl | SO₂Me | CH₂ | Et | Q12 | |
| 632. | Cl | SO₂Me | CH₂ | Et | Q13 | |
| 633. | Cl | SO₂Me | CH₂ | n-Pr | Q1a | |
| 634. | Cl | SO₂Me | CH₂ | n-Pr | Q1b | |
| 635. | Cl | SO₂Me | CH₂ | n-Pr | Q1c | |
| 636. | Cl | SO₂Me | CH₂ | n-Pr | Q1d | |
| 637. | Cl | SO₂Me | CH₂ | n-Pr | Q1e | |
| 638. | Cl | SO₂Me | CH₂ | n-Pr | Q2a | |
| 639. | Cl | SO₂Me | CH₂ | n-Pr | Q2b | |
| 640. | Cl | SO₂Me | CH₂ | n-Pr | Q3a | |
| 641. | Cl | SO₂Me | CH₂ | n-Pr | Q3c | |
| 642. | Cl | SO₂Me | CH₂ | n-Pr | Q4a | |
| 643. | Cl | SO₂Me | CH₂ | n-Pr | Q5a | |
| 644. | Cl | SO₂Me | CH₂ | n-Pr | Q6a | |
| 645. | Cl | SO₂Me | CH₂ | n-Pr | Q8a | |
| 646. | Cl | SO₂Me | CH₂ | n-Pr | Q8d | |
| 647. | Cl | SO₂Me | CH₂ | n-Pr | Q9a | |
| 648. | Cl | SO₂Me | CH₂ | n-Pr | Q12 | |
| 649. | Cl | SO₂Me | CH₂ | n-Pr | Q13 | |
| 650. | Cl | SO₂Me | CH₂ | SMe | Q1a | 92-97 |
| 651. | Cl | SO₂Me | CH₂ | SMe | Q1b | |
| 652. | Cl | SO₂Me | CH₂ | SMe | Q1c | |
| 653. | Cl | SO₂Me | CH₂ | SMe | Q1d | |
| 654. | Cl | SO₂Me | CH₂ | SMe | Q1e | |
| 655. | Cl | SO₂Me | CH₂ | SMe | Q2a | |
| 656. | Cl | SO₂Me | CH₂ | SMe | Q2b | |
| 657. | Cl | SO₂Me | CH₂ | SMe | Q3a | |
| 658. | Cl | SO₂Me | CH₂ | SMe | Q3c | |
| 659. | Cl | SO₂Me | CH₂ | SMe | Q4a | |
| 660. | Cl | SO₂Me | CH₂ | SMe | Q5a | |
| 661. | Cl | SO₂Me | CH₂ | SMe | Q6a | |
| 662. | Cl | SO₂Me | CH₂ | SMe | Q8a | |
| 663. | Cl | SO₂Me | CH₂ | SMe | Q8d | |
| 664. | Cl | SO₂Me | CH₂ | SMe | Q9a | |
| 665. | Cl | SO₂Me | CH₂ | SMe | Q12 | |
| 666. | Cl | SO₂Me | CH₂ | SMe | Q13 | |
| 667. | Cl | SO₂Me | CH₂ | n-Bu | Q1a | |
| 668. | Cl | SO₂Me | CH₂ | n-Pen | Q1a | |
| 669. | Cl | SO₂Me | CH₂ | n-Hex | Q1a | |
| 670. | Cl | SO₂Me | CH₂ | cyclo-Pr | Q1a | |
| 671. | Cl | SO₂Me | CH₂ | cyclo-Bu | Q1a | |
| 672. | Cl | SO₂Me | CH₂ | cyclo-Pen | Q1a | |
| 673. | Cl | SO₂Me | CH₂ | cyclo-Hex | Q1a | |
| 674. | Cl | SO₂Me | CH₂ | CH₂CH₂ | Q1a | |
| 675. | Cl | SO₂Me | CH₂ | CH₂CH∇CH₂ | Q1a | |
| 676. | Cl | SO₂Me | CH₂ | C≡CH | Q1a | |
| 677. | Cl | SO₂Me | CH₂ | CF₃ | Q1a | |
| 678. | Cl | SO₂Me | CH₂ | CH₂CH₂Cl | Q1a | |
| 679. | Cl | SO₂Me | CH₂ | CH₂CH₂CH₂ | Q1a | |
| | | | | Br | | |
| 680. | Cl | SO₂Me | CH₂ | SEt | Q1a | |
| 681. | Cl | SO₂Me | CH₂ | S-n-Pr | Q1a | |
| 682. | Cl | SO₂Me | CH₂ | Ph | Q1a | |
| 683. | Cl | SO₂Me | CH₂ | 2-Cl-Ph | Q1a | |
| 684. | Cl | SO₂Me | CH₂ | 3-Cl-Ph | Q1a | |
| 685. | Cl | SO₂Me | CH₂ | 2-Me-Ph | Q1a | |
| 686. | Cl | SO₂Me | CH₂ | 4-Me-Ph | Q1a | |
| 687. | Cl | SO₂Me | CH₂ | 3-Et-Ph | Q1a | |
| 688. | Cl | SO₂Me | CH₂ | 4-n-Pr-Ph | Q1a | |
| 689. | Cl | SO₂Me | CH₂ | 3-CF₃-Ph | Q1a | |
| 690. | Cl | SO₂Me | CH₂ | 3-CH₂CF₃- | Q1a | |
| | | | | Ph | | |
| 691. | Cl | SO₂Me | CH₂ | 3-NO₂-Ph | Q1a | |
| 692. | Cl | SO₂Me | CH₂CH₂ | H | Q1a | |
| 693. | Cl | SO₂Me | CH₂CH₂ | H | Q1b | |
| 694. | Cl | SO₂Me | CH₂CH₂ | H | Q1c | |
| 695. | Cl | SO₂Me | CH₂CH₂ | H | Q1d | |
| 696. | Cl | SO₂Me | CH₂CH₂ | H | Q1e | |
| 697. | Cl | SO₂Me | CH₂CH₂ | H | Q2a | |
| 698. | Cl | SO₂Me | CH₂CH₂ | H | Q2b | |
| 699. | Cl | SO₂Me | CH₂CH₂ | H | Q3a | |
| 700. | Cl | SO₂Me | CH₂CH₂ | H | Q3c | |
| 701. | Cl | SO₂Me | CH₂CH₂ | H | Q4a | |
| 702. | Cl | SO₂Me | CH₂CH₂ | H | Q5a | |
| 703. | Cl | SO₂Me | CH₂CH₂ | H | Q6a | |
| 704. | Cl | SO₂Me | CH₂CH₂ | H | Q8a | |
| 705. | Cl | SO₂Me | CH₂CH₂ | H | Q8d | |
| 706. | Cl | SO₂Me | CH₂CH₂ | H | Q9a | |
| 707. | Cl | SO₂Me | CH₂CH₂ | H | Q12 | |
| 708. | Cl | SO₂Me | CH₂CH₂ | H | Q13 | |
| 709. | Cl | SO₂Me | CH₂CH₂ | Me | Q1a | |
| 710. | Cl | SO₂Me | CH₂CH₂ | Me | Q2a | |
| 711. | Cl | SO₂Me | CH₂CH₂ | Me | Q2b | |
| 712. | Cl | SO₂Me | CH₂CH₂ | Me | Q3a | |
| 713. | Cl | SO₂Me | CH₂CH₂ | Me | Q6a | |
| 714. | Cl | SO₂Me | CH₂CH₂ | Me | Q8d | |
| 715. | Cl | SO₂Me | OCH₂CH₂ | H | Q1a | 78-81 |
| 716. | Cl | SO₂Me | OCH₂CH₂ | H | Q1b | |
| 717. | Cl | SO₂Me | OCH₂CH₂ | H | Q1c | |
| 718. | Cl | SO₂Me | OCH₂CH₂ | H | Q1d | |
| 719. | Cl | SO₂Me | OCH₂CH₂ | H | Q1e | |
| 720. | Cl | SO₂Me | OCH₂CH₂ | H | Q2a | |
| 721. | Cl | SO₂Me | OCH₂CH₂ | H | Q2b | |
| 722. | Cl | SO₂Me | OCH₂CH₂ | H | Q3a | |
| 723. | Cl | SO₂Me | OCH₂CH₂ | H | Q3b | |
| 724. | Cl | SO₂Me | OCH₂CH₂ | H | Q3c | |
| 725. | Cl | SO₂Me | OCH₂CH₂ | H | Q3d | |
| 726. | Cl | SO₂Me | OCH₂CH₂ | H | Q3e | |
| 727. | Cl | SO₂Me | OCH₂CH₂ | H | Q3f | |
| 728. | Cl | SO₂Me | OCH₂CH₂ | H | Q3g | |
| 729. | Cl | SO₂Me | OCH₂CH₂ | H | Q3h | |
| 730. | Cl | SO₂Me | OCH₂CH₂ | H | Q3i | |
| 731. | Cl | SO₂Me | OCH₂CH₂ | H | Q3j | |
| 732. | Cl | SO₂Me | OCH₂CH₂ | H | Q3k | |
| 733. | Cl | SO₂Me | OCH₂CH₂ | H | Q31 | |
| 734. | Cl | SO₂Me | OCH₂CH₂ | H | Q3m | |
| 735. | Cl | SO₂Me | OCH₂CH₂ | H | Q3n | |
| 736. | Cl | SO₂Me | OCH₂CH₂ | H | Q3o | |
| 737. | Cl | SO₂Me | OCH₂CH₂ | H | Q3p | |
| 738. | Cl | SO₂Me | OCH₂CH₂ | H | Q3q | |
| 739. | Cl | SO₂Me | OCH₂CH₂ | H | Q3r | |
| 740. | Cl | SO₂Me | OCH₂CH₂ | H | Q3s | |
| 741. | Cl | SO₂Me | OCH₂CH₂ | H | Q3t | |
| 742. | Cl | SO₂Me | OCH₂CH₂ | H | Q3u | |
| 743. | Cl | SO₂Me | OCH₂CH₂ | H | Q3v | |
| 744. | Cl | SO₂Me | OCH₂CH₂ | H | Q3w | |
| 745. | Cl | SO₂Me | OCH₂CH₂ | H | Q3x | |
| 746. | Cl | SO₂Me | OCH₂CH₂ | H | Q3y | |
| 747. | Cl | SO₂Me | OCH₂CH₂ | H | Q3z | |
| 748. | Cl | SO₂Me | OCH₂CH₂ | H | Q3za | |
| 749. | Cl | SO₂Me | OCH₂CH₂ | H | Q3zb | |
| 750. | Cl | SO₂Me | OCH₂CH₂ | H | Q3zc | |
| 751. | Cl | SO₂Me | OCH₂CH₂ | H | Q3zd | |
| 752. | Cl | SO₂Me | OCH₂CH₂ | H | Q4a | |
| 753. | Cl | SO₂Me | OCH₂CH₂ | H | Q4b | |
| 754. | Cl | SO₂Me | OCH₂CH₂ | H | Q4c | |
| 755. | Cl | SO₂Me | OCH₂CH₂ | H | Q4d | |
| 756. | Cl | SO₂Me | OCH₂CH₂ | H | Q5a | |
| 757. | Cl | SO₂Me | OCH₂CH₂ | H | Q5b | |
| 758. | Cl | SO₂Me | OCH₂CH₂ | H | Q5c | |
| 759. | Cl | SO₂Me | OCH₂CH₂ | H | Q5d | |
| 760. | Cl | SO₂Me | OCH₂CH₂ | H | Q5e | |
| 761. | Cl | SO₂Me | OCH₂CH₂ | H | Q6a | |
| 762. | Cl | SO₂Me | OCH₂CH₂ | H | Q6b | |
| 763. | Cl | SO₂Me | OCH₂CH₂ | H | Q7 | |
| 764. | Cl | SO₂Me | OCH₂CH₂ | H | Q8a | |
| 765. | Cl | SO₂Me | OCH₂CH₂ | H | Q8b | |
| 766. | Cl | SO₂Me | OCH₂CH₂ | H | Q8c | |
| 767. | Cl | SO₂Me | OCH₂CH₂ | H | Q8d | |
| 768. | Cl | SO₂Me | OCH₂CH₂ | H | Q9a | |
| 769. | Cl | SO₂Me | OCH₂CH₂ | H | Q9b | |
| 770. | Cl | SO₂Me | OCH₂CH₂ | H | Q9c | |
| 771. | Cl | SO₂Me | OCH₂CH₂ | H | Q9d | |
| 772. | Cl | SO₂Me | OCH₂CH₂ | H | Q10a | |
| 773. | Cl | SO₂Me | OCH₂CH₂ | H | Q11a | |
| 774. | Cl | SO₂Me | OCH₂CH₂ | H | Q12 | |
| 775. | Cl | SO₂Me | OCH₂CH₂ | H | Q13 | |
| 776. | Cl | SO₂Me | OCH₂CH₂ | Me | Q1a | 75-80 |
| 777. | Cl | SO₂Me | OCH₂CH₂ | Me | Q1b | |
| 778. | Cl | SO₂Me | OCH₂CH₂ | Me | Q1c | |
| 779. | Cl | SO₂Me | OCH₂CH₂ | Me | Q1d | |
| 780. | Cl | SO₂Me | OCH₂CH₂ | Me | Q1e | |
| 781. | Cl | SO₂Me | OCH₂CH₂ | Me | Q2a | |
| 782. | Cl | SO₂Me | OCH₂CH₂ | Me | Q2b | |
| 783. | Cl | SO₂Me | OCH₂CH₂ | Me | Q3a | |
| 784. | Cl | SO₂Me | OCH₂CH₂ | Me | Q3b | |
| 785. | Cl | SO₂Me | OCH₂CH₂ | Me | Q3c | |
| 786. | Cl | SO₂Me | OCH₂CH₂ | Me | Q3d | |
| 787. | Cl | SO₂Me | OCH₂CH₂ | Me | Q3e | |
| 788. | Cl | SO₂Me | OCH₂CH₂ | Me | Q3f | |
| 789. | Cl | SO₂Me | OCH₂CH₂ | Me | Q3g | |
| 790. | Cl | SO₂Me | OCH₂CH₂ | Me | Q3h | |
| 791. | Cl | SO₂Me | OCH₂CH₂ | Me | Q31 | |
| 792. | Cl | SO₂Me | OCH₂CH₂ | Me | Q3j | |
| 793. | Cl | SO₂Me | OCH₂CH₂ | Me | Q3k | |
| 794. | Cl | SO₂Me | OCH₂CH₂ | Me | Q31 | |
| 795. | Cl | SO₂Me | OCH₂CH₂ | Me | Q3m | |
| 796. | Cl | SO₂Me | OCH₂CH₂ | Me | Q3n | |
| 797. | Cl | SO₂Me | OCH₂CH₂ | Me | Q3o | |
| 798. | Cl | SO₂Me | OCH₂CH₂ | Me | Q3p | |
| 799. | Cl | SO₂Me | OCH₂CH₂ | Me | Q3q | |
| 800. | Cl | SO₂Me | OCH₂CH₂ | Me | Q3r | |
| 801. | Cl | SO₂Me | OCH₂CH₂ | Me | Q3s | |
| 802. | Cl | SO₂Me | OCH₂CH₂ | Me | Q3t | |
| 803. | Cl | SO₂Me | OCH₂CH₂ | Me | Q3u | |
| 804. | Cl | SO₂Me | OCH₂CH₂ | Me | Q3v | |
| 805. | Cl | SO₂Me | OCH₂CH₂ | Me | Q3w | |
| 806. | Cl | SO₂Me | OCH₂CH₂ | Me | Q3x | |
| 807. | Cl | SO₂Me | OCH₂CH₂ | Me | Q3y | |
| 808. | Cl | SO₂Me | OCH₂CH₂ | Me | Q3z | |
| 809. | Cl | SO₂Me | OCH₂CH₂ | Me | Q3za | |
| 810. | Cl | SO₂Me | OCH₂CH₂ | Me | Q3zb | |
| 811. | Cl | SO₂Me | OCH₂CH₂ | Me | Q3zc | |
| 812. | Cl | SO₂Me | OCH₂CH₂ | Me | Q3zd | |
| 813. | Cl | SO₂Me | OCH₂CH₂ | Me | Q4a | |
| 814. | Cl | SO₂Me | OCH₂CH₂ | Me | Q4b | |
| 815. | Cl | SO₂Me | OCH₂CH₂ | Me | Q4c | |
| 816. | Cl | SO₂Me | OCH₂CH₂ | Me | Q4d | |
| 817. | Cl | SO₂Me | OCH₂CH₂ | Me | Q5a | |
| 818. | Cl | SO₂Me | OCH₂CH₂ | Me | Q5b | |
| 819. | Cl | SO₂Me | OCH₂CH₂ | Me | Q5c | |
| 820. | Cl | SO₂Me | OCH₂CH₂ | Me | Q6a | |
| 821. | Cl | SO₂Me | OCH₂CH₂ | Me | Q6b | |
| 822. | Cl | SO₂Me | OCH₂CH₂ | Me | Q7 | |
| 823. | Cl | SO₂Me | OCH₂CH₂ | Me | Q8a | |
| 824. | Cl | SO₂Me | OCH₂CH₂ | Me | Q8b | |
| 825. | Cl | SO₂Me | OCH₂CH₂ | Me | Q8c | |
| 826. | Cl | SO₂Me | OCH₂CH₂ | Me | Q8d | |
| 827. | Cl | SO₂Me | OCH₂CH₂ | Me | Q9a | |
| 828. | Cl | SO₂Me | OCH₂CH₂ | Me | Q9b | |
| 829. | Cl | SO₂Me | OCH₂CH₂ | Me | Q9c | |
| 830. | Cl | SO₂Me | OCH₂CH₂ | Me | Q9d | |
| 831. | Cl | SO₂Me | OCH₂CH₂ | Me | Q10a | |
| 832. | Cl | SO₂Me | OCH₂CH₂ | Me | Q11a | |
| 833. | Cl | SO₂Me | OCH₂CH₂ | Me | Q12 | |
| 834. | Cl | SO₂Me | OCH₂CH₂ | Et | Q1a | |
| 835. | Cl | SO₂Me | OCH₂CH₂ | Et | Q1b | |
| 836. | Cl | SO₂Me | OCH₂CH₂ | Et | Q1c | |
| 837. | Cl | SO₂Me | OCH₂CH₂ | Et | Q1d | |
| 838. | Cl | SO₂Me | OCH₂CH₂ | Et | Q1e | |
| 839. | Cl | SO₂Me | OCH₂CH₂ | Et | Q2a | |
| 840. | Cl | SO₂Me | OCH₂CH₂ | Et | Q2b | |
| 841. | Cl | SO₂Me | OCH₂CH₂ | Et | Q3a | |
| 842. | Cl | SO₂Me | OCH₂CH₂ | Et | Q3c | |
| 843. | Cl | SO₂Me | OCH₂CH₂ | Et | Q4a | |
| 844. | Cl | SO₂Me | OCH₂CH₂ | Et | Q5a | |
| 845. | Cl | SO₂Me | OCH₂CH₂ | Et | Q6a | |
| 846. | Cl | SO₂Me | OCH₂CH₂ | Et | Q8a | |
| 847. | Cl | SO₂Me | OCH₂CH₂ | Et | Q8d | |
| 848. | Cl | SO₂Me | OCH₂CH₂ | Et | Q9a | |
| 849. | Cl | SO₂Me | OCH₂CH₂ | Et | Q12 | |
| 850. | Cl | SO₂Me | OCH₂CH₂ | Et | Q13 | |
| 851. | Cl | SO₂Me | OCH₂CH₂ | n-Pr | Q1a | |
| 852. | Cl | SO₂Me | OCH₂CH₂ | n-Pr | Q3a | |
| 853. | Cl | SO₂Me | OCH₂CH₂ | n-Pr | Q6a | |
| 854. | Cl | SO₂Me | OCH₂CH₂ | SMe | Q1a | |
| 855. | Cl | SO₂Me | OCH₂CH₂ | SMe | Q3a | |
| 856. | Cl | SO₂Me | OCH₂CH₂ | SMe | Q6a | |
| 857. | Cl | SO₂Me | OCH₂CH₂ | n-Bu | Q1a | |
| 858. | Cl | SO₂Me | OCH₂CH₂ | n-Pen | Q1a | |
| 859. | Cl | SO₂Me | OCH₂CH₂ | n-Hex | Q1a | |
| 860. | Cl | SO₂Me | OCH₂CH₂ | cyclo-Pr | Q1a | |
| 861. | Cl | SO₂Me | OCH₂CH₂ | cyclo-Bu | Q1a | |
| 862. | Cl | SO₂Me | OCH₂CH₂ | cyclo-Pen | Q1a | |
| 863. | Cl | SO₂Me | OCH₂CH₂ | cyclo-Hex | Q1a | |
| 864. | Cl | SO₂Me | OCH₂CH₂ | SEt | Q1a | |
| 865. | Cl | SO₂Me | OCH₂CH₂ | S-n-Pr | Q1a | |
| 866. | Cl | SO₂Me | OCH₂CH₂ | Ph | Q1a | |
| 867. | Cl | SO₂Me | OCH₂CH₂ | 2-Cl-Ph | Q1a | |
| 868. | Cl | SO₂Me | OCH₂CH₂ | 3-Cl-Ph | Q1a | |
| 869. | Cl | SO₂Me | OCH₂CH₂ | 2-Me-Ph | Q1a | |
| 870. | Cl | SO₂Me | OCH₂CH₂ | 4-Me-Ph | Q1a | |
| 871. | Cl | SO₂Me | OCH₂CH₂ | 3-Et-Ph | Q1a | |
| 872. | Cl | SO₂Me | OCH₂CH₂ | 4-n-Pr-Ph | Q1a | |
| 873. | Cl | SO₂Me | OCH₂CH₂ | 3-CF₃-Ph | Q1a | |
| 874. | Cl | SO₂Me | OCH₂CH₂ | 3-CH₂CF₃- | Q1a | |
| | | | | Ph | | |
| 875. | Cl | SO₂Me | OCH₂CH₂ | 3-NO₂-Ph | Q1a | |
| 876. | Cl | SO₂Me | OCH₂CH(Me) | H | Q1a | |
| 877. | Cl | SO₂Me | OCH₂CH(Me) | H | Q1b | |
| 878. | Cl | SO₂Me | OCH₂CH(Me) | H | Q1c | |
| 879. | Cl | SO₂Me | OCH₂CH(Me) | H | Q1d | |
| 880. | Cl | SO₂Me | OCH₂CH(Me) | H | Q1e | |
| 881. | Cl | SO₂Me | OCH₂CH(Me) | H | Q2a | |
| 882. | Cl | SO₂Me | OCH₂CH(Me) | H | Q2b | |
| 883. | Cl | SO₂Me | OCH₂CH(Me) | H | Q3a | |
| 884. | Cl | SO₂Me | OCH₂CH(Me) | H | Q3c | |
| 885. | Cl | SO₂Me | OCH₂CH(Me) | H | Q4a | |
| 886. | Cl | SO₂Me | OCH₂CH(Me) | H | Q5a | |
| 887. | Cl | SO₂Me | OCH₂CH(Mc) | H | Q6a | |
| 888. | Cl | SO₂Me | OCH₂CH(Me) | H | Q8a | |
| 889. | Cl | SO₂Me | OCH₂CH(Me) | H | Q8d | |
| 890. | Cl | SO₂Me | OCH₂CH(Me) | H | Q9a | |
| 891. | Cl | SO₂Me | OCH₂CH(Me) | H | Q12 | |
| 892. | Cl | SO₂Me | OCH₂CH(Me) | H | Q13 | |
| 893. | Cl | SO₂Me | OCH₂CH(Me) | Me | Q1a | |
| 894. | Cl | SO₂Me | OCH₂CH(Me) | Me | Q3a | |
| 895. | Cl | SO₂Me | OCH₂CH(Me) | Me | Q6a | |
| 896. | Cl | SO₂Me | OCH₂CH₂CH₂ | H | Q1a | |
| 897. | Cl | SO₂Me | OCH₂CH₂CH₂ | H | Q1b | |
| 898. | Cl | SO₂Me | OCH₂CH₂CH₂ | H | Q1c | |
| 899. | Cl | SO₂Me | OCH₂CH₂CH₂ | H | Q1d | |
| 900. | Cl | SO₂Me | OCH₂CH₂CH₂ | H | Q1e | |
| 901. | Cl | SO₂Me | OCH₂CH₂CH₂ | H | Q2a | |
| 902. | Cl | SO₂Me | OCH₂CH₂CH₂ | H | Q2b | |
| 903. | Cl | SO₂Me | OCH₂CH₂CH₂ | H | Q3a | |
| 904. | Cl | SO₂Me | OCH₂CH₂CH₂ | H | Q3c | |
| 905. | Cl | SO₂Me | OCH₂CH₂CH₂ | H | Q4a | |
| 906. | Cl | SO₂Me | OCH₂CH₂CH₂ | H | Q5a | |
| 907. | Cl | SO₂Me | OCH₂CH₂CH₂ | H | Q6a | |
| 908. | Cl | SO₂Me | OCH₂CH₂CH₂ | H | Q8a | |
| 909. | Cl | SO₂Me | OCH₂CH₂CH₂ | H | Q8d | |
| 910. | Cl | SO₂Me | OCH₂CH₂CH₂ | H | Q9a | |
| 911. | Cl | SO₂Me | OCH₂CH₂CH₂ | H | Q12 | |
| 912. | Cl | SO₂Me | OCH₂CH₂CH₂ | H | Q13 | |
| 913. | Cl | SO₂Me | OCH₂CH₂CH₂ | Me | Q1a | |
| 914. | Cl | SO₂Me | OCH₂CH₂CH₂ | Me | Q2a | |
| 915. | Cl | SO₂Me | OCH₂CH₂CH₂ | Me | Q2b | |
| 916. | Cl | SO₂Me | OCH₂CH₂CH₂ | Me | Q3a | |
| 917. | Cl | SO₂Me | OCH₂CH₂CH₂ | Me | Q6a | |
| 918. | Cl | SO₂Me | OCH₂CH₂CH₂ | Me | Q13 | |
| 919. | Cl | SO₂Me | CH(Me) | H | Q1a | |
| 920. | Cl | SO₂Me | CH(Me)CH₂ | H | Q1a | |
| 921. | Cl | SO₂Me | CH₂CH₂CH₂ | H | Q1a | |
| 922. | Cl | SO₂Me | OCH₂ | H | Q1a | |
| 923. | Cl | SO₂Me | OCH₂ | Me | Q1a | |
| 924. | Cl | SO₂Me | OCH(Me)CH₂ | H | Q1a | |
| 925. | Cl | SO₂Me | OCH(Me)CH₂ | Me | Q1a | |
| 926. | Cl | SO₂Et | CH₂ | H | Q1a | |
| 927. | Cl | SO₂Et | CH₂ | H | Q1b | |
| 928. | Cl | SO₂Et | CH₂ | H | Q1c | |
| 929. | Cl | SO₂Et | CH₂ | H | Q1d | |
| 930. | Cl | SO₂Et | CH₂ | H | Q1e | |
| 931. | Cl | SO₂Et | CH₂ | H | Q2a | |
| 932. | Cl | SO₂Et | CH₂ | H | Q2b | |
| 933. | Cl | SO₂Et | CH₂ | H | Q3a | |
| 934. | Cl | SO₂Et | CH₂ | H | Q3c | |
| 935. | Cl | SO₂Et | CH₂ | H | Q4a | |
| 936. | Cl | SO₂Et | CH₂ | H | Q5a | |
| 937. | Cl | SO₂Et | CH₂ | H | Q6a | |
| 938. | Cl | SO₂Et | CH₂ | H | Q8a | |
| 939. | Cl | SO₂Et | CH₂ | H | Q8d | |
| 940. | Cl | SO₂Et | CH₂ | H | Q9a | |
| 941. | Cl | SO₂Et | CH₂ | H | Q12 | |
| 942. | Cl | SO₂Et | CH₂ | H | Q13 | |
| 943. | Cl | SO₂Et | CH₂ | Me | Q1a | |
| 944. | Cl | SO₂Et | CH₂ | Me | Q2a | |
| 945. | Cl | SO₂Et | CH₂ | Me | Q2b | |
| 946. | Cl | SO₂Et | CH₂ | Me | Q3a | |
| 947. | Cl | SO₂Et | CH₂ | Me | Q6a | |
| 948. | Cl | SO₂Et | CH₂ | Me | Q8d | |
| 949. | Cl | SO₂Et | CH₂CH₂ | H | Q1a | |
| 950. | Cl | SO₂Et | CH₂CH₂ | Me | Q1a | |
| 951. | Cl | SO₂Et | OCH₂CH₂ | H | Q1a | |
| 952. | Cl | SO₂Et | OCH₂CH₂ | H | Q1b | |
| 953. | Cl | SO₂Et | OCH₂CH₂ | H | Q1c | |
| 954. | Cl | SO₂Et | OCH₂CH₂ | H | Q1d | |
| 955. | Cl | SO₂Et | OCH₂CH₂ | H | Q1e | |
| 956. | Cl | SO₂Et | OCH₂CH₂ | H | Q2a | |
| 957. | Cl | SO₂Et | OCH₂CH₂ | H | Q2b | |
| 958. | Cl | SO₂Et | OCH₂CH₂ | H | Q3a | |
| 959. | Cl | SO₂Et | OCH₂CH₂ | H | Q3c | |
| 960. | Cl | SO₂Et | OCH₂CH₂ | H | Q4a | |
| 961. | Cl | SO₂Et | OCH₂CH₂ | H | Q5a | |
| 962. | Cl | SO₂Et | OCH₂CH₂ | H | Q6a | |
| 963. | Cl | SO₂Et | OCH₂CH₂ | H | Q8a | |
| 964. | Cl | SO₂Et | OCH₂CH₂ | H | Q8d | |
| 965. | Cl | SO₂Et | OCH₂CH₂ | H | Q9a | |
| 966. | Cl | SO₂Et | OCH₂CH₂ | H | Q12 | |
| 967. | Cl | SO₂Et | OCH₂CH₂ | H | Q13 | |
| 968. | Cl | SO₂Et | OCH₂CH₂ | Me | Q1a | |
| 969. | Cl | SO₂Et | OCH₂CH₂ | Me | Q2a | |
| 970. | Cl | SO₂Et | OCH₂CH₂ | Me | Q2b | |
| 971. | Cl | SO₂Et | OCH₂CH₂ | Me | Q3a | |
| 972. | Cl | SO₂Et | OCH₂CH₂ | Me | Q6a | |
| 973. | Cl | SO₂Et | OCH₂CH₂ | Me | Q8d | |
| 974. | Cl | SO₂Et | OCH₂CH(Me) | H | Q1a | |
| 975. | Cl | SO₂Et | OCH₂CH₂CH₂ | H | Q1a | |
| 976. | Cl | SO₂Pr-n | CH₂ | H | Q1a | |
| 977. | Cl | SO₂Pr-n | OCH₂CH₂ | H | Q1a | |
| 978. | Cl | SO₂Pr-n | OCH₂CH₂ | Me | Q1a | |
| 979. | Cl | NO₂ | CH₂ | H | Q1a | |
| 980. | Cl | NO₂ | CH₂ | H | Q3a | |
| 981. | Cl | NO₂ | CH₂ | Me | Q1a | |
| 982. | Cl | NO₂ | OCH₂CH₂ | H | Q1a | |
| 983. | Cl | NO₂ | OCH₂CH₂ | H | Q1b | |
| 984. | Cl | NO₂ | OCH₂CH₂ | H | Q1c | |
| 985. | Cl | NO₂ | OCH₂CH₂ | H | Q1d | |
| 986. | Cl | NO₂ | OCH₂CH₂ | H | Q1e | |
| 987. | Cl | NO₂ | OCH₂CH₂ | H | Q2a | |
| 988. | Cl | NO₂ | OCH₂CH₂ | H | Q2b | |
| 989. | Cl | NO₂ | OCH₂CH₂ | H | Q3a | |
| 990. | Cl | NO₂ | OCH₂CH₂ | H | Q3c | |
| 991. | Cl | NO₂ | OCH₂CH₂ | H | Q4a | |
| 992. | Cl | NO₂ | OCH₂CH₂ | H | Q5a | |
| 993. | Cl | NO₂ | OCH₂CH₂ | H | Q6a | |
| 994. | Cl | NO₂ | OCH₂CH₂ | H | Q8a | |
| 995. | Cl | NO₂ | OCH₂CH₂ | H | Q8d | |
| 996. | Cl | NO₂ | OCH₂CH₂ | H | Q9a | |
| 997. | Cl | NO₂ | OCH₂CH₂ | H | Q12 | |
| 998. | Cl | NO₂ | OCH₂CH₂ | H | Q13 | |
| 999. | Cl | NO₂ | OCH₂CH₂ | Me | Q1a | |
| 1000. | Cl | NO₂ | OCH₂CH₂ | Me | Q2a | |
| 1001. | Cl | NO₂ | OCH₂CH₂ | Me | Q2b | |
| 1002. | Cl | NO₂ | OCH₂CH₂ | Me | Q3a | |
| 1003. | Cl | NO₂ | OCH₂CH₂ | Me | Q6a | |
| 1004. | Cl | NO₂ | OCH₂CH₂ | Me | Q8d | |
| 1005. | Cl | NO₂ | OCH₂CH(Me) | H | Q1a | |
| 1006. | Cl | NO₂ | OCH₂CH₂CH₂ | H | Q1a | |
| 1007. | Br | F | OCH₂CH₂ | H | Q1a | |
| 1008. | Br | F | OCH₂CH₂ | H | Q1b | |
| 1009. | Br | F | OCH₂CH₂ | H | Q1c | |
| 1010. | Br | F | OCH₂CH₂ | H | Q1d | |
| 1011. | Br | F | OCH₂CH₂ | H | Q1e | |
| 1012. | Br | F | OCH₂CH₂ | H | Q2a | |
| 1013. | Br | F | OCH₂CH₂ | H | Q2b | |
| 1014. | Br | F | OCH₂CH₂ | H | Q3a | |
| 1015. | Br | F | OCH₂CH₂ | H | Q3c | |
| 1016. | Br | F | OCH₂CH₂ | H | Q4a | |
| 1017. | Br | F | OCH₂CH₂ | H | Q5a | |
| 1018. | Br | F | OCH₂CH₂ | H | Q6a | |
| 1019. | Br | F | OCH₂CH₂ | H | Q8a | |
| 1020. | Br | F | OCH₂CH₂ | H | Q8d | |
| 1021. | Br | F | OCH₂CH₂ | H | Q9a | |
| 1022. | Br | F | OCH₂CH₂ | H | Q12 | |
| 1023. | Br | F | OCH₂CH₂ | H | Q13 | |
| 1024. | Br | F | OCH₂CH₂ | Me | Q1a | |
| 1025. | Br | F | OCH₂CH₂ | Me | Q2a | |
| 1026. | Br | F | OCH₂CH₂ | Me | Q2b | |
| 1027. | Br | F | OCH₂CH₂ | Me | Q3a | |
| 1028. | Br | F | OCH₂CH₂ | Me | Q6a | |
| 1029. | Br | F | OCH₂CH₂ | Me | Q8d | |
| 1030. | Br | F | OCH₂CH(Me) | H | Q1a | |
| 1031. | Br | F | OCH₂CH₂CH₂ | H | Q1a | |
| 1032. | Br | F | OCH₂CH₂CH₂ | H | Q3a | |
| 1033. | Br | Cl | OCH₂CH₂ | H | Q1a | |
| 1034. | Br | Cl | OCH₂CH₂ | H | Q1b | |
| 1035. | Br | Cl | OCH₂CH₂ | H | Q1c | |
| 1036. | Br | Cl | OCH₂CH₂ | H | Q1d | |
| 1037. | Br | Cl | OCH₂CH₂ | H | Q1e | |
| 1038. | Br | Cl | OCH₂CH₂ | H | Q2a | |
| 1039. | Br | Cl | OCH₂CH₂ | H | Q2b | |
| 1040. | Br | Cl | OCH₂CH₂ | H | Q3a | |
| 1041. | Br | Cl | OCH₂CH₂ | H | Q3c | |
| 1042. | Br | Cl | OCH₂CH₂ | H | Q4a | |
| 1043. | Br | Cl | OCH₂CH₂ | H | Q5a | |
| 1044. | Br | Cl | OCH₂CH₂ | H | Q6a | |
| 1045. | Br | Cl | OCH₂CH₂ | H | Q8a | |
| 1046. | Br | Cl | OCH₂CH₂ | H | Q8d | |
| 1047. | Br | Cl | OCH₂CH₂ | H | Q9a | |
| 1048. | Br | Cl | OCH₂CH₂ | H | Q12 | |
| 1049. | Br | Cl | OCH₂CH₂ | H | Q13 | |
| 1050. | Br | Cl | OCH₂CH₂ | Me | Q1a | |
| 1051. | Br | Cl | OCH₂CH₂ | Me | Q2a | |
| 1052. | Br | Cl | OCH₂CH₂ | Me | Q2b | |
| 1053. | Br | Cl | OCH₂CH₂ | Me | Q3a | |
| 1054. | Br | Cl | OCH₂CH₂ | Me | Q6a | |
| 1055. | Br | Cl | OCH₂CH₂ | Me | Q8d | |
| 1056. | Br | Cl | OCH₂CH(Me) | H | Q1a | |
| 1057. | Br | Cl | OCH₂CH₂CH₂ | H | Q1a | |
| 1058. | Br | Cl | OCH₂CH₂CH₂ | H | Q3a | |
| 1059. | Br | Br | CH₂ | H | Q1a | 70-74 |
| 1060. | Br | Br | CH₂ | H | Q1b | |
| 1061. | Br | Br | CH₂ | H | Q1c | |
| 1062. | Br | Br | CH₂ | H | Q1d | |
| 1063. | Br | Br | CH₂ | H | Q1e | |
| 1064. | Br | Br | CH₂ | H | Q2a | |
| 1065. | Br | Br | CH₂ | H | Q2b | |
| 1066. | Br | Br | CH₂ | H | Q3a | 163-168 |
| 1067. | Br | Br | CH₂ | H | Q3b | |
| 1068. | Br | Br | CH₂ | H | Q3c | |
| 1069. | Br | Br | CH₂ | H | Q3d | |
| 1070. | Br | Br | CH₂ | H | Q3e | |
| 1071. | Br | Br | CH₂ | H | Q3f | |
| 1072. | Br | Br | CH₂ | H | Q3g | |
| 1073. | Br | Br | CH₂ | H | Q3h | |
| 1074. | Br | Br | CH₂ | H | Q3i | |
| 1075. | Br | Br | CH₂ | H | Q3j | |
| 1076. | Br | Br | CH₂ | H | Q3k | |
| 1077. | Br | Br | CH₂ | H | Q31 | |
| 1078. | Br | Br | CH₂ | H | Q3m | |
| 1079. | Br | Br | CH₂ | H | Q3n | |
| 1080. | Br | Br | CH₂ | H | Q3o | |
| 1081. | Br | Br | CH₂ | H | Q3p | |
| 1082. | Br | Br | CH₂ | H | Q3q | |
| 1083. | Br | Br | CH₂ | H | Q3r | |
| 1084. | Br | Br | CH₂ | H | Q3s | |
| 1085. | Br | Br | CH₂ | H | Q3t | |
| 1086. | Br | Br | CH₂ | H | Q3u | |
| 1087. | Br | Br | CH₂ | H | Q3v | |
| 1088. | Br | Br | CH₂ | H | Q3w | |
| 1089. | Br | Br | CH₂ | H | Q3x | |
| 1090. | Br | Br | CH₂ | H | Q3y | |
| 1091. | Br | Br | CH₂ | H | Q3z | |
| 1092. | Br | Br | CH₂ | H | Q3za | |
| 1093. | Br | Br | CH₂ | H | Q3zb | |
| 1094. | Br | Br | CH₂ | H | Q3zc | |
| 1095. | Br | Br | CH₂ | H | Q3zd | |
| 1096. | Br | Br | CH₂ | H | Q4a | |
| 1097. | Br | Br | CH₂ | H | Q4b | |
| 1098. | Br | Br | CH₂ | H | Q4c | |
| 1099. | Br | Br | CH₂ | H | Q4d | |
| 1100. | Br | Br | CH₂ | H | Q5a | |
| 1101. | Br | Br | CH₂ | H | Q5b | |
| 1102. | Br | Br | CH₂ | H | Q5c | |
| 1103. | Br | Br | CH₂ | H | Q6a | |
| 1104. | Br | Br | CH₂ | H | Q6b | |
| 1105. | Br | Br | CH₂ | H | Q7 | |
| 1106. | Br | Br | CH₂ | H | Q8a | |
| 1107. | Br | Br | CH₂ | H | Q8b | |
| 1108. | Br | Br | CH₂ | H | Q8c | |
| 1109. | Br | Br | CH₂ | H | Q8d | |
| 1110. | Br | Br | CH₂ | H | Q9a | |
| 1111. | Br | Br | CH₂ | H | Q9b | |
| 1112. | Br | Br | CH₂ | H | Q9c | |
| 1113. | Br | Br | CH₂ | H | Q9d | |
| 1114. | Br | Br | CH₂ | H | Q10a | |
| 1115. | Br | Br | CH₂ | H | Q11a | |
| 1116. | Br | Br | CH₂ | H | Q12 | |
| 1117. | Br | Br | CH₂ | H | Q13 | |
| 1118. | Br | Br | CH₂ | Me | Q1a | |
| 1119. | Br | Br | CH₂ | Me | Q1b | |
| 1120. | Br | Br | CH₂ | Me | Q1c | |
| 1121. | Br | Br | CH₂ | Me | Q1d | |
| 1122. | Br | Br | CH₂ | Me | Q1e | |
| 1123. | Br | Br | CH₂ | Me | Q2a | |
| 1124. | Br | Br | CH₂ | Me | Q2b | |
| 1125. | Br | Br | CH₂ | Me | Q3a | |
| 1126. | Br | Br | CH₂ | Me | Q3b | |
| 1127. | Br | Br | CH₂ | Me | Q3c | |
| 1128. | Br | Br | CH₂ | Me | Q3d | |
| 1129. | Br | Br | CH₂ | Me | Q3e | |
| 1130. | Br | Br | CH₂ | Me | Q3f | |
| 1131. | Br | Br | CH₂ | Me | Q3g | |
| 1132. | Br | Br | CH₂ | Me | Q3h | |
| 1133. | Br | Br | CH₂ | Me | Q3i | |
| 1134. | Br | Br | CH₂ | Me | Q3j | |
| 1135. | Br | Br | CH₂ | Me | Q3k | |
| 1136. | Br | Br | CH₂ | Me | Q31 | |
| 1137. | Br | Br | CH₂ | Me | Q3m | |
| 1138. | Br | Br | CH₂ | Me | Q3n | |
| 1139. | Br | Br | CH₂ | Me | Q3o | |
| 1140. | Br | Br | CH₂ | Me | Q3p | |
| 1141. | Br | Br | CH₂ | Me | Q3q | |
| 1142. | Br | Br | CH₂ | Me | Q3r | |
| 1143. | Br | Br | CH₂ | Me | Q3s | |
| 1144. | Br | Br | CH₂ | Me | Q3t | |
| 1145. | Br | Br | CH₂ | Me | Q3u | |
| 1146. | Br | Br | CH₂ | Me | Q3v | |
| 1147. | Br | Br | CH₂ | Me | Q3w | |
| 1148. | Br | Br | CH₂ | Me | Q3x | |
| 1149. | Br | Br | CH₂ | Me | Q3y | |
| 1150. | Br | Br | CH₂ | Me | Q3z | |
| 1151. | Br | Br | CH₂ | Me | Q3za | |
| 1152. | Br | Br | CH₂ | Me | Q3zb | |
| 1153. | Br | Br | CH₂ | Me | Q3zc | |
| 1154. | Br | Br | CH₂ | Me | Q3zd | |
| 1155. | Br | Br | CH₂ | Me | Q4a | |
| 1156. | Br | Br | CH₂ | Me | Q4b | |
| 1157. | Br | Br | CH₂ | Me | Q4c | |
| 1158. | Br | Br | CH₂ | Me | Q4d | |
| 1159. | Br | Br | CH₂ | Me | QSa | |
| 1160. | Br | Br | CH₂ | Me | Q6a | |
| 1161. | Br | Br | CH₂ | Me | Q6b | |
| 1162. | Br | Br | CH₂ | Me | Q7 | |
| 1163. | Br | Br | CH₂ | Me | Q8a | |
| 1164. | Br | Br | CH₂ | Me | Q8b | |
| 1165. | Br | Br | CH₂ | Me | Q8c | |
| 1166. | Br | Br | CH₂ | Me | Q8d | |
| 1167. | Br | Br | CH₂ | Me | Q9a | |
| 1168. | Br | Br | CH₂ | Me | Q9b | |
| 1169. | Br | Br | CH₂ | Me | Q9c | |
| 1170. | Br | Br | CH₂ | Me | Q9d | |
| 1171. | Br | Br | CH₂ | Me | Q10a | |
| 1172. | Br | Br | CH₂ | Me | Q11a | |
| 1173. | Br | Br | CH₂ | Me | Q12 | |
| 1174. | Br | Br | CH₂ | Me | Q13 | |
| 1175. | Br | Br | CH₂ | Et | Q1a | |
| 1176. | Br | Br | CH₂ | Et | Q1b | |
| 1177. | Br | Br | CH₂ | Et | Q1c | |
| 1178. | Br | Br | CH₂ | Et | Q1d | |
| 1179. | Br | Br | CH₂ | Et | Q1e | |
| 1180. | Br | Br | CH₂ | Et | Q2a | |
| 1181. | Br | Br | CH₂ | Et | Q2b | |
| 1182. | Br | Br | CH₂ | Et | Q3a | |
| 1183. | Br | Br | CH₂ | Et | Q3c | |
| 1184. | Br | Br | CH₂ | Et | Q4a | |
| 1185. | Br | Br | CH₂ | Et | Q5a | |
| 1186. | Br | Br | CH₂ | Et | Q6a | |
| 1187. | Br | Br | CH₂ | Et | Q8a | |
| 1188. | Br | Br | CH₂ | Et | Q8d | |
| 1189. | Br | Br | CH₂ | Et | Q9a | |
| 1190. | Br | Br | CH₂ | Et | Q12 | |
| 1191. | Br | Br | CH₂ | Et | Q13 | |
| 1192. | Br | Br | CH₂ | n-Pr | Q1a | |
| 1193. | Br | Br | CH₂ | n-Pr | Q3a | |
| 1194. | Br | Br | CH₂ | n-Pr | Q6a | |
| 1195. | Br | Br | CH₂ | SMe | Q1a | |
| 1196. | Br | Br | CH₂ | SMe | Q1b | |
| 1197. | Br | Br | CH₂ | SMe | Q1c | |
| 1198. | Br | Br | CH₂ | SMe | Q1d | |
| 1199. | Br | Br | CH₂ | SMe | Q1e | |
| 1200. | Br | Br | CH₂ | SMe | Q2a | |
| 1201. | Br | Br | CH₂ | SMe | Q2b | |
| 1202. | Br | Br | CH₂ | SMe | Q3a | |
| 1203. | Br | Br | CH₂ | SMe | Q3c | |
| 1204. | Br | Br | CH₂ | SMe | Q4a | |
| 1205. | Br | Br | CH₂ | SMe | Q5a | |
| 1206. | Br | Br | CH₂ | SMe | Q6a | |
| 1207. | Br | Br | CH₂ | SMe | Q8a | |
| 1208. | Br | Br | CH₂ | SMe | Q8d | |
| 1209. | Br | Br | CH₂ | SMe | Q9a | |
| 1210. | Br | Br | CH₂ | SMe | Q12 | |
| 1211. | Br | Br | CH₂ | SMe | Q13 | |
| 1212. | Br | Br | CH₂CH₂ | H | Q1a | |
| 1213. | Br | Br | CH₂CH₂ | H | Q1b | |
| 1214. | Br | Br | CH₂CH₂ | H | Q1c | |
| 1215. | Br | Br | CH₂CH₂ | H | Q1d | |
| 1216. | Br | Br | CH₂CH₂ | H | Q1e | |
| 1217. | Br | Br | CH₂CH₂ | H | Q2a | |
| 1218. | Br | Br | CH₂CH₂ | H | Q2b | |
| 1219. | Br | Br | CH₂CH₂ | H | Q3a | |
| 1220. | Br | Br | CH₂CH₂ | H | Q3c | |
| 1221. | Br | Br | CH₂CH₂ | H | Q4a | |
| 1222. | Br | Br | CH₂CH₂ | H | Q5a | |
| 1223. | Br | Br | CH₂CH₂ | H | Q6a | |
| 1224. | Br | Br | CH₂CH₂ | H | Q8a | |
| 1225. | Br | Br | CH₂CH₂ | H | Q8d | |
| 1226. | Br | Br | CH₂CH₂ | H | Q9a | |
| 1227. | Br | Br | CH₂CH₂ | H | Q12 | |
| 1228. | Br | Br | CH₂CH₂ | H | Q13 | |
| 1229. | Br | Br | CH₂CH₂ | Me | Q1a | |
| 1230. | Br | Br | CH₂CH₂ | Me | Q2a | |
| 1231. | Br | Br | CH₂CH₂ | Me | Q2b | |
| 1232. | Br | Br | CH₂CH₂ | Me | Q3a | |
| 1233. | Br | Br | CH₂CH₂ | Me | Q6a | |
| 1234. | Br | Br | OCH₂CH₂ | H | Q1a | 64-68 |
| 1235. | Br | Br | OCH₂CH₂ | H | Q1b | |
| 1236. | Br | Br | OCH₂CH₂ | H | Q1c | |
| 1237. | Br | Br | OCH₂CH₂ | H | Q1d | |
| 1238. | Br | Br | OCH₂CH₂ | H | Q1e | |
| 1239. | Br | Br | OCH₂CH₂ | H | Q2a | 1.6035 |
| 1240. | Br | Br | OCH₂CH₂ | H | Q2b | |
| 1241. | Br | Br | OCH₂CH₂ | H | Q3a | |
| 1242. | Br | Br | OCH₂CH₂ | H | Q3b | |
| 1243. | Br | Br | OCH₂CH₂ | H | Q3c | |
| 1244. | Br | Br | OCH₂CH₂ | H | Q3d | |
| 1245. | Br | Br | OCH₂CH₂ | H | Q3e | |
| 1246. | Br | Br | OCH₂CH₂ | H | Q3f | |
| 1247. | Br | Br | OCH₂CH₂ | H | Q3g | |
| 1248. | Br | Br | OCH₂CH₂ | H | Q3h | |
| 1249. | Br | Br | OCH₂CH₂ | H | Q3i | |
| 1250. | Br | Br | OCH₂CH₂ | H | Q3j | |
| 1251. | Br | Br | OCH₂CH₂ | H | Q3k | |
| 1252. | Br | Br | OCH₂CH₂ | H | Q31 | |
| 1253. | Br | Br | OCH₂CH₂ | H | Q3m | |
| 1254. | Br | Br | OCH₂CH₂ | H | Q3n | |
| 1255. | Br | Br | OCH₂CH₂ | H | Q3o | |
| 1256. | Br | Br | OCH₂CH₂ | H | Q3p | |
| 1257. | Br | Br | OCH₂CH₂ | H | Q3q | |
| 1258. | Br | Br | OCH₂CH₂ | H | Q3r | |
| 1259. | Br | Br | OCH₂CH₂ | H | Q3s | |
| 1260. | Br | Br | OCH₂CH₂ | H | Q3t | |
| 1261. | Br | Br | OCH₂CH₂ | H | Q3u | |
| 1262. | Br | Br | OCH₂CH₂ | H | Q3v | |
| 1263. | Br | Br | OCH₂CH₂ | H | Q3w | |
| 1264. | Br | Br | OCH₂CH₂ | H | Q3x | |
| 1265. | Br | Br | OCH₂CH₂ | H | Q3y | |
| 1266. | Br | Br | OCH₂CH₂ | H | Q3z | |
| 1267. | Br | Br | OCH₂CH₂ | H | Q3za | |
| 1268. | Br | Br | OCH₂CH₂ | H | Q3zb | |
| 1269. | Br | Br | OCH₂CH₂ | H | Q3zc | |
| 1270. | Br | Br | OCH₂CH₂ | H | Q3zd | |
| 1271. | Br | Br | OCH₂CH₂ | H | Q4a | |
| 1272. | Br | Br | OCH₂CH₂ | H | Q4b | |
| 1273. | Br | Br | OCH₂CH₂ | H | Q4c | |
| 1274. | Br | Br | OCH₂CH₂ | H | Q4d | |
| 1275. | Br | Br | OCH₂CH₂ | H | Q5a | |
| 1276. | Br | Br | OCH₂CH₂ | H | Q5b | |
| 1277. | Br | Br | OCH₂CH₂ | H | Q5c | |
| 1278. | Br | Br | OCH₂CH₂ | H | Q5d | |
| 1279. | Br | Br | OCH₂CH₂ | H | Q5e | |
| 1280. | Br | Br | OCH₂CH₂ | H | Q6a | |
| 1281. | Br | Br | OCH₂CH₂ | H | Q6b | |
| 1282. | Br | Br | OCH₂CH₂ | H | Q7 | |
| 1283. | Br | Br | OCH₂CH₂ | H | Q8a | |
| 1284. | Br | Br | OCH₂CH₂ | H | Q8b | |
| 1285. | Br | Br | OCH₂CH₂ | H | Q8c | |
| 1286. | Br | Br | OCH₂CH₂ | H | Q8d | |
| 1287. | Br | Br | OCH₂CH₂ | H | Q9a | |
| 1288. | Br | Br | OCH₂CH₂ | H | Q9b | |
| 1289. | Br | Br | OCH₂CH₂ | H | Q9c | |
| 1290. | Br | Br | OCH₂CH₂ | H | Q9d | |
| 1291. | Br | Br | OCH₂CH₂ | H | Q10a | |
| 1292. | Br | Br | OCH₂CH₂ | H | Q11a | |
| 1293. | Br | Br | OCH₂CH₂ | H | Q12 | |
| 1294. | Br | Br | OCH₂CH₂ | H | Q13 | |
| 1295. | Br | Br | OCH₂CH₂ | Me | Q1a | |
| 1296. | Br | Br | OCH₂CH₂ | Me | Q1b | |
| 1297. | Br | Br | OCH₂CH₂ | Me | Q1c | |
| 1298. | Br | Br | OCH₂CH₂ | Me | Q1d | |
| 1299. | Br | Br | OCH₂CH₂ | Me | Q1e | |
| 1300. | Br | Br | OCH₂CH₂ | Me | Q2a | |
| 1301. | Br | Br | OCH₂CH₂ | Me | Q2b | |
| 1302. | Br | Br | OCH₂CH₂ | Me | Q3a | |
| 1303. | Br | Br | OCH₂CH₂ | Me | Q3b | |
| 1304. | Br | Br | OCH₂CH₂ | Me | Q3c | |
| 1305. | Br | Br | OCH₂CH₂ | Me | Q3d | |
| 1306. | Br | Br | OCH₂CH₂ | Me | Q3e | |
| 1307. | Br | Br | OCH₂CH₂ | Me | Q3f | |
| 1308. | Br | Br | OCH₂CH₂ | Me | Q3g | |
| 1309. | Br | Br | OCH₂CH₂ | Me | Q3h | |
| 1310. | Br | Br | OCH₂CH₂ | Me | Q3i | |
| 1311. | Br | Br | OCH₂CH₂ | Me | Q3j | |
| 1312. | Br | Br | OCH₂CH₂ | Me | Q3k | |
| 1313. | Br | Br | OCH₂CH₂ | Me | Q31 | |
| 1314. | Br | Br | OCH₂CH₂ | Me | Q3m | |
| 1315. | Br | Br | OCH₂CH₂ | Me | Q3n | |
| 1316. | Br | Br | OCH₂CH₂ | Me | Q3o | |
| 1317. | Br | Br | OCH₂CH₂ | Me | Q3p | |
| 1318. | Br | Br | OCH₂CH₂ | Me | Q3q | |
| 1319. | Br | Br | OCH₂CH₂ | Me | Q3r | |
| 1320. | Br | Br | OCH₂CH₂ | Me | Q3s | |
| 1321. | Br | Br | OCH₂CH₂ | Me | Q3t | |
| 1322. | Br | Br | OCH₂CH₂ | Me | Q3u | |
| 1323. | Br | Br | OCH₂CH₂ | Me | Q3v | |
| 1324. | Br | Br | OCH₂CH₂ | Me | Q3w | |
| 1325. | Br | Br | OCH₂CH₂ | Me | Q3x | |
| 1326. | Br | Br | OCH₂CH₂ | Me | Q3y | |
| 1327. | Br | Br | OCH₂CH₂ | Me | Q3z | |
| 1328. | Br | Br | OCH₂CH₂ | Me | Q3za | |
| 1329. | Br | Br | OCH₂CH₂ | Me | Q3zb | |
| 1330. | Br | Br | OCH₂CH₂ | Me | Q3zc | |
| 1331. | Br | Br | OCH₂CH₂ | Me | Q3zd | |
| 1332. | Br | Br | OCH₂CH₂ | Me | Q4a | |
| 1333. | Br | Br | OCH₂CH₂ | Me | Q4b | |
| 1334. | Br | Br | OCH₂CH₂ | Me | Q4c | |
| 1335. | Br | Br | OCH₂CH₂ | Me | Q4d | |
| 1336. | Br | Br | OCH₂CH₂ | Me | Q5a | |
| 1337. | Br | Br | OCH₂CH₂ | Me | Q5b | |
| 1338. | Br | Br | OCH₂CH₂ | Me | Q5c | |
| 1339. | Br | Br | OCH₂CH₂ | Me | Q6a | |
| 1340. | Br | Br | OCH₂CH₂ | Me | Q6b | |
| 1341. | Br | Br | OCH₂CH₂ | Me | Q7 | |
| 1342. | Br | Br | OCH₂CH₂ | Me | Q8a | |
| 1343. | Br | Br | OCH₂CH₂ | Me | Q8b | |
| 1344. | Br | Br | OCH₂CH₂ | Me | Q8c | |
| 1345. | Br | Br | OCH₂CH₂ | Me | Q8d | |
| 1346. | Br | Br | OCH₂CH₂ | Me | Q9a | |
| 1347. | Br | Br | OCH₂CH₂ | Me | Q9b | |
| 1348. | Br | Br | OCH₂CH₂ | Me | Q9c | |
| 1349. | Br | Br | OCH₂CH₂ | Me | Q9d | |
| 1350. | Br | Br | OCH₂CH₂ | Me | Q10a | |
| 1351. | Br | Br | OCH₂CH₂ | Me | Q11a | |
| 1352. | Br | Br | OCH₂CH₂ | Me | Q12 | |
| 1353. | Br | Br | OCH₂CH₂ | Me | Q13 | |
| 1354. | Br | Br | OCH₂CH₂ | Et | Q1a | |
| 1355. | Br | Br | OCH₂CH₂ | Et | Q1b | |
| 1356. | Br | Br | OCH₂CH₂ | Et | Q1c | |
| 1357. | Br | Br | OCH₂CH₂ | Et | Q1d | |
| 1358. | Br | Br | OCH₂CH₂ | Et | Q1e | |
| 1359. | Br | Br | OCH₂CH₂ | Et | Q2a | |
| 1360. | Br | Br | OCH₂CH₂ | Et | Q2b | |
| 1361. | Br | Br | OCH₂CH₂ | Et | Q3a | |
| 1362. | Br | Br | OCH₂CH₂ | Et | Q3c | |
| 1363. | Br | Br | OCH₂CH₂ | Et | Q4a | |
| 1364. | Br | Br | OCH₂CH₂ | Et | Q5a | |
| 1365. | Br | Br | OCH₂CH₂ | Et | Q6a | |
| 1366. | Br | Br | OCH₂CH₂ | Et | Q8a | |
| 1367. | Br | Br | OCH₂CH₂ | Et | Q8d | |
| 1368. | Br | Br | OCH₂CH₂ | Et | Q9a | |
| 1369. | Br | Br | OCH₂CH₂ | Et | Q12 | |
| 1370. | Br | Br | OCH₂CH₂ | Et | Q13 | |
| 1371. | Br | Br | OCH₂CH₂ | n-Pr | Q1a | |
| 1372. | Br | Br | OCH₂CH₂ | n-Pr | Q3a | |
| 1373. | Br | Br | OCH₂CH₂ | n-Pr | Q6a | |
| 1374. | Br | Br | OCH₂CH₂ | SMe | Q1a | |
| 1375. | Br | Br | OCH₂CH₂ | SMe | Q3a | |
| 1376. | Br | Br | OCH₂CH₂ | SMe | Q6a | |
| 1377. | Br | Br | OCH₂CH(Me) | H | Q1a | |
| 1378. | Br | Br | OCH₂CH(Me) | H | Q1b | |
| 1379. | Br | Br | OCH₂CH(Me) | H | Q1c | |
| 1380. | Br | Br | OCH₂CH(Me) | H | Q1d | |
| 1381. | Br | Br | OCH₂CH(Me) | H | Q1e | |
| 1382. | Br | Br | OCH₂CH(Me) | H | Q2a | |
| 1383. | Br | Br | OCH₂CH(Me) | H | Q2b | |
| 1384. | Br | Br | OCH₂CH(Me) | H | Q3a | |
| 1385. | Br | Br | OCH₂CH(Me) | H | Q3c | |
| 1386. | Br | Br | OCH₂CH(Me) | H | Q4a | |
| 1387. | Br | Br | OCH₂CH(Me) | H | Q5a | |
| 1388. | Br | Br | OCH₂CH(Me) | H | Q6a | |
| 1389. | Br | Br | OCH₂CH(Me) | H | Q8a | |
| 1390. | Br | Br | OCH₂CH(Me) | H | Q8d | |
| 1391. | Br | Br | OCH₂CH(Me) | H | Q9a | |
| 1392. | Br | Br | OCH₂CH(Me) | H | Q12 | |
| 1393. | Br | Br | OCH₂CH(Me) | H | Q13 | |
| 1394. | Br | Br | OCH₂CH(Me) | Me | Q1a | |
| 1395. | Br | Br | OCH₂CH(Me) | Me | Q3a | |
| 1396. | Br | Br | OCH₂CH(Me) | Me | Q6a | |
| 1397. | Br | Br | OCH₂CH₂CH₂ | H | Q1a | |
| 1398. | Br | Br | OCH₂CH₂CH₂ | H | Q1b | |
| 1399. | Br | Br | OCH₂CH₂CH₂ | H | Q1c | |
| 1400. | Br | Br | OCH₂CH₂CH₂ | H | Q1d | |
| 1401. | Br | Br | OCH₂CH₂CH₂ | H | Q1e | |
| 1402. | Br | Br | OCH₂CH₂CH₂ | H | Q2a | |
| 1403. | Br | Br | OCH₂CH₂CH₂ | H | Q2b | |
| 1404. | Br | Br | OCH₂CH₂CH₂ | H | Q3a | |
| 1405. | Br | Br | OCH₂CH₂CH₂ | H | Q3c | |
| 1406. | Br | Br | OCH₂CH₂CH₂ | H | Q4a | |
| 1407. | Br | Br | OCH₂CH₂CH₂ | H | Q5a | |
| 1408. | Br | Br | OCH₂CH₂CH₂ | H | Q6a | |
| 1409. | Br | Br | OCH₂CH₂CH₂ | H | Q8a | |
| 1410. | Br | Br | OCH₂CH₂CH₂ | H | Q8d | |
| 1411. | Br | Br | OCH₂CH₂CH₂ | H | Q9a | |
| 1412. | Br | Br | OCH₂CH₂CH₂ | H | Q12 | |
| 1413. | Br | Br | OCH₂CH₂CH₂ | H | Q13 | |
| 1414. | Br | Br | OCH₂CH₂CH₂ | Me | Q1a | |
| 1415. | Br | Br | OCH₂CH₂CH₂ | Me | Q3a | |
| 1416. | Br | Br | OCH₂CH₂CH₂ | Me | Q6a | |
| 1417. | Br | Br | CH(Me) | H | Q1a | |
| 1418. | Br | Br | CH(Me)CH₂ | H | Q1a | |
| 1419. | Br | Br | OCH₂ | H | Q1a | |
| 1420. | Br | Br | OCH(Me)CH₂ | H | Q1a | |
| 1421. | Br | Br(Me) | OCH₂CH₂ | H | Q1a | |
| 1422. | Br | Br(Me) | OCH₂CH₂ | H | Q2a | |
| 1423. | Br | Br(Me) | OCH₂CH₂ | H | Q2b | |
| 1424. | Br | Br(Me) | OCH₂CH₂ | H | Q3a | |
| 1425. | Br | Br(Me) | OCH₂CH₂ | H | Q6a | |
| 1426. | Br | Br(Me) | OCH₂CH₂ | H | Q8d | |
| 1427. | Br | Br(Me) | OCH₂CH₂ | Me | Q1a | |
| 1428. | Br | Br(Me) | OCH₂CH₂ | Me | Q2a | |
| 1429. | Br | Br(Me) | OCH₂CH₂ | Me | Q2b | |
| 1430. | Br | Br(Me) | OCH₂CH₂ | Me | Q3a | |
| 1431. | Br | Br(Me) | OCH₂CH₂ | Me | Q6a | |
| 1432. | Br | Br(Me) | OCH₂CH₂ | Me | Q8d | |
| 1433. | Br | I | OCH₂CH₂ | H | Q1a | |
| 1434. | Br | I | OCH₂CH₂ | H | Q2a | |
| 1435. | Br | I | OCH₂CH₂ | H | Q2b | |
| 1436. | Br | I | OCH₂CH₂ | H | Q3a | |
| 1437. | Br | I | OCH₂CH₂ | H | Q6a | |
| 1438. | Br | I | OCH₂CH₂ | H | Q8d | |
| 1439. | Br | I | OCH₂CH₂ | Me | Q1a | |
| 1440. | Br | I | OCH₂CH₂ | Me | Q2a | |
| 1441. | Br | I | OCH₂CH₂ | Me | Q2b | |
| 1442. | Br | I | OCH₂CH₂ | Me | Q6a | |
| 1443. | Br | I | OCH(Me)CH₂ | H | Q1a | |
| 1444. | Br | I | OCH₂CH₂CH₂ | H | Q1a | |
| 1445. | Br | Me | OCH₂CH₂ | H | Q1a | |
| 1446. | Br | Me | OCH₂CH₂ | H | Q1b | |
| 1447. | Br | Me | OCH₂CH₂ | H | Q1c | |
| 1448. | Br | Me | OCH₂CH₂ | H | Q1d | |
| 1449. | Br | Me | OCH₂CH₂ | H | Q1e | |
| 1450. | Br | Me | OCH₂CH₂ | H | Q2a | |
| 1451. | Br | Me | OCH₂CH₂ | H | Q2b | |
| 1452. | Br | Me | OCH₂CH₂ | H | Q3a | |
| 1453. | Br | Me | OCH₂CH₂ | H | Q3c | |
| 1454. | Br | Me | OCH₂CH₂ | H | Q4a | |
| 1455. | Br | Me | OCH₂CH₂ | H | Q5a | |
| 1456. | Br | Me | OCH₂CH₂ | H | Q6a | |
| 1457. | Br | Me | OCH₂CH₂ | H | Q8a | |
| 1458. | Br | Me | OCH₂CH₂ | H | Q8d | |
| 1459. | Br | Me | OCH₂CH₂ | H | Q9a | |
| 1460. | Br | Me | OCH₂CH₂ | H | Q12 | |
| 1461. | Br | Me | OCH₂CH₂ | H | Q13 | |
| 1462. | Br | Me | OCH₂CH₂ | Me | Q1a | |
| 1463. | Br | Me | OCH₂CH₂ | Me | Q2a | |
| 1464. | Br | Me | OCH₂CH₂ | Me | Q2b | |
| 1465. | Br | Me | OCH₂CH₂ | Me | Q3a | |
| 1466. | Br | Me | OCH₂CH₂ | Me | Q6a | |
| 1467. | Br | Me | OCH₂CH₂ | Me | Q8d | |
| 1468. | Br | Me | OCH₂CH(Me) | H | Q1a | |
| 1469. | Br | Me | OCH₂CH₂CH₂ | H | Q1a | |
| 1470. | Br | CN | OCH₂CH₂ | H | Q1a | |
| 1471. | Br | CN | OCH₂CH₂ | H | Q3a | |
| 1472. | Br | CN | OCH₂CH₂ | H | Q4a | |
| 1473. | Br | CN | OCH₂CH₂ | Me | Q1a | |
| 1474. | Br | CN | OCH₂CH₂ | Me | Q3a | |
| 1475. | Br | CN | OCH₂CH₂ | Me | Q4a | |
| 1476. | Br | CF₃ | OCH₂CH₂ | H | Q1a | |
| 1477. | Br | CF₃ | OCH₂CH₂ | H | Q2a | |
| 1478. | Br | CF₃ | OCH₂CH₂ | H | Q2b | |
| 1479. | Br | CF₃ | OCH₂CH₂ | H | Q3a | |
| 1480. | Br | CF₃ | OCH₂CH₂ | Me | Q1a | |
| 1481. | Br | CF₃ | OCH₂CH₂ | Me | Q3a | |
| 1482. | Br | CF₃ | OCH₂CH₂CH₂ | H | Q1a | |
| 1483. | Br | CF₃ | OCH₂CH₂CH₂ | Me | Q1a | |
| 1484. | Br | SO₂Me | CH₂ | H | Q1a | |
| 1485. | Br | SO₂Me | CH₂ | H | Q1b | |
| 1486. | Br | SO₂Me | CH₂ | H | Q1c | |
| 1487. | Br | SO₂Me | CH₂ | H | Q1d | |
| 1488. | Br | SO₂Me | CH₂ | H | Q1e | |
| 1489. | Br | SO₂Me | CH₂ | H | Q2a | |
| 1490. | Br | SO₂Me | CH₂ | H | Q2b | |
| 1491. | Br | SO₂Me | CH₂ | H | Q3a | |
| 1492. | Br | SO₂Me | CH₂ | H | Q3c | |
| 1493. | Br | SO₂Me | CH₂ | H | Q4a | |
| 1494. | Br | SO₂Me | CH₂ | H | Q5a | |
| 1495. | Br | SO₂Me | CH₂ | H | Q6a | |
| 1496. | Br | SO₂Me | CH₂ | H | Q8a | |
| 1497. | Br | SO₂Me | CH₂ | H | Q8d | |
| 1498. | Br | SO₂Me | CH₂ | H | Q9a | |
| 1499. | Br | SO₂Me | CH₂ | H | Q12 | |
| 1500. | Br | SO₂Me | CH₂ | H | Q13 | |
| 1501. | Br | SO₂Me | CH₂ | Me | Q1a | |
| 1502. | Br | SO₂Me | CH₂ | Me | Q2a | |
| 1503. | Br | SO₂Me | CH₂ | Me | Q2b | |
| 1504. | Br | SO₂Me | CH₂ | Me | Q3a | |
| 1505. | Br | SO₂Me | CH₂ | Me | Q6a | |
| 1506. | Br | SO₂Me | CH₂ | Me | Q8d | |
| 1507. | Br | SO₂Me | CH₂ | Et | Q1a | |
| 1508. | Br | SO₂Me | CH₂ | n-Pr | Q1a | |
| 1509. | Br | SO₂Me | CH₂ | SMe | Q1a | |
| 1510. | Br | SO₂Me | CH₂CH₂ | H | Q1a | |
| 1511. | Br | SO₂Me | CH₂CH₂ | Me | Q1a | |
| 1512. | Br | SO₂Me | OCH₂CH₂ | H | Q1a | |
| 1513. | Br | SO₂Me | OCH₂CH₂ | H | Q1b | |
| 1514. | Br | SO₂Me | OCH₂CH₂ | H | Q1c | |
| 1515. | Br | SO₂Me | OCH₂CH₂ | H | Q1d | |
| 1516. | Br | SO₂Me | OCH₂CH₂ | H | Q1e | |
| 1517. | Br | SO₂Me | OCH₂CH₂ | H | Q2a | |
| 1518. | Br | SO₂Me | OCH₂CH₂ | H | Q2b | |
| 1519. | Br | SO₂Me | OCH₂CH₂ | H | Q3a | |
| 1520. | Br | SO₂Me | OCH₂CH₂ | H | Q3c | |
| 1521. | Br | SO₂Me | OCH₂CH₂ | H | Q4a | |
| 1522. | Br | SO₂Me | OCH₂CH₂ | H | Q5a | |
| 1523. | Br | SO₂Me | OCH₂CH₂ | H | Q6a | |
| 1524. | Br | SO₂Me | OCH₂CH₂ | H | Q8a | |
| 1525. | Br | SO₂Me | OCH₂CH₂ | H | Q8d | |
| 1526. | Br | SO₂Me | OCH₂CH₂ | H | Q9a | |
| 1527. | Br | SO₂Me | OCH₂CH₂ | H | Q12 | |
| 1528. | Br | SO₂Me | OCH₂CH₂ | H | Q13 | |
| 1529. | Br | SO₂Me | OCH₂CH₂ | Me | Q1a | |
| 1530. | Br | SO₂Me | OCH₂CH₂ | Me | Q2a | |
| 1531. | Br | SO₂Me | OCH₂CH₂ | Me | Q2b | |
| 1532. | Br | SO₂Me | OCH₂CH₂ | Me | Q3a | |
| 1533. | Br | SO₂Me | OCH₂CH₂ | Me | Q6a | |
| 1534. | Br | SO₂Me | OCH₂CH₂ | Me | Q8d | |
| 1535. | Br | SO₂Me | OCH₂CH₂ | Et | Q1a | |
| 1536. | Br | SO₂Me | OCH₂CH₂ | n-Pr | Q1a | |
| 1537. | Br | SO₂Me | OCH₂CH₂ | SMe | Q1a | |
| 1538. | Br | SO₂Me | OCH₂CH(Me) | H | Q1a | |
| 1539. | Br | SO₂Me | OCH₂CH₂CH₂ | H | Q1a | |
| 1540. | Br | SO₂Me | OCH₂CH₂CH₂ | Me | Q1a | |
| 1541. | I | I | OCH₂CH₂ | H | Q1a | |
| 1542. | I | I | OCH₂CH₂ | H | Q2a | |
| 1543. | I | I | OCH₂CH₂ | H | Q2b | |
| 1544. | I | I | OCH₂CH₂ | H | Q3a | |
| 1545. | I | I | OCH₂CH₂ | H | Q6a | |
| 1546. | I | I | OCH₂CH₂ | H | Q8d | |
| 1547. | I | I | OCH₂CH₂ | Me | Q1a | |
| 1548. | I | I | OCH₂CH₂ | Me | Q3a | |
| 1549. | I | I | OCH₂CH₂ | Me | Q6a | |
| 1550. | I | I | OCH₂CH(Me) | H | Q1a | |
| 1551. | I | I | OCH₂CH₂CH₂ | H | Q1a | |
| 1552. | Me | Cl | OCH₂CH₂ | H | Q1a | |
| 1553. | Me | Cl | OCH₂CH₂ | H | Q1b | |
| 1554. | Me | Cl | OCH₂CH₂ | H | Q1c | |
| 1555. | Me | Cl | OCH₂CH₂ | H | Q1d | |
| 1556. | Me | Cl | OCH₂CH₂ | H | Q1e | |
| 1557. | Me | Cl | OCH₂CH₂ | H | Q2a | |
| 1558. | Me | Cl | OCH₂CH₂ | H | Q2b | |
| 1559. | Me | Cl | OCH₂CH₂ | H | Q3a | |
| 1560. | Me | Cl | OCH₂CH₂ | H | Q3c | |
| 1561. | Me | Cl | OCH₂CH₂ | H | Q4a | |
| 1562. | Me | Cl | OCH₂CH₂ | H | QSa | |
| 1563. | Me | Cl | OCH₂CH₂ | H | Q6a | |
| 1564. | Me | Cl | OCH₂CH₂ | H | Q8a | |
| 1565. | Me | Cl | OCH₂CH₂ | H | Q8d | |
| 1566. | Me | Cl | OCH₂CH₂ | H | Q9a | |
| 1567. | Me | Cl | OCH₂CH₂ | H | Q12 | |
| 1568. | Me | Cl | OCH₂CH₂ | H | Q13 | |
| 1569. | Me | Cl | OCH₂CH₂ | Me | Q1a | |
| 1570. | Me | Cl | OCH₂CH₂ | Me | Q2a | |
| 1571. | Me | Cl | OCH₂CH₂ | Me | Q2b | |
| 1572. | Me | Cl | OCH₂CH₂ | Me | Q3a | |
| 1573. | Me | Cl | OCH₂CH₂ | Me | Q6a | |
| 1574. | Me | Cl | OCH₂CH₂ | Me | Q8d | |
| 1575. | Me | Cl | OCH₂CH(Me) | H | Q1a | |
| 1576. | Me | Cl | OCH₂CH₂CH₂ | H | Q1a | |
| 1577. | Me | Br | OCH₂CH₂ | H | Q1a | 1.5938 |
| 1578. | Me | Br | OCH₂CH₂ | H | Q1b | |
| 1579. | Me | Br | OCH₂CH₂ | H | Q1c | |
| 1580. | Me | Br | OCH₂CH₂ | H | Q1d | |
| 1581. | Me | Br | OCH₂CH₂ | H | Q1e | |
| 1582. | Me | Br | OCH₂CH₂ | H | Q2a | 63-66 |
| 1583. | Me | Br | OCH₂CH₂ | H | Q2b | |
| 1584. | Me | Br | OCH₂CH₂ | H | Q3a | 68-70 |
| 1585. | Me | Br | OCH₂CH₂ | H | Q3c | |
| 1586. | Me | Br | OCH₂CH₂ | H | Q4a | |
| 1587. | Me | Br | OCH₂CH₂ | H | Q5a | |
| 1588. | Me | Br | OCH₂CH₂ | H | Q6a | |
| 1589. | Me | Br | OCH₂CH₂ | H | Q8a | |
| 1590. | Me | Br | OCH₂CH₂ | H | Q8d | |
| 1591. | Me | Br | OCH₂CH₂ | H | Q9a | |
| 1592. | Me | Br | OCH₂CH₂ | H | Q12 | |
| 1593. | Me | Br | OCH₂CH₂ | H | Q13 | 154-155 |
| 1594. | Me | Br | OCH₂CH₂ | Me | Q1a | 65-70 |
| 1595. | Me | Br | OCH₂CH₂ | Me | Q2a | |
| 1596. | Me | Br | OCH₂CH₂ | Me | Q2b | |
| 1597. | Me | Br | OCH₂CH₂ | Me | Q3a | |
| 1598. | Me | Br | OCH₂CH₂ | Me | Q6a | |
| 1599. | Me | Br | OCH₂CH₂ | Me | Q8d | |
| 1600. | Me | Br | OCH₂CH(Me) | H | Q1a | |
| 1601. | Me | Br | OCH₂CH₂CH₂ | H | Q1a | |
| 1602. | Me | I | OCH₂CH₂ | H | Q1a | |
| 1603. | Me | I | OCH₂CH₂ | H | Q3a | |
| 1604. | Me | I | OCH₂CH₂ | H | Q6a | |
| 1605. | Me | I | OCH₂CH₂ | H | Q8d | |
| 1606. | Me | I | OCH₂CH₂ | Me | Q1a | |
| 1607. | Me | I | OCH₂CH₂ | Me | Q3a | |
| 1608. | Me | I | OCH₂CH₂ | Me | Q6a | |
| 1609. | Me | I | OCH₂CH(Me) | H | Q1a | |
| 1610. | Me | I | OCH₂CH₂CH₂ | H | Q1a | |
| 1611. | Me | SMe | OCH₂CH₂ | H | Q1a | |
| 1612. | Me | SMe | OCH₂CH₂ | H | Q3a | |
| 1613. | Me | SMe | OCH₂CH₂ | H | Q6a | |
| 1614. | Me | SMe | OCH₂CH₂ | H | Q8d | |
| 1615. | Me | SMe | OCH₂CH₂ | Me | Q1a | |
| 1616. | Me | SMe | OCH₂CH₂ | Me | Q3a | |
| 1617. | Me | SMe | OCH₂CH₂ | Me | Q6a | |
| 1618. | Me | SMe | OCH₂CH(Me) | H | Q1a | |
| 1619. | Me | SMe | OCH₂CH₂CH₂ | H | Q1a | |
| 1620. | Me | SO₂Me | CH₂ | H | Q1a | |
| 1621. | Me | SO₂Me | CH₂ | H | Q1b | |
| 1622. | Me | SO₂Me | CH₂ | H | Q1c | |
| 1623. | Me | SO₂Me | CH₂ | H | Q1d | |
| 1624. | Me | SO₂Me | CH₂ | H | Q1e | |
| 1625. | Me | SO₂Me | CH₂ | H | Q2a | |
| 1626. | Me | SO₂Me | CH₂ | H | Q2b | |
| 1627. | Me | SO₂Me | CH₂ | H | Q3a | |
| 1628. | Me | SO₂Me | CH₂ | H | Q3c | |
| 1629. | Me | SO₂Me | CH₂ | H | Q4a | |
| 1630. | Me | SO₂Me | CH₂ | H | Q5a | |
| 1631. | Me | SO₂Me | CH₂ | H | Q6a | |
| 1632. | Me | SO₂Me | CH₂ | H | Q8a | |
| 1633. | Me | SO₂Me | CH₂ | H | Q8d | |
| 1634. | Me | SO₂Me | CH₂ | H | Q9a | |
| 1635. | Me | SO₂Me | CH₂ | H | Q12 | |
| 1636. | Me | SO₂Me | CH₂ | H | Q13 | |
| 1637. | Me | SO₂Me | CH₂ | Me | Q1a | |
| 1638. | Me | SO₂Me | CH₂ | Me | Q2a | |
| 1639. | Me | SO₂Me | CH₂ | Me | Q2b | |
| 1640. | Me | SO₂Me | CH₂ | Me | Q3a | |
| 1641. | Me | SO₂Me | CH₂ | Me | Q6a | |
| 1642. | Me | SO₂Me | CH₂ | Me | Q8d | |
| 1643. | Me | SO₂Me | CH₂ | Et | Q1a | |
| 1644. | Me | SO₂Me | CH₂ | n-Pr | Q1a | |
| 1645. | Me | SO₂Me | CH₂ | SMe | Q1a | |
| 1646. | Me | SO₂Me | CH₂CH₂ | H | Q1a | |
| 1647. | Me | SO₂Me | CH₂CH₂ | Me | Q1a | |
| 1648. | Me | SO₂Me | OCH₂CH₂ | H | Q1a | |
| 1649. | Me | SO₂Me | OCH₂CH₂ | H | Q1b | |
| 1650. | Me | SO₂Me | OCH₂CH₂ | H | Q1c | |
| 1651. | Me | SO₂Me | OCH₂CH₂ | H | Q1d | |
| 1652. | Me | SO₂Me | OCH₂CH₂ | H | Q1e | |
| 1653. | Me | SO₂Me | OCH₂CH₂ | H | Q2a | |
| 1654. | Me | SO₂Me | OCH₂CH₂ | H | Q2b | |
| 1655. | Me | SO₂Me | OCH₂CH₂ | H | Q3a | |
| 1656. | Me | SO₂Me | OCH₂CH₂ | H | Q3c | |
| 1657. | Me | SO₂Me | OCH₂CH₂ | H | Q4a | |
| 1658. | Me | SO₂Me | OCH₂CH₂ | H | Q5a | |
| 1659. | Me | SO₂Me | OCH₂CH₂ | H | Q6a | |
| 1660. | Me | SO₂Me | OCH₂CH₂ | H | Q8a | |
| 1661. | Me | SO₂Me | OCH₂CH₂ | H | Q8d | |
| 1662. | Me | SO₂Me | OCH₂CH₂ | H | Q9a | |
| 1663. | Me | SO₂Me | OCH₂CH₂ | H | Q12 | |
| 1664. | Me | SO₂Me | OCH₂CH₂ | H | Q13 | |
| 1665. | Me | SO₂Me | OCH₂CH₂ | Me | Q1a | |
| 1666. | Me | SO₂Me | OCH₂CH₂ | Me | Q2a | |
| 1667. | Me | SO₂Me | OCH₂CH₂ | Me | Q2b | |
| 1668. | Me | SO₂Me | OCH₂CH₂ | Me | Q3a | |
| 1669. | Me | SO₂Me | OCH₂CH₂ | Me | Q6a | |
| 1670. | Me | SO₂Me | OCH₂CH₂ | Me | Q8d | |
| 1671. | Me | SO₂Me | OCH₂CH₂ | Et | Q1a | |
| 1672. | Me | SO₂Me | OCH₂CH₂ | n-Pr | Q1a | |
| 1673. | Me | SO₂Me | OCH₂CH₂ | SMe | Q1a | |
| 1674. | Me | SO₂Me | OCH₂CH(Me) | H | Q1a | |
| 1675. | Me | SO₂Me | OCH₂CH₂CH₂ | H | Q1a | |
| 1676. | Me | SO₂Me | OCH₂CH₂CH₂ | Me | Q1a | |
| 1677. | Me | NO₂ | OCH₂CH₂ | H | Q1a | |
| 1678. | Me | NO₂ | OCH₂CH₂ | H | Q1b | |
| 1679. | Me | NO₂ | OCH₂CH₂ | H | Q1c | |
| 1680. | Me | NO₂ | OCH₂CH₂ | H | Q1d | |
| 1681. | Me | NO₂ | OCH₂CH₂ | H | Q1e | |
| 1682. | Me | NO₂ | OCH₂CH₂ | H | Q2a | |
| 1683. | Me | NO₂ | OCH₂CH₂ | H | Q2b | |
| 1684. | Me | NO₂ | OCH₂CH₂ | H | Q3a | |
| 1685. | Me | NO₂ | OCH₂CH₂ | H | Q3c | |
| 1686. | Me | NO₂ | OCH₂CH₂ | H | Q4a | |
| 1687. | Me | NO₂ | OCH₂CH₂ | H | Q5a | |
| 1688. | Me | NO₂ | OCH₂CH₂ | H | Q6a | |
| 1689. | Me | NO₂ | OCH₂CH₂ | H | Q8a | |
| 1690. | Me | NO₂ | OCH₂CH₂ | H | Q8d | |
| 1691. | Me | NO₂ | OCH₂CH₂ | H | Q9a | |
| 1692. | Me | NO₂ | OCH₂CH₂ | H | Q12 | |
| 1693. | Me | NO₂ | OCH₂CH₂ | H | Q13 | |
| 1694. | Me | NO₂ | OCH₂CH₂ | Me | Q1a | |
| 1695. | Me | NO₂ | OCH₂CH₂ | Me | Q2a | |
| 1696. | Me | NO₂ | OCH₂CH₂ | Me | Q2b | |
| 1697. | Me | NO₂ | OCH₂CH₂ | Me | Q3a | |
| 1698. | Me | NO₂ | OCH₂CH₂ | Me | Q6a | |
| 1699. | Me | NO₂ | OCH₂CH₂ | Me | Q8d | |
| 1700. | Me | NO₂ | OCH₂CH₂ | Et | Q1a | |
| 1701. | Me | NO₂ | OCH₂CH₂ | n-Pr | Q1a | |
| 1702. | Me | NO₂ | OCH₂CH₂ | SMe | Q1a | |
| 1703. | Me | NO₂ | OCH₂CH(Me) | H | Q1a | |
| 1704. | Me | NO₂ | OCH₂CH₂CH₂ | H | Q1a | |
| 1705. | Me | NO₂ | OCH₂CH₂CH₂ | Me | Q1a | |
| 1706. | OMe | Cl | CH₂ | H | Q1a | |
| 1707. | OMe | Cl | CH₂ | H | Q3a | |
| 1708. | OMe | Cl | CH₂ | H | Q8d | |
| 1709. | OMe | Cl | CH₂ | Me | Q1a | |
| 1710. | OMe | Cl | CH₂ | Me | Q3a | |
| 1711. | OMe | Cl | CH₂ | Me | Q8d | |
| 1712. | SMe | Cl | OCH₂CH₂ | H | Q1a | |
| 1713. | SMe | Cl | OCH₂CH₂ | H | Q3a | |
| 1714. | SMe | Cl | OCH₂CH₂ | H | Q8d | |
| 1715. | SMe | Cl | OCH₂CH₂ | Me | Q1a | |
| 1716. | SMe | Cl | OCH₂CH₂ | Me | Q3a | |
| 1717. | SMe | Cl | OCH₂CH₂ | Me | Q8d | |
| 1718. | SMe | SMe | CH₂ | H | Q1a | |
| 1719. | SMe | SMe | CH₂ | H | Q3a | |
| 1720. | SO₂Me | Cl | OCH₂CH₂ | H | Q1a | |
| 1721. | SO₂Me | Cl | OCH₂CH₂ | H | Q3a | |
| 1722. | SO₂Me | Cl | OCH₂CH₂ | H | Q8d | |
| 1723. | SO₂Me | Cl | OCH₂CH₂ | Me | Q1a | |
| 1724. | SO₂Me | Cl | OCH₂CH₂ | Me | Q3a | |
| 1725. | SO₂Me | Cl | OCH₂CH₂ | Me | Q8d | |
| 1726. | NO₂ | Me | OCH₂CH₂ | H | Q1a | |
| 1727. | NO₂ | Me | OCH₂CH₂ | H | Q3a | |
| 1728. | NO₂ | Me | OCH₂CH₂ | H | Q8d | |
| 1729. | NO₂ | Me | OCH₂CH₂ | Me | Q1a | |
| 1730. | NO₂ | Me | OCH₂CH₂ | Me | Q3a | |
| 1731. | NO₂ | Me | OCH₂CH₂ | Me | Q8d | |
| 1732. | NO₂ | CH₂OMe | OCH₂CH₂ | H | Q1a | |
| 1733. | NO₂ | CH₂SMe | OCH₂CH₂ | H | Q1a | |
| 1734. | NO₂ | CH₂SO₂Me | OCH₂CH₂ | H | Q1a | |
| 1735. | CN | Me | OCH₂CH₂ | H | Q1a | |
| 1736. | CN | Me | OCH₂CH₂ | H | Q3a | |
| 1737. | CN | Me | OCH₂CH₂ | H | Q8d | |
| 1738. | CN | Me | OCH₂CH₂ | Me | Q1a | |
| 1739. | CN | Me | OCH₂CH₂ | Me | Q3a | |
| 1740. | CN | Me | OCH₂CH₂ | Me | Q8d | |
| 1741. | Cl | Cl | CH₂ | H | Q4e | |
| 1742. | Cl | Cl | CH₂ | H | Q4f | |
| 1743. | Cl | Cl | CH₂ | H | Q4g | |
| 1744. | Cl | Cl | CH₂ | H | Q10b | |
| 1745. | Cl | Cl | CH₂ | H | Q16 | |
| 1746. | Cl | Cl | CH₂ | H | Q22 | |
| 1747. | Cl | Cl | OCH₂CH₂ | H | Q4e | |
| 1748. | Cl | Cl | OCH₂CH₂ | H | Q4f | |
| 1749. | Cl | Cl | OCH₂CH₂ | H | Q4g | |
| 1750. | Cl | Cl | OCH₂CH₂ | H | Q4h | |
| 1751. | Cl | Cl | OCH₂CH₂ | H | Q4i | |
| 1752. | Cl | Cl | OCH₂CH₂ | H | Q4j | |
| 1753. | Cl | Cl | OCH₂CH₂ | H | Q10b | |
| 1754. | Cl | Cl | OCH₂CH₂ | H | Q10c | |
| 1755. | Cl | Cl | OCH₂CH₂ | H | Q10d | |
| 1756. | Cl | Cl | OCH₂CH₂ | H | Q10c | |
| 1757. | Cl | Cl | OCH₂CH₂ | H | Q10f | |
| 1758. | Cl | Cl | OCH₂CH₂ | H | Q16 | |
| 1759. | Cl | Cl | OCH₂CH₂ | H | Q17 | |
| 1760. | Cl | Cl | OCH₂CH₂ | H | Q18 | |
| 1761. | Cl | Cl | OCH₂CH₂ | H | Q19 | |
| 1762. | Cl | Cl | OCH₂CH₂ | H | Q20 | |
| 1763. | Cl | Cl | OCH₂CH₂ | H | Q21 | |
| 1764. | Cl | Cl | OCH₂CH₂ | H | Q22 | |
| 1765. | Cl | Cl | OCH₂CH₂ | H | Q23 | |
| 1766. | Cl | Cl | OCH₂CH₂ | H | Q24 | |
| 1767. | Cl | Cl | OCH₂CH₂ | H | Q25 | |
| 1768. | Cl | Cl | OCH₂CH₂ | H | Q26 | |
| 1769. | Cl | Cl | OCH₂CH₂ | Me | Q4e | |
| 1770. | Cl | Cl | OCH₂CH₂ | Me | Q4f | |
| 1771. | Cl | Cl | OCH₂CH₂ | Me | Q10b | |
| 1772. | Cl | Cl | OCH₂CH₂ | Me | Q16 | |
| 1773. | Cl | Cl | OCH₂CH₂ | Me | Q19 | |
| 1774. | Cl | Cl | OCH₂CH₂ | Me | Q22 | |
| 1775. | Cl | SO₂Me | CH₂ | H | Q4e | |
| 1776. | Cl | SO₂Me | CH₂ | H | Q4f | |
| 1777. | Cl | SO₂Me | CH₂ | H | Q4g | |
| 1778. | Cl | SO₂Me | CH₂ | H | Q4h | |
| 1779. | Cl | SO₂Me | CH₂ | H | Q4i | |
| 1780. | Cl | SO₂Me | CH₂ | H | Q4j | |
| 1781. | Cl | SO₂Me | CH₂ | H | Q10b | |
| 1782. | Cl | SO₂Me | CH₂ | H | Q10c | |
| 1783. | Cl | SO₂Me | CH₂ | H | Q10d | |
| 1784. | Cl | SO₂Me | CH₂ | H | Q10e | |
| 1785. | Cl | SO₂Me | CH₂ | H | Q10f | |
| 1786. | Cl | SO₂Me | CH₂ | H | Q11b | |
| 1787. | Cl | SO₂Me | CH₂ | H | Q11c | |
| 1788. | Cl | SO₂Me | CH₂ | H | Q1ld | |
| 1789. | Cl | SO₂Me | CH₂ | H | Q16 | |
| 1790. | Cl | SO₂Me | CH₂ | H | Q17 | |
| 1791. | Cl | SO₂Me | CH₂ | H | Q18 | |
| 1792. | Cl | SO₂Me | CH₂ | H | Q19 | |
| 1793. | Cl | SO₂Me | CH₂ | H | Q20 | |
| 1794. | Cl | SO₂Me | CH₂ | H | Q21 | |
| 1795. | Cl | SO₂Me | CH₂ | H | Q22 | |
| 1796. | Cl | SO₂Me | CH₂ | H | Q23 | |
| 1797. | Cl | SO₂Me | CH₂ | H | Q24 | |
| 1798. | Cl | SO₂Me | CH₂ | H | Q25 | |
| 1799. | Cl | SO₂Me | CH₂ | H | Q26 | |
| 1800. | Cl | SO₂Me | CH₂ | Me | Q4e | |
| 1801. | Cl | SO₂Me | CH₂ | Me | Q4f | |
| 1802. | Cl | SO₂Me | CH₂ | Me | Q10b | |
| 1803. | Cl | SO₂Me | CH₂ | Me | Q16 | |
| 1804. | Cl | SO₂Me | CH₂ | Me | Q19 | |
| 1805. | Cl | SO₂Me | CH₂ | Me | Q22 | |

*Substituent R^{1c} is mentioned in parenthesis in the column of the group R^{1b}, only when it is other than a hydrogen atom (H).

TABLE 2

| Compound | | | | | | Mp (° C.) |
|---|---|---|---|---|---|---|
| No. | R^{1a} | R^{1b} (R^{1c}) | (O)ₙ—A | R² | Q | or n_{D}²⁰ |
| 1806. | H | Cl | OCH₂CH₂ | H | Q1a | |
| 1807. | H | Cl | OCH₂CH₂ | H | Q2a | |
| 1808. | F | Cl | CH₂ | H | Q1a | |
| 1809. | F | Cl | OCH₂CH₂ | H | Q1a | |
| 1810. | F | Cl | CH₂ | Me | Q1a | |
| 1811. | Cl | F | CH₂ | H | Q1a | |
| 1812. | Cl | F | CH₂ | Me | Q1a | |
| 1813. | Cl | F | OCH₂CH₂ | H | Q1a | |
| 1814. | Cl | F | OCH₂CH₂ | H | Q1b | |
| 1815. | Cl | F | OCH₂CH₂ | H | Q1c | |
| 1816. | Cl | F | OCH₂CH₂ | H | Q1d | |
| 1817. | Cl | F | OCH₂CH₂ | H | Q1e | |
| 1818. | Cl | F | OCH₂CH₂ | H | Q2a | |
| 1819. | Cl | F | OCH₂CH₂ | H | Q2b | |
| 1820. | Cl | F | OCH₂CH₂ | H | Q3a | |
| 1821. | Cl | F | OCH₂CH₂ | H | Q3c | |
| 1822. | Cl | F | OCH₂CH₂ | H | Q4a | |
| 1823. | Cl | F | OCH₂CH₂ | H | QSa | |
| 1824. | Cl | F | OCH₂CH₂ | H | Q6a | |
| 1825. | Cl | F | OCH₂CH₂ | H | Q8a | |
| 1826. | Cl | F | OCH₂CH₂ | H | Q8d | |
| 1827. | Cl | F | OCH₂CH₂ | H | Q9a | |
| 1828. | Cl | F | OCH₂CH₂ | H | Q12 | |
| 1829. | Cl | F | OCH₂CH₂ | H | Q13 | |
| 1830. | Cl | F | OCH₂CH₂ | Me | Q1a | |
| 1831. | Cl | F | OCH₂CH₂ | Me | Q2a | |
| 1832. | Cl | F | OCH₂CH₂ | Me | Q2b | |
| 1833. | Cl | F | OCH₂CH₂ | Me | Q3a | |
| 1834. | Cl | F | OCH₂CH₂ | Me | Q6a | |
| 1835. | Cl | F | OCH₂CH₂ | Me | Q8d | |
| 1836. | Cl | F | OCH₂CH(Me) | H | Q1a | |
| 1837. | Cl | F | OCH₂CH₂CH₂ | H | Q1a | |
| 1838. | Cl | Cl | CH₂ | H | Q1a | 113-114 |
| 1839. | Cl | Cl | CH₂ | H | Q1b | |
| 1840. | Cl | Cl | CH₂ | H | Q1c | |
| 1841. | Cl | Cl | CH₂ | H | Q1d | |
| 1842. | Cl | Cl | CH₂ | H | Q1e | |
| 1843. | Cl | Cl | CH₂ | H | Q2a | |
| 1844. | Cl | Cl | CH₂ | H | Q2b | |
| 1845. | Cl | Cl | CH₂ | H | Q3a | 146-147 |
| 1846. | Cl | Cl | CH₂ | H | Q3b | |
| 1847. | Cl | Cl | CH₂ | H | Q3c | |
| 1848. | Cl | Cl | CH₂ | H | Q3d | |
| 1849. | Cl | Cl | CH₂ | H | Q3e | |
| 1850. | Cl | Cl | CH₂ | H | Q3f | |
| 1851. | Cl | Cl | CH₂ | H | Q3g | |
| 1852. | Cl | Cl | CH₂ | H | Q3h | |
| 1853. | Cl | Cl | CH₂ | H | Q3i | |
| 1854. | Cl | Cl | CH₂ | H | Q3j | |
| 1855. | Cl | Cl | CH₂ | H | Q3k | |
| 1856. | Cl | Cl | CH₂ | H | Q31 | |
| 1857. | Cl | Cl | CH₂ | H | Q3m | |
| 1858. | Cl | Cl | CH₂ | H | Q3n | |
| 1859. | Cl | Cl | CH₂ | H | Q3o | |
| 1860. | Cl | Cl | CH₂ | H | Q3p | |
| 1861. | Cl | Cl | CH₂ | H | Q3q | |
| 1862. | Cl | Cl | CH₂ | H | Q3r | |
| 1863. | Cl | Cl | CH₂ | H | Q3s | |
| 1864. | Cl | Cl | CH₂ | H | Q3t | |
| 1865. | Cl | Cl | CH₂ | H | Q3u | |
| 1866. | Cl | Cl | CH₂ | H | Q3v | |
| 1867. | Cl | Cl | CH₂ | H | Q3w | |
| 1868. | Cl | Cl | CH₂ | H | Q3x | |
| 1869. | Cl | Cl | CH₂ | H | Q3y | |
| 1870. | Cl | Cl | CH₂ | H | Q3z | |
| 1871. | Cl | Cl | CH₂ | H | Q3za | |
| 1872. | Cl | Cl | CH₂ | H | Q3zb | |
| 1873. | Cl | Cl | CH₂ | H | Q3zc | |
| 1874. | Cl | Cl | CH₂ | H | Q3zd | |
| 1875. | Cl | Cl | CH₂ | H | Q4a | |
| 1876. | Cl | Cl | CH₂ | H | Q4b | |
| 1877. | Cl | Cl | CH₂ | H | Q4c | |
| 1878. | Cl | Cl | CH₂ | H | Q4d | |
| 1879. | Cl | Cl | CH₂ | H | Q5a | |
| 1880. | Cl | Cl | CH₂ | H | Q5b | |
| 1881. | Cl | Cl | CH₂ | H | Q5c | |
| 1882. | Cl | Cl | CH₂ | H | Q6a | |
| 1883. | Cl | Cl | CH₂ | H | Q6b | |
| 1884. | Cl | Cl | CH₂ | H | Q7 | |
| 1885. | Cl | Cl | CH₂ | H | Q8a | |
| 1886. | Cl | Cl | CH₂ | H | Q8b | |
| 1887. | Cl | Cl | CH₂ | H | Q8c | |
| 1888. | Cl | Cl | CH₂ | H | Q8d | |
| 1889. | Cl | Cl | CH₂ | H | Q9a | |
| 1890. | Cl | Cl | CH₂ | H | Q9b | |
| 1891. | Cl | Cl | CH₂ | H | Q9c | |
| 1892. | Cl | Cl | CH₂ | H | Q9d | |
| 1893. | Cl | Cl | CH₂ | H | Q10a | |
| 1894. | Cl | Cl | CH₂ | H | Q11a | |
| 1895. | Cl | Cl | CH₂ | H | Q12 | |
| 1896. | Cl | Cl | CH₂ | H | Q13 | |
| 1897. | Cl | Cl | CH₂ | H | Q14 | |
| 1898. | Cl | Cl | CH₂ | H | Q15 | |
| 1899. | Cl | Cl | CH₂ | Me | Q1a | |
| 1900. | Cl | Cl | CH₂ | Me | Q1b | |
| 1901. | Cl | Cl | CH₂ | Me | Q1c | |
| 1902. | Cl | Cl | CH₂ | Me | Q1d | |
| 1903. | Cl | Cl | CH₂ | Me | Q1e | |
| 1904. | Cl | Cl | CH₂ | Me | Q2a | |
| 1905. | Cl | Cl | CH₂ | Me | Q2b | |
| 1906. | Cl | Cl | CH₂ | Me | Q3a | |
| 1907. | Cl | Cl | CH₂ | Me | Q3b | |
| 1908. | Cl | Cl | CH₂ | Me | Q3c | |
| 1909. | Cl | Cl | CH₂ | Me | Q3d | |
| 1910. | Cl | Cl | CH₂ | Me | Q3e | |
| 1911. | Cl | Cl | CH₂ | Me | Q3f | |
| 1912. | Cl | Cl | CH₂ | Me | Q3g | |
| 1913. | Cl | Cl | CH₂ | Me | Q3h | |
| 1914. | Cl | Cl | CH₂ | Me | Q3i | |
| 1915. | Cl | Cl | CH₂ | Me | Q3j | |
| 1916. | Cl | Cl | CH₂ | Me | Q3k | |
| 1917. | Cl | Cl | CH₂ | Me | Q31 | |
| 1918. | Cl | Cl | CH₂ | Me | Q3m | |
| 1919. | Cl | Cl | CH₂ | Me | Q3n | |
| 1920. | Cl | Cl | CH₂ | Me | Q3o | |
| 1921. | Cl | Cl | CH₂ | Me | Q3p | |
| 1922. | Cl | Cl | CH₂ | Me | Q3q | |
| 1923. | Cl | Cl | CH₂ | Me | Q3r | |
| 1924. | Cl | Cl | GB2 | Me | Q3s | |
| 1925. | Cl | Cl | GB2 | Me | Q3t | |
| 1926. | Cl | Cl | GB2 | Me | Q3u | |
| 1927. | Cl | Cl | GB2 | Me | Q3v | |
| 1928. | Cl | Cl | GB2 | Me | Q3w | |
| 1929. | Cl | Cl | CH₂ | Me | Q3x | |
| 1930. | Cl | Cl | GB2 | Me | Q3y | |
| 1931. | Cl | Cl | CH₂ | Me | Q3z | |
| 1932. | Cl | Cl | CH₂ | Me | Q3za | |
| 1933. | Cl | Cl | GB2 | Me | Q3zb | |
| 1934. | Cl | Cl | CH₂ | Me | Q3zc | |
| 1935. | Cl | Cl | CH₂ | Me | Q3zd | |
| 1936. | Cl | Cl | CH₂ | Me | Q4a | |
| 1937. | Cl | Cl | CH₂ | Me | Q4b | |
| 1938. | Cl | Cl | CH₂ | Me | Q4c | |
| 1939. | Cl | Cl | CH₂ | Me | Q4d | |
| 1940. | Cl | Cl | CH₂ | Me | Q5a | |
| 1941. | Cl | Cl | CH₂ | Me | Q6a | |
| 1942. | Cl | Cl | CH₂ | Me | Q6b | |
| 1943. | Cl | Cl | CH₂ | Me | Q7 | |
| 1944. | Cl | Cl | CH₂ | Me | Q8a | |
| 1945. | Cl | Cl | CH₂ | Me | Q8b | |
| 1946. | Cl | Cl | CH₂ | Me | Q8c | |
| 1947. | Cl | Cl | CH₂ | Me | Q8d | |
| 1948. | Cl | Cl | CH₂ | Me | Q9a | |
| 1949. | Cl | Cl | CH₂ | Me | Q9b | |
| 1950. | Cl | Cl | CH₂ | Me | Q9c | |
| 1951. | Cl | Cl | CH₂ | Me | Q9d | |
| 1952. | Cl | Cl | CH₂ | Me | Q10a | |
| 1953. | Cl | Cl | CH₂ | Me | Q11a | |
| 1954. | Cl | Cl | CH₂ | Me | Q12 | |
| 1955. | Cl | Cl | CH₂ | Me | Q13 | |
| 1956. | Cl | Cl | CH₂ | Me | Q14 | |
| 1957. | Cl | Cl | CH₂ | Me | Q15 | |
| 1958. | Cl | Cl | CH₂ | Et | Q1a | |
| 1959. | Cl | Cl | CH₂ | Et | Q1b | |
| 1960. | Cl | Cl | CH₂ | Et | Q1c | |
| 1961. | Cl | Cl | CH₂ | Et | Q1d | |
| 1962. | Cl | Cl | CH₂ | Et | Q1e | |
| 1963. | Cl | Cl | CH₂ | Et | Q2a | |
| 1964. | Cl | Cl | CH₂ | Et | Q2b | |
| 1965. | Cl | Cl | CH₂ | Et | Q3a | |
| 1966. | Cl | Cl | CH₂ | Et | Q3c | |
| 1967. | Cl | Cl | CH₂ | Et | Q4a | |
| 1968. | Cl | Cl | CH₂ | Et | Q5a | |
| 1969. | Cl | Cl | CH₂ | Et | Q6a | |
| 1970. | Cl | Cl | CH₂ | Et | Q8a | |
| 1971. | Cl | Cl | CH₂ | Et | Q8d | |
| 1972. | Cl | Cl | CH₂ | Et | Q9a | |
| 1973. | Cl | Cl | CH₂ | Et | Q12 | |
| 1974. | Cl | Cl | CH₂ | Et | Q13 | |
| 1975. | Cl | Cl | CH₂ | n-Pr | Q1a | |
| 1976. | Cl | Cl | CH₂ | n-Pr | Q3a | |
| 1977. | Cl | Cl | CH₂ | n-Pr | Q6a | |
| 1978. | Cl | Cl | CH₂ | SMe | Q1a | 106-109 |
| 1979. | Cl | Cl | CH₂ | SMe | Q1b | |
| 1980. | Cl | Cl | CH₂ | SMe | Q1c | |
| 1981. | Cl | Cl | CH₂ | SMe | Q1d | |
| 1982. | Cl | Cl | CH₂ | SMe | Q1e | |
| 1983. | Cl | Cl | CH₂ | SMe | Q2a | |
| 1984. | Cl | Cl | CH₂ | SMe | Q2b | |
| 1985. | GI | Cl | CH₂ | SMe | Q3a | |
| 1986. | Cl | Cl | CH₂ | SMe | Q3c | |
| 1987. | Cl | Cl | CH₂ | SMe | Q4a | |
| 1988. | Cl | Cl | CH₂ | SMe | Q5a | |
| 1989. | Cl | Cl | CH₂ | SMe | Q6a | |
| 1990. | Cl | Cl | CH₂ | SMe | Q8a | |
| 1991. | Cl | Cl | CH₂ | SMe | Q8d | |
| 1992. | Cl | Cl | CH₂ | SMe | Q9a | |
| 1993. | Cl | Cl | CH₂ | SMe | Q12 | |
| 1994. | Cl | Cl | CH₂ | SMe | Q13 | |
| 1995. | Cl | Cl | CH₂CH₂ | H | Q1a | |
| 1996. | Cl | Cl | CH₂CH₂ | H | Q1b | |
| 1997. | Cl | Cl | CH₂CH₂ | H | Q1c | |
| 1998. | Cl | Cl | CH₂CH₂ | H | Q1d | |
| 1999. | Cl | Cl | CH₂CH₂ | H | Q1e | |
| 2000. | Cl | Cl | CH₂CH₂ | H | Q2a | |
| 2001. | Cl | Cl | CH₂CH₂ | H | Q2b | |
| 2002. | Cl | Cl | CH₂CH₂ | H | Q3a | |
| 2003. | Cl | Cl | CH₂CH₂ | H | Q3c | |
| 2004. | Cl | Cl | CH₂CH₂ | H | Q4a | |
| 2005. | Cl | Cl | CH₂CH₂ | H | Q5a | |
| 2006. | Cl | Cl | CH₂CH₂ | H | Q6a | |
| 2007. | Cl | Cl | CH₂CH₂ | H | Q8a | |
| 2008. | Cl | Cl | CH₂CH₂ | H | Q8d | |
| 2009. | Cl | Cl | CH₂CH₂ | H | Q9a | |
| 2010. | Cl | Cl | CH₂CH₂ | H | Q12 | |
| 2011. | Cl | Cl | CH₂CH₂ | H | Q13 | |
| 2012. | Cl | Cl | CH₂CH₂ | Me | Q1a | |
| 2013. | Cl | Cl | CH₂CH₂ | Me | Q2a | |
| 2014. | Cl | Cl | CH₂CH₂ | Me | Q2b | |
| 2015. | Cl | Cl | CH₂CH₂ | Me | Q3a | |
| 2016. | Cl | Cl | CH₂CH₂ | Me | Q6a | |
| 2017. | Cl | Cl | OCH₂CH₂ | H | Q1a | 1.5892 |
| 2018. | Cl | Cl | OCH₂CH₂ | H | Q1b | 1.5725 |
| 2019. | Cl | Cl | OCH₂CH₂ | H | Q1c | |
| 2020. | Cl | Cl | OCH₂CH₂ | H | Q1d | 1.5735 |
| 2021. | Cl | Cl | OCH₂CH₂ | H | Q1e | |
| 2022. | Cl | Cl | OCH₂CH₂ | H | Q2a | 1.5860 |
| 2023. | Cl | Cl | OCH₂CH₂ | H | Q2b | 1.5779 |
| 2024. | Cl | Cl | OCH₂CH₂ | H | Q3a | 1.6290 |
| 2025. | Cl | Cl | OCH₂CH₂ | H | Q3b | |
| 2026. | Cl | Cl | OCH₂CH₂ | H | Q3c | |
| 2027. | Cl | Cl | OCH₂CH₂ | H | Q3d | |
| 2028. | Cl | Cl | OCH₂CH₂ | H | Q3e | |
| 2029. | Cl | Cl | OCH₂CH₂ | H | Q3f | |
| 2030. | Cl | Cl | OCH₂CH₂ | H | Q3g | |
| 2031. | Cl | Cl | OCH₂CH₂ | H | Q3h | |
| 2032. | Cl | Cl | OCH₂CH₂ | H | Q3i | |
| 2033. | Cl | Cl | OCH₂CH₂ | H | Q3j | |
| 2034. | Cl | Cl | OCH₂CH₂ | H | Q3k | |
| 2035. | Cl | Cl | OCH₂CH₂ | H | Q31 | |
| 2036. | Cl | Cl | OCH₂CH₂ | H | Q3m | |
| 2037. | Cl | Cl | OCH₂CH₂ | H | Q3n | |
| 2038. | Cl | Cl | OCH₂CH₂ | H | Q3o | |
| 2039. | Cl | Cl | OCH₂CH₂ | H | Q3p | |
| 2040. | Cl | Cl | OCH₂CH₂ | H | Q3q | |
| 2041. | Cl | Cl | OCH₂CH₂ | H | Q3r | |
| 2042. | Cl | Cl | OCH₂CH₂ | H | Q3s | |
| 2043. | Cl | Cl | OCH₂CH₂ | H | Q3t | |
| 2044. | Cl | Cl | OCH₂CH₂ | H | Q3u | |
| 2045. | Cl | Cl | OCH₂CH₂ | H | Q3v | |
| 2046. | Cl | Cl | OCH₂CH₂ | H | Q3w | |
| 2047. | Cl | Cl | OCH₂CH₂ | H | Q3x | |
| 2048. | Cl | Cl | OCH₂CH₂ | H | Q3y | |
| 2049. | Cl | Cl | OCH₂CH₂ | H | Q3z | |
| 2050. | Cl | Cl | OCH₂CH₂ | H | Q3za | |
| 2051. | Cl | Cl | OCH₂CH₂ | H | Q3zb | |
| 2052. | Cl | Cl | OCH₂CH₂ | H | Q3zc | |
| 2053. | Cl | Cl | OCH₂CH₂ | H | Q3zd | |
| 2054. | Cl | Cl | OCH₂CH₂ | H | Q4a | |
| 2055. | Cl | Cl | OCH₂CH₂ | H | Q4b | |
| 2056. | Cl | Cl | OCH₂CH₂ | H | Q4c | |
| 2057. | Cl | Cl | OCH₂CH₂ | H | Q4d | |
| 2058. | Cl | Cl | OCH₂CH₂ | H | Q5a | |
| 2059. | Cl | Cl | OCH₂CH₂ | H | Q5b | |
| 2060. | Cl | Cl | OCH₂CH₂ | H | Q5c | |
| 2061. | Cl | Cl | OCH₂CH₂ | H | Q5d | |
| 2062. | Cl | Cl | OCH₂CH₂ | H | Q5e | |
| 2063. | Cl | Cl | OCH₂CH₂ | H | Q6a | 1.5783 |
| 2064. | Cl | Cl | OCH₂CH₂ | H | Q6b | |
| 2065. | Cl | Cl | OCH₂CH₂ | H | Q7 | |
| 2066. | Cl | Cl | OCH₂CH₂ | H | Q8a | |
| 2067. | Cl | Cl | OCH₂CH₂ | H | Q8b | |
| 2068. | Cl | Cl | OCH₂CH₂ | H | Q8c | |
| 2069. | Cl | Cl | OCH₂CH₂ | H | Q8d | |
| 2070. | Cl | Cl | OCH₂CH₂ | H | Q9a | |
| 2071. | Cl | Cl | OCH₂CH₂ | H | Q9b | |
| 2072. | Cl | Cl | OCH₂CH₂ | H | Q9c | |
| 2073. | Cl | Cl | OCH₂CH₂ | H | Q9d | |
| 2074. | Cl | Cl | OCH₂CH₂ | H | Q10a | |
| 2075. | Cl | Cl | OCH₂CH₂ | H | Q11a | |
| 2076. | Cl | Cl | OCH₂CH₂ | H | Q12 | |
| 2077. | Cl | Cl | OCH₂CH₂ | H | Q13 | 1.5620 |
| 2078. | Cl | Cl | OCH₂CH₂ | H | Q14 | |
| 2079. | Cl | Cl | OCH₂CH₂ | H | Q15 | |
| 2080. | Cl | Cl | OCH₂CH₂ | Me | Q1a | 1.5839 |
| 2081. | Cl | Cl | OCH₂CH₂ | Me | Q1b | 1.5560 |
| 2082. | Cl | Cl | OCH₂CH₂ | Me | Q1c | |
| 2083. | Cl | Cl | OCH₂CH₂ | Me | Q1d | 1.5637 |
| 2084. | Cl | Cl | OCH₂CH₂ | Me | Q1e | |
| 2085. | Cl | Cl | OCH₂CH₂ | Me | Q2a | |
| 2086. | Cl | Cl | OCH₂CH₂ | Me | Q2b | |
| 2087. | Cl | Cl | OCH₂CH₂ | Me | Q3a | |
| 2088. | Cl | Cl | OCH₂CH₂ | Me | Q3b | |
| 2089. | Cl | Cl | OCH₂CH₂ | Me | Q3c | |
| 2090. | Cl | Cl | OCH₂CH₂ | Me | Q3d | |
| 2091. | Cl | Cl | OCH₂CH₂ | Me | Q3e | |
| 2092. | Cl | Cl | OCH₂CH₂ | Me | Q3f | |
| 2093. | Cl | Cl | OCH₂CH₂ | Me | Q3g | |
| 2094. | Cl | Cl | OCH₂CH₂ | Me | Q3h | |
| 2095. | Cl | Cl | OCH₂CH₂ | Me | Q3i | |
| 2096. | Cl | Cl | OCH₂CH₂ | Me | Q3j | |
| 2097. | Cl | Cl | OCH₂CH₂ | Me | Q3k | |
| 2098. | Cl | Cl | OCH₂CH₂ | Me | Q31 | |
| 2099. | Cl | Cl | OCH₂CH₂ | Me | Q3m | |
| 2100. | Cl | Cl | OCH₂CH₂ | Me | Q3n | |
| 2101. | Cl | Cl | OCH₂CH₂ | Me | Q3o | |
| 2102. | Cl | Cl | OCH₂CH₂ | Me | Q3p | |
| 2103. | Cl | Cl | OCH₂CH₂ | Me | Q3q | |
| 2104. | Cl | Cl | OCH₂CH₂ | Me | Q3r | |
| 2105. | Cl | Cl | OCH₂CH₂ | Me | Q3s | |
| 2106. | Cl | Cl | OCH₂CH₂ | Me | Q3t | |
| 2107. | Cl | Cl | OCH₂CH₂ | Me | Q3u | |
| 2108. | Cl | Cl | OCH₂CH₂ | Me | Q3v | |
| 2109. | Cl | Cl | OCH₂CH₂ | Me | Q3w | |
| 2110. | Cl | Cl | OCH₂CH₂ | Me | Q3x | |
| 2111. | Cl | Cl | OCH₂CH₂ | Me | Q3y | |
| 2112. | Cl | Cl | OCH₂CH₂ | Me | Q3z | |
| 2113. | Cl | Cl | OCH₂CH₂ | Me | Q3za | |
| 2114. | Cl | Cl | OCH₂CH₂ | Me | Q3zb | |
| 2115. | Cl | Cl | OCH₂CH₂ | Me | Q3zc | |
| 2116. | Cl | Cl | OCH₂CH₂ | Me | Q3zd | |
| 2117. | Cl | Cl | OCH₂CH₂ | Me | Q4a | |
| 2118. | Cl | Cl | OCH₂CH₂ | Me | Q4b | |
| 2119. | Cl | Cl | OCH₂CH₂ | Me | Q4c | |
| 2120. | Cl | Cl | OCH₂CH₂ | Me | Q4d | |
| 2121. | Cl | Cl | OCH₂CH₂ | Me | Q5a | |
| 2122. | Cl | Cl | OCH₂CH₂ | Me | Q5b | |
| 2123. | Cl | Cl | OCH₂CH₂ | Me | Q5c | |
| 2124. | Cl | Cl | OCH₂CH₂ | Me | Q6a | |
| 2125. | Cl | Cl | OCH₂CH₂ | Me | Q6b | |
| 2126. | Cl | Cl | OCH₂CH₂ | Me | Q7 | |
| 2127. | Cl | Cl | OCH₂CH₂ | Me | Q8a | |
| 2128. | Cl | Cl | OCH₂CH₂ | Me | Q8b | |
| 2129. | Cl | Cl | OCH₂CH₂ | Me | Q8c | |
| 2130. | Cl | Cl | OCH₂CH₂ | Me | Q8d | |
| 2131. | Cl | Cl | OCH₂CH₂ | Me | Q9a | |
| 2132. | Cl | Cl | OCH₂CH₂ | Me | Q9b | |
| 2133. | Cl | Cl | OCH₂CH₂ | Me | Q9c | |
| 2134. | Cl | Cl | OCH₂CH₂ | Me | Q9d | |
| 2135. | Cl | Cl | OCH₂CH₂ | Me | Q10a | |
| 2136. | Cl | Cl | OCH₂CH₂ | Me | Q11a | |
| 2137. | Cl | Cl | OCH₂CH₂ | Me | Q12 | |
| 2138. | Cl | Cl | OCH₂CH₂ | Me | Q13 | |
| 2139. | Cl | Cl | OCH₂CH₂ | Me | Q14 | |
| 2140. | Cl | Cl | OCH₂CH₂ | Me | Q15 | |
| 2141. | Cl | Cl | OCH₂CH₂ | Et | Q1a | |
| 2142. | Cl | Cl | OCH₂CH₂ | Et | Q1b | |
| 2143. | Cl | Cl | OCH₂CH₂ | Et | Q1c | |
| 2144. | Cl | Cl | OCH₂CH₂ | Et | Q1d | |
| 2145. | Cl | Cl | OCH₂CH₂ | Et | Q1e | |
| 2146. | Cl | Cl | OCH₂CH₂ | Et | Q2a | |
| 2147. | Cl | Cl | OCH₂CH₂ | Et | Q2b | |
| 2148. | Cl | Cl | OCH₂CH₂ | Et | Q3a | |
| 2149. | Cl | Cl | OCH₂CH₂ | Et | Q3c | |
| 2150. | Cl | Cl | OCH₂CH₂ | Et | Q4a | |
| 2151. | Cl | Cl | OCH₂CH₂ | Et | Q5a | |
| 2152. | Cl | Cl | OCH₂CH₂ | Et | Q6a | |
| 2153. | Cl | Cl | OCH₂CH₂ | Et | Q8a | |
| 2154. | Cl | Cl | OCH₂CH₂ | Et | Q8d | |
| 2155. | Cl | Cl | OCH₂CH₂ | Et | Q9a | |
| 2156. | Cl | Cl | OCH₂CH₂ | Et | Q12 | |
| 2157. | Cl | Cl | OCH₂CH₂ | Et | Q13 | |
| 2158. | Cl | Cl | OCH₂CH₂ | n-Pr | Q1a | |
| 2159. | Cl | Cl | OCH₂CH₂ | n-Pr | Q3a | |
| 2160. | Cl | Cl | OCH₂CH₂ | n-Pr | Q6a | |
| 2161. | Cl | Cl | OCH₂CH₂ | SMe | Q1a | |
| 2162. | Cl | Cl | OCH₂CH₂ | SMe | Q3a | |
| 2163. | Cl | Cl | OCH₂CH₂ | SMe | Q6a | |
| 2164. | Cl | Cl | OCH₂CH(Me) | H | Q1a | |
| 2165. | Cl | Cl | OCH₂CH(Me) | H | Q1b | |
| 2166. | Cl | Cl | OCH₂CH(Me) | H | Q1c | |
| 2167. | Cl | Cl | OCH₂CH(Me) | H | Q1d | |
| 2168. | Cl | Cl | OCH₂CH(Me) | H | Q1e | |
| 2169. | Cl | Cl | OCH₂CH(Me) | H | Q2a | |
| 2170. | Cl | Cl | OCH₂CH(Me) | H | Q2b | |
| 2171. | Cl | Cl | OCH₂CH(Me) | H | Q3a | |
| 2172. | Cl | Cl | OCH₂CH(Me) | H | Q3c | |
| 2173. | Cl | Cl | OCH₂CH(Me) | H | Q4a | |
| 2174. | Cl | Cl | OCH₂CH(Me) | H | Q5a | |
| 2175. | Cl | Cl | OCH₂CH(Me) | H | Q6a | |
| 2176. | Cl | Cl | OCH₂CH(Me) | H | Q8a | |
| 2177. | Cl | Cl | OCH₂CH(Me) | H | Q8d | |
| 2178. | Cl | Cl | OCH₂CH(Me) | H | Q9a | |
| 2179. | Cl | Cl | OCH₂CH(Me) | H | Q12 | |
| 2180. | Cl | Cl | OCH₂CH(Me) | H | Q13 | |
| 2181. | Cl | Cl | OCH₂CH(Me) | Me | Q1a | |
| 2182. | Cl | Cl | OCH₂CH(Me) | Me | Q3a | |
| 2183. | Cl | Cl | OCH₂CH(Me) | Me | Q6a | |
| 2184. | Cl | Cl | OCH₂CH₂CH₂ | H | Q1a | |
| 2185. | Cl | Cl | OCH₂CH₂CH₂ | H | Q1b | |
| 2186. | Cl | Cl | OCH₂CH₂CH₂ | H | Q1c | |
| 2187. | Cl | Cl | OCH₂CH₂CH₂ | H | Q1d | |
| 2188. | Cl | Cl | OCH₂CH₂CH₂ | H | Q1e | |
| 2189. | Cl | Cl | OCH₂CH₂CH₂ | H | Q2a | |
| 2190. | Cl | Cl | OCH₂CH₂CH₂ | H | Q2b | |
| 2191. | Cl | Cl | OCH₂CH₂CH₂ | H | Q3a | |
| 2192. | Cl | Cl | OCH₂CH₂CH₂ | H | Q3c | |
| 2193. | Cl | Cl | OCH₂CH₂CH₂ | H | Q4a | |
| 2194. | Cl | Cl | OCH₂CH₂CH₂ | H | Q5a | |
| 2195. | Cl | Cl | OCH₂CH₂CH₂ | H | Q6a | |
| 2196. | Cl | Cl | OCH₂CH₂CH₂ | H | Q8a | |
| 2197. | Cl | Cl | OCH₂CH₂CH₂ | H | Q8d | |
| 2198. | Cl | Cl | OCH₂CH₂CH₂ | H | Q9a | |
| 2199. | Cl | Cl | OCH₂CH₂CH₂ | H | Q12 | |
| 2200. | Cl | Cl | OCH₂CH₂CH₂ | H | Q13 | |
| 2201. | Cl | Cl | OCH₂CH₂CH₂ | Me | Q1a | |
| 2202. | Cl | Cl | OCH₂CH₂CH₂ | Me | Q3a | |
| 2203. | Cl | Cl | OCH₂CH₂CH₂ | Me | Q6a | |
| 2204. | Cl | Cl | CH(Me) | H | Q1a | |
| 2205. | Cl | Cl | CH(Me)CH₂ | H | Q1a | |
| 2206. | Cl | Cl | CH₂CH₂CH₂ | H | Q1a | |
| 2207. | Cl | Cl | OCH₂ | H | Q1a | |
| 2208. | Cl | Cl | OCH(Me)CH₂ | H | Q1a | |
| 2209. | Cl | Cl(Me) | OCH₂CH₂ | H | Q1a | |
| 2210. | Cl | Cl(Me) | OCH₂CH₂ | H | Q2a | |
| 2211. | Cl | Cl(Me) | OCH₂CH₂ | H | Q2b | |
| 2212. | Cl | Cl(Me) | OCH₂CH₂ | H | Q3a | |
| 2213. | Cl | Cl(Me) | OCH₂CH₂ | H | Q6a | |
| 2214. | Cl | Cl(Me) | OCH₂CH₂ | H | Q8d | |
| 2215. | Cl | C1(Me) | OCH₂CH₂ | Me | Q1a | |
| 2216. | Cl | Cl(Me) | OC.H₂CH₂ | Me | Q2a | |
| 2217. | Cl | Cl(Me) | OCH₂CH₂ | Me | Q2b | |
| 2218. | Cl | Cl(Me) | OCH₂CH₂ | Me | Q3a | |
| 2219. | Cl | Cl(Me) | OCH₂CH₂ | Me | Q6a | |
| 2220. | Cl | Cl(Me) | OCH₂CH₂ | Me | Q8d | |
| 2221. | Cl | Br | CH₂ | H | Q1a | |
| 2222. | Cl | Br | CH₂ | Me | Q1a | |
| 2223. | Cl | Br | OCH₂CH₂ | H | Q1a | |
| 2224. | Cl | Br | OCH₂CH₂ | H | Q1b | |
| 2225. | Cl | Br | OCH₂CH₂ | H | Q1c | |
| 2226. | Cl | Br | OCH₂CH₂ | H | Q1d | |
| 2227. | Cl | Br | OCH₂CH₂ | H | Q1e | |
| 2228. | Cl | Br | OCH₂CH₂ | H | Q2a | |
| 2229. | Cl | Br | OCH₂CH₂ | H | Q2b | |
| 2230. | Cl | Br | OCH₂CH₂ | H | Q3a | |
| 2231. | Cl | Br | OCH₂CH₂ | H | Q3c | |
| 2232. | Cl | Br | OCH₂CH₂ | H | Q4a | |
| 2233. | Cl | Br | OCH₂CH₂ | H | Q5a | |
| 2234. | Cl | Br | OCH₂CH₂ | H | Q6a | |
| 2235. | Cl | Br | OCH₂CH₂ | H | Q8a | |
| 2236. | Cl | Br | OCH₂CH₂ | H | Q8d | |
| 2237. | Cl | Br | OCH₂CH₂ | H | Q9a | |
| 2238. | Cl | Br | OCH₂CH₂ | H | Q12 | |
| 2239. | Cl | Br | OCH₂CH₂ | H | Q13 | |
| 2240. | Cl | Br | OCH₂CH₂ | Me | Q1a | |
| 2241. | Cl | Br | OCH₂CH₂ | Me | Q3a | |
| 2242. | Cl | Br | OCH₂CH₂ | Me | Q6a | |
| 2243. | Cl | Br | OCH₂CH(Me) | H | Q1a | |
| 2244. | Cl | Br | OCH₂CH₂CH₂ | H | Q1a | |
| 2245. | Cl | I | OCH₂CH₂ | H | Q1a | |
| 2246. | Cl | I | OCH₂CH₂ | Me | Q1a | |
| 2247. | Cl | Me | OCH₂CH₂ | H | Q1a | |
| 2248. | Cl | Me | OCH₂CH₂ | H | Q1b | |
| 2249. | Cl | Me | OCH₂CH₂ | H | Q1c | |
| 2250. | Cl | Me | OCH₂CH₂ | H | Q1d | |
| 2251. | Cl | Me | OCH₂CH₂ | H | Q1e | |
| 2252. | Cl | Me | OCH₂CH₂ | H | Q2a | |
| 2253. | Cl | Me | OCH₂CH₂ | H | Q2b | |
| 2254. | Cl | Me | OCH₂CH₂ | H | Q3a | |
| 2255. | Cl | Me | OCH₂CH₂ | H | Q3c | |
| 2256. | Cl | Me | OCH₂CH₂ | H | Q4a | |
| 2257. | Cl | Me | OCH₂CH₂ | H | Q5a | |
| 2258. | Cl | Me | OCH₂CH₂ | H | Q6a | |
| 2259. | Cl | Me | OCH₂CH₂ | H | Q8a | |
| 2260. | Cl | Me | OCH₂CH₂ | H | Q8d | |
| 2261. | Cl | Me | OCH₂CH₂ | H | Q9a | |
| 2262. | Cl | Me | OCH₂CH₂ | H | Q12 | |
| 2263. | Cl | Me | OCH₂CH₂ | H | Q13 | |
| 2264. | Cl | Me | OCH₂CH₂ | Me | Q1a | |
| 2265. | Cl | Me | OCH₂CH₂ | Me | Q2a | |
| 2266. | Cl | Me | OCH₂CH₂ | Me | Q2b | |
| 2267. | Cl | Me | OCH₂CH₂ | Me | Q3a | |
| 2268. | Cl | Me | OCH₂CH₂ | Me | Q6a | |
| 2269. | Cl | Me | OCH₂CH₂ | Me | Q8d | |
| 2270. | Cl | Me | OCH₂CH(Me) | H | Q1a | |
| 2271. | Cl | Me | OCH₂CH₂CH₂ | H | Q1a | |
| 2272. | Cl | CF3 | OCH₂CH₂ | H | Q1a | |
| 2273. | Cl | CF3 | OCH₂CH₂ | H | Q2a | |
| 2274. | Cl | CF3 | OCH₂CH₂ | H | Q2b | |
| 2275. | Cl | CF3 | OCH₂CH₂ | H | Q3a | |
| 2276. | Cl | CF3 | OCH₂CH₂ | Me | Q1a | |
| 2277. | Cl | CF3 | OCH₂CH₂ | Me | Q3a | |
| 2278. | Cl | CF3 | OCH₂CH₂CH₂ | H | Q1a | |
| 2279. | Cl | CF3 | OCH₂CH₂CH₂ | Me | Q1a | |
| 2280. | Cl | OMe | CH₂ | H | Q1a | 114-116 |
| 2281. | Cl | OMe | CH₂ | H | Q3a | |
| 2282. | Cl | OMe | CH₂ | Me | Q1a | |
| 2283. | Cl | OSO₂Me | CH₂ | H | Q1a | |
| 2284. | Cl | OSO₂Et | CH₂ | H | Q1a | |
| 2285. | Cl | SMe | CH₂ | H | Q1a | |
| 2286. | Cl | SMe | CH₂ | H | Q3a | |
| 2287. | Cl | SMe | CH₂ | Me | Q1a | |
| 2288. | Cl | SMe | OCH₂CH₂ | H | Q1a | |
| 2289. | Cl | SMe | OCH₂CH₂ | H | Q2a | |
| 2290. | Cl | SMe | OCH₂CH₂ | H | Q2b | |
| 2291. | Cl | SMe | OCH₂CH₂ | H | Q3a | |
| 2292. | Cl | SMe | OCH₂CH₂ | H | Q6a | |
| 2293. | Cl | SMe | OCH₂CH₂ | H | Q8d | |
| 2294. | Cl | SMe | OCH₂CH₂ | Me | Q1a | |
| 2295. | Cl | SMe | OCH₂CH₂ | Me | Q3a | |
| 2296. | Cl | SMe | OCH₂CH(Me) | H | Q1a | |
| 2297. | Cl | SMe | OCH₂CH₂CH₂ | H | Q1a | |
| 2298. | Cl | SEt | CH₂ | H | Q1a | |
| 2299. | Cl | SEt | OCH₂CH₂ | H | Q1a | |
| 2300. | Cl | SEt | OCH₂CH₂ | Me | Q1a | |
| 2301. | Cl | SO₂Me | CH₂ | H | Q1a | 194-197 |
| 2302. | Cl | SO₂Me | CH₂ | H | Q1b | |
| 2303. | Cl | SO₂Me | CH₂ | H | Q1c | |
| 2304. | Cl | SO₂Me | CH₂ | H | Q1d | |
| 2305. | Cl | SO₂Me | CH₂ | H | Q1e | |
| 2306. | Cl | SO₂Me | CH₂ | H | Q2a | |
| 2307. | Cl | SO₂Me | CH₂ | H | Q2b | |
| 2308. | Cl | SO₂Me | CH₂ | H | Q3a | 93-97 |
| 2309. | Cl | SO₂Me | CH₂ | H | Q3b | |
| 2310. | Cl | SO₂Me | CH₂ | H | Q3c | 182-183 |
| 2311. | Cl | SO₂Me | CH₂ | H | Q3d | |
| 2312. | Cl | SO₂Me | CH₂ | H | Q3e | 165-167 |
| 2313. | Cl | SO₂Me | CH₂ | H | Q3f | |
| 2314. | Cl | SO₂Me | CH₂ | H | Q3g | |
| 2315. | Cl | SO₂Me | CH₂ | H | Q3h | |
| 2316. | Cl | SO₂Me | CH₂ | H | Q3i | |
| 2317. | Cl | SO₂Me | CH₂ | H | Q3j | |
| 2318. | Cl | SO₂Me | CH₂ | H | Q3k | |
| 2319. | Cl | SO₂Me | CH₂ | H | Q31 | |
| 2320. | Cl | SO₂Me | CH₂ | H | Q3m | |
| 2321. | Cl | SO₂Me | CH₂ | H | Q3n | |
| 2322. | Cl | SO₂Me | CH₂ | H | Q3o | |
| 2323. | Cl | SO₂Me | CH₂ | H | Q3p | |
| 2324. | Cl | SO₂Me | CH₂ | H | Q3q | |
| 2325. | Cl | SO₂Me | CH₂ | H | Q3r | |
| 2326. | Cl | SO₂Me | CH₂ | H | Q3s | |
| 2327. | Cl | SO₂Me | CH₂ | H | Q3t | 222-227 |
| 2328. | Cl | SO₂Me | CH₂ | H | Q3u | |
| 2329. | Cl | SO₂Me | CH₂ | H | Q3v | 172-174 |
| 2330. | Cl | SO₂Me | CH₂ | H | Q3w | |
| 2331. | Cl | SO₂Me | CH₂ | H | Q3x | |
| 2332. | Cl | SO₂Me | CH₂ | H | Q3y | |
| 2333. | Cl | SO₂Me | CH₂ | H | Q3z | |
| 2334. | Cl | SO₂Me | CH₂ | H | Q3za | |
| 2335. | Cl | SO₂Me | CH₂ | H | Q3zb | 213-214 |
| 2336. | Cl | SO₂Me | CH₂ | H | Q3zc | |
| 2337. | Cl | SO₂Me | CH₂ | H | Q3zd | |
| 2338. | Cl | SO₂Me | CH₂ | H | Q4a | 146-148 |
| 2339. | Cl | SO₂Me | CH₂ | H | Q4b | |
| 2340. | Cl | SO₂Me | CH₂ | H | Q4c | |
| 2341. | Cl | SO₂Me | CH₂ | H | Q4d | |
| 2342. | Cl | SO₂Me | CH₂ | H | Q5a | |
| 2343. | Cl | SO₂Me | CH₂ | H | Q5b | |
| 2344. | Cl | SO₂Me | CH₂ | H | Q5c | |
| 2345. | Cl | SO₂Me | CH₂ | H | Q5d | |
| 2346. | Cl | SO₂Me | CH₂ | H | Q5e | |
| 2347. | Cl | SO₂Me | CH₂ | H | Q6a | 70-75 |
| 2348. | Cl | SO₂Me | CH₂ | H | Q6b | |
| 2349. | Cl | SO₂Me | CH₂ | H | Q7 | |
| 2350. | Cl | SO₂Me | CH₂ | H | Q8a | |
| 2351. | Cl | SO₂Me | CH₂ | H | Q8b | |
| 2352. | Cl | SO₂Me | CH₂ | H | Q8c | |
| 2353. | Cl | SO₂Me | CH₂ | H | Q8d | |
| 2354. | Cl | SO₂Me | CH₂ | H | Q9a | |
| 2355. | Cl | SO₂Me | CH₂ | H | Q9b | |
| 2356. | Cl | SO₂Me | CH₂ | H | Q9c | |
| 2357. | Cl | SO₂Me | CH₂ | H | Q9d | |
| 2358. | Cl | SO₂Me | CH₂ | H | Q10a | |
| 2359. | Cl | SO₂Me | CH₂ | H | Q11a | |
| 2360. | Cl | SO₂Me | GEl2 | H | Q12 | |
| 2361. | Cl | SO₂Me | CH₂ | H | Q13 | |
| 2362. | Cl | SO₂Me | CH₂ | H | Q14 | |
| 2363. | Cl | SO₂Me | CH₂ | H | Q15 | |
| 2364. | Cl | SO₂Me | CH₂ | Me | Q1a | 63-66 |
| 2365. | Cl | SO₂Me | CH₂ | Me | Q1b | |
| 2366. | Cl | SO₂Me | CH₂ | Me | Q1c | |
| 2367. | Cl | SO₂Me | CH₂ | Me | Q1d | |
| 2368. | Cl | SO₂Me | CH₂ | Me | Q1e | |
| 2369. | Cl | SO₂Me | CH₂ | Me | Q2a | |
| 2370. | Cl | SO₂Me | CH₂ | Me | Q2b | |
| 2371. | Cl | SO₂Me | CH₂ | Me | Q3a | |
| 2372. | Cl | SO₂Me | CH₂ | Me | Q3b | |
| 2373. | Cl | SO₂Me | CH₂ | Me | Q3c | |
| 2374. | Cl | SO₂Me | CH₂ | Me | Q3d | |
| 2375. | Cl | SO₂Me | CH₂ | Me | Q3e | |
| 2376. | Cl | SO₂Me | CH₂ | Me | Q3f | |
| 2377. | Cl | SO₂Me | CH₂ | Me | Q3g | |
| 2378. | Cl | SO₂Me | CH₂ | Me | Q3h | |
| 2379. | Cl | SO₂Me | CH₂ | Me | Q3i | |
| 2380. | Cl | SO₂Me | CH₂ | Me | Q3j | |
| 2381. | Cl | SO₂Me | CH₂ | Me | Q3k | |
| 2382. | Cl | SO₂Me | CH₂ | Me | Q31 | |
| 2383. | Cl | SO₂Me | CH₂ | Me | Q3m | |
| 2384. | Cl | SO₂Me | CH₂ | Me | Q3n | |
| 2385. | Cl | SO₂Me | CH₂ | Me | Q3o | |
| 2386. | Cl | SO₂Me | CH₂ | Me | Q3p | |
| 2387. | Cl | SO₂Me | CH₂ | Me | Q3q | |
| 2388. | Cl | SO₂Me | CH₂ | Me | Q3r | |
| 2389. | Cl | SO₂Me | CH₂ | Me | Q3s | |
| 2390. | Cl | SO₂Me | CH₂ | Me | Q3t | |
| 2391. | Cl | SO₂Me | CH₂ | Me | Q3u | |
| 2392. | Cl | SO₂Me | CH₂ | Me | Q3v | |
| 2393. | Cl | SO₂Me | CH₂ | Me | Q3w | |
| 2394. | Cl | SO₂Me | CH₂ | Me | Q3x | |
| 2395. | Cl | SO₂Me | CH₂ | Me | Q3y | |
| 2396. | Cl | SO₂Me | CH₂ | Me | Q3z | |
| 2397. | Cl | SO₂Me | CH₂ | Me | Q3za | |
| 2398. | Cl | SO₂Me | CH₂ | Me | Q3zb | |
| 2399. | Cl | SO₂Me | CH₂ | Me | Q3zc | |
| 2400. | Cl | SO₂Me | CH₂ | Me | Q3zd | |
| 2401. | Cl | SO₂Me | CH₂ | Me | Q4a | |
| 2402. | Cl | SO₂Me | CH₂ | Me | Q4b | |
| 2403. | Cl | SO₂Me | CH₂ | Me | Q4c | |
| 2404. | Cl | SO₂Me | CH₂ | Me | Q4d | |
| 2405. | Cl | SO₂Me | CH₂ | Me | Q5a | |
| 2406. | Cl | SO₂Me | CH₂ | Me | Q5b | |
| 2407. | Cl | SO₂Me | CH₂ | Me | Q5c | |
| 2408. | Cl | SO₂Me | CH₂ | Me | Q6a | |
| 2409. | Cl | SO₂Me | CH₂ | Me | Q6b | |
| 2410. | Cl | SO₂Me | CH₂ | Me | Q7 | |
| 2411. | Cl | SO₂Me | CH₂ | Me | Q8a | |
| 2412. | Cl | SO₂Me | CH₂ | Me | Q8b | |
| 2413. | Cl | SO₂Me | CH₂ | Me | Q8c | |
| 2414. | Cl | SO₂Me | CH₂ | Me | Q8d | |
| 2415. | Cl | SO₂Me | CH₂ | Me | Q9a | |
| 2416. | Cl | SO₂Me | CH₂ | Me | Q9b | |
| 2417. | Cl | SO₂Me | CH₂ | Me | Q9c | |
| 2418. | Cl | SO₂Me | CH₂ | Me | Q9d | |
| 2419. | Cl | SO₂Me | CH₂ | Me | Q10a | |
| 2420. | Cl | SO₂Me | CH₂ | Me | Q11a | |
| 2421. | Cl | SO₂Me | CH₂ | Me | Q12 | |
| 2422. | Cl | SO₂Me | CH₂ | Me | Q13 | |
| 2423. | Cl | SO₂Me | CH₂ | Et | Q1a | |
| 2424. | Cl | SO₂Me | CH₂ | Et | Q1b | |
| 2425. | Cl | SO₂Me | CH₂ | Et | Q1c | |
| 2426. | Cl | SO₂Me | CH₂ | Et | Q1d | |
| 2427. | Cl | SO₂Me | CH₂ | Et | Q1e | |
| 2428. | Cl | SO₂Me | CH₂ | Et | Q2a | |
| 2429. | Cl | SO₂Me | CH₂ | Et | Q2b | |
| 2430. | Cl | SO₂Me | CH₂ | Et | Q3a | |
| 2431. | Cl | SO₂Me | CH₂ | Et | Q3c | |
| 2432. | Cl | SO₂Me | CH₂ | Et | Q4a | |
| 2433. | Cl | SO₂Me | CH₂ | Et | Q5a | |
| 2434. | Cl | SO₂Me | CH₂ | Et | Q6a | |
| 2435. | Cl | SO₂Me | CH₂ | Et | Q8a | |
| 2436. | Cl | SO₂Me | CH₂ | Et | Q8d | |
| 2437. | Cl | SO₂Me | CH₂ | Et | Q9a | |
| 2438. | Cl | SO₂Me | CH₂ | Et | Q12 | |
| 2439. | Cl | SO₂Me | CH₂ | Et | Q13 | |
| 2440. | Cl | SO₂Me | CH₂ | n-Pr | Q1a | |
| 2441. | Cl | SO₂Me | CH₂ | n-Pr | Q1b | |
| 2442. | Cl | SO₂Me | CH₂ | n-Pr | Q1c | |
| 2443. | Cl | SO₂Me | CH₂ | n-Pr | Q1d | |
| 2444. | Cl | SO₂Me | CH₂ | n-Pr | Q1e | |
| 2445. | Cl | SO₂Me | CH₂ | n-Pr | Q2a | |
| 2446. | Cl | SO₂Me | CH₂ | n-Pr | Q2b | |
| 2447. | Cl | SO₂Me | CH₂ | n-Pr | Q3a | |
| 2448. | Cl | SO₂Me | CH₂ | n-Pr | Q3c | |
| 2449. | Cl | SO₂Me | CH₂ | n-Pr | Q4a | |
| 2450. | Cl | SO₂Me | CH₂ | n-Pr | Q5a | |
| 2451. | Cl | SO₂Me | CH₂ | n-Pr | Q6a | |
| 2452. | Cl | SO₂Me | CH₂ | n-Pr | Q8a | |
| 2453. | Cl | SO₂Me | CH₂ | n-Pr | Q8d | |
| 2454. | Cl | SO₂Me | CH₂ | n-Pr | Q9a | |
| 2455. | Cl | SO₂Me | CF2 | n-Pr | Q12 | |
| 2456. | Cl | SO₂Me | CH₂ | n-Pr | Q13 | |
| 2457. | Cl | SO₂Me | CF2 | SMe | Q1a | 76-81 |
| 2458. | Cl | SO₂Me | CF2 | SMe | Q1b | |
| 2459. | Cl | SO₂Me | CH₂ | SMe | Q1c | |
| 2460. | Cl | SO₂Me | CF2 | SMe | Q1d | |
| 2461. | Cl | SO₂Me | CF2 | SMe | Q1e | |
| 2462. | Cl | SO₂Me | CF2 | SMe | Q2a | |
| 2463. | Cl | SO₂Me | CH₂ | SMe | Q2b | |
| 2464. | Cl | SO₂Me | CH₂ | SMe | Q3a | |
| 2465. | Cl | SO₂Me | CH₂ | SMe | Q3c | |
| 2466. | Cl | SO₂Me | CF2 | SMe | Q4a | |
| 2467. | Cl | SO₂Me | CF2 | SMe | Q5a | |
| 2468. | Cl | SO₂Me | CF2 | SMe | Q6a | |
| 2469. | Cl | SO₂Me | CF2 | SMe | Q8a | |
| 2470. | Cl | SO₂Me | CH₂ | SMe | Q8d | |
| 2471. | Cl | SO₂Me | CH₂ | SMe | Q9a | |
| 2472. | Cl | SO₂Me | CF2 | SMe | Q12 | |
| 2473. | Cl | SO₂Me | CH₂ | SMe | Q13 | |
| 2474. | Cl | SO₂Me | CH₂ | n-Bu | Q1a | |
| 2475. | Cl | SO₂Me | CF2 | n-Pen | Q1a | |
| 2476. | Cl | SO₂Me | CH₂ | n-Hex | Q1a | |
| 2477. | Cl | SO₂Me | CH₂ | cyclo-Pr | Q1a | |
| 2478. | Cl | SO₂Me | CH₂ | cyclo-Bu | Q1a | |
| 2479. | Cl | SO₂Me | CH₂ | cyclo-pen | Q1a | |
| 2480. | Cl | SO₂Me | CF2 | cyclo-Hex | Q1a | |
| 2481. | Cl | SO₂Me | CH₂ | CH═CH₂ | Q1a | |
| 2482. | Cl | SO₂Me | CF2 | CF2CF═CH₂ | Q1a | |
| 2483. | Cl | SO₂Me | CF2 | C≡CH | Q1a | |
| 2484. | Cl | SO₂Me | CF2 | CF3 | Q1a | |
| 2485. | Cl | SO₂Me | CH₂ | CH₂CH₂C1 | Q1a | |
| 2486. | Cl | SO₂Me | CH₂ | CH₂CH₂CH₂Br | Q1a | |
| 2487. | Cl | SO₂Me | CH₂ | S-n-Pr | Q1a | |
| 2488. | Cl | SO₂Me | CH₂ | Ph | Q1a | |
| 2489. | Cl | SO₂Me | CH₂ | 2-Cl-Ph | Q1a | |
| 2490. | Cl | SO₂Me | CH₂ | 3-Cl-Ph | Q1a | |
| 2491. | Cl | SO₂Me | CH₂ | 2-Me-Ph | Q1a | |
| 2492. | Cl | SO₂Me | CH₂ | 4-Me-Ph | Q1a | |
| 2493. | Cl | SO₂Me | CH₂ | 3-Et-Ph | Q1a | |
| 2494. | Cl | SO₂Me | CH₂ | 4-n-Pr-Ph | Q1 a | |
| 2495. | Cl | SO₂Me | CH₂ | 3-CF3-Ph | Q1a | |
| 2496. | Cl | SO₂Me | CH₂ | 3-CH₂CF3-Ph | Q1a | |
| 2497. | Cl | SO₂Me | CH₂ | 3-NO₂-Ph | Q1a | |
| 2498. | Cl | SO₂Me | CH₂CH₂ | H | Q1a | |
| 2499. | Cl | SO₂Me | CH₂CH₂ | H | Q1b | |
| 2500. | Cl | SO₂Me | CH₂CH₂ | H | Q1c | |
| 2501. | Cl | SO₂Me | CH₂CH₂ | H | Q1d | |
| 2502. | Cl | SO₂Me | CH₂CH₂ | H | Q1e | |
| 2503. | Cl | SO₂Me | CH₂CH₂ | H | Q2a | |
| 2504. | Cl | SO₂Me | CH₂CH₂ | H | Q2b | |
| 2505. | Cl | SO₂Me | CH₂CH₂ | H | Q3a | |
| 2506. | Cl | SO₂Me | CH₂CH₂ | H | Q3c | |
| 2507. | Cl | SO₂Me | CH₂CH₂ | H | Q4a | |
| 2508. | Cl | SO₂Me | CH₂CH₂ | H | Q5a | |
| 2509. | Cl | SO₂Me | CH₂CH₂ | H | Q6a | |
| 2510. | Cl | SO₂Me | CH₂CH₂ | H | Q8a | |
| 2511. | Cl | SO₂Me | CH₂CH₂ | H | Q8d | |
| 2512. | Cl | SO₂Me | CH₂CH₂ | H | Q9a | |
| 2513. | Cl | SO₂Me | CH₂CH₂ | H | Q12 | |
| 2514. | Cl | SO₂Me | CH₂CH₂ | H | Q13 | |
| 2515. | Cl | SO₂Me | CH₂CH₂ | Me | Q1a | |
| 2516. | Cl | SO₂Me | CH₂CH₂ | Me | Q2a | |
| 2517. | Cl | SO₂Me | CH₂CH₂ | Me | Q2b | |
| 2518. | Cl | SO₂Me | CH₂CH₂ | Me | Q3a | |
| 2519. | Cl | SO₂Me | CH₂CH₂ | Me | Q6a | |
| 2520. | Cl | SO₂Me | CHZCH₂ | Me | Q8d | |
| 2521. | Cl | SO₂Me | OCH₂CH₂ | H | Q1a | 69-73 |
| 2522. | Cl | SO₂Me | OCH₂CH₂ | H | Q1b | |
| 2523. | Cl | SO₂Me | OCH₂CH₂ | H | Q1c | |
| 2524. | Cl | SO₂Me | OCH₂CH₂ | H | Q1d | |
| 2525. | Cl | SO₂Me | OCH₂CH₂ | H | Q1e | |
| 2526. | Cl | SO₂Me | OCH₂CH₂ | H | Q2a | |
| 2527. | Cl | SO₂Me | OCH₂CH₂ | H | Q2b | |
| 2528. | Cl | SO₂Me | OCH₂CH₂ | H | Q3a | |
| 2529. | Cl | SO₂Me | OCH₂CH₂ | H | Q3b | |
| 2530. | Cl | SO₂Me | OCH₂CH₂ | H | Q3c | |
| 2531. | Cl | SO₂Me | OCH₂CH₂ | H | Q3d | |
| 2532. | Cl | SO₂Me | OCH₂CH₂ | H | Q3e | |
| 2533. | Cl | SO₂Me | OCH₂CH₂ | H | Q3f | |
| 2534. | Cl | SO₂Me | OCH₂CH₂ | H | Q3g | |
| 2535. | Cl | SO₂Me | OCH₂CH₂ | H | Q3h | |
| 2536. | Cl | SO₂Me | OCH₂CH₂ | H | Q3i | |
| 2537. | Cl | SO₂Me | OCH₂CH₂ | H | Q3j | |
| 2538. | Cl | SO₂Me | OCH₂CH₂ | H | Q3k | |
| 2539. | Cl | SO₂Me | OCH₂CH₂ | H | Q31 | |
| 2540. | Cl | SO₂Me | OCH₂CH₂ | H | Q3m | |
| 2541. | Cl | SO₂Me | OCH₂CH₂ | H | Q3n | |
| 2542. | Cl | SO₂Me | OCH₂CH₂ | H | Q3o | |
| 2543. | Cl | SO₂Me | OCH₂CH₂ | H | Q3p | |
| 2544. | Cl | SO₂Me | OCH₂CH₂ | H | Q3q | |
| 2545. | Cl | SO₂Me | OCH₂CH₂ | H | Q3r | |
| 2546. | Cl | SO₂Me | OCH₂CH₂ | H | Q3s | |
| 2547. | Cl | SO₂Me | OCH₂CH₂ | H | Q3t | |
| 2548. | Cl | SO₂Me | OCH₂CH₂ | H | Q3u | |
| 2549. | Cl | SO₂Me | OCH₂CH₂ | H | Q3v | |
| 2550. | Cl | SO₂Me | OCH₂CH₂ | H | Q3w | |
| 2551. | Cl | SO₂Me | OCH₂CH₂ | H | Q3x | |
| 2552. | Cl | SO₂Me | OCH₂CH₂ | H | Q3y | |
| 2553. | Cl | SO₂Me | OCH₂CH₂ | H | Q3z | |
| 2554. | Cl | SO₂Me | OCH₂CH₂ | H | Q3za | |
| 2555. | Cl | SO₂Me | OCH₂CH₂ | H | Q3zb | |
| 2556. | Cl | SO₂Me | OCH₂CH₂ | H | Q3zc | |
| 2557. | Cl | SO₂Me | OCH₂CH₂ | H | Q3zd | |
| 2558. | Cl | SO₂Me | OCH₂CH₂ | H | Q4a | |
| 2559. | Cl | SO₂Me | OCH₂CH₂ | H | Q4b | |
| 2560. | Cl | SO₂Me | OCH₂CH₂ | H | Q4c | |
| 2561. | Cl | SO₂Me | OCH₂CH₂ | H | Q4d | |
| 2562. | Cl | SO₂Me | OCH₂CH₂ | H | Q5a | |
| 2563. | Cl | SO₂Me | OCH₂CH₂ | H | Q5b | |
| 2564. | Cl | SO₂Me | OCH₂CH₂ | H | Q5c | |
| 2565. | Cl | SO₂Me | OCH₂CH₂ | H | QSd | |
| 2566. | Cl | SO₂Me | OCH₂CH₂ | H | QSe | |
| 2567. | Cl | SO₂Me | OCH₂CH₂ | H | Q6a | |
| 2568. | Cl | SO₂Me | OCH₂CH₂ | H | Q6b | |
| 2569. | Cl | SO₂Me | OCH₂CH₂ | H | Q7 | |
| 2570. | Cl | SO₂Me | OCH₂CH₂ | H | Q8a | |
| 2571. | Cl | SO₂Me | OCH₂CH₂ | H | QSb | |
| 2572. | Cl | SO₂Me | OCH₂CH₂ | H | Q8c | |
| 2573. | Cl | SO₂Me | OCH₂CH₂ | H | Q8d | |
| 2574. | Cl | SO₂Me | OCH₂CH₂ | H | Q9a | |
| 2575. | Cl | SO₂Me | OCH₂CH₂ | H | Q9b | |
| 2576. | Cl | SO₂Me | OCH₂CH₂ | H | Q9c | |
| 2577. | Cl | SO₂Me | OCH₂CH₂ | H | Q9d | |
| 2578. | Cl | SO₂Me | OCH₂CH₂ | H | Q10a | |
| 2579. | Cl | SO₂Me | OCH₂CH₂ | H | Q11a | |
| 2580. | Cl | SO₂Me | OCH₂CH₂ | H | Q12 | |
| 2581. | Cl | SO₂Me | OCH₂CH₂ | H | Q13 | |
| 2582. | Cl | SO₂Me | OCH₂CH₂ | Me | Q1a | 68-69 |
| 2583. | Cl | SO₂Me | OCH₂CH₂ | Me | Q1b | |
| 2584. | Cl | SO₂Me | OCH₂CH₂ | Me | Q1c | |
| 2585. | Cl | SO₂Me | OCH₂CH₂ | Me | Q1d | |
| 2586. | Cl | SO₂Me | OCH₂CH₂ | Me | Q1e | |
| 2587. | Cl | SO₂Me | OCH₂CH₂ | Me | Q2a | |
| 2588. | Cl | SO₂Me | OCH₂CH₂ | Me | Q2b | |
| 2589. | Cl | SO₂Me | OCH₂CH₂ | Me | Q3a | |
| 2590. | Cl | SO₂Me | OCH₂CH₂ | Me | Q3b | |
| 2591. | Cl | SO₂Me | OCH₂CH₂ | Me | Q3c | |
| 2592. | Cl | SO₂Me | OCH₂CH₂ | Me | Q3d | |
| 2593. | Cl | SO₂Me | OCH₂CH₂ | Me | Q3e | |
| 2594. | Cl | SO₂Me | OCH₂CH₂ | Me | Q3f | |
| 2595. | Cl | SO₂Me | OCH₂CH₂ | Me | Q3g | |
| 2596. | Cl | SO₂Me | OCH₂CH₂ | Me | Q3h | |
| 2597. | Cl | SO₂Me | OCH₂CH₂ | Me | Q3i | |
| 2598. | Cl | SO₂Me | OCH₂CH₂ | Me | Q3j | |
| 2599. | Cl | SO₂Me | OCH₂CH₂ | Me | Q3k | |
| 2600. | Cl | SO₂Me | OCH₂CH₂ | Me | Q31 | |
| 2601. | Cl | SO₂Me | OCH₂CH₂ | Me | Q3m | |
| 2602. | Cl | SO₂Me | OCH₂CH₂ | Me | Q3n | |
| 2603. | Cl | SO₂Me | OCH₂CH₂ | Me | Q3o | |
| 2604. | Cl | SO₂Me | OCH₂CH₂ | Me | Q3p | |
| 2605. | Cl | SO₂Me | OCH₂CH₂ | Me | Q3q | |
| 2606. | Cl | SO₂Me | OCH₂CH₂ | Me | Q3r | |
| 2607. | Cl | SO₂Me | OCH₂CH₂ | Me | Q3s | |
| 2608. | Cl | SO₂Me | OCH₂CH₂ | Me | Q3t | |
| 2609. | Cl | SO₂Me | OCH₂CH₂ | Me | Q3u | |
| 2610. | Cl | SO₂Me | OCH₂CH₂ | Me | Q3v | |
| 2611. | Cl | SO₂Me | OCH₂CH₂ | Me | Q3w | |
| 2612. | Cl | SO₂Me | OCH₂CH₂ | Me | Q3x | |
| 2613. | Cl | SO₂Me | OCH₂CH₂ | Me | Q3y | |
| 2614. | Cl | SO₂Me | OCH₂CH₂ | Me | Q3z | |
| 2615. | Cl | SO₂Me | OCH₂CH₂ | Me | Q3za | |
| 2616. | Cl | SO₂Me | OCH₂CH₂ | Me | Q3zb | |
| 2617. | Cl | SO₂Me | OCH₂CH₂ | Me | Q3zc | |
| 2618. | Cl | SO₂Me | OCH₂CH₂ | Me | Q3zd | |
| 2619. | Cl | SO₂Me | OCH₂CH₂ | Me | Q4a | |
| 2620. | Cl | SO₂Me | OCH₂CH₂ | Me | Q4b | |
| 2621. | Cl | SO₂Me | OCH₂CH₂ | Me | Q4c | |
| 2622. | Cl | SO₂Me | OCH₂CH₂ | Me | Q4d | |
| 2623. | Cl | SO₂Me | OCH₂CH₂ | Me | Q5a | |
| 2624. | Cl | SO₂Me | OCH₂CH₂ | Me | QSb | |
| 2625. | Cl | SO₂Me | OCH₂CH₂ | Me | Q5c | |
| 2626. | Cl | SO₂Me | OCH₂CH₂ | Me | Q6a | |
| 2627. | Cl | SO₂Me | OCH₂CH₂ | Me | Q6b | |
| 2628. | Cl | SO₂Me | OCH₂CH₂ | Me | Q7 | |
| 2629. | Cl | SO₂Me | OCH₂CH₂ | Me | Q8a | |
| 2630. | Cl | SO₂Me | OCH₂CH₂ | Me | Q8b | |
| 2631. | Cl | SO₂Me | OCH₂CH₂ | Me | Q8c | |
| 2632. | Cl | SO₂Me | OCH₂CH₂ | Me | Q8d | |
| 2633. | Cl | SO₂Me | OCH₂CH₂ | Me | Q9a | |
| 2634. | Cl | SO₂Me | OCH₂CH₂ | Me | Q9b | |
| 2635. | Cl | SO₂Me | OCH₂CH₂ | Me | Q9c | |
| 2636. | Cl | SO₂Me | OCH₂CH₂ | Me | Q9d | |
| 2637. | Cl | SO₂Me | OCH₂CH₂ | Me | Q10a | |
| 2638. | Cl | SO₂Me | OCH₂CH₂ | Me | Q11a | |
| 2639. | Cl | SO₂Me | OCH₂CH₂ | Me | Q12 | |
| 2640. | Cl | SO₂Me | OCH₂CH₂ | Et | Q1a | |
| 2641. | Cl | SO₂Me | OCH₂CH₂ | Et | Q1b | |
| 2642. | Cl | SO₂Me | OCH₂CH₂ | Et | Q1c | |
| 2643. | Cl | SO₂Me | OCH₂CH₂ | Et | Q1d | |
| 2644. | Cl | SO₂Me | OCH₂CH₂ | Et | Q1e | |
| 2645. | Cl | SO₂Me | OCH₂CH₂ | Et | Q2a | |
| 2646. | Cl | SO₂Me | OCH₂CH₂ | Et | Q2b | |
| 2647. | Cl | SO₂Me | OCH₂CH₂ | Et | Q3a | |
| 2648. | Cl | SO₂Me | OCH₂CH₂ | Et | Q3c | |
| 2649. | Cl | SO₂Me | OCH₂CH₂ | Et | Q4a | |
| 2650. | Cl | SO₂Me | OCH₂CH₂ | Et | Q5a | |
| 2651. | Cl | SO₂Me | OCH₂CH₂ | Et | Q6a | |
| 2652. | Cl | SO₂Me | OCH₂CH₂ | Et | Q8a | |
| 2653. | Cl | SO₂Me | OCH₂CH₂ | Et | Q8d | |
| 2654. | Cl | SO₂Me | OCH₂CH₂ | Et | Q9a | |
| 2655. | Cl | SO₂Me | OCH₂CH₂ | Et | Q12 | |
| 2656. | Cl | SO₂Me | OCH₂CH₂ | Et | Q13 | |
| 2657. | Cl | SO₂Me | OCH₂CH₂ | n-Pr | Q1a | |
| 2658. | Cl | SO₂Me | OCH₂CH₂ | n-Pr | Q3a | |
| 2659. | Cl | SO₂Me | OCH₂CH₂ | n-Pr | Q6a | |
| 2660. | Cl | SO₂Me | OCH₂CH₂ | SMe | Q1a | |
| 2661. | Cl | SO₂Me | OCH₂CH₂ | SMe | Q3a | |
| 2662. | Cl | SO₂Me | OCH₂CH₂ | SMe | Q6a | |
| 2663. | Cl | SO₂Me | OCH₂CH₂ | n-Bu | Q1a | |
| 2664. | Cl | SO₂Me | OCH₂CH₂ | n-Pen | Q1a | |
| 2665. | Cl | SO₂Me | OCH₂CH₂ | n-Hex | Q1a | |
| 2666. | Cl | SO₂Me | OCH₂CH₂ | cyclo-Pr | Q1a | |
| 2667. | Cl | SO₂Me | OCH₂CH₂ | cyclo-Bu | Q1a | |
| 2668. | Cl | SO₂Me | OCH₂CH₂ | cyclo-Pen | Q1a | |
| 2669. | Cl | SO₂Me | OCH₂CH₂ | cyclo-Hex | Q1a | |
| 2670. | Cl | SO₂Me | OCH₂CH₂ | SEt | Q1a | |
| 2671. | Cl | SO₂Me | OCH₂CH₂ | S-n-Pr | Q1a | |
| 2672. | Cl | SO₂Me | OCH₂CH₂ | Ph | Q1a | |
| 2673. | Cl | SO₂Me | OCH₂CH₂ | 2-Cl-Ph | Q1a | |
| 2674. | Cl | SO₂Me | OCH₂CH₂ | 3-Cl-Ph | Q1a | |
| 2675. | Cl | SO₂Me | OCH₂CH₂ | 2-Me-Ph | Q1a | |
| 2676. | Cl | SO₂Me | OCH₂CH₂ | 4-Me-Ph | Q1a | |
| 2677. | Cl | SO₂Me | OCH₂CH₂ | 3-Et-Ph | Q1a | |
| 2678. | Cl | SO₂Me | OCH₂CH₂ | 4-n-Pr-Ph | Q1a | |
| 2679. | Cl | SO₂Me | OCH₂CH₂ | 3-CF3-Ph | Q1a | |
| 2680. | Cl | SO₂Me | OCH₂CH₂ | 3-CH₂CF3-Ph | Q1a | |
| 2681. | Cl | SO₂Me | OCH₂CH₂ | 3-NO₂-Ph | Q1a | |
| 2682. | Cl | SO₂Me | OCH₂CH(Me) | H | Q1a | |
| 2683. | Cl | SO₂Me | OCH₂CH(Me) | H | Q1b | |
| 2684. | Cl | SO₂Me | OCH₂CH(Me) | H | Q1c | |
| 2685. | Cl | SO₂Me | OCH₂CH(Me) | H | Q1d | |
| 2686. | Cl | SO₂Me | OCH₂CH(Me) | H | Q1e | |
| 2687. | Cl | SO₂Me | OCH₂CH(Me) | H | Q2a | |
| 2688. | Cl | SO₂Me | OCH₂CH(Me) | H | Q2b | |
| 2689. | Cl | SO₂Me | OCH₂CH(Me) | H | Q3a | |
| 2690. | Cl | SO₂Me | OCH₂CH(Me) | H | Q3c | |
| 2691. | Cl | SO₂Me | OCH₂CH(Me) | H | Q4a | |
| 2692. | Cl | SO₂Me | OCH₂CH(Me) | H | Q5a | |
| 2693. | Cl | SO₂Me | OCH₂CH(Me) | H | Q6a | |
| 2694. | Cl | SO₂Me | OCH₂CH(Me) | H | Q8a | |
| 2695. | Cl | SO₂Me | OCH₂CH(Me) | H | Q8d | |
| 2696. | Cl | SO₂Me | OCH₂CH(Me) | H | Q9a | |
| 2697. | Cl | SO₂Me | OCH₂CH(Me) | H | Q12 | |
| 2698. | Cl | SO₂Me | OCH₂CH(Me) | H | Q13 | |
| 2699. | Cl | SO₂Me | OCH₂CH(Me) | Me | Q1a | |
| 2700. | Cl | SO₂Me | OCH₂CH(Me) | Me | Q3a | |
| 2701. | Cl | SO₂Me | OCH₂CH(Me) | Me | Q6a | |
| 2702. | Cl | SO₂Me | OCH₂CH₂CH₂ | H | Q1a | |
| 2703. | Cl | SO₂Me | OCH₂CH₂CH₂ | H | Q1b | |
| 2704. | Cl | SO₂Me | OCH₂CH₂CH₂ | H | Q1c | |
| 2705. | Cl | SO₂Me | OCH₂CH₂CH₂ | H | Q1d | |
| 2706. | Cl | SO₂Me | OCH₂CH₂CH₂ | H | Q1e | |
| 2707. | Cl | SO₂Me | OCH₂CH₂CH₂ | H | Q2a | |
| 2708. | Cl | SO₂Me | OCH₂CH₂CH₂ | H | Q2b | |
| 2709. | Cl | SO₂Me | OCH₂CH₂CH₂ | H | Q3a | |
| 2710. | Cl | SO₂Me | OCH₂CH₂CH₂ | H | Q3c | |
| 2711. | Cl | SO₂Me | OCH₂CH₂CH₂ | H | Q4a | |
| 2712. | Cl | SO₂Me | OCH₂CH₂CH₂ | H | Q5a | |
| 2713. | Cl | SO₂Me | OCH₂CH₂CH₂ | H | Q6a | |
| 2714. | Cl | SO₂Me | OCH₂CH₂CH₂ | H | Q8a | |
| 2715. | Cl | SO₂Me | OCH₂CH₂CH₂ | H | Q8d | |
| 2716. | Cl | SO₂Me | OCH₂CH₂CH₂ | H | Q9a | |
| 2717. | Cl | SO₂Me | OCH₂CH₂CH₂ | H | Q12 | |
| 2718. | Cl | SO₂Me | OCH₂CH₂CH₂ | H | Q13 | |
| 2719. | Cl | SO₂Me | OCH₂CH₂CH₂ | Me | Q1a | |
| 2720. | Cl | SO₂Me | OCH₂CH₂CH₂ | Me | Q2a | |
| 2721. | Cl | SO₂Me | OCH₂CH₂CH₂ | Me | Q2b | |
| 2722. | Cl | SO₂Me | OCH₂CH₂CH₂ | Me | Q3a | |
| 2723. | Cl | SO₂Me | OCH₂CH₂CH₂ | Me | Q6a | |
| 2724. | Cl | SO₂Me | OCH₂CH₂CH₂ | Me | Q13 | |
| 2725. | Cl | SO₂Me | CH(Me) | H | Q1a | |
| 2726. | Cl | SO₂Me | CH(Me)CH₂ | H | Q1a | |
| 2727. | Cl | SO₂Me | CH₂CH₂CH₂ | H | Q1a | |
| 2728. | Cl | SO₂Me | OCH₂ | H | Q1a | |
| 2729. | Cl | SO₂Me | OCH₂ | Me | Q1a | |
| 2730. | Cl | SO₂Me | OCH(Me)CH₂ | H | Q1a | |
| 2731. | Cl | SO₂Me | OCH(Me)CH₂ | Me | Q1a | |
| 2732. | Cl | SO₂Et | CH₂ | H | Q1a | |
| 2733. | Cl | SO₂Et | CH₂ | H | Q1b | |
| 2734. | Cl | SO₂Et | CH₂ | H | Q1c | |
| 2735. | Cl | SO₂Et | CH₂ | H | Q1d | |
| 2736. | Cl | SO₂Et | CH₂ | H | Q1e | |
| 2737. | Cl | SO₂Et | CH₂ | H | Q2a | |
| 2738. | Cl | SO₂Et | CH₂ | H | Q2b | |
| 2739. | Cl | SO₂Et | CH₂ | H | Q3a | |
| 2740. | Cl | SO₂Et | CH₂ | H | Q3c | |
| 2741. | Cl | SO₂Et | CH₂ | H | Q4a | |
| 2742. | Cl | SO₂Et | CH₂ | H | Q5a | |
| 2743. | Cl | SO₂Et | CH₂ | H | Q6a | |
| 2744. | Cl | SO₂Et | CH₂ | H | Q8a | |
| 2745. | Cl | SO₂Et | CH₂ | H | Q8d | |
| 2746. | Cl | SO₂Et | CH₂ | H | Q9a | |
| 2747. | Cl | SO₂Et | CH₂ | H | Q12 | |
| 2748. | Cl | SO₂Et | CH₂ | H | Q13 | |
| 2749. | Cl | SO₂Et | CH₂ | Me | Q1a | |
| 2750. | Cl | SO₂Et | CH₂ | Me | Q2a | |
| 2751. | Cl | SO₂Et | CH₂ | Me | Q2b | |
| 2752. | Cl | SO₂Et | CH₂ | Me | Q3a | |
| 2753. | Cl | SO₂Et | CH₂ | Me | Q6a | |
| 2754. | Cl | SO₂Et | CH₂ | Me | Q8d | |
| 2755. | Cl | SO₂Et | CH₂CH₂ | H | Q1a | |
| 2756. | Cl | SO₂Et | CH₂CH₂ | Me | Q1a | |
| 2757. | Cl | SO₂Et | OCH₂CH₂ | H | Q1a | |
| 2758. | Cl | SO₂Et | OCH₂CH₂ | H | Q1b | |
| 2759. | Cl | SO₂Et | OCH₂CH₂ | H | Q1c | |
| 2760. | Cl | SO₂Et | OCH₂CH₂ | H | Q1d | |
| 2761. | Cl | SO₂Et | OCH₂CH₂ | H | Q1e | |
| 2762. | Cl | SO₂Et | OCH₂CH₂ | H | Q2a | |
| 2763. | Cl | SO₂Et | OCH₂CH₂ | H | Q2b | |
| 2764. | Cl | SO₂Et | OCH₂CH₂ | H | Q3a | |
| 2765. | Cl | SO₂Et | OCH₂CH₂ | H | Q3c | |
| 2766. | Cl | SO₂Et | OCH₂CH₂ | H | Q4a | |
| 2767. | Cl | SO₂Et | OCH₂CH₂ | H | Q5a | |
| 2768. | Cl | SO₂Et | OCH₂CH₂ | H | Q6a | |
| 2769. | Cl | SO₂Et | OCH₂CH₂ | H | Q8a | |
| 2770. | Cl | SO₂Et | OCH₂CH₂ | H | Q8d | |
| 2771. | Cl | SO₂Et | OCH₂CH₂ | H | Q9a | |
| 2772. | Cl | SO₂Et | OCH₂CH₂ | H | Q12 | |
| 2773. | Cl | SO₂Et | OCH₂CH₂ | H | Q13 | |
| 2774. | Cl | SO₂Et | OCH₂CH₂ | Me | Q1a | |
| 2775. | Cl | SO₂Et | OCH₂CH₂ | Me | Q2a | |
| 2776. | Cl | SO₂Et | OCH₂CH₂ | Me | Q2b | |
| 2777. | Cl | SO₂Et | OCH₂CH₂ | Me | Q3a | |
| 2778. | Cl | SO₂Et | OCH₂CH₂ | Me | Q6a | |
| 2779. | Cl | SO₂Et | OCH₂CH₂ | Me | Q8d | |
| 2780. | Cl | SO₂Et | OCH₂CH(Me) | H | Q1a | |
| 2781. | Cl | SO₂Et | OCH₂CH₂CH₂ | H | Q1a | |
| 2782. | Cl | SO₂Pr-n | CH₂ | H | Q1a | |
| 2783. | Cl | SO₂Pr-n | OCH₂CH₂ | H | Q1a | |
| 2784. | Cl | SO₂Pr-n | OCH₂CH₂ | Me | Q1a | |
| 2785. | Cl | NO₂ | CH₂ | H | Q1a | |
| 2786. | Cl | NO₂ | CH₂ | H | Q3a | |
| 2787. | Cl | NO₂ | CH₂ | Me | Q1a | |
| 2788. | Cl | NO₂ | OCH₂CH₂ | H | Q1a | |
| 2789. | Cl | NO₂ | 0C112C112 | H | Q1b | |
| 2790. | Cl | NO₂ | OCH₂CH₂ | H | Q1c | |
| 2791. | Cl | NO₂ | OCH₂CH₂ | H | Q1d | |
| 2792. | Cl | NO₂ | OCH₂CH₂ | H | Q1e | |
| 2793. | Cl | NO₂ | OCH₂CH₂ | H | Q2a | |
| 2794. | Cl | NO₂ | OCH₂CH₂ | H | Q2b | |
| 2795. | Cl | NO₂ | OCH₂CH₂ | H | Q3a | |
| 2796. | Cl | NO₂ | OCH₂CH₂ | H | Q3c | |
| 2797. | Cl | NO₂ | OCH₂CH₂ | H | Q4a | |
| 2798. | Cl | NO₂ | OCH₂CH₂ | H | Q5a | |
| 2799. | Cl | NO₂ | OCH₂CH₂ | H | Q6a | |
| 2800. | Cl | NO₂ | OCH₂CH₂ | H | Q8a | |
| 2801. | Cl | NO₂ | OCH₂CH₂ | H | Q8d | |
| 2802. | Cl | NO₂ | OCH₂CH₂ | H | Q9a | |
| 2803. | Cl | NO₂ | OCH₂CH₂ | H | Q12 | |
| 2804. | Cl | NO₂ | OCH₂CH₂ | H | Q13 | |
| 2805. | Cl | NO₂ | OCH₂CH₂ | Me | Q1a | |
| 2806. | Cl | NO₂ | OCH₂CH₂ | Me | Q2a | |
| 2807. | Cl | NO₂ | OCH₂CH₂ | Me | Q2b | |
| 2808. | Cl | NO₂ | OCH₂CH₂ | Me | Q3a | |
| 2809. | Cl | NO₂ | OCH₂CH₂ | Me | Q6a | |
| 2810. | Cl | NO₂ | OCH₂CH₂ | Me | Q8d | |
| 2811. | Cl | NO₂ | OCH₂CH(Me) | H | Q1a | |
| 2812. | Cl | NO₂ | OCH₂CH₂CH₂ | H | Q1a | |
| 2813. | Br | F | OCH₂CH₂ | H | Q1a | |
| 2814. | Br | F | OCH₂CH₂ | H | Q1b | |
| 2815. | Br | F | OCH₂CH₂ | H | Q1c | |
| 2816. | Br | F | OCH₂CH₂ | H | Q1d | |
| 2817. | Br | F | OCH₂CH₂ | H | Q1e | |
| 2818. | Br | F | OCH₂CH₂ | H | Q2a | |
| 2819. | Br | F | OCH₂CH₂ | H | Q2b | |
| 2820. | Br | F | OCH₂CH₂ | H | Q3a | |
| 2821. | Br | F | OCH₂CH₂ | H | Q3c | |
| 2822. | Br | F | OCH₂CH₂ | H | Q4a | |
| 2823. | Br | F | OCH₂CH₂ | H | Q5a | |
| 2824. | Br | F | OCH₂CH₂ | H | Q6a | |
| 2825. | Br | F | OCH₂CH₂ | H | Q8a | |
| 2826. | Br | F | OCH₂CH₂ | H | Q8d | |
| 2827. | Br | F | OCH₂CH₂ | H | Q9a | |
| 2828. | Br | F | OCH₂CH₂ | H | Q12 | |
| 2829. | Br | F | OCH₂CH₂ | H | Q13 | |
| 2830. | Br | F | OCH₂CH₂ | Me | Q1a | |
| 2831. | Br | F | OCH₂CH₂ | Me | Q2a | |
| 2832. | Br | F | OCH₂CH₂ | Me | Q2b | |
| 2833. | Br | F | OCH₂CH₂ | Me | Q3a | |
| 2834. | Br | F | OCH₂CH₂ | Me | Q6a | |
| 2835. | Br | F | OCH₂CH₂ | Me | Q8d | |
| 2836. | Br | F | OCH₂CH(Me) | H | Q1a | |
| 2837. | Br | F | OCH₂CH₂CH₂ | H | Q1a | |
| 2838. | Br | F | OCH₂CH₂CH₂ | H | Q3a | |
| 2839. | Br | Cl | OCH₂CH₂ | H | Q1a | |
| 2840. | Br | Cl | OCH₂CH₂ | H | Q1b | |
| 2841. | Br | Cl | OCH₂CH₂ | H | Q1c | |
| 2842. | Br | Cl | OCH₂CH₂ | H | Q1d | |
| 2843. | Br | Cl | OCH₂CH₂ | H | Q1e | |
| 2844. | Br | Cl | OCH₂CH₂ | H | Q2a | |
| 2845. | Br | Cl | OCH₂CH₂ | H | Q2b | |
| 2846. | Br | Cl | OCH₂CH₂ | H | Q3a | |
| 2847. | Br | Cl | OCH₂CH₂ | H | Q3c | |
| 2848. | Br | Cl | OCH₂CH₂ | H | Q4a | |
| 2849. | Br | Cl | OCH₂CH₂ | H | Q5a | |
| 2850. | Br | Cl | OCH₂CH₂ | H | Q6a | |
| 2851. | Br | Cl | OCH₂CH₂ | H | Q8a | |
| 2852. | Br | Cl | OCH₂CH₂ | H | Q8d | |
| 2853. | Br | Cl | OCH₂CH₂ | H | Q9a | |
| 2854. | Br | Cl | OCH₂CH₂ | H | Q12 | |
| 2855. | Br | Cl | OCH₂CH₂ | H | Q13 | |
| 2856. | Br | Cl | OCH₂CH₂ | Me | Q1a | |
| 2857. | Br | Cl | OCH₂CH₂ | Me | Q2a | |
| 2858. | Br | Cl | OCH₂CH₂ | Me | Q2b | |
| 2859. | Br | Cl | OCH₂CH₂ | Me | Q3a | |
| 2860. | Br | Cl | OCH₂CH₂ | Me | Q6a | |
| 2861. | Br | Cl | OCH₂CH₂ | Me | Q8d | |
| 2862. | Br | Cl | OCH₂CH(Me) | H | Q1a | |
| 2863. | Br | Cl | OCH₂CH₂CH₂ | H | Q1a | |
| 2864. | Br | Cl | OCH₂CH₂CH₂ | H | Q3a | |
| 2865. | Br | Br | CH₂ | H | Q1a | 128-130 |
| 2866. | Br | Br | CH₂ | H | Q1b | |
| 2867. | Br | Br | CH₂ | H | Q1c | |
| 2868. | Br | Br | CH₂ | H | Q1d | |
| 2869. | Br | Br | CH₂ | H | Q1e | |
| 2870. | Br | Br | CH₂ | H | Q2a | |
| 2871. | Br | Br | CH₂ | H | Q2b | |
| 2872. | Br | Br | CH₂ | H | Q3a | 133-139 |
| 2873. | Br | Br | CH₂ | H | Q3b | |
| 2874. | Br | Br | CH₂ | H | Q3c | |
| 2875. | Br | Br | CH₂ | H | Q3d | |
| 2876. | Br | Br | CH₂ | H | Q3e | |
| 2877. | Br | Br | CH₂ | H | Q3f | |
| 2878. | Br | Br | CH₂ | H | Q3g | |
| 2879. | Br | Br | CH₂ | H | Q3h | |
| 2880. | Br | Br | CH₂ | H | Q3i | |
| 2881. | Br | Br | CH₂ | H | Q3j | |
| 2882. | Br | Br | CH₂ | H | Q3k | |
| 2883. | Br | Br | CH₂ | H | Q31 | |
| 2884. | Br | Br | CH₂ | H | Q3m | |
| 2885. | Br | Br | CH₂ | H | Q3n | |
| 2886. | Br | Br | CH₂ | H | Q3o | |
| 2887. | Br | Br | CH₂ | H | Q3p | |
| 2888. | Br | Br | CH₂ | H | Q3q | |
| 2889. | Br | Br | CH₂ | H | Q3r | |
| 2890. | Br | Br | CH₂ | H | Q3s | |
| 2891. | Br | Br | CH₂ | H | Q3t | |
| 2892. | Br | Br | CH₂ | H | Q3u | |
| 2893. | Br | Br | CH₂ | H | Q3v | |
| 2894. | Br | Br | CH₂ | H | Q3w | |
| 2895. | Br | Br | CH₂ | H | Q3x | |
| 2896. | Br | Br | CH₂ | H | Q3y | |
| 2897. | Br | Br | CH₂ | H | Q3z | |
| 2898. | Br | Br | CH₂ | H | Q3za | |
| 2899. | Br | Br | CH₂ | H | Q3zb | |
| 2900. | Br | Br | CH₂ | H | Q3zc | |
| 2901. | Br | Br | CH₂ | H | Q3zd | |
| 2902. | Br | Br | CH₂ | H | Q4a | |
| 2903. | Br | Br | CH₂ | H | Q4b | |
| 2904. | Br | Br | CH₂ | H | Q4c | |
| 2905. | Br | Br | CH₂ | H | Q4d | |
| 2906. | Br | Br | CH₂ | Me | Q5a | |
| 2907. | Br | Br | CH₂ | H | Q6a | |
| 2908. | Br | Br | CH₂ | H | Q6b | |
| 2909. | Br | Br | CH₂ | H | Q7 | |
| 2910. | Br | Br | CH₂ | H | Q8a | |
| 2911. | Br | Br | CH₂ | H | Q8b | |
| 2912. | Br | Br | CH₂ | H | Q8c | |
| 2913. | Br | Br | CH₂ | H | Q8d | |
| 2914. | Br | Br | CH₂ | H | Q9a | |
| 2915. | Br | Br | CH₂ | H | Q9b | |
| 2916. | Br | Br | CH₂ | H | Q9c | |
| 2917. | Br | Br | CH₂ | H | Q9d | |
| 2918. | Br | Br | CH₂ | H | Q10a | |
| 2919. | Br | Br | CH₂ | H | Q11a | |
| 2920. | Br | Br | CH₂ | H | Q12 | |
| 2921. | Br | Br | CH₂ | H | Q13 | |
| 2922. | Br | Br | CH₂ | Me | Q1a | |
| 2923. | Br | Br | CH₂ | Me | Q1b | |
| 2924. | Br | Br | CH₂ | Me | Q1c | |
| 2925. | Br | Br | CH₂ | Me | Q1d | |
| 2926. | Br | Br | CH₂ | Me | Q1e | |
| 2927. | Br | Br | CH₂ | Me | Q2a | |
| 2928. | Br | Br | CH₂ | Me | Q2b | |
| 2929. | Br | Br | CH₂ | Me | Q3a | |
| 2930. | Br | Br | CH₂ | Me | Q3b | |
| 2931. | Br | Br | CH₂ | Me | Q3c | |
| 2932. | Br | Br | CH₂ | Me | Q3d | |
| 2933. | Br | Br | CH₂ | Me | Q3e | |
| 2934. | Br | Br | CH₂ | Me | Q3f | |
| 2935. | Br | Br | CH₂ | Me | Q3g | |
| 2936. | Br | Br | CH₂ | Me | Q3h | |
| 2937. | Br | Br | CH₂ | Me | Q3i | |
| 2938. | Br | Br | CH₂ | Me | Q3j | |
| 2939. | Br | Br | CH₂ | Me | Q3k | |
| 2940. | Br | Br | CH₂ | Me | Q31 | |
| 2941. | Br | Br | CH₂ | Me | Q3m | |
| 2942. | Br | Br | CH₂ | Me | Q3n | |
| 2943. | Br | Br | CH₂ | Me | Q3o | |
| 2944. | Br | Br | CH₂ | Me | Q3p | |
| 2945. | Br | Br | CH₂ | Me | Q3q | |
| 2946. | Br | Br | CH₂ | Me | Q3r | |
| 2947. | Br | Br | CH₂ | Me | Q3s | |
| 2948. | Br | Br | CH₂ | Me | Q3t | |
| 2949. | Br | Br | CH₂ | Me | Q3u | |
| 2950. | Br | Br | CH₂ | Me | Q3v | |
| 2951. | Br | Br | CH₂ | Me | Q3w | |
| 2952. | Br | Br | CH₂ | Me | Q3x | |
| 2953. | Br | Br | CH₂ | Me | Q3y | |
| 2954. | Br | Br | CH₂ | Me | Q3z | |
| 2955. | Br | Br | CH₂ | Me | Q3za | |
| 2956. | Br | Br | CH₂ | Me | Q3zb | |
| 2957. | Br | Br | CH₂ | Me | Q3zc | |
| 2958. | Br | Br | CH₂ | Me | Q3zd | |
| 2959. | Br | Br | CH₂ | Me | Q4a | |
| 2960. | Br | Br | CH₂ | Me | Q4b | |
| 2961. | Br | Br | CH₂ | Me | Q4c | |
| 2962. | Br | Br | CH₂ | Me | Q4d | |
| 2963. | Br | Br | CH₂ | Me | Q5a | |
| 2964. | Br | Br | CH₂ | Me | Q6a | |
| 2965. | Br | Br | CH₂ | Me | Q6b | |
| 2966. | Br | Br | CH₂ | Me | Q7 | |
| 2967. | Br | Br | CH₂ | Me | Q8a | |
| 2968. | Br | Br | CH₂ | Me | Q8b | |
| 2969. | Br | Br | CH₂ | Me | Q8c | |
| 2970. | Br | Br | CH₂ | Me | Q8d | |
| 2971. | Br | Br | CH₂ | Me | Q9a | |
| 2972. | Br | Br | CH₂ | Me | Q9b | |
| 2973. | Br | Br | CH₂ | Me | Q9c | |
| 2974. | Br | Br | CH₂ | Me | Q9d | |
| 2975. | Br | Br | CH₂ | Me | Q10a | |
| 2976. | Br | Br | CH₂ | Me | Q11a | |
| 2977. | Br | Br | CH₂ | Me | Q12 | |
| 2978. | Br | Br | CH₂ | Me | Q13 | |
| 2979. | Br | Br | CH₂ | Et | Q1a | |
| 2980. | Br | Br | CH₂ | Et | Q1b | |
| 2981. | Br | Br | CH₂ | Et | Q1c | |
| 2982. | Br | Br | CH₂ | Et | Q1d | |
| 2983. | Br | Br | CH₂ | Et | Q1e | |
| 2984. | Br | Br | CH₂ | Et | Q2a | |
| 2985. | Br | Br | CH₂ | Et | Q2b | |
| 2986. | Br | Br | CH₂ | Et | Q3a | |
| 2987. | Br | Br | CH₂ | Et | Q3c | |
| 2988. | Br | Br | CH₂ | Et | Q4a | |
| 2989. | Br | Br | CH₂ | Et | Q5a | |
| 2990. | Br | Br | CH₂ | Et | Q6a | |
| 2991. | Br | Br | CH₂ | Et | Q8a | |
| 2992. | Br | Br | CH₂ | Et | Q8d | |
| 2993. | Br | Br | CH₂ | Et | Q9a | |
| 2994. | Br | Br | CH₂ | Et | Q12 | |
| 2995. | Br | Br | CH₂ | Et | Q13 | |
| 2996. | Br | Br | CH₂ | n-Pr | Q1a | |
| 2997. | Br | Br | CH₂ | n-Pr | Q3a | |
| 2998. | Br | Br | CH₂ | n-Pr | Q6a | |
| 2999. | Br | Br | CH₂ | SMe | Q1a | |
| 3000. | Br | Br | CH₂ | SMe | Q1b | |
| 3001. | Br | Br | CH₂ | SMe | Q1c | |
| 3002. | Br | Br | CH₂ | SMe | Q1d | |
| 3003. | Br | Br | CH₂ | SMe | Q1e | |
| 3004. | Br | Br | CH₂ | SMe | Q2a | |
| 3005. | Br | Br | CH₂ | SMe | Q2b | |
| 3006. | Br | Br | CH₂ | SMe | Q3a | |
| 3007. | Br | Br | CH₂ | SMe | Q3c | |
| 3008. | Br | Br | CH₂ | SMe | Q4a | |
| 3009. | Br | Br | CH₂ | SMe | Q5a | |
| 3010. | Br | Br | CH₂ | SMe | Q6a | |
| 3011. | Br | Br | CH₂ | SMe | Q8a | |
| 3012. | Br | Br | CH₂ | SMe | Q8d | |
| 3013. | Br | Br | CH₂ | SMe | Q9a | |
| 3014. | Br | Br | CH₂ | SMe | Q12 | |
| 3015. | Br | Br | CH₂ | SMe | Q13 | |
| 3016. | Br | Br | CH₂CH₂ | H | Q1a | |
| 3017. | Br | Br | CH₂CH₂ | H | Q1b | |
| 3018. | Br | Br | CH₂CH₂ | H | Q1c | |
| 3019. | Br | Br | CH₂CH₂ | H | Q1d | |
| 3020. | Br | Br | CH₂CH₂ | H | Q1e | |
| 3021. | Br | Br | CH₂CH₂ | H | Q2a | |
| 3022. | Br | Br | CH₂CH₂ | H | Q2b | |
| 3023. | Br | Br | CH₂CH₂ | H | Q3a | |
| 3024. | Br | Br | CH₂CH₂ | H | Q3c | |
| 3025. | Br | Br | CH₂CH₂ | H | Q4a | |
| 3026. | Br | Br | CH₂CH₂ | H | Q5a | |
| 3027. | Br | Br | CH₂CH₂ | H | Q6a | |
| 3028. | Br | Br | CH₂CH₂ | H | Q8a | |
| 3029. | Br | Br | CH₂CH₂ | H | Q8d | |
| 3030. | Br | Br | CH₂CH₂ | H | Q9a | |
| 3031. | Br | Br | CH₂CH₂ | H | Q12 | |
| 3032. | Br | Br | CH₂CH₂ | H | Q13 | |
| 3033. | Br | Br | CH₂CH₂ | Me | Q1a | |
| 3034. | Br | Br | CH₂CH₂ | Me | Q2a | |
| 3035. | Br | Br | CH₂CH₂ | Me | Q2b | |
| 3036. | Br | Br | CH₂CH₂ | Me | Q3a | |
| 3037. | Br | Br | CH₂CH₂ | Me | Q6a | |
| 3038. | Br | Br | OCH₂CH₂ | H | Q1a | 1.6008 |
| 3039. | Br | Br | OCH₂CH₂ | H | Q1b | |
| 3040. | Br | Br | OCH₂CH₂ | H | Q1c | |
| 3041. | Br | Br | OCH₂CH₂ | H | Q1d | |
| 3042. | Br | Br | OCH₂CH₂ | H | Q1e | |
| 3043. | Br | Br | OCH₂CH₂ | H | Q2a | 54-58 |
| 3044. | Br | Br | OCH₂CH₂ | H | Q2b | |
| 3045. | Br | Br | OCH₂CH₂ | H | Q3a | |
| 3046. | Br | Br | OCH₂CH₂ | H | Q3b | |
| 3047. | Br | Br | OCH₂CH₂ | H | Q3c | |
| 3048. | Br | Br | OCH₂CH₂ | H | Q3d | |
| 3049. | Br | Br | OCH₂CH₂ | H | Q3e | |
| 3050. | Br | Br | OCH₂CH₂ | H | Q3f | |
| 3051. | Br | Br | OCH₂CH₂ | H | Q3g | |
| 3052. | Br | Br | OCH₂CH₂ | H | Q3h | |
| 3053. | Br | Br | OCH₂CH₂ | H | Q3i | |
| 3054. | Br | Br | OCH₂CH₂ | H | Q3j | |
| 3055. | Br | Br | OCH₂CH₂ | H | Q3k | |
| 3056. | Br | Br | OCH₂CH₂ | H | Q31 | |
| 3057. | Br | Br | OCH₂CH₂ | H | Q3m | |
| 3058. | Br | Br | OCH₂CH₂ | H | Q3n | |
| 3059. | Br | Br | OCH₂CH₂ | H | Q3o | |
| 3060. | Br | Br | OCH₂CH₂ | H | Q3p | |
| 3061. | Br | Br | OCH₂CH₂ | H | Q3q | |
| 3062. | Br | Br | OCH₂CH₂ | H | Q3r | |
| 3063. | Br | Br | OCH₂CH₂ | H | Q3s | |
| 3064. | Br | Br | OCH₂CH₂ | H | Q3t | |
| 3065. | Br | Br | OCH₂CH₂ | H | Q3u | |
| 3066. | Br | Br | OCH₂CH₂ | H | Q3v | |
| 3067. | Br | Br | OCH₂CH₂ | H | Q3w | |
| 3068. | Br | Br | OCH₂CH₂ | H | Q3x | |
| 3069. | Br | Br | OCH₂CH₂ | H | Q3y | |
| 3070. | Br | Br | OCH₂CH₂ | H | Q3z | |
| 3071. | Br | Br | OCH₂CH₂ | H | Q3za | |
| 3072. | Br | Br | OCH₂CH₂ | H | Q3zb | |
| 3073. | Br | Br | OCH₂CH₂ | H | Q3zc | |
| 3074. | Br | Br | OCH₂CH₂ | H | Q3zd | |
| 3075. | Br | Br | OCH₂CH₂ | H | Q4a | |
| 3076. | Br | Br | OCH₂CH₂ | H | Q4b | |
| 3077. | Br | Br | OCH₂CH₂ | H | Q4c | |
| 3078. | Br | Br | OCH₂CH₂ | H | Q4d | |
| 3079. | Br | Br | OCH₂CH₂ | H | Q5a | |
| 3080. | Br | Br | OCH₂CH₂ | H | Q5b | |
| 3081. | Br | Br | OCH₂CH₂ | H | Q5c | |
| 3082. | Br | Br | OCH₂CH₂ | H | Q5d | |
| 3083. | Br | Br | OCH₂CH₂ | H | Q5e | |
| 3084. | Br | Br | OCH₂CH₂ | H | Q6a | |
| 3085. | Br | Br | OCH₂CH₂ | H | Q6b | |
| 3086. | Br | Br | OCH₂CH₂ | H | Q7 | |
| 3087. | Br | Br | OCH₂CH₂ | H | Q8a | |
| 3088. | Br | Br | OCH₂CH₂ | H | Q8b | |
| 3089. | Br | Br | OCH₂CH₂ | H | Q8c | |
| 3090. | Br | Br | OCH₂CH₂ | H | Q8d | |
| 3091. | Br | Br | OCH₂CH₂ | H | Q9a | |
| 3092. | Br | Br | OCH₂CH₂ | H | Q9b | |
| 3093. | Br | Br | OCH₂CH₂ | H | Q9c | |
| 3094. | Br | Br | OCH₂CH₂ | H | Q9d | |
| 3095. | Br | Br | OCH₂CH₂ | H | Q10a | |
| 3096. | Br | Br | OCH₂CH₂ | H | Q11a | |
| 3097. | Br | Br | OCH₂CH₂ | H | Q12 | |
| 3098. | Br | Br | OCH₂CH₂ | H | Q13 | |
| 3099. | Br | Br | OCH₂CH₂ | Me | Q1a | |
| 3100. | Br | Br | OCH₂CH₂ | Me | Q1b | |
| 3101. | Br | Br | OCH₂CH₂ | Me | Q1c | |
| 3102. | Br | Br | OCH₂CH₂ | Me | Q1d | |
| 3103. | Br | Br | OCH₂CH₂ | Me | Q1e | |
| 3104. | Br | Br | OCH₂CH₂ | Me | Q2a | |
| 3105. | Br | Br | OCH₂CH₂ | Me | Q2b | |
| 3106. | Br | Br | OCH₂CH₂ | Me | Q3a | |
| 3107. | Br | Br | OCH₂CH₂ | Me | Q3b | |
| 3108. | Br | Br | OCH₂CH₂ | Me | Q3c | |
| 3109. | Br | Br | OCH₂CH₂ | Me | Q3d | |
| 3110. | Br | Br | OCH₂CH₂ | Me | Q3e | |
| 3111. | Br | Br | OCH₂CH₂ | Me | Q3f | |
| 3112. | Br | Br | OCH₂CH₂ | Me | Q3g | |
| 3113. | Br | Br | OCH₂CH₂ | Me | Q3h | |
| 3114. | Br | Br | OCH₂CH₂ | Me | Q3i | |
| 3115. | Br | Br | OCH₂CH₂ | Me | Q3j | |
| 3116. | Br | Br | OCH₂CH₂ | Me | Q3k | |
| 3117. | Br | Br | OCH₂CH₂ | Me | Q31 | |
| 3118. | Br | Br | OCH₂CH₂ | Me | Q3m | |
| 3119. | Br | Br | OCH₂CH₂ | Me | Q3n | |
| 3120. | Br | Br | OCH₂CH₂ | Me | Q3o | |
| 3121. | Br | Br | OCH₂CH₂ | Me | Q3p | |
| 3122. | Br | Br | OCH₂CH₂ | Me | Q3q | |
| 3123. | Br | Br | OCH₂CH₂ | Me | Q3r | |
| 3124. | Br | Br | OCH₂CH₂ | Me | Q3s | |
| 3125. | Br | Br | OCH₂CH₂ | Me | Q3t | |
| 3126. | Br | Br | OCH₂CH₂ | Me | Q3u | |
| 3127. | Br | Br | OCH₂CH₂ | Me | Q3v | |
| 3128. | Br | Br | OCH₂CH₂ | Me | Q3w | |
| 3129. | Br | Br | OCH₂CH₂ | Me | Q3x | |
| 3130. | Br | Br | OCH₂CH₂ | Me | Q3y | |
| 3131. | Br | Br | OCH₂CH₂ | Me | Q3z | |
| 3132. | Br | Br | OCH₂CH₂ | Me | Q3za | |
| 3133. | Br | Br | OCH₂CH₂ | Me | Q3zb | |
| 3134. | Br | Br | OCH₂CH₂ | Me | Q3zc | |
| 3135. | Br | Br | OCH₂CH₂ | Me | Q3zd | |
| 3136. | Br | Br | OCH₂CH₂ | Me | Q4a | |
| 3137. | Br | Br | OCH₂CH₂ | Me | Q4b | |
| 3138. | Br | Br | OCH₂CH₂ | Me | Q4c | |
| 3139. | Br | Br | OCH₂CH₂ | Me | Q4d | |
| 3140. | Br | Br | OCH₂CH₂ | Me | Q5a | |
| 3141. | Br | Br | OCH₂CH₂ | Me | Q5b | |
| 3142. | Br | Br | OCH₂CH₂ | Me | Q5c | |
| 3143. | Br | Br | OCH₂CH₂ | Me | Q6a | |
| 3144. | Br | Br | OCH₂CH₂ | Me | Q6b | |
| 3145. | Br | Br | OCH₂CH₂ | Me | Q7 | |
| 3146. | Br | Br | OCH₂CH₂ | Me | Q8a | |
| 3147. | Br | Br | OCH₂CH₂ | Me | Q8b | |
| 3148. | Br | Br | OCH₂CH₂ | Me | Q8c | |
| 3149. | Br | Br | OCH₂CH₂ | Me | Q8d | |
| 3150. | Br | Br | OCH₂CH₂ | Me | Q9a | |
| 3151. | Br | Br | OCH₂CH₂ | Me | Q9b | |
| 3152. | Br | Br | OCH₂CH₂ | Me | Q9c | |
| 3153. | Br | Br | OCH₂CH₂ | Me | Q9d | |
| 3154. | Br | Br | OCH₂CH₂ | Me | Q10a | |
| 3155. | Br | Br | OCH₂CH₂ | Me | Q11a | |
| 3156. | Br | Br | OCH₂CH₂ | Me | Q12 | |
| 3157. | Br | Br | OCH₂CH₂ | Me | Q13 | |
| 3158. | Br | Br | OCH₂CH₂ | Et | Q1a | |
| 3159. | Br | Br | OCH₂CH₂ | Et | Q1b | |
| 3160. | Br | Br | OCH₂CH₂ | Et | Q1c | |
| 3161. | Br | Br | OCH₂CH₂ | Et | Q1d | |
| 3162. | Br | Br | OCH₂CH₂ | Et | Q1e | |
| 3163. | Br | Br | OCH₂CH₂ | Et | Q2a | |
| 3164. | Br | Br | OCH₂CH₂ | Et | Q2b | |
| 3165. | Br | Br | OCH₂CH₂ | Et | Q3a | |
| 3166. | Br | Br | OCH₂CH₂ | Et | Q3c | |
| 3167. | Br | Br | OCH₂CH₂ | Et | Q4a | |
| 3168. | Br | Br | OCH₂CH₂ | Et | Q5a | |
| 3169. | Br | Br | OCH₂CH₂ | Et | Q6a | |
| 3170. | Br | Br | OCH₂CH₂ | Et | Q8a | |
| 3171. | Br | Br | OCH₂CH₂ | Et | Q8d | |
| 3172. | Br | Br | OCH₂CH₂ | Et | Q9a | |
| 3173. | Br | Br | OCH₂CH₂ | Et | Q12 | |
| 3174. | Br | Br | OCH₂CH₂ | Et | Q13 | |
| 3175. | Br | Br | OCH₂CH₂ | n-Pr | Q1a | |
| 3176. | Br | Br | OCH₂CH₂ | n-Pr | Q3a | |
| 3177. | Br | Br | OCH₂CH₂ | n-Pr | Q6a | |
| 3178. | Br | Br | OCH₂CH₂ | SMe | Q1a | |
| 3179. | Br | Br | OCH₂CH₂ | SMe | Q3a | |
| 3180. | Br | Br | OCH₂CH₂ | SMe | Q6a | |
| 3181. | Br | Br | OCH₂CH(Me) | H | Q1a | |
| 3182. | Br | Br | OCH₂CH(Me) | H | Q1b | |
| 3183. | Br | Br | OCH₂CH(Me) | H | Q1c | |
| 3184. | Br | Br | OCH₂CH(Me) | H | Q1d | |
| 3185. | Br | Br | OCH₂CH(Me) | H | Q1e | |
| 3186. | Br | Br | OCH₂CH(Me) | H | Q2a | |
| 3187. | Br | Br | OCH₂CH(Me) | H | Q2b | |
| 3188. | Br | Br | OCH₂CH(Me) | H | Q3a | |
| 3189. | Br | Br | OCH₂CH(Me) | H | Q3c | |
| 3190. | Br | Br | OCH₂CH(Me) | H | Q4a | |
| 3191. | Br | Br | OCH₂CH(Me) | H | Q5a | |
| 3192. | Br | Br | OCH₂CH(Me) | H | Q6a | |
| 3193. | Br | Br | OCH₂CH(Me) | H | Q8a | |
| 3194. | Br | Br | OCH₂CH(Me) | H | Q8d | |
| 3195. | Br | Br | OCH₂CH(Me) | H | Q9a | |
| 3196. | Br | Br | OCH₂CH(Me) | H | Q12 | |
| 3197. | Br | Br | OCH₂CH(Me) | H | Q13 | |
| 3198. | Br | Br | OCH₂CH(Me) | Me | Q1a | |
| 3199. | Br | Br | OCH₂CH(Me) | Me | Q3a | |
| 3200. | Br | Br | OCH₂CH(Me) | Me | Q6a | |
| 3201. | Br | Br | OCH₂CH₂CH₂ | H | Q1a | |
| 3202. | Br | Br | OCH₂CH₂CH₂ | H | Q1b | |
| 3203. | Br | Br | OCH₂CH₂CH₂ | H | Q1c | |
| 3204. | Br | Br | OCH₂CH₂CH₂ | H | Q1d | |
| 3205. | Br | Br | OCH₂CH₂CH₂ | H | Q1e | |
| 3206. | Br | Br | OCH₂CH₂CH₂ | H | Q2a | |
| 3207. | Br | Br | OCH₂CH₂CH₂ | H | Q2b | |
| 3208. | Br | Br | OCH₂CH₂CH₂ | H | Q3a | |
| 3209. | Br | Br | OCH₂CH₂CH₂ | H | Q3c | |
| 3210. | Br | Br | OCH₂CH₂CH₂ | H | Q4a | |
| 3211. | Br | Br | OCH₂CH₂CH₂ | H | Q5a | |
| 3212. | Br | Br | OCH₂CH₂CH₂ | H | Q6a | |
| 3213. | Br | Br | OCH₂CH₂CH₂ | H | Q8a | |
| 3214. | Br | Br | OCH₂CH₂CH₂ | H | Q8d | |
| 3215. | Br | Br | OCH₂CH₂CH₂ | H | Q9a | |
| 3216. | Br | Br | OCH₂CH₂CH₂ | H | Q12 | |
| 3217. | Br | Br | OCH₂CH₂CH₂ | H | Q13 | |
| 3218. | Br | Br | OCH₂CH₂CH₂ | Me | Q1a | |
| 3219. | Br | Br | OCH₂CH₂CH₂ | Me | Q3a | |
| 3220. | Br | Br | OCH₂CH₂CH₂ | Me | Q6a | |
| 3221. | Br | Br | CH(Me) | H | Q1a | |
| 3222. | Br | Br | CH(Me)CH₂ | H | Q1a | |
| 3223. | Br | Br | OCH₂ | H | Q1a | |
| 3224. | Br | Br | OCH(Me)CH₂ | H | Q1a | |
| 3225. | Br | Br(Me) | OCH₂CH₂ | H | Q1a | |
| 3226. | Br | Br(Me) | OCH₂CH₂ | H | Q2a | |
| 3227. | Br | Br(Me) | OCH₂CH₂ | H | Q2b | |
| 3228. | Br | Br(Me) | OCH₂CH₂ | H | Q3a | |
| 3229. | Br | Br(Me) | OCH₂CH₂ | H | Q6a | |
| 3230. | Br | Br(Me) | OCH₂CH₂ | H | Q8d | |
| 3231. | Br | Br(Me) | OCH₂CH₂ | Me | Q1a | |
| 3232. | Br | Br(Me) | OCH₂CH₂ | Me | Q2a | |
| 3233. | Br | Br(Me) | OCH₂CH₂ | Me | Q2b | |
| 3234. | Br | Br(Me) | OCH₂CH₂ | Me | Q3a | |
| 3235. | Br | Br(Me) | OCH₂CH₂ | Me | Q6a | |
| 3236. | Br | Br(Me) | OCH₂CH₂ | Me | Q8d | |
| 3237. | Br | I | OCH₂CH₂ | H | Q1a | |
| 3238. | Br | I | OCH₂CH₂ | H | Q2a | |
| 3239. | Br | I | OCH₂CH₂ | H | Q2b | |
| 3240. | Br | I | OCH₂CH₂ | H | Q3a | |
| 3241. | Br | I | OCH₂CH₂ | H | Q6a | |
| 3242. | Br | I | OCH₂CH₂ | H | Q8d | |
| 3243. | Br | I | OCH₂CH₂ | Me | Q1a | |
| 3244. | Br | I | OCH₂CH₂ | Me | Q2a | |
| 3245. | Br | I | OCH₂CH₂ | Me | Q2b | |
| 3246. | Br | I | OCH₂CH₂ | Me | Q6a | |
| 3247. | Br | I | OCH(Me)CH₂ | H | Q1a | |
| 3248. | Br | I | OCH₂CH₂CH₂ | H | Q1a | |
| 3249. | Br | Me | OCH₂CH₂ | H | Q1a | |
| 3250. | Br | Me | OCH₂CH₂ | H | Q1b | |
| 3251. | Br | Me | OCH₂CH₂ | H | Q1c | |
| 3252. | Br | Me | OCH₂CH₂ | H | Q1d | |
| 3253. | Br | Me | OCH₂CH₂ | H | Q1e | |
| 3254. | Br | Me | OCH₂CH₂ | H | Q2a | |
| 3255. | Br | Me | OCH₂CH₂ | H | Q2b | |
| 3256. | Br | Me | OCH₂CH₂ | H | Q3a | |
| 3257. | Br | Me | OCH₂CH₂ | H | Q3c | |
| 3258. | Br | Me | OCH₂CH₂ | H | Q4a | |
| 3259. | Br | Me | OCH₂CH₂ | H | Q5a | |
| 3260. | Br | Me | OCH₂CH₂ | H | Q6a | |
| 3261. | Br | Me | OCH₂CH₂ | H | Q8a | |
| 3262. | Br | Me | OCH₂CH₂ | H | Q8d | |
| 3263. | Br | Me | OCH₂CH₂ | H | Q9a | |
| 3264. | Br | Me | OCH₂CH₂ | H | Q12 | |
| 3265. | Br | Me | OCH₂CH₂ | H | Q13 | |
| 3266. | Br | Me | OCH₂CH₂ | Me | Q1a | |
| 3267. | Br | Me | OCH₂CH₂ | Me | Q2a | |
| 3268. | Br | Me | OCH₂CH₂ | Me | Q2b | |
| 3269. | Br | Me | OCH₂CH₂ | Me | Q3a | |
| 3270. | Br | Me | OCH₂CH₂ | Me | Q6a | |
| 3271. | Br | Me | OCH₂CH₂ | Me | Q8d | |
| 3272. | Br | Me | OCH₂CH(Me) | H | Q1a | |
| 3273. | Br | Me | OCH₂CH₂CH₂ | H | Q1a | |
| 3274. | Br | CN | OCH₂CH₂ | H | Q1a | |
| 3275. | Br | CN | OCH₂CH₂ | H | Q3a | |
| 3276. | Br | CN | OCH₂CH₂ | H | Q4a | |
| 3277. | Br | CN | OCH₂CH₂ | Me | Q1a | |
| 3278. | Br | CN | OCH₂CH₂ | Me | Q3a | |
| 3279. | Br | CN | OCH₂CH₂ | Me | Q4a | |
| 3280. | Br | CF3 | OCH₂CH₂ | H | Q1a | |
| 3281. | Br | CF3 | OCH₂CH₂ | H | Q2a | |
| 3282. | Br | CF3 | OCH₂CH₂ | H | Q2b | |
| 3283. | Br | CF3 | OCH₂CH₂ | H | Q3a | |
| 3284. | Br | CF3 | OCH₂CH₂ | Me | Q1a | |
| 3285. | Br | CF3 | OCH₂CH₂ | Me | Q3a | |
| 3286. | Br | CF3 | OCH₂CH₂CH₂ | H | Q1a | |
| 3287. | Br | CF3 | OCH₂CH₂CH₂ | Me | Q1a | |
| 3288. | Br | SO₂Me | CH₂ | H | Q1a | |
| 3289. | Br | SO₂Me | CH₂ | H | Q1b | |
| 3290. | Br | SO₂Me | CH₂ | H | Q1c | |
| 3291. | Br | SO₂Me | CH₂ | H | Q1d | |
| 3292. | Br | SO₂Me | CH₂ | H | Q1e | |
| 3293. | Br | SO₂Me | CH₂ | H | Q2a | |
| 3294. | Br | SO₂Me | CH₂ | H | Q2b | |
| 3295. | Br | SO₂Me | CH₂ | H | Q3a | |
| 3296. | Br | SO₂Me | CH₂ | H | Q3c | |
| 3297. | Br | SO₂Me | CH₂ | H | Q4a | |
| 3298. | Br | SO₂Me | CH₂ | H | Q5a | |
| 3299. | Br | SO₂Me | CH₂ | H | Q6a | |
| 3300. | Br | SO₂Me | CH₂ | H | Q8a | |
| 3301. | Br | SO₂Me | CH₂ | H | Q8d | |
| 3302. | Br | SO₂Me | CH₂ | H | Q9a | |
| 3303. | Br | SO₂Me | CH₂ | H | Q12 | |
| 3304. | Br | SO₂Me | CH₂ | H | Q13 | |
| 3305. | Br | SO₂Me | CH₂ | Me | Q1a | |
| 3306. | Br | SO₂Me | CH₂ | Me | Q2a | |
| 3307. | Br | SO₂Me | CH₂ | Me | Q2b | |
| 3308. | Br | SO₂Me | CH₂ | Me | Q3a | |
| 3309. | Br | SO₂Me | CH₂ | Me | Q6a | |
| 3310. | Br | SO₂Me | CH₂ | Me | Q8d | |
| 3311. | Br | SO₂Me | CH₂ | Et | Q1a | |
| 3312. | Br | SO₂Me | CH₂ | n-Pr | Q1a | |
| 3313. | Br | SO₂Me | CH₂ | SMe | Q1a | |
| 3314. | Br | SO₂Me | CH₂CH₂ | H | Q1a | |
| 3315. | Br | SO₂Me | CH₂CH₂ | Me | Q1a | |
| 3316. | Br | SO₂Me | OCH₂CH₂ | H | Q1a | |
| 3317. | Br | SO₂Me | OCH₂CH₂ | H | Q1b | |
| 3318. | Br | SO₂Me | OCH₂CH₂ | H | Q1c | |
| 3319. | Br | SO₂Me | OCH₂CH₂ | H | Q1d | |
| 3320. | Br | SO₂Me | OCH₂CH₂ | H | Q1e | |
| 3321. | Br | SO₂Me | OCH₂CH₂ | H | Q2a | |
| 3322. | Br | SO₂Me | OCH₂CH₂ | H | Q2b | |
| 3323. | Br | SO₂Me | OCH₂CH₂ | H | Q3a | |
| 3324. | Br | SO₂Me | OCH₂CH₂ | H | Q3c | |
| 3325. | Br | SO₂Me | OCH₂CH₂ | H | Q4a | |
| 3326. | Br | SO₂Me | OCH₂CH₂ | H | Q5a | |
| 3327. | Br | SO₂Me | OCH₂CH₂ | H | Q6a | |
| 3328. | Br | SO₂Me | OCH₂CH₂ | H | Q8a | |
| 3329. | Br | SO₂Me | OCH₂CH₂ | H | Q8d | |
| 3330. | Br | SO₂Me | OCH₂CH₂ | H | Q9a | |
| 3331. | Br | SO₂Me | OCH₂CH₂ | H | Q12 | |
| 3332. | Br | SO₂Me | OCH₂CH₂ | H | Q13 | |
| 3333. | Br | SO₂Me | OCH₂CH₂ | Me | Q1a | |
| 3334. | Br | SO₂Me | OCH₂CH₂ | Me | Q2a | |
| 3335. | Br | SO₂Me | OCH₂CH₂ | Me | Q2b | |
| 3336. | Br | SO₂Me | OCH₂CH₂ | Me | Q3a | |
| 3337. | Br | SO₂Me | OCH₂CH₂ | Me | Q6a | |
| 3338. | Br | SO₂Me | OCH₂CH₂ | Me | Q8d | |
| 3339. | Br | SO₂Me | OCH₂CH₂ | Et | Q1a | |
| 3340. | Br | SO₂Me | OCH₂CH₂ | n-Pr | Q1a | |
| 3341. | Br | SO₂Me | OCH₂CH₂ | SMe | Q1a | |
| 3342. | Br | SO₂Me | OCH₂CH(Me) | H | Q1a | |
| 3343. | Br | SO₂Me | OCH₂CH₂CH₂ | H | Q1a | |
| 3344. | Br | SO₂Me | OCH₂CH₂CH₂ | Me | Q1a | |
| 3345. | I | I | OCH₂CH₂ | H | Q1a | |
| 3346. | I | I | OCH₂CH₂ | H | Q2a | |
| 3347. | I | I | OCH₂CH₂ | H | Q2b | |
| 3348. | I | I | OCH₂CH₂ | H | Q3a | |
| 3349. | I | I | OCH₂CH₂ | H | Q6a | |
| 3350. | I | I | OCH₂CH₂ | H | Q8d | |
| 3351. | I | I | OCH₂CH₂ | Me | Q1a | |
| 3352. | I | I | OCH₂CH₂ | Me | Q3a | |
| 3353. | I | I | OCH₂CH₂ | Me | Q6a | |
| 3354. | I | I | OCH₂CH₂(Me) | H | Q1a | |
| 3355. | I | I | OCH₂CH₂CH₂ | H | Q1a | |
| 3356. | Me | Cl | OCH₂CH₂ | H | Q1a | |
| 3357. | Me | Cl | OCH₂CH₂ | H | Q1b | |
| 3358. | Me | Cl | OCH₂CH₂ | H | Q1c | |
| 3359. | Me | Cl | OCH₂CH₂ | H | Q1d | |
| 3360. | Me | Cl | OCH₂CH₂ | H | Q1e | |
| 3361. | Me | Cl | OCH₂CH₂ | H | Q2a | |
| 3362. | Me | Cl | OCH₂CH₂ | H | Q2b | |
| 3363. | Me | Cl | OCH₂CH₂ | H | Q3a | |
| 3364. | Me | Cl | OCH₂CH₂ | H | Q3c | |
| 3365. | Me | Cl | OCH₂CH₂ | H | Q4a | |
| 3366. | Me | Cl | OCH₂CH₂ | H | Q5a | |
| 3367. | Me | Cl | OCH₂CH₂ | H | Q6a | |
| 3368. | Me | Cl | OCH₂CH₂ | H | Q8a | |
| 3369. | Me | Cl | OCH₂CH₂ | H | Q8d | |
| 3370. | Me | Cl | OCH₂CH₂ | H | Q9a | |
| 3371. | Me | Cl | OCH₂CH₂ | H | Q12 | |
| 3372. | Me | Cl | OCH₂CH₂ | H | Q13 | |
| 3373. | Me | Cl | OCH₂CH₂ | Me | Q1a | |
| 3374. | Me | Cl | OCH₂CH₂ | Me | Q2a | |
| 3375. | Me | Cl | OCH₂CH₂ | Me | Q2b | |
| 3376. | Me | Cl | OCH₂CH₂ | Me | Q3a | |
| 3377. | Me | Cl | OCH₂CH₂ | Me | Q6a | |
| 3378. | Me | Cl | OCH₂CH₂ | Me | Q8d | |
| 3379. | Me | Cl | OCH₂CH₂(Me) | H | Q1a | |
| 3380. | Me | Cl | OCH₂CH₂CH₂ | H | Q1a | |
| 3381. | Me | Br | OCH₂CH₂ | H | Q1a | 1.5475 |
| 3382. | Me | Br | OCH₂CH₂ | H | Q1b | |
| 3383. | Me | Br | OCH₂CH₂ | H | Q1c | |
| 3384. | Me | Br | OCH₂CH₂ | H | Q1d | |
| 3385. | Me | Br | OCH₂CH₂ | H | Q1e | |
| 3386. | Me | Br | OCH₂CH₂ | H | Q2a | 1.5915 |
| 3387. | Me | Br | OCH₂CH₂ | H | Q2b | |
| 3388. | Me | Br | OCH₂CH₂ | H | Q3a | 1.6328 |
| 3389. | Me | Br | OCH₂CH₂ | H | Q3c | |
| 3390. | Me | Br | OCH₂CH₂ | H | Q4a | |
| 3391. | Me | Br | OCH₂CH₂ | H | Q5a | |
| 3392. | Me | Br | OCH₂CH₂ | H | Q6a | |
| 3393. | Me | Br | OCH₂CH₂ | H | Q8a | |
| 3394. | Me | Br | OCH₂CH₂ | H | Q8d | |
| 3395. | Me | Br | OCH₂CH₂ | H | Q9a | |
| 3396. | Me | Br | OCH₂CH₂ | H | Q12 | |
| 3397. | Me | Br | OCH₂CH₂ | H | Q13 | 1.5688 |
| 3398. | Me | Br | OCH₂CH₂ | Me | Q1a | 1.5692 |
| 3399. | Me | Br | OCH₂CH₂ | Me | Q2a | |
| 3400. | Me | Br | OCH₂CH₂ | Me | Q2b | |
| 3401. | Me | Br | OCH₂CH₂ | Me | Q3a | |
| 3402. | Me | Br | OCH₂CH₂ | Me | Q6a | |
| 3403. | Me | Br | OCH₂CH₂ | Me | Q8d | |
| 3404. | Me | Br | OCH₂CH(Me) | H | Q1a | |
| 3405. | Me | Br | OCH₂CH₂CH₂ | H | Q1a | |
| 3406. | Me | I | OCH₂CH₂ | H | Q1a | |
| 3407. | Me | I | OCH₂CH₂ | H | Q3a | |
| 3408. | Me | 1 | OCH₂CH₂ | H | Q6a | |
| 3409. | Me | I | OCH₂CH₂ | H | Q8d | |
| 3410. | Me | I | OCH₂CH₂ | Me | Q1a | |
| 3411. | Me | I | OCH₂CH₂ | Me | Q3a | |
| 3412. | Me | I | OCH₂CH₂ | Me | Q6a | |
| 3413. | Me | I | OCH₂CH(Me) | H | Q1a | |
| 3414. | Me | I | OCH₂CH₂CH₂ | H | Q1a | |
| 3415. | Me | SMe | OCH₂CH₂ | H | Q1a | |
| 3416. | Me | SMe | OCH₂CH₂ | H | Q3a | |
| 3417. | Me | SMe | OCH₂CH₂ | H | Q6a | |
| 3418. | Me | SMe | OCH₂CH₂ | H | Q8d | |
| 3419. | Me | SMe | OCH₂CH₂ | Me | Q1a | |
| 3420. | Me | SMe | OCH₂CH₂ | Me | Q3a | |
| 3421. | Me | SMe | OCH₂CH₂ | Me | Q6a | |
| 3422. | Me | SMe | OCH₂CH(Me) | H | Q1a | |
| 3423. | Me | SMe | OCH₂CH₂CH₂ | H | Q1a | |
| 3424. | Me | SO₂Me | CH₂ | H | Q1a | |
| 3425. | Me | SO₂Me | CH₂ | H | Q1b | |
| 3426. | Me | SO₂Me | CH₂ | H | Q1c | |
| 3427. | Me | SO₂Me | CH₂ | H | Q1d | |
| 3428. | Me | SO₂Me | CH₂ | H | Q1e | |
| 3429. | Me | SO₂Me | CH₂ | H | Q2a | |
| 3430. | Me | SO₂Me | CH₂ | H | Q2b | |
| 3431. | Me | SO₂Me | CH₂ | H | Q3a | |
| 3432. | Me | SO₂Me | CH₂ | H | Q3c | |
| 3433. | Me | SO₂Me | CH₂ | H | Q4a | |
| 3434. | Me | SO₂Me | CH₂ | H | Q5a | |
| 3435. | Me | SO₂Me | CH₂ | H | Q6a | |
| 3436. | Me | SO₂Me | CH₂ | H | Q8a | |
| 3437. | Me | SO₂Me | CH₂ | H | Q8d | |
| 3438. | Me | SO₂Me | CH₂ | H | Q9a | |
| 3439. | Me | SO₂Me | CH₂ | H | Q12 | |
| 3440. | Me | SO₂Me | CH₂ | H | Q13 | |
| 3441. | Me | SO₂Me | CH₂ | Me | Q1a | |
| 3442. | Me | SO₂Me | CH₂ | Me | Q2a | |
| 3443. | Me | SO₂Me | CH₂ | Me | Q2b | |
| 3444. | Me | SO₂Me | CH₂ | Me | Q3a | |
| 3445. | Me | SO₂Me | CH₂ | Me | Q6a | |
| 3446. | Me | SO₂Me | CH₂ | Me | Q8d | |
| 3447. | Me | SO₂Me | CH₂ | Et | Q1a | |
| 3448. | Me | SO₂Me | CH₂ | n-Pr | Q1a | |
| 3449. | Me | SO₂Me | CH₂ | SMe | Q1a | |
| 3450. | Me | SO₂Me | CH₂CH₂ | H | Q1a | |
| 3451. | Me | SO₂Me | CH₂CH₂ | Me | Q1a | |
| 3452. | Me | SO₂Me | OCH₂CH₂ | H | Q1a | |
| 3453. | Me | SO₂Me | OCH₂CH₂ | H | Q1b | |
| 3454. | Me | SO₂Me | OCH₂CH₂ | H | Q1c | |
| 3455. | Me | SO₂Me | OCH₂CH₂ | H | Q1d | |
| 3456. | Me | SO₂Me | OCH₂CH₂ | H | Q1e | |
| 3457. | Me | SO₂Me | OCH₂CH₂ | H | Q2a | |
| 3458. | Me | SO₂Me | OCH₂CH₂ | H | Q2b | |
| 3459. | Me | SO₂Me | OCH₂CH₂ | H | Q3a | |
| 3460. | Me | SO₂Me | OCH₂CH₂ | H | Q3c | |
| 3461. | Me | SO₂Me | OCH₂CH₂ | H | Q4a | |
| 3462. | Me | SO₂Me | OCH₂CH₂ | H | Q5a | |
| 3463. | Me | SO₂Me | OCH₂CH₂ | H | Q6a | |
| 3464. | Me | SO₂Me | OCH₂CH₂ | H | Q8a | |
| 3465. | Me | SO₂Me | OCH₂CH₂ | H | Q8d | |
| 3466. | Me | SO₂Me | OCH₂CH₂ | H | Q9a | |
| 3467. | Me | SO₂Me | OCH₂CH₂ | H | Q12 | |
| 3468. | Me | SO₂Me | OCH₂CH₂ | H | Q13 | |
| 3469. | Me | SO₂Me | OCH₂CH₂ | Me | Q1a | |
| 3470. | Me | SO₂Me | OCH₂CH₂ | Me | Q2a | |
| 3471. | Me | SO₂Me | OCH₂CH₂ | Me | Q2b | |
| 3472. | Me | SO₂Me | OCH₂CH₂ | Me | Q3a | |
| 3473. | Me | SO₂Me | OCH₂CH₂ | Me | Q6a | |
| 3474. | Me | SO₂Me | OCH₂CH₂ | Me | Q8d | |
| 3475. | Me | SO₂Me | OCH₂CH₂ | Et | Q1a | |
| 3476. | Me | SO₂Me | OCH₂CH₂ | n-Pr | Q1a | |
| 3477. | Me | SO₂Me | OCH₂CH₂ | SMe | Q1a | |
| 3478. | Me | SO₂Me | OCH₂CH(Me) | H | Q1a | |
| 3479. | Me | SO₂Me | OCH₂CH₂CH₂ | H | Q1a | |
| 3480. | Me | SO₂Me | OCH₂CH₂CH₂ | Me | Q1a | |
| 3481. | Me | NO₂ | OCH₂CH₂ | H | Q1a | |
| 3482. | Me | NO₂ | OClI2CH₂ | H | Q1b | |
| 3483. | Me | NO₂ | OCH₂CH₂ | H | Q1c | |
| 3484. | Me | NO₂ | OCH₂CH₂ | H | Q1d | |
| 3485. | Me | NO₂ | OCH₂CH₂ | H | Q1e | |
| 3486. | Me | NO₂ | OCH₂CH₂ | H | Q2a | |
| 3487. | Me | NO₂ | OCH₂CH₂ | H | Q2b | |
| 3488. | Me | NO₂ | OCH₂CH₂ | H | Q3a | |
| 3489. | Me | NO₂ | OCH₂CH₂ | H | Q3c | |
| 3490. | Me | NO₂ | OCH₂CH₂ | H | Q4a | |
| 3491. | Me | NO₂ | OCH₂CH₂ | H | Q5a | |
| 3492. | Me | NO₂ | OCH₂CH₂ | H | Q6a | |
| 3493. | Me | NO₂ | OCH₂CH₂ | H | Q8a | |
| 3494. | Me | NO₂ | OCH₂CH₂ | H | Q8d | |
| 3495. | Me | NO₂ | OCH₂CH₂ | H | Q9a | |
| 3496. | Me | NO₂ | OCH₂CH₂ | H | Q12 | |
| 3497. | Me | NO₂ | OCH₂CH₂ | H | Q13 | |
| 3498. | Me | NO₂ | OCH₂CH₂ | Me | Q1a | |
| 3499. | Me | NO₂ | OCH₂CH₂ | Me | Q2a | |
| 3500. | Me | NO₂ | OCH₂CH₂ | Me | Q2b | |
| 3501. | Me | NO₂ | OCH₂CH₂ | Me | Q3a | |
| 3502. | Me | NO₂ | OCH₂CH₂ | Me | Q6a | |
| 3503. | Me | NO₂ | OCH₂CH₂ | Me | Q8d | |
| 3504. | Me | NO₂ | OCH₂CH₂ | Et | Q1a | |
| 3505. | Me | NO₂ | OCH₂CH₂ | n-Pr | Q1a | |
| 3506. | Me | NO₂ | OCH₂CH₂ | SMe | Q1a | |
| 3507. | Me | NO₂ | OCH₂CH(Me) | H | Q1a | |
| 3508. | Me | NO₂ | OCH₂CH₂CH₂ | H | Q1a | |
| 3509. | Me | NO₂ | OCH₂CH₂CH₂ | Me | Q1a | |
| 3510. | OMe | Cl | CH₂ | H | Q1a | |
| 3511. | OMe | Cl | CH₂ | H | Q3a | |
| 3512. | OMe | Cl | CH₂ | H | Q8d | |
| 3513. | OMe | Cl | CH₂ | Me | Q1a | |
| 3514. | OMe | Cl | CH₂ | Me | Q3a | |
| 3515. | OMe | Cl | CH₂ | Me | Q8d | |
| 3516. | SMe | Cl | OCH₂CH₂ | H | Q1a | |
| 3517. | SMe | Cl | OCH₂CH₂ | H | Q3a | |
| 3518. | SMe | Cl | OCH₂CH₂ | H | Q8d | |
| 3519. | SMe | Cl | OCH₂CH₂ | Me | Q1a | |
| 3520. | SMe | Cl | OCH₂CH₂ | Me | Q3a | |
| 3521. | SMe | Cl | OCH₂CH₂ | Me | Q8d | |
| 3522. | SMe | SMe | CH₂ | H | Q1a | |
| 3523. | SMe | SMe | CH₂ | H | Q3a | |
| 3524. | SO₂Me | Cl | OCH₂CH₂ | H | Q1a | |
| 3525. | SO₂Me | Cl | OCH₂CH₂ | H | Q3a | |
| 3526. | SO₂Me | Cl | OCH₂CH₂ | H | Q8d | |
| 3527. | SO₂Me | Cl | OCH₂CH₂ | Me | Q1a | |
| 3528. | SO₂Me | Cl | OCH₂CH₂ | Me | Q3a | |
| 3529. | SO₂Me | Cl | OCH₂CH₂ | Me | Q8d | |
| 3530. | NO₂ | Me | OCH₂CH₂ | H | Q1a | |
| 3531. | NO₂ | Me | OCH₂CH₂ | H | Q3a | |
| 3532. | NO₂ | Me | OCH₂CH₂ | H | Q8d | |
| 3533. | NO₂ | Me | OCH₂CH₂ | Me | Q1a | |
| 3534. | NO₂ | Me | OCH₂CH₂ | Me | Q3a | |
| 3535. | NO₂ | Me | OCH₂CH₂ | Me | Q8d | |
| 3536. | NO₂ | CH₂OMe | OCH₂CH₂ | H | Q1a | |
| 3537. | NO₂ | CH₂SMe | OCH₂CH₂ | H | Q1a | |
| 3538. | NO₂ | CH₂SO₂Me | OCH₂CH₂ | H | Q1a | |
| 3539. | CN | Me | OCH₂CH₂ | H | Q1a | |
| 3540. | CN | Me | OCH₂CH₂ | H | Q3a | |
| 3541. | CN | Me | OCH₂CH₂ | H | Q8d | |
| 3542. | CN | Me | OCH₂CH₂ | Me | Q1a | |
| 3543. | CN | Me | OCH₂CH₂ | Me | Q3a | |
| 3544. | CN | Me | OCH₂CH₂ | Me | Q8d | |
| 3545. | Cl | Cl | CH₂ | H | Q4e | |
| 3546. | Cl | Cl | CH₂ | H | Q4f | |
| 3547. | Cl | Cl | CH₂ | H | Q4g | |
| 3548. | Cl | Cl | CH₂ | H | Q10b | |
| 3549. | Cl | Cl | CH₂ | H | Q16 | |
| 3550. | Cl | Cl | CH₂ | H | Q22 | |
| 3551. | Cl | Cl | OCH₂CH₂ | H | Q4e | |
| 3552. | Cl | Cl | OCH₂CH₂ | H | Q4f | |
| 3553. | Cl | Cl | OCH₂CH₂ | H | Q4g | |
| 3554. | Cl | Cl | OCH₂CH₂ | H | Q4h | |
| 3555. | Cl | Cl | OCH₂CH₂ | H | Q4i | |
| 3556. | Cl | Cl | OCH₂CH₂ | H | Q4j | |
| 3557. | Cl | Cl | OCH₂CH₂ | H | Q10b | |
| 3558. | Cl | Cl | OCH₂CH₂ | H | Q10c | |
| 3559. | Cl | Cl | OCH₂CH₂ | H | Q10d | |
| 3560. | Cl | Cl | OCH₂CH₂ | H | Q1Qe | |
| 3561. | Cl | Cl | OCH₂CH₂ | H | Q10f | |
| 3562. | Cl | Cl | OCH₂CH₂ | H | Q16 | |
| 3563. | Cl | Cl | OCH₂CH₂ | H | Q17 | |
| 3564. | Cl | Cl | OCH₂CH₂ | H | Q18 | |
| 3565. | Cl | Cl | OCH₂CH₂ | H | Q19 | |
| 3566. | Cl | Cl | OCH₂CH₂ | H | Q20 | |
| 3567. | Cl | Cl | OCH₂CH₂ | H | Q21 | |
| 3568. | Cl | Cl | OCH₂CH₂ | H | Q22 | |
| 3569. | Cl | Cl | OCH₂CH₂ | H | Q23 | |
| 3570. | Cl | Cl | OCH₂CH₂ | H | Q24 | |
| 3571. | Cl | Cl | OCH₂CH₂ | H | Q25 | |
| 3572. | Cl | Cl | OCH₂CH₂ | H | Q26 | |
| 3573. | Cl | Cl | OCH₂CH₂ | Me | Q4e | |
| 3574. | Cl | Cl | OCH₂CH₂ | Me | Q4f | |
| 3575. | Cl | Cl | OCH₂CH₂ | Me | Q10b | |
| 3576. | Cl | Cl | OCH₂CH₂ | Me | Q16 | |
| 3577. | Cl | Cl | OCH₂CH₂ | Me | Q19 | |
| 3578. | Cl | Cl | OCH₂CH₂ | Me | Q22 | |
| 3579. | Cl | SO₂Me | CH₂ | H | Q4e | |
| 3580. | Cl | SO₂Me | CH₂ | H | Q4f | |
| 3581. | Cl | SO₂Me | Cl2 | H | Q4g | |
| 3582. | Cl | SO₂Me | CH₂ | H | Q4h | |
| 3583. | Cl | SO₂Me | CH₂ | H | Q4i | |
| 3584. | Cl | SO₂Me | CH₂ | H | Q4j | |
| 3585. | Cl | SO₂Me | CH₂ | H | Q10b | |
| 3586. | Cl | SO₂Me | CH₂ | H | Q10c | |
| 3587. | Cl | SO₂Me | CH₂ | H | Q10d | |
| 3588. | Cl | SO₂Me | CH₂ | H | Q10e | |
| 3589. | Cl | SO₂Me | CH₂ | H | Q10f | |
| 3590. | Cl | SO₂Me | CH₂ | H | Q1lb | |
| 3591. | Cl | SO₂Me | CH₂ | H | Q1lc | |
| 3592. | Cl | SO₂Me | CH₂ | H | Q1ld | |
| 3593. | Cl | SO₂Me | CH₂ | H | Q16 | |
| 3594. | Cl | SO₂Me | CH₂ | H | Q17 | |
| 3595. | Cl | SO₂Me | CH₂ | H | Q15 | |
| 3596. | Cl | SO₂Me | CH₂ | H | Q19 | |
| 3597. | Cl | SO₂Me | CH₂ | H | Q20 | |
| 3598. | Cl | SO₂Me | CH₂ | H | Q21 | |
| 3599. | Cl | SO₂Me | CH₂ | H | Q22 | |
| 3600. | Cl | SO₂Me | CH₂ | H | Q23 | |
| 3601. | Cl | SO₂Me | CH₂ | H | Q24 | |
| 3602. | Cl | SO₂Me | CH₂ | H | Q25 | |
| 3603. | Cl | SO₂Me | CH₂ | H | Q26 | |
| 3604. | Cl | SO₂Me | CH₂ | Me | Q4e | |
| 3605. | Cl | SO₂Me | CH₂ | Me | Q4f | |
| 3606. | Cl | SO₂Me | CH₂ | Me | Q10b | |
| 3607. | Cl | SO₂Me | CH₂ | Me | Q16 | |
| 3608. | Cl | SO₂Me | CH₂ | Me | Q19 | |
| 3609. | Cl | SO₂Me | CH₂ | Me | Q22 | |

*Substituent R^{1c} is mentioned in parenthesis in the column of the group R^{1b}, only when it is other than a hydrogen atom (H).

### SYNTHESIS EXAMPLE 5

### Intermediate Synthesis

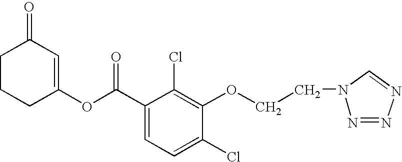

2,4-Dichloro-3-[2-(1H-tetrazol-1-yl)ethoxy]benzoic acid (0.68 g) and thionyl chloride (0.40 g) were added in 1,2-dichloroethane (20 ml) and after addition of 2 drops of N,N-dimethylformamide the mixture was refluxed for 5 hours. After cooling the solvent was distilled off. The obtained residue was dissolved in dichloromethane (10 ml) and added into a solution of 1,3-cyclohexanedione (0.18 g) and triethylamine (0.18 g) in dichloromethane (10 ml) drop by drop at 5° C. and the mixture was stirred at room temperature for 6 hours. After the reaction it was extracted with dichloromethane (100 ml), washed with diluted hydrochloric acid and an aqueous solution of sodium hydrogen carbonate and dried with anhydrous magnesium sulfate. After dichloromethane was distilled off, the obtained residue was purified by silica gel column chromatography (eluent:ethyl acetate:hexane=3:2) to obtain the objective 3-oxo-1-cyclohexenyl 2,4-dichloro-3-[2-(1H-tetrazol-1-yl)ethoxy]benzoate (0.51 g).

n^{D}₂₀: 1.5835.

### SYNTHESIS EXAMPLE 6

### Intermediate Synthesis

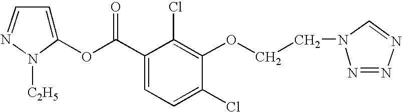

2,4-Dichloro-3-[2-(1H-tetrazol-1-yl)ethoxy]benzoic acid (0.46 g) and thionyl chloride (0.36 g) were added in 1,2-dichloroethane (10 ml) and after addition of 2 drops of N,N-dimethylformamide the mixture was refluxed for 5 hours. After cooling the solvent was distilled off. The obtained residue was dissolved in dichloromethane (5 ml) and added into a solution of 1-ethyl-5-hydroxypyrazole (0.18 g) and triethylamine (0.19 g) in dichloromethane (5 ml) drop by drop at 5° C. and the mixture was stirred at room temperature for 6 hours. After the reaction it was extracted with dichloromethane (100 ml), washed with diluted hydrochloric acid and an aqueous solution of sodium hydrogen carbonate and dried with anhydrous magnesium sulfate. Dichloromethane was distilled off to obtain the objective 5-{2,4-dichloro-3-[2-(1H-tetrazol-1-yl)ethoxy]benzoyloxy}-1-ethylpyrazole (0.58 g) (viscous oily substance), which was used as the intermediate for preparation in the aforementioned Synthesis Example 4.

### SYNTHESIS EXAMPLE 7

### Intermediate Synthesis

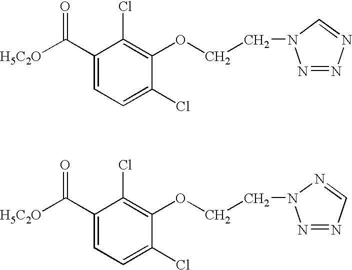

In N,N-dimethylformamide (15 ml) 1H-tetrazole (0.45 g) and ethyl 3-(2-bromo-ethoxy)-2,4-dichlorobenzoate (2.00 g) were suspended and potassium carbonate (1.21 g) and sodium iodide (0.04 g) were added thereto and the mixture was stirred at 80° C. for 3 hours. After the reaction cold water was added to the mixture and it was extracted with ethyl acetate (100 ml) and dried with anhydrous magnesium sulfate. After ethyl acetate was distilled off, the obtained residue was purified by silica gel column chromatography (eluent:ethyl acetate:hexane=1:1) to obtain the objective ethyl 2,4-dichloro-3-[2-(1H-tetrazol-1-yl)ethoxy]benzoate (0.88 g, n^{D}₂₀: 1.5535) and ethyl 2,4-dichloro-3-[2-(1H-tetrazol-2-yl)ethoxy]benzoate (0.99 g), n^{D}₂₀: 1.5483.

### SYNTHESIS EXAMPLE 8

### Intermediate Synthesis

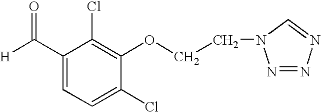

To a solution of ethyl 2,4-dichloro-3-[2-(1H-tetrazol-1-yl)ethoxy]benzoate (0.88 g) in methanol (15 ml) sodium hydroxide (0.16 g) and water (5 ml) were added and the mixture was stirred at room temperature for 7 hours. After addition of water (20 ml) and concentration under reduced pressure, an aqueous solution of sodium hydroxide was added and washed with ethyl acetate (50 ml). The aqueous layer was acidified with hydrochloric acid and extracted with ethyl acetate. The organic layer was washed with saturated aqueous solution of sodium chloride and dried with anhydrous magnesium sulfate. Ethyl acetate was distilled off to obtain the objective 2,4-dichloro-3-[2-(1H-tetrazol-1-yl)ethoxy]benzoic acid (0.69 g).

Mp: 145-146° C.

Biological Tests

### EXAMPLE 1

### Test for Herbicidal Effect Against Paddy Field Weeds

Preparation of formulation of the active compound
Carrier: Acetone 5 parts by weight
Emulsifier: Benzyloxy polyglycol ether 1 part by weight

A formulation of an active substance is obtained as an emulsifiable concentrate by mixing 1 part by weight of the active compound with the above-mentioned amount of the carrier and emulsifier. A prescribed amount of the formulation is diluted with water.

Test Method

In a green house 3 seedlings of paddy rice (variety: Nihonbare) of 2.5 leaf stage (15 cm tall) were transplanted in a 500 cm² pot filled with paddy field soil. Then seeds or tubers of Echinochloa crusgalli, Cyperus difformis L., Scirpus juncoides Roxburgh, Monochoria vaginalis Presl, broad-leaved weeds (Lindernia Procumbens Philcox, Rotala indica Koehne, Elatine triandra Schk, Ammannia multiflora Roxb., Dopatrium junceum Hammilt etc.), Cyperus serotinus Rottboel and Sagittaria pygmaea Miq were inoculated and water was poured to a depth of about 2-3 cm. 5 days after the rice transplantation a prescribed, diluted solution of each formulation of active compound prepared according to the aforementioned preparation method was applied to the surface of water. After the treatment the water depth of 3 cm was maintained and the herbicidal effect was examined after 3 weeks from the treatment. The herbicidal effect was rated 100% in the case of complete death and as 0% in the case of no herbicidal effect.

Results

As a result, as specific examples the compounds No. 217, 219, 558, 1838, 2024, 2329, 2335 and 2364 showed a sufficient herbicidal effect against paddy field weeds at the chemical amount (amount of the active component) of 0.25 kg/ha and a safety to the transplanted paddy rice.

### EXAMPLE 2

### Test of Pre-Emergence Soil Treatment Against Field Weeds

Test Method

In a green house, on the surface layer of a 120 cm² pot filled with field soil, seeds of Echinochloa crusgalli, Setaria viridis, Amaranthus retroflexus and Polygonum were sown and covered with soil. A diluted solution of the prescribed chemical amount prepared in the same manner as in the above-mentioned Test Example 1 was spread uniformly on the soil surface layer of each test pot. The herbicidal effect was examined 4 weeks after the treatment.

Results

As a result, as specific examples the compounds No. 213, 215, 258, 272, 275, 278, 715, 1234, 1239, 1593, 2077, 2080, 2081, 2301, 2347, 2521 and 3043 showed a herbicidal activity of higher than 90% against Echinochloa crusgalli, Setaria viridis, Amaranthus retroflexus and Polygonum at the chemical amount (amount of the active component) of 2.0 kg/ha.

### EXAMPLE 3

### Test of Post-Emergence Foliage Treatment Against Field Weeds

Test Method

In a green house, seeds of Echinochloa crusgalli, Setaria viridis, Amaranthus retro-flexus and Polygonum were sown in a 120 cm² pot filled with field soil and covered with soil. 10 days after the sowing and soil covering (weeds were 2-leaf stage in average) a diluted solution of the prescribed chemical amount prepared in the same manner as in the above-mentioned Test Example 1 was spread uniformly on the foliage of the test plants in each test pot. The herbicidal effect was examined 3 weeks after the treatment.

Results

As a result, as specific examples the compounds No. 212, 213, 215, 258, 272, 275, 355, 495, 650, 715, 1234, 1239, 1593, 1693, 2017, 2022, 2063, 2077, 2080, 2081, 2301, 2347, 2364, 2521, 3038 and 3043 showed a herbicidal activity of 90% against Echinochloa crusgalli, Setaria viridis, Amaranthus retroflexus and Polygonum at the chemical amount (amount of the effective component) of 2.0 kg/ha.

Formulations

### EXAMPLE 1

### Granule

To a mixture of the compound of the present invention No. 2024 (10 parts), bentonite (montmorillonite) (30 parts), talc (58 parts) and ligninsulfonate salt (2 parts), water (25 parts) were added, well kneaded, made into granules of 10-40 mesh by an extrusion granulator and dried at 40-50° C. to obtain granules.

### EXAMPLE 2

### Granule

Clay mineral particles (95 parts) having particle diameter distribution in the range of 0.2-2 mm are put in a rotary mixer. While rotating it, the compound of the present invention No. 217 (5 parts) are sprayed together with a liquid diluent, wetted uniformly and dried at 40-50° C. to obtain granules.

### EXAMPLE 3

### Emulsifiable Concentrate

The compound of the present invention No. 212 (30 parts), xylene (5 parts), polyoxyethylene alkyl phenyl ether (8 parts) and calcium alkylbenzenesulfonate (7 parts) are mixed and stirred to obtain an emulsifiable concentrate.

### EXAMPLE 4

### Wettable Powder

The compound of the present invention No. 258 (15 Parts), a mixture of white carbon (hydrous amorphous silicon oxide fine powders) and powder clay (1:5) (80 parts), sodium alkylbenzenesulfonate (2 parts) and sodium alkylnaphthalenesulfonate-formalin-condensate (3 parts) are crushed and mixed to make a wettable powder.

### EXAMPLE 5

### Water Dispersible Granule

The compound of the present invention No. 258 (20 Parts), sodium ligninsulfonate (30 parts), bentonite (15 parts) and calcined diatomaceous earth (35 parts) are well mixed, added with water, extruded with 0.3 mm screen and dried to obtain water dispersible granules.

1) Compounds of formula (I) 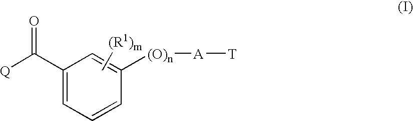
wherein
R¹ represents halogen, alkyl, haloalkyl, alkoxy, alkylthio, alkylsulfonyl, alkylsulfonyloxy, alkoxyalkyl, alkylthioalkyl, alkylsulfonylalkyl, nitro or cyano,
m represents 0, 1, 2 or 3,
R¹ may be identical or different to each other, when m represents 2 or 3,
n represents 0 or 1,
A represents alkylene,
T represents a group 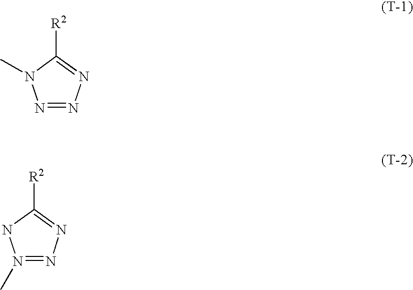
in which
R² represents hydrogen, alkyl or cycloalkyl, which may be optionally substituted by alogen and alkyl, or
represents alkenyl, alkynyl, haloalkyl, alkylthio, or phenyl which may be optionally substituted by halogen, alkyl, haloalkyl and nitro, and
Q represents a group 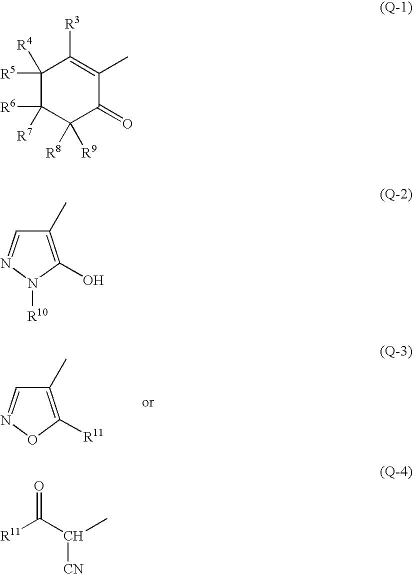
wherein
R³ represents hydroxy, halogen or alkylcarbonyloxy, or
represents alkylthio which may be optionally substituted by hydroxy, cyano, carboxy, alkoxycarbonyl and phenyl, or
represents 5- or 6-membered heteroarylthio containing 1-2 hetero atom(s) selected from the group consisting of nitrogen, oxygen and sulfur, which may be optionally substituted by halogen and alkyl, and when R³ represents pyridylthio, said pyridylthio may form N-oxide, or represents phenylthio which may be optionally substituted by halogen, alkyl, alkoxy, haloalkyl and nitro, or
represents phenylcarbonyloxy which may be optionally substituted by halogen and alkyl, or
represents 1-pyrazolyl which may be optionally substituted or 1-imidazolyl which may be optionally substituted by halogen and alkyl, or
represents 1,2,4-triazol-1-yl or 1H-tetrazol-1-yl,
R⁴, R⁵, R⁶, R⁷, R⁸ and R⁹ each independently represent hydrogen or alkyl, or
R⁴ may, together with R⁹, form an ethylene chain,
R¹⁰ represents alkyl, and
R¹¹ represents alkyl or cycloalkyl.

2) The compounds of the formula(I) according to claim 1 wherein
R¹ represents fluoro, chloro, bromo, iodo, C₁₋₆ alkyl, C₁₋₆ haloalkyl, C₁₋₆ alkoxy, C₁₋₄ alkylthio, C₁₋₆ alkylsulfonyl, C₁₋₆ alkylsulfonyloxy, C₂₋₆ alkoxyalkyl, C₂₋₆ alkylthioalkyl, C₂₋₆ alkylsulfonylalkyl, nitro or cyano,
m represents 2 or 3,
A represents C₁₋₄ alkylene,
R² represents hydrogen, C₁₋₆ alkyl, C₃₋₆ cycloalkyl, C₂₋₆ alkenyl, C₂₋₆ alkynyl, C₁₋₆ haloalkyl, C₁₋₆ alkylthio, or phenyl which may be optionally substituted by chloro, C₁₋₄ alkyl, C₁₋₄ haloalkyl and nitro, and
R³ represents hydroxy, chloro, bromo, C₂₋₅ alkylcarbonyloxy, or
C₁₋₆ alkylthio which may be optionally substituted by hydroxy, cyano, carboxy, C₂₋₅ alkoxycarbonyl and phenyl, or
thienylthio, thiazolylthio, oxazolylthio, pyridylthio, 1-oxidopyridylthio or pyrimidylthio, optionally substituted by chloro-, bromo-, or C₁-C₄ alkyl, or
phenylthio which may be optionally substituted by one or two substituents selected from the group consisting of fluoro, chloro, bromo, C₁₋₄ alkyl, C₁₋₄ alkoxy, C₁₋₄ haloalkyl and nitro, or
phenylcarbonyloxy which may be optionally substituted by one or two substituents selected from the group consisting of chloro and C₁₋₄ alkyl, or
1-pyrazolyl or 1-imidazolyl which may be optionally substituted by one or two substituents selected from the group consisting of chloro, bromo and C₁₋₄ alkyl, or
1,2,4-triazol-1-yl or 1H-tetrazol-1-yl,
R⁴, R⁵, R⁶, R⁷, R⁸ and R⁹ each independently preferably represent hydrogen or C₁₋₄ alkyl, or
R⁴ may, together with R⁹, form an ethylene chain,
R¹⁰ preferably represents C₁₋₄ alkyl,
R¹¹ preferably represents C₃₋₆ cycloalkyl.
R¹ particularly preferably represents chloro, bromo, methyl, trifluoromethyl, methoxy, methylthio, ethylthio, methylsulfonyl, ethylsulfonyl, n-propylsulfonyl, methylsulfonyloxy, methoxymethyl, methylthiomethyl, methylsulfonylmethyl, nitro or cyano.

3) The compounds of the formula (I) according to claims 1 or 2 wherein
m represents 2,
A represents C₁₋₄ alkylene,
R² represents hydrogen, methyl, ethyl, n-propyl, cyclopropyl, cyclopentyl, vinyl, allyl, ethynyl, trifluoromethyl, 2-chloroethyl, 3-bromopropyl, methylthio, ethylthio, n-propylthio, or phenyl which may be optionally substituted by chloro, methyl, ethyl, n-propyl, trifluoromethyl and nitro, and
R³ represents hydroxy, chloro, acetoxy, tert-butylcarbonyloxy, methylthio, ethylthio, n-propylthio, 2-hydroxyethylthio, 2-cyanoethylthio, carboxymethylthio, methoxycarbonylmethylthio, 2-(ethoxycarbonyl)ethylthio, benzylthio, or
2-thienylthio, 2-thiazolylthio, 2-oxazolylthio, 2-pyridylthio, 1-oxido-2-pyridylthio or 2-pyrimidylthio, optionally substituted by one or more substituents selected from the group consisting of chloro and methyl, or
phenylthio which may be optionally substituted by a substituent selected from the group consisting of fluoro, chloro, methyl, ethyl, n-propyl, methoxy, trifluoromethyl and nitro, or
represents phenylcarbonyloxy which may be optionally substituted by a substituent selected from the group consisting of chloro and methyl, or
represents1-pyrazolyl or 1-imidazolyl which may be optionally substituted by one or two substituents selected from the group consisting of chloro and methyl, or
represents 1,2,4-triazol-1-yl or 1H-tetrazol-1-yl,
R⁴, R⁵, R⁶, R⁷, R⁸ and R⁹ each independently, represent hydrogen or methyl, or
R⁴ may, together with R⁹, form an ethylene chain,
R¹⁰ represents methyl or ethyl,
R¹¹ particularly preferably represents cyclopropyl.

4) Process for preparing compounds according to claim 1, 2 or 3 characterized in that
b) in case of preparing a compound of the formula (I) in which
Q represents the group (Q-1) and
R³ represents hydroxy
compounds of the formula (II) 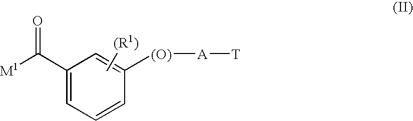
wherein
R¹, m, n, A and T have the same definition as aforementioned, and
M¹ represents a group 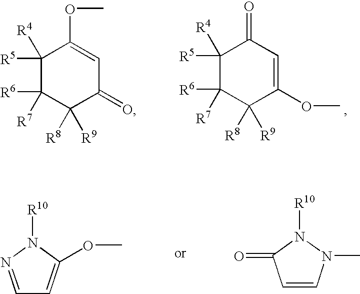
in which
R⁴, R⁵, R⁶, R⁷, R⁸, R⁹ and R¹⁰ have the same definition as aforementioned,
are reacted to a rearrangement in the presence of inert solvents, and if appropriate, in the presence of a base and a cyanide, and if appropriate, in the presence of a phase-transfer catalyst, or
c) in case of preparing a compound of the formula (I) in which
Q represents the group (Q-1) and
R³ represents halogen, preferably chloro or bromo:
compounds of the formula (Ib) 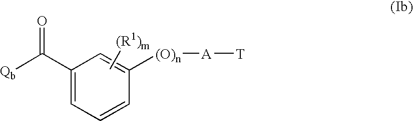
wherein
R¹, m, n, A and T have the same definition as aforementioned, and
Q_{b} represents group 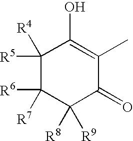
in which
R⁴, R⁵, R⁶, R⁷, R⁸ and R⁹ have the same definition as aforementioned,
are reacted with a halogenating agent in the presence of inert solvents, or
c) in case of preparing a compound of the formula (I) in which
Q represents the group (Q-1) and
R³ represents alkylthio which may be optionally substituted or
represents 5- or 6-membered heteroarylthio, or
represents phenylthio which may be optionally substituted, or
represents 1-pyrazolyl which may be optionally substituted or
represents 1-imidazolyl which may be optionally substituted, or
represents 1,2,4-triazol-1-yl or 1H-tetrazol-1-yl:
compounds of the formula (Ic) 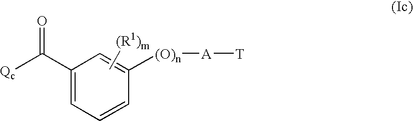
wherein
R¹, m, n, A and T have the same definition as aforementioned, and
Q_{c} represents group 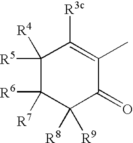
in which
R⁴, R⁵, R⁶, R⁷, R⁸ and R⁹ have the same definition as aforementioned, and
R^{3c} represents chloro or bromo,
are reacted with compounds of the formula (III)
R¹²—H (III)
wherein
R¹² has the same definition as the above-mentioned R³ in the preparation process (c),
in the presence of inert solvents, and if appropriate, in the presence of an acid binding agent, or
e) in case of preparing a compound of the formula (I) in which
Q represents the group (Q-1) and
R³ represents alkylcarbonyloxy or phenylcarbonyloxy which may be optionally substituted:
compounds of the formula (Ib) 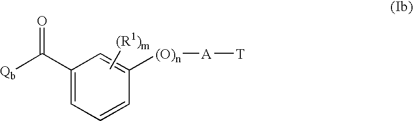
are reacted with compounds of the formula (IV) 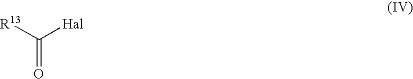
wherein
R¹³ represents alkyl or phenyl which may be optionally substituted, and
Hal represents halogen, preferably chloro or bromo,
in the presence of inert solvents, and if appropriate, in the presence of an acid binding agent, or
f) in case of preparing a compound of the formula (I) in which Q represents the group (Q-2):
compounds of the formula (IIe) 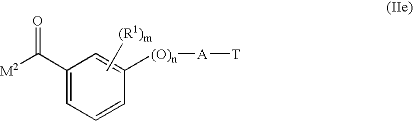
wherein
R¹, m, n, A and T have the same definition as aforementioned, and
M² represents group 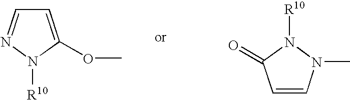
in which
R¹⁰ has the same definition as aforementioned,
are reacted to a rearrangement in the presence of inert solvents,
and if appropriate, in the presence of a base, or
f) in case of preparing a compound of the formula (I) in which Q represents the group (Q-3):
compounds of the formula (V) 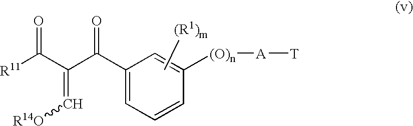
wherein
R¹, m, n, A, T and R¹¹ have the same definition as aforementioned, and
R¹⁴ represents C₁₋₄ alkyl, preferably methyl or ethyl,
are reacted with hydroxylamine in the presence of inert solvents, and if appropriate, in the presence of a base, or
h) in case of preparing a compound of the formula (I) in which Q represents the group (Q-4):
compounds of the formula (Ig) 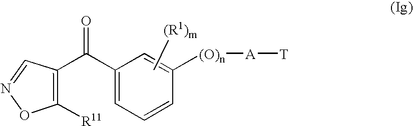
wherein
R¹, m, n, A, T and R¹¹ have the same definition as aforementioned,
are subject to a ring—opening reaction in the presence of inert solvents, and if appropriate, in the presence of a base.

5) Herbicidal compositions, characterized in that they contain at least one compound of the formula (I) according to claim 1.

6) Process for combating weeds, characterized in that compounds of formula (I) according to claim 1 are allowed to act on weeds and/or their habitat.

7) Use of compounds of formula (I) according to claim 1 for combating weeds.

8) Process for the preparation of herbicidal compositions, characterized in that compounds of formula (I) according to claim 1 are mixed with extenders and/or surface active agents.

9) Compounds of the formula (XVI) 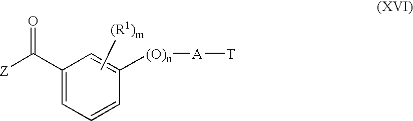
wherein
Z represents a group 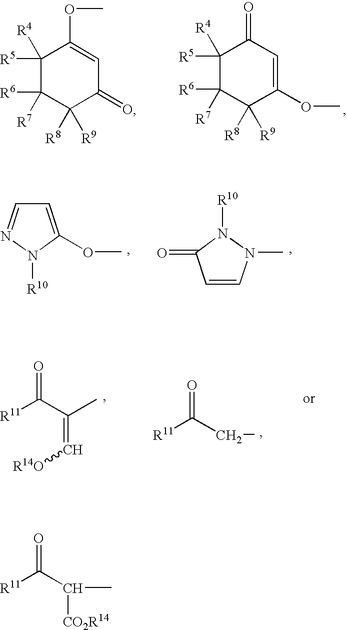
and R¹, R⁴, R⁵, R⁶, R⁷, R⁸, R⁹, R¹⁰, R¹¹ and R¹⁴ m, n, A, T are as defined in claim 1.
Compounds of the formula (I) 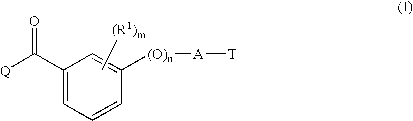
wherein T represents a group 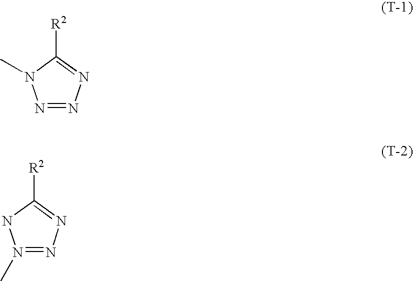
Q represents a group 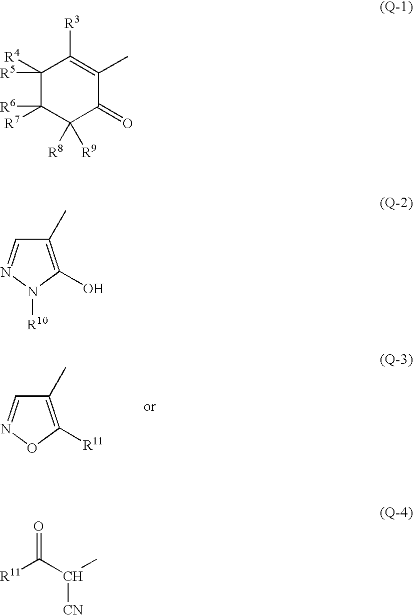
R¹, R², R³, R⁴, R⁵, R⁶, R⁷, R⁸, R^{9,} R¹⁰, R¹¹ are as defined in the description,
m represents 0, 1, 2 or 3,
n represents 0 or 1,
A represents alkylene,
processes for their preparation, their intermediates and their use in agriculture are described.
